(19) United States
(12) Patent Application Publication
(10) Pub. No.: US 2022/0089768 A1
(43) Pub. Date: Mar. 24, 2022

(54) MULTI-SPECIFIC PROTEIN MOLECULES AND USES THEREOF

(71) Applicant: Trio Pharmaceuticals, Inc., San Diego, CA (US)

(72) Inventors: Shivarupam BHOWMIK, San Diego, CA (US); William A. BRADY, San Diego, CA (US)

(21) Appl. No.: 17/420,000

(22) PCT Filed: Jan. 3, 2020
(86) PCT No.: PCT/US2020/012139

§ 371 Date: Jun. 30, 2021

Related U.S. Application Data
(60) Provisional application No. 62/788,495, filed on Jan. 4, 2019.

(51) Int. Cl. C07K 16/30; C07K 16/28; A61K 47/68; A61P 35/00

(52) U.S. Cl. CPC C07K 16/30; A61K 2039/505; A61K 47/6817; A61P 35/00; A61K 47/6849; A61K 47/6851; A61K 47/6879; C07K 16/303; C07K 16/28; C07K 16/2896; C07K 16/2866; C07K 2317/31; C07K 2317/24; C07K 2317/92; C07K 2317/732; C07K 2317/53; C07K 16/2803

(57) ABSTRACT

Disclosed herein are multi-specific binding polypeptide molecules (e.g., multi-specific antibodies) that can interact with a tumor cell and an immunosuppressive cell. In some embodiments, also described herein are methods for treating one or more diseases or conditions (e.g., cancers) using the multi-specific binding polypeptides (e.g., multi-specific antibodies).

### CROSS REFERENCE TO RELATED APPLICATIONS

This application claims the benefit under 35 U.S.C. § 119(e) of U.S. Provisional Application Ser. No. 62/788,495, filed Jan. 4, 2019, the contents of which are incorporated by reference herein in its entirety including all figures and tables.

### SUMMARY OF THE DISCLOSURE

Described herein are multi-specific binding polypeptides useful for the treatment of cancers. The multi-specific binding polypeptides comprise at least two distinct binding moieties with one of the binding moieties specific for a tumor-associated antigen, and the other specific for an antigen expressed on an immunosuppressive cell. In certain embodiments, the immunosuppressive cell is a myeloid derived suppressor cell (MDSC). In certain embodiments, the immunosuppressive cell is a tumor-associated macrophage, optionally a M2-tumor associated macrophage (M2-TAM). In certain embodiments, the multi-specific binding polypeptide further comprises a cytotoxic moiety.

In certain aspects, described herein is a multi-specific binding polypeptide comprising a tumor binding moiety that specifically binds to a tumor-associated antigen and an immune cell binding moiety that specifically binds to an antigen expressed on an immunosuppressive cell. In some embodiments, the multi-specific binding polypeptide is a multi-specific antibody. In some embodiments, the multi-specific antibody comprises a tumor binding moiety that specifically binds to a tumor-associated antigen and an immune cell binding moiety that specifically binds to an antigen expressed on an immunosuppressive cell.

In certain embodiments, described herein is a method of treating a disease or condition in a subject in need thereof, comprising administering to the subject a therapeutically effective amount of a multi-specific binding polypeptide (e.g., a multi-specific antibody), a pharmaceutical composition comprising, consisting essentially of, or consisting of the multi-specific binding polypeptide (e.g., the multi-specific antibody), or a nucleic acid encoding the multi-specific binding polypeptide (e.g., the multi-specific antibody). In some aspects, also described herein is a method of treating an individual with a cancer, the method comprising administering to the individual with cancer the multi-specific binding polypeptide (e.g., the multi-specific antibody), the pharmaceutical composition comprising, consisting essentially of, or consisting of the multi-specific binding polypeptide (e.g., the multi-specific antibody), or a nucleic acid encoding the multi-specific binding polypeptide (e.g., the multi-specific antibody). In certain embodiments, the individual has previously been treated with an immune checkpoint inhibitor treatment.

In certain embodiments, described herein is a method of inducing tumor and immunosuppressive cell killing effect in a target cell population, the method comprises contacting the target cell population comprising at least one tumor cell and at least one immunosuppressive cell with a multi-specific binding polypeptide (e.g., a multi-specific antibody), a pharmaceutical composition comprising, consisting essentially of, or consisting of the multi-specific binding polypeptide (e.g., the multi-specific antibody), or a nucleic acid encoding the multi-specific binding polypeptide (e.g., the multi-specific antibody) for a time sufficient to induce cell kill effect, thereby killing the at least one tumor cell and the at least one immunosuppressive cell in the target cell population.

In certain embodiments, additionally described herein is a method for making a cancer treatment comprising contacting the nucleic acid encoding the multi-specific binding polypeptide to a suitable cell line to establish a transfected cell line, culturing the transfected cell line under conditions that promote secretion of the multi-specific binding polypeptide, and harvesting the multi-specific binding polypeptide from the supernatant of the transfected cell line. In certain embodiments, the method further comprises purifying the multi-specific polypeptide from the supernatant of the transfected cell line. In certain embodiments, the transfected cell line is stably transfected. In certain embodiments, described herein is a method for making a cancer treatment comprising, consisting essentially of, or consisting of admixing the multi-specific binding polypeptide and a pharmaceutically acceptable excipient, carrier, or diluent.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A illustrates a non-limiting embodiment of a multi-specific binding polypeptide of the current disclosure.

FIG. 1B illustrates a non-limiting embodiment of a multi-specific binding polypeptide of the current disclosure with a cytotoxic moiety.

FIG. 1C illustrates a cartoon representation of an exemplary single action bispecific antibody which binds and kills cancer cells but does not bind or kill immunosuppressor cells. The exemplary single action bispecific antibody is a non-applicant bispecific antibody.

FIG. 1D illustrates a cartoon representation of a multi-action multi-specific antibody described herein that binds and modulates killing of a cancer cell and an immunosuppressor cell.

FIG. 2 depicts an expression heat map of receptors expressed on Triple Negative Breast Cancer (TNBC) cells and immunosuppressive cells detected in the same TNBC biopsy. As shown in this figure, the TNBC cells are expressing a high level of TROP2. Select receptors are noted in the heat map chart as: TROP2=TACSTD2; TRAIL-R2=TNFRSF10B; PD1=PDCD1; PDL1=CD274.

FIG. 3 depicts an expression heat map of receptors expressed on Lung Adenocarcinoma (LUAD) cells and immunosuppressive cells detected in the same LUAD biopsy. As shown in this figure, the LUAD cells are expressing high levels of TROP2.

FIG. 4 depicts an expression heat map of receptors expressed on Lung Squamous Cell Carcinoma (LUSC) cells and immunosuppressive cells detected in the same LUSC biopsy. As shown in this figure, the LUSC cells are expressing high levels of TROP2.

FIG. 5 depicts an expression heat map of receptors expressed on Prostate Adenocarcinoma (PRAD) cells and immunosuppressive cells detected in the same PRAD biopsy. As shown in this figure, the PRAD cells are expressing high levels of TROP2.

FIG. 6 depicts an expression heat map of receptors expressed on Liver Hepatocellular Carcinoma (LIHC) cells and immunosuppressive cells detected in the same LIHC biopsy. As shown in this figure, the LIHC cells are expressing high levels of GPC3.

FIG. 7 depicts an expression heat map of receptors expressed on Lung Adenocarcinoma (LUAD) cells and immunosuppressive cells detected in the same LUAD biopsy. As shown in this figure, the LUAD cells are expressing high levels of GPC3.

FIG. 8 depicts an expression heat map of receptors expressed on Lung Adenocarcinoma (LUAD) cells and immunosuppressive cells detected in the same LUAD biopsy. As shown in this figure, the LUAD cells are expressing high levels of FOLR1.

FIG. 9 depicts an expression heat map of receptors expressed on Lung Squamous Cell carcinoma (LUSC) cells and immunosuppressive cells detected in the same LUSC biopsy. As shown in this figure, the LUSC cells are expressing high levels of FOLR1.

FIG. 10 depicts an expression heat map of receptors expressed on Ovarian Cystadenocarcinoma (OV) cells and immunosuppressive cells detected in the same OV biopsy. As shown in this figure, the OV cells are expressing high levels of FOLR1.

FIG. 11 depicts an expression heat map of receptors expressed on Prostate Adenocarcinoma (PRAD) cells and immunosuppressive cells detected in the same PRAD biopsy. As shown in this figure, the PRAD cells are expressing high levels of FOLH1.

FIG. 12A-FIG. 12E illustrate expression and analytical characterization of αTROP2×αTRAIL-R2 bispecific antibody. FIG. 12A shows transient expression titer of αTROP2×αTRAIL-R2 bispecific antibody. The titer estimate is based on the net amount of bispecific antibody generated after Protein A purification. FIG. 12B shows solution profile analysis of Protein A purified αTROP2×αTRAIL-R2 bispecific antibody by Size Exclusion Ultra-Performance Liquid Chromatography (SE-UPLC). The major peak with retention time=3.596 corresponds to monomeric form of the intact bispecific antibody constructs and is ˜85% of all the species noted. Peaks with retention time 2.980 and 3.142 are higher molecular weight species of the bispecific antibody construct. FIG. 12C shows SDS-PAGE analysis of Protein A purified αTROP2×αTRAIL-R2 bispecific antibody. The samples were analyzed under reducing (R) and non-reducing (NR) conditions. Two bands are observed under reducing conditions, top band corresponding to the molecular weight of the heavy chain and lower band corresponding to the molecular weight of the light chain. Under non-reducing conditions only one band was observed corresponding to the molecular weight of the intact bispecific antibody construct. FIG. 12D shows intact-mass analysis of Protein A purified αTROP2×αTRAIL-R2 bispecific antibody. The sample was reduced and de-glycosylated before analyzing the mass. Both the heavy chain (upper panel) and light chain (lower panel) exhibited a major peak corresponding to theoretical molecular mass based on the amino acid sequence. The second peak observed in the upper panel corresponds to a glycosylated heavy chain species arising from in-complete de-glycosylation. FIG. 12 E shows binding affinity measurement of αTROP2×αTRAIL-R2 bispecific antibody toward TROP2 and TRAIL-R2 antigen. The binding affinity was measured by ELISA. The binding affinity for TROP2 antigen expressed on cancer cell was K_{D}=0.0024 nM and binding affinity for TRAIL-R2 expressed on immunosuppressor cell was K_{D}=0.22 nM. Rabbit anti-TROP2 and anti-TRAIL-R2 antibodies bound TROP2 and TRAIL-R2 antigens, respectively with binding affinity, K_{D}, 0.004 nM and 0.0009 nM. The non-binding human IgG1 isotype control did not bind to TROP2 and TRAIL-R2 antigens.

FIG. 13A-FIG. 13G illustrate target mediated selective binding of αTROP2×αTRAIL-R2 bispecific antibody on the surface of cancer cells measured by Fluorescent Activated Cell Sorting method (FACS). FIG. 13A shows selective binding of αTROP2×αTRAIL-R2 bispecific antibody on the surface of a TROP2+ cancer cell line, SKBR3 with a binding affinity, K_{D}=2.4 nM (0.463 μg/ml). The non-binding IgG1 isotype control did not bind on the surface of SKBR3. FIG. 13B shows high levels of TROP2 expressed on the surface of SKBR3 cell line. The non-binding rabbit antibody isotype control did not bind on the surface of SKBR3 cell line as detected by no fluorescent signal. FIG. 13C shows low expression of TRAIL-R2 in SKBR3 cell line. The non-binding rabbit antibody isotype control did not bind on the surface of SKBR3 cell line. FIG. 13D shows selective binding of αTROP2×αTRAIL-R2 bispecific antibody on the surface of a TRAIL-R2+ cancer cell line, U937. There was a concentration dependent increase in fluorescence intensity by αTROP2×αTRAIL-R2 in U937. In contrast, there was no concentration dependent increase in fluorescence intensity by Herceptin in U937 suggesting it did not bind U937. Herceptin, a therapeutic antibody which is known to bind HER2 antigen did not bind on the surface of U937, which is known not to express HER2. FIG. 13E shows no binding of αTROP2×αTRAIL-R2 bispecific antibody on the surface of a TROP2⁻and TRAIL-R2⁻cell line, THP1. Herceptin, a therapeutic antibody known to bind HER2 did not bind on the surface of THP1. There was no concentration dependent increase in fluorescence intensity suggesting neither αTROP2×αTRAIL-R2 nor Herceptin bound THP1. FIG. 13F shows low levels of TRAIL-R2 and no detectable levels of expression of TROP2 in U937 cell line. Rabbit anti-TRAIL-R2 antibody bound the surface of U937 as measured by detectable level of fluorescent signal. Mouse anti-TROP2 antibody did not bind the surface of U937 as detected by no fluorescent signal. Both rabbit and mouse antibody isotype controls did not bind the surface of U937. FIG. 13G shows there is no detectable expression of TRAIL-R2 and TROP2 in U937. Rabbit anti-TRAIL-R2 antibody and mouse anti-TROP2 antibody did not bind the surface of U937 as detected by no fluorescent signal. Both rabbit and mouse antibody isotype controls did not bind the surface of U937.

FIG. 14 shows dose dependent TRAIL-R2 mediated apoptosis and cell death in U937 by αTROP2×αTRAIL-R2 bispecific antibody with a maximum cell killing of 11% at cell passage number 7 and maximum cell killing of 15% at cell passage number 10 at IC₅₀=0.65 nM (0.13 μg/ml).

FIG. 15 shows solution profile analysis of Protein A and preparative Size Exclusion Chromatography (SEC) purified αTROP2×αCD33, αTROP2×αCSF1R and α0TROP2×αCD163, bispecific antibody by Size Exclusion Ultra-Performance Liquid Chromatography (SE-UPLC). The samples are analysed on a Waters Acquity Protein BEH SEC 125 A 1.7 μm column using PBS with 10% isopropanol as the mobile phase. A single major peak corresponding to the molecular weight of the monomer bispecific antibody assembly was observed for both αTROP2×αCD33 and αTROP2×αCSF1R suggesting there is no presence of low molecular and high molecular aggregates indicating stability. The major peak for αTROP2×αCD163 corresponding to the molecular weight of the monomer bispecific antibody assembly was 81% with a preceding shoulder corresponding to possible high molecular weight forms.

FIG. 16 shows SDS-PAGE analysis of Protein A purified αTROP2×αCD33, αTROP2×αCSF1R and αTROP2×αCD163 bispecific antibody. The samples were analyzed under reducing (R) and non-reducing (NR) conditions. Two bands are observed under reducing conditions, top band corresponding to the molecular weight of the heavy chain and lower band corresponding to the molecular weight of the light chain. Under non-reducing conditions only one band was observed corresponding to the molecular weight of the intact bispecific antibody construct. 2 μg of each antibody sample +/−β-mercaptoethanol (reduced/non-reduced respectively) is loaded on a NuPage 4-12% Bis-Tris gel (ThermoFisher, Loughborough, UK) and run at 200 V for 40 minutes. Gels are stained with InstantBlue (Expedeon, Swavesey, UK). M: PAGERuler™ Plus pre-stained protein ladder (ThermoFisher, Loughborough, UK). Lanes 1,2; αTROP2×αCSF1R bispecific antibody, reduced and non-reduced, respectively; Lanes 3, 4; αTROP2×αCD163 bispecific antibody, reduced and non-reduced, respectively; Lanes 5,6; αTROP2×αCD33 bispecific antibody, reduced and non-reduced, respectively; Lanes 7,8; αTROP2 monospecific antibody, reduced and non-reduced, respectively.

FIG. 17 shows Biacore multi-cycle kinetics sensorgrams of αTROP2×αCD33 binding to TROP2 and CD33 following protein A and preparative SEC purification. A 1 to 1 model was used for fitting.

FIG. 18 shows Biacore multi-cycle kinetics sensorgrams of αTROP2×αCD163 binding to TROP2 and CD33 following protein A and preparative SEC purification. A 1 to 1 model was used for fitting.

FIG. 19 shows Biacore multi-cycle kinetics sensorgrams of αTROP2×αCSF1R binding to TROP2 and CSF1R following protein A purification and preparative SEC purification. A 1 to 1 model was used for fitting.

FIG. 20A-FIG. 20D show target mediated selective binding of αTROP2×αCD33 bispecific antibody on the surface of cancer cells measured by Fluorescent Activated Cell Sorting method (FACS). FIG. 20A shows selective binding of αTROP2×αCD33 bispecific antibody on the surface of a TROP2+ cancer cell line, SKBR3, with a binding affinity, K_{D}=2.7 nM (0.55 μg/ml). The non-binding IgG1 isotype control did not bind on the surface of SKBR3. As a system control Herceptin bound on the surface of SKBR3 with a binding affinity, K_{D}=4.1 nM (0.62 μg/ml) due to high expression of HER2 as manifested by high fluorescence intensity compared to the binding of αTROP2×αCD33. FIG. 20B shows selective binding of αTROP2×αCD33 bispecific antibody on the surface of a CD33+ cancer cell line, THP1, with a binding affinity, K_{D}=6.7 nM (1.33 μg/ml). There was a concentration dependent increase in fluorescence intensity for αTROP2×αCD33. In contrast neither the non-binding IgG1 isotype control nor Herceptin exhibited any concentration dependent increase in fluorescence intensity in THP1 suggestion neither of the antibodies bound THP1. FIG. 20C shows high levels of TROP2 expressed on the surface of SKBR3 cell line. The non-binding rabbit antibody isotype control did not bind on the surface of SKBR3 cell line as detected by low fluorescent signal. FIG. 20D shows high levels of CD33 expressed on the surface of THP1 cell line. The non-binding rabbit antibody isotype control did not bind on the surface of THP1 cell line as detected by no fluorescent signal.

FIG. 21A illustrates a cartoon representation of a TRAIL-R2 mediated activation on a target cancer cell which is selectively activated by a multi-specific antibody disclosed herein. As shown in this figure, the multi-specific antibody binds to a tumor specific antigen located in the lipid raft which recruits and facilitates oligomerization (e.g., dimerization) of the TRAIL-R2 and subsequent activation of the TRAIL-R2 mediated apoptosis within the target cancer cell.

FIG. 21B illustrates a cartoon representation of an exemplary multi-specific antibody that binds to a tumor specific antigen located outside of the lipid raft of a target cancer cell. The exemplary multi-specific antibody, upon binding to the tumor specific antigen, does not activate the TRAIL-R2 mediated apoptosis within the target cancer cell.

FIG. 21C illustrates a cartoon representation of a conventional antibody such as the single action bispecific antibody illustrated in FIG. 1C which generates non-tumor toxicity due to leakage of inflammatory cytokines, granzymes, and/or performs to nearby normal cells and tissues. As shown in this figure, upon recruiting an immune effector cell to a cancer cell and activation of the immune effector cell by the conventional antibody, the activated immune effector cells not only secretes soluble TRAILs that can lead to activation of apoptosis within the cancer cell via the TRAIL-R2 pathway, but can also release cytokine, granzymes, and/or performs which leakage to nearby normal cells leads to non-tumor toxicity.

FIG. 21D illustrates a cartoon representation of an exemplary multi-specific antibody which modulates TRAIL-R2 mediated apoptosis of the cancer cell and does not activate immune effector cells. As shown in this figure, the lack of immune effector cell activation minimizes or prevents the release of inflammatory cytokines, granzymes, and/or performs, and as such, reduces or inhibits toxicity to nearby normal cells or tissues.

FIG. 22 illustrates no cell killing by an exemplary multi-specific antibody in a TRAIL-sensitive MDAMB231 cell line.

### DETAILED DESCRIPTION OF THE DISCLOSURE

### Tumor Microenvironment and Immune Suppression

The tumor microenvironment (TME) plays an integral part in tumor progression, tumor cell adaptation, and resistance to anti-cancer therapy. Components of the tumor microenvironments include myeloid-derived suppressor cells (MDSCs), antigen-presenting cells (APCs), lymphocytes, neutrophils, tumor-associated macrophages (TAMs), fibroblasts, extracellular matrix composed of collagen and proteoglycans, and soluble factors (e.g., cytokines and growth factors), all of which may assist or hinder antitumor immune responses. Immunotherapy harnesses the immune system, both innate and adaptive, to attack and destroy tumor cells. However, multiple mechanisms used by tumor cells, including alteration of the antigen presentation machinery, secretion of immune suppressive factors that can induce apoptosis of lymphocytes, or activate negative regulatory pathways, resulting in evasion from anti-tumor immune response, thereby limiting the effectiveness of the immune response.

Exemplary immune suppressive factors and cells include CD4+ T regulatory (Treg) cells, MDSCs, TAMs, and inhibitory immune checkpoint molecules. In some embodiments, Treg cells are characterized with expression of FOXP3, CD4, and CD25. In some embodiments, Treg cells suppress T-effector cell responses via secretion of inhibitory cytokines (e.g., IL-10, IL-35, and TGF-β) or via direct cell contact.

Myeloid-derived suppressor cells (MDSCs) are a heterogeneous group of immune cells originated from bone marrow stem cells. MDSCs normally differentiate into granulocytes, macrophages, or dendritic cells. However, in the tumor microenvironment, MDSCs become activated, expand rapidly, but remain undifferentiated. In some embodiments, MDSCs have been shown in clinical studies to be associated with reduced survival in several human tumors including colorectal cancer and breast cancer.

Macrophages, a plentiful myeloid derived phagocytic cell, can be classified as pro-inflammatory or anti-inflammatory, also known as classic (M1) and alternative (M2). TAMs are a subset of macrophages that affect responses to immunotherapy and coordinate tumor-promoting angiogenesis, fibrous stroma deposition, and metastasis formation. TAMs display an alternatively activated M2 phenotype known to control tissue homeostasis and wound healing. However, in a tumor setting, this phenotype enables T cell inhibition via cytokines (e.g., IL-10), depletion of metabolites (e.g., expression of arginase, IDO), and/or by contact inhibition (e.g., via PD-L1).

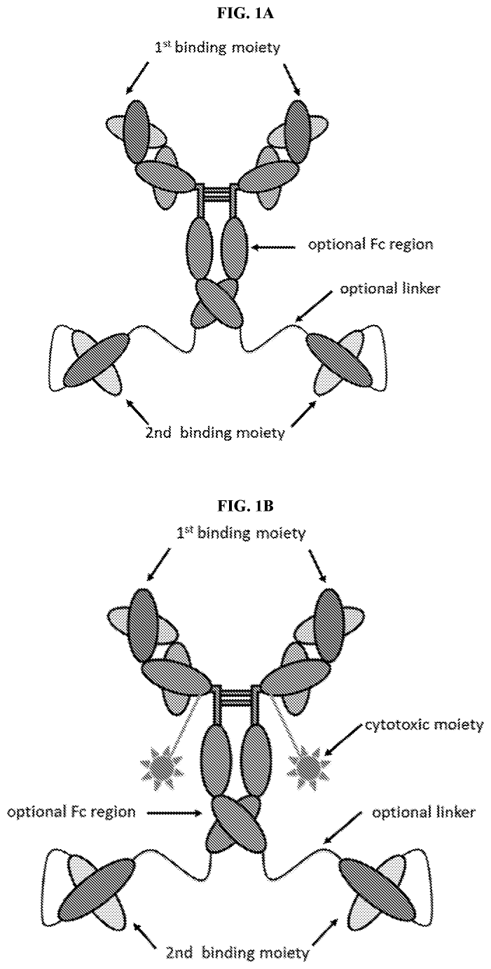

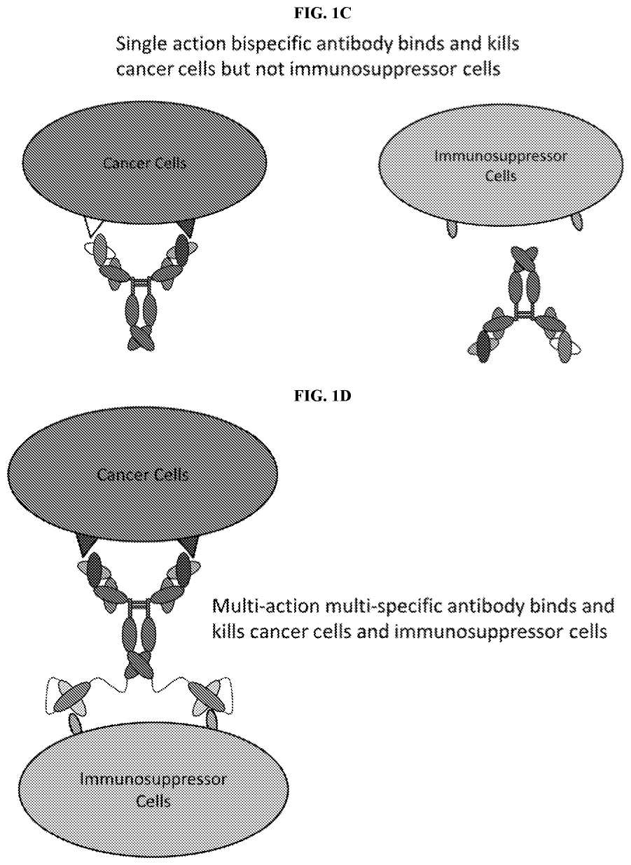

Cancer therapies focus in eliminating cancer cells either directly or by engaging the body's immune system (also see FIG. 1C). For example, therapies such as chemotherapy, radiotherapy, antibody drug conjugates, CAR-Ts, or BiTEs are designed to directly eliminate cancer cells while immunotherapies such as immunecheckpoint inhibitors block certain immunecheckpoint receptors on cancer cells or T-cells to activate T-cells to eliminate cancer cells. Immuno-oncology strategies involving bispecific antibodies are also designed to eliminate cancer cells by engaging or harnessing T-cells or NK-cells. Indeed, there are 86 clinical trials evaluating bispecific antibodies and all of them are designed to engage and/or activate the immune system. As such, these cancer treatment modalities only target and kill tumor cells and do not address the immunosuppressive cells.

Disclosed herein, in certain embodiments, are multi-specific polypeptide molecules (e.g., multi-specific antibodies) that comprise a first binding moiety specific for a tumor-associated antigen or receptor expressed on a tumor cell, and a second binding moiety specific for a receptor or antigen expressed on an immunosuppressive cell. In some embodiments, the immunosuppressive cell comprises a myeloid-derived suppressor cell (MDSC), a tumor-associated macrophage, or a CD4+ T regulator (Treg) cell. In some embodiments, also described herein are multi-specific polypeptide molecules (e.g., multi-specific antibodies) that comprise a first binding moiety specific for a tumor-associated antigen or receptor expressed on a tumor cell and a second binding moiety specific for a receptor or antigen expressed on a MDSC or a tumor-associated macrophage. In additional embodiments, described herein are multi-specific polypeptide molecules (e.g., multi-specific antibodies) that comprise a first binding moiety specific for a tumor-associated antigen or receptor expressed on a tumor cell and a second binding moiety specific for a receptor or antigen expressed on Treg cell.

In some embodiments, the multi-specific polypeptide molecules (e.g., multi-specific antibodies) described herein modulate an immunosuppressive cell population. In some instances, the multi-specific polypeptide molecules (e.g., multi-specific antibodies) described herein decreases an immunosuppressive cell population, induces a cell-kill effect on the immunosuppressive cell population, reduces immunosuppressive cell expansion, reduces immunosuppressive cell activation, and/or decreases immunosuppressive cell proliferation. In some cases, the multi-specific polypeptide molecules (e.g., multi-specific antibodies) described herein ameliorates, eliminates, and/or blocks an escape mechanism that the cancer cells utilize to evade an anti-tumor immune response. In some cases, the multi-specific polypeptide molecules (e.g., multi-specific antibodies) described herein does not target or specifically binds to an immune checkpoint pathway receptor.

In some embodiments, the multi-specific polypeptide molecules (e.g., multi-specific antibodies) described herein modulates a tumor-infiltrating lymphocyte (TIL) expansion by eliminating immunosuppressive factors such as, immunosuppressive cells and immunosuppressive cytokines. In some instances, the multi-specific polypeptide molecules (e.g., multi-specific antibodies) described herein enhances TIL population expansion, cytotoxic T-cell proliferation, and/or cytotoxic-T cell activation. In some cases, the multi-specific polypeptide molecules (e.g., multi-specific antibodies) described herein increases a ratio of TIL to immunosuppressive cells.

In some embodiments, the multi-specific polypeptide molecules (e.g., multi-specific antibodies) described herein modulates a population of Treg. In some instances, the multi-specific polypeptide molecules (e.g., multi-specific antibodies) described herein decreases Tregs within a target population, reduces Treg proliferation, reduces Treg expansion, and/or reduces Treg activation. In some cases, the multi-specific polypeptide molecules (e.g., multi-specific antibodies) described herein decreases a ratio of Tregs to TILs in a target population.

In some embodiments, the multi-specific polypeptide molecules (e.g., multi-specific antibodies) described herein modulates a population of MDSCs. In some instances, the multi-specific polypeptide molecules (e.g., multi-specific antibodies) described herein reduces MDSC proliferation, reduces MDSC activation, and/or reduces MDSC expansion. In some instances, the multi-specific polypeptide molecules (e.g., multi-specific antibodies) described herein reduces MDSCs in a target population. In some cases, the multi-specific polypeptide molecules (e.g., multi-specific antibodies) described herein reduces a ratio of MDSCs to TILs in a target population.

In some embodiments, the multi-specific polypeptide molecules (e.g., multi-specific antibodies) described herein modulates a M1 polarization of macrophages. In some instances, the multi-specific polypeptide molecules (e.g., multi-specific antibodies) described herein increases M1 polarization. In some instances, the multi-specific polypeptide molecules (e.g., multi-specific antibodies) described herein decreases M2 polarization. In some cases, the multi-specific polypeptide molecules (e.g., multi-specific antibodies) described herein decreases TAM (e.g., M2-TAM) proliferation, expansion, and/or activation. In some cases, the multi-specific polypeptide molecules (e.g., multi-specific antibodies) described herein increases a ratio of macrophages presenting a M1 phenotype over macrophages presenting a M2 phenotype. In some cases, the multi-specific polypeptide molecules (e.g., multi-specific antibodies) described herein increases an anti-tumor macrophage proliferation and/or activation. In some cases, the multi-specific polypeptide molecules (e.g., multi-specific antibodies) described herein enhances expansion of a population of anti-tumor macrophages.

In certain embodiments, further described herein are methods of treating a subject with a multi-specific protein molecule (e.g., a multi-specific antibody) that comprise administering a multi-specific polypeptide (e.g., a multi-specific antibody) comprising a first binding moiety specific for a tumor-associated antigen or receptor expressed on a tumor cell and a second binding moiety specific for a receptor or antigen expressed on an immunosuppressive cell.

In certain embodiments, additionally described herein are methods of inducing tumor and immunosuppressive cell killing in a target cell population with use of a multi-specific protein molecule (e.g., a multi-specific antibody) that comprises a first binding moiety specific for a tumor-associated antigen or receptor expressed on a tumor cell and a second binding moiety specific for a receptor or antigen expressed on an immunosuppressive cell.

In certain embodiments, additionally described herein are methods of inducing tumor and immunosuppressive cell killing in a target cell population via induce direct cell death of both cancer cells and immunosuppressive cells by activating apoptotic pathways by a multi-specific protein molecule (e.g., a multi-specific antibody) that comprises a first binding moiety specific for a tumor-associated antigen or receptor expressed on a tumor cell and a second binding moiety specific for a receptor or antigen expressed on an immunosuppressive cell. In some instances, the method comprises induction of cell death via a cytotoxic payload conjugated to the multi-specific protein molecule (e.g., the multi-specific antibody). In other instances, the method comprises utilizing antibody-dependent cellular cytotoxicity to induce cell kill effect. In additional instances, the method comprises a combination of cytotoxic payload associated cytotoxicity and antibody-dependent cellular cytotoxicity to induce cell kill effect.

In certain embodiments, further described herein are antibody-cytotoxin conjugates that specifically binds to an antigen expressed on an immunosuppressive cell. In some instances, the antibody-cytotoxin conjugates are used in a method of inducing immunosuppressive cell killing in a subject in need thereof, comprising administering the antibody-cytotoxin conjugate that specifically binds to an antigen expressed on an immunosuppressive cell, thereby killing the immunosuppressive cell in the subject. In other instances, the antibody-cytotoxin conjugates are used in a method of activating tumor cell-killing immune cells in a subject in need thereof, comprising administering an antibody-cytotoxin conjugate that specifically binds to an antigen expressed on an immunosuppressive cell, thereby killing the immunosuppressive cell and activating the tumor cell-killing immune cells in the subject. In additional instances, the antibody-cytotoxin conjugates are used in a method of reducing suppression of tumor cell-killing immune cells in a subject in need thereof, comprising administering an antibody-cytotoxin conjugate that specifically binds to an antigen expressed on an immunosuppressive cell, thereby killing the immunosuppressive cell and reducing suppression of the tumor cell-killing immune cells in the subject. In some cases, the antibody-cytotoxin conjugate further comprises a tumor-specific binding moiety. In some cases, the antibody-cytotoxin conjugate is a multi-specific binding polypeptide (e.g., a multi-specific antibody) described herein.

### Multi-Specific Binding Polypeptide Structures

Disclosed herein are multi-specific binding polypeptides (e.g., multi-specific antibodies) that comprise a tumor binding moiety that specifically binds to a tumor-associated antigen and an immune cell binding moiety that specifically binds to an antigen expressed on an immunosuppressive cell. In some instances, the multi-specific binding polypeptide (e.g., the multi-specific antibody) binds to and modulate killing of a cancer cell and binds to and modulate killing of an immunosuppressor cell. Also see FIG. 1D. In some instances, the multi-specific binding polypeptide comprises a single polypeptide complex comprising at least two distinct non-overlapping binding domains, each of the distinct binding domains being specific for distinct non-overlapping antigens or epitopes. The antigen or epitope can be on different molecules (e.g., different proteins), or non-overlapping portions of the same molecule. A single polypeptide complex encompasses single polypeptides, polypeptides that dimerize or multimerize to form a binding unit (e.g., heavy chain and light chain of an IgG, two heavy chain and light chain pairs forming a mature IgG), polypeptides that are distinct but held together by covalent (e.g., disulfide bonds, linker molecules) or non-covalent interactions (e.g., biotin-streptavidin; affinity interactions; charge interactions, knob-in-hole).

The multi-specific binding polypeptides described herein, in certain embodiments, comprise antibodies or binding fragments of antibodies. Among the provided multi-specific binding polypeptides are monoclonal antibodies, multi-specific antibodies, for example, bispecific antibodies, and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region.

In some embodiments, a multi-specific antibody described herein comprises a full-length antibody, an appended antibody, a bispecific fusion protein, or a bispecific antibody conjugate. In some cases, the multi-specific antibody comprises a nanobody, BiTE, diabody, DART, TandAb, scDiabody, scDiabody-CH3, triple body, miniantibody, minibody, TriBi minibody, scFv-CH3 KIH, Fab-scFv, scFv-CH-CL-scFv, F(ab′)2, F(ab′)2-scFv2. scFv-KIH, Fab-scFv-Fc, tetravalent HCAb, scDiabody-Fc, diabody-Fc, tandem scFv-Fc, or intrabody. In some cases, the multi-specific antibody comprises an appended antibody. In some cases, the multi-specific antibody comprises a bispecific fusion protein. In some cases, the multi-specific antibody comprises a bispecific antibody conjugate. In some cases, the multi-specific antibody comprises an appended antibody further comprising a conjugate (e.g., a payload such as a cytotoxic moiety).

The multi-specific binding polypeptides (e.g., the multi-specific antibody) of the current disclosure have certain structural attributes. FIG. 1A shows a specific non-limiting embodiment of one such multi-specific binding polypeptide (e.g., a multi-specific antibody). The polypeptide minimally comprises a first binding moiety that binds a tumor-associated antigen. This binding moiety comprises, for example, a VH/VL pair of a monoclonal antibody specific for the tumor-associated antigen. The polypeptide also comprises a second binding moiety comprising a single-chain variable region (scFv) that binds to an antigen expressed on an immunosuppressive cell. While the first binding moiety is described as being specific for a tumor-associated antigen, and the second binding moiety is described as being specific for an antigen expressed on an immunosuppressive cell, this order can be reversed with the first binding moiety binding to an antigen expressed on an immunosuppressive cell and the second binding moiety binding to a tumor-associated antigen. FIG. 1A also shows optional features for the polypeptides described herein, including an Fc region and a polypeptide linker (e.g., a flexible polypeptide linker) coupling the first binding moiety and the second binding moiety. In one embodiment, the multi-specific binding polypeptide (e.g., the multi-specific antibody) comprises a first binding moiety that comprises a monoclonal IgG antibody and a second binding moiety that comprises an scFv, wherein the scFv antibody is coupled to the C-terminus of one or both of the Fc regions of the monoclonal antibody. In certain embodiments, the IgG comprises an IgG₁ isotype. In certain embodiments, the IgG comprises an IgG₂ isotype. In certain embodiments, the IgG comprises an IgG₄ isotype. In certain embodiments, the polypeptide linker comprises a poly-Ala linker, a poly-Gly linker, or a combination of poly-Ala and poly-Gly residues. In some instances, the polypeptide linker comprises (Gly4Ser)ₙ, wherein n is an integer from 1 to 10. In some instances, the n is an integer from 1 to 6, 1 to 4, or 1 to 2. In some cases, n is 1, 2, 3, 4, 5, 6, 7, 8, 9, or 10. In some cases, n is 1, 2, 3, 4, 5, or 6. In some cases, n is 1, 2, 3, or 4. In some cases, the polypeptide linker comprises GGGSGGGS (SEQ ID NO: 78). In some cases, the polypeptide linker comprises GGGSGGGSGGGS (SEQ ID NO: 140). In some cases, the polypeptide linker comprises GGGSGGGSGGGSGGGS (SEQ ID NO: 141). In certain embodiments, the Fc region has been modified to increase half-life in circulation. In certain embodiments, the half-life is extended to at least 2, 3, 4, 5, 6, 7, 8, 9, 10, 11, 12, 13, 14, 15, 16, 17, 18, 19, 20, 21, 25, 28, 30, 60, or more days. In certain embodiments, the half-life is extended to at least 7, 14 or 21 days. In certain embodiments, the Fc region has been modified to increase antibody dependent cell-cytotoxicity (ADCC). In certain embodiments, the Fc region that has been modified to reduce the affinity for human neonatal Fc receptor (FcRn). In certain embodiments, the Fc region that has been modified to reduce neutropenia. In certain embodiments, one or both hinge regions have been modified.

In addition to the configuration shown in FIG. 1A other configurations are envisioned. Exemplary multi-specific antibody configurations include, but are not limited to: (1) a Light chain c-terminal scFv format, wherein an scFv is coupled to the C-terminus of one or more light chains of an antibody, as in US 2013/0216543; (2) a dual-variable domain (DVD) antibody, wherein the light chain and heavy chain variable regions comprise a distinct second binding domain, as in U.S. Pat. No. 8,258,268; (3) a knob-in-hole bispecific, wherein one heavy chain constant region comprises an engineered hole, and one heavy chain constant region comprises an engineered knob, as in Merchant et al. “An efficient route to human bispecific IgG.” Nat Biotechnol. 1998 July;16(7):677-81, and WO 2016/172485; (4) dual scFvs, wherein two scFvs with different binding specificities are joined by a linker; and (5) dual F(ab′)₂ wherein two F(ab′)s with different binding specificities are joined by a linker or chemical crosslinking. Any bispecific antibody format can be used as a multi-specific binding polypeptide of the current disclosure. See Spiess, et al., Molecular Immunology, 67(2): 95-106 (2015). In certain embodiments, the multi-specific binding polypeptide is a bispecific of a format selected from the list consisting of: IgG-scFv, nanobody, diabody, Dual-affinity Re-targeting Antibody (DART), TandAb, scDiabody, scDiabody-CH3, triple body, mini-antibody, minibody, TriBi minibody, scFv-CH3 KIH, Cross-mab Knob-in-hole (KIH), Fab-scFv, scFv-CH-CL-scFv, F(ab′)2, F(ab′)2-scFv2. scFv-KIH, Fab-scFv-Fc, tetravalent HCAb, scDiabody-Fc, diabody-Fc, tandem scFv-Fc, and intrabody.

In some embodiments, the multi-specific binding polypeptide (e.g., the multi-specific antibody) comprises a bispecific format described in Godar, et al., “Therapeutic bispecific antibody formats: a patent applications review (1994-2017),” Expert Opinion on Therapeutic Patents, 28(3): 251-276 (2018).

In some embodiments, the multi-specific binding polypeptide (e.g., the multi-specific antibody) comprises a bispecific format described in Labrijn, et al., “Bispecific antibodies: a mechanistic review of the pipeline,” Nature Reviews 18: 585-608 (2019).

### Target Antigens

In certain embodiments, a multi-specific binding polypeptide (e.g., a multi-specific antibody) of the current disclosure possesses distinct binding moieties that bind to a tumor associated antigen and an immunosuppressive cell expressed antigen. In certain embodiments, a multi-specific binding polypeptide (e.g., a multi-specific antibody) of the current disclosure contributes to killing of both tumor/cancer cells and immunosuppressive cells.

Tumor-associated antigens (TAAs) are cell-surface antigens accessible to a multi-specific binding polypeptide (e.g., a multi-specific antibody). The multi-specific binding polypeptides (e.g., multi-specific antibodies) of the current disclosure can contribute to cell killing directly by an ADCC mechanism or by target mediated delivery of a cytotoxic payload. In certain embodiments, the tumor associated antigen is TROP2 (also referred to as TACSTD2), HER2 (also referred to as ERBB2), GPC3 (also referred to as Glypican 3), GD2, FOLR1 (also referred to as Folate Receptor 1), FLT3, BCMA (also referred to as B-cell maturation antigen), MUC16, SLC4A4, STEAP1, CD19, CD20, CD22, CD25, CD33, CD38, CD30, CD47, CD123, mesothelin, MT1-MMP (also referred to as MMP14), or PSMA (also referred to as FOLH1). In certain embodiments, the tumor associated antigen is TROP2. In certain embodiments, the tumor-associated antigen is HER2. In certain embodiments, the tumor-associated antigen is CD30. In certain embodiments, the tumor-associated antigen is CD22. In certain embodiments, the tumor-associated antigen is GD2. In certain embodiments, the tumor-associated antigen is FOLR1. In certain embodiments, the tumor-associated antigen is CD33. In certain embodiments, the tumor-associated antigen is GPC3. In certain embodiments, the tumor-associated antigen is CD38. In certain embodiments, the tumor-associated antigen is FLT3. In certain embodiments, the tumor-associated antigen is CD47. In certain embodiments, the tumor-associated antigen is CD22. In certain embodiments, the tumor-associated antigen is FOLH1.

In certain embodiments, a multi-specific binding polypeptide (e.g., a multi-specific antibody) comprises a tumor binding moiety that binds to TROP2, HER2, GPC3, GD2, FOLR1, FLT3, BCMA, MUC16, SLC4A4, STEAP1, CD19, CD20, CD22, CD25, CD33, CD38, CD30, CD47, CD123, mesothelin, MT1-MMP, or PSMA. In some embodiments, the tumor binding moiety binds to TROP2, GPC3, HER2, FOLR1, CD33, CD38, FLT3, CD30, CD22, or GD2. In some embodiments, the tumor binding moiety binds to TROP2, GPC3, FOLR1, CD33, CD38, or FLT3. In some embodiments, the tumor binding moiety binds to TROP2, CD47, HER2, CD30, CD22, GD2, or FOLR1. In some cases, the tumor binding moiety binds to TROP2. In some cases, the tumor binding moiety binds to CD30. In some cases, the tumor binding moiety binds to CD22. In some cases, the tumor binding moiety binds to GD2. In some cases, the tumor binding moiety binds to FOLR1. In some cases, the tumor binding moiety binds to CD33. In some cases, the tumor binding moiety binds to GPC3. In some cases, the tumor binding moiety binds to HER2. In some cases, the tumor binding moiety binds to CD38. In some cases, the tumor binding moiety binds to FLT3. In some cases, the tumor binding moiety binds to CD47. In some cases, the tumor binding moiety binds to CD22.

Immunosuppressive cells infiltrate tumor sites and suppress the endogenous immune response to cancer cells. Killing immunosuppressive cells by a cytotoxic payload or ADCC can contribute to increased tumor killing by natural killer cells, and cytotoxic T lymphocytes. In certain embodiments, the immunosuppressive cell comprises a myeloid derived suppressor cell (MDSC), a CD4+ T regulatory cell, a CD8+ T regulatory cell, or a tumor-associated macrophage. Exemplary MDSC receptors include, but are not limited to, TNF-related apoptosis-inducing ligand receptor 2 (TRAIL-R2) and colony stimulating factor 1 receptor (CSF1R). Exemplary tumor-associated macrophage receptors include, but are not limited to, Cluster of Differentiation 163 (CD163) and macrophage receptor with collagenous structure (MARCO). Exemplary Treg receptors include, but are not limited to, tumor necrosis factor receptor 2 (TNFR2) and semaphorin 4A (SEMA4A).

In certain embodiments, exemplary antigens expressed by an immunosuppressive cell include, but are not limited to, TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the antigen expressed by an immunosuppressive cell comprises TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), CD200R, or SELPLG (also referred to as PSLG-1 or CD162). In some instances, the antigen expressed by an immunosuppressive cell comprises TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the antigen expressed by an immunosuppressive cell comprises TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the antigen expressed by an immunosuppressive cell comprises TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the antigen expressed by an immunosuppressive cell comprises TRAIL-R2, CSF1R, TREM2, C5AR1, LYVE1, MRC1, STAB1, MERTK, SIGLEC1, or IL4R. In some instances, the antigen expressed by an immunosuppressive cell comprises MARCO, SELPLG, CD163, MS4A7, CD200R, MGL1, or MGL2. In some instances, the antigen expressed by an immunosuppressive cell comprises CD33, ABCC3, LILRB4, TMEM37, TMEM119, SIGLEC7, or SIGLEC9. In some instances, the antigen expressed by an immunosuppressive cell comprises SEMA4A, SEMA4D, or TNFR2. In some instances, the antigen expressed by an immunosuppressive cell comprises TRAIL-R2, CD33, CD163, or CSF1R. In some instances, the antigen expressed by an immunosuppressive cell comprises CD33, CD163, or CSF1R. In some instances, the antigen expressed on the immunosuppressive cell is CSF1R. In some instances, the antigen expressed on the immunosuppressive cell is SEMA4A. In some instances, the antigen expressed on the immunosuppressive cell is CD163. In some instances, the antigen expressed on the immunosuppressive cell is TNFR2 (or TNFRSF1B). In some instances, the antigen expressed on the immunosuppressive cell is TRAIL-R2 (or TNFRSF10B or DR5). In some instances, the antigen expressed on the immunosuppressive cell is CD33. In some instances, the antigen expressed on the immunosuppressive cell is SEMA4D. In some instances, the antigen expressed on the immunosuppressive cell is LILRB4. In some instances, the antigen expressed on the immunosuppressive cell is TREM2. In some instances, the antigen expressed on the immunosuppressive cell is C5AR1. In some instances, the antigen expressed on the immunosuppressive cell is SIGLEC1. In some instances, the antigen expressed on the immunosuppressive cell is TMEM37. In some instances, the antigen expressed on the immunosuppressive cell is TMEM119. In some instances, the antigen expressed on the immunosuppressive cell is IL4R. In some instances, the antigen expressed on the immunosuppressive cell is SIGLEC7.

In some embodiments, a multi-specific binding polypeptide (e.g., a multi-specific antibody) comprises an immune cell binding moiety that binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the immune cell binding moiety binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), CD200R, or SELPLG (also referred to as PSLG-1 or CD162). In some instances, the immune cell binding moiety binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the immune cell binding moiety binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the immune cell binding moiety binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the immune cell binding moiety binds to TRAIL-R2, CSF1R, TREM2, C5AR1, LYVE1, MRC1, STAB1, MERTK, SIGLEC1, or IL4R. In some instances, the immune cell binding moiety binds to MARCO, SELPLG, CD163, MS4A7, CD200R, MGL1, or MGL2. In some instances, the immune cell binding moiety binds to CD33, ABCC3, LILRB4, TMEM37, TMEM119, SIGLEC7, or SIGLEC9. In some instances, the immune cell binding moiety binds to SEMA4A, SEMA4D, or TNFR2. In some instances, the immune cell binding moiety binds to TRAIL-R2, CD33, CD163, or CSF1R. In some instances, the immune cell binding moiety binds to CD33, CD163, or CSF1R. In some cases, the immune cell binding moiety binds to CSF1R. In some cases, the immune cell binding moiety binds to SEMA4A. In some cases, the immune cell binding moiety binds to CD163. In some cases, the immune cell binding moiety binds to TNFR2 (or TNFRSF1B). In some cases, the immune cell binding moiety binds to TRAIL-R2 (or TNFRSF10B or DR5). In some cases, the immune cell binding moiety binds to CD33. In some cases, the immune cell binding moiety binds to SEMA4D. In some cases, the immune cell binding moiety binds to TREM2. In some cases, the immune cell binding moiety binds to C5AR1. In some cases, the immune cell binding moiety binds to LYVE1. In some cases, the immune cell binding moiety binds to ABCC3. In some cases, the immune cell binding moiety binds to LILRB4. In some cases, the immune cell binding moiety binds to MRC1. In some cases, the immune cell binding moiety binds to SIGLEC1. In some cases, the immune cell binding moiety binds to STAB1. In some cases, the immune cell binding moiety binds to TMEM37. In some cases, the immune cell binding moiety binds to MERTK. In some cases, the immune cell binding moiety binds to TMEM119. In some cases, the immune cell binding moiety binds to SIGLEC7. In some cases, the immune cell binding moiety binds to SIGLEC9. In some cases, the immune cell binding moiety binds to IL4R. In some cases, the immune cell binding moiety binds to MARCO. In some cases, the immune cell binding moiety binds to SELPLG (also referred to as PSLG-1 or CD162). In some cases, the immune cell binding moiety binds to CD163. In some cases, the immune cell binding moiety binds to MS4A7. In some cases, the immune cell binding moiety binds to CD200R. In some cases, the immune cell binding moiety binds to MGL1.

In certain embodiments, the multi-specific binding polypeptide is a multispecific antibody (optionally a bispecific antibody) comprising an immune cell binding moiety that binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the immune cell binding moiety binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), CD200R, or SELPLG (also referred to as PSLG-1 or CD162). In some instances, the immune cell binding moiety binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the immune cell binding moiety binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the immune cell binding moiety binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the immune cell binding moiety binds to TRAIL-R2, CSF1R, TREM2, C5AR1, LYVE1, MRC1, STAB1, MERTK, SIGLEC1, or IL4R. In some instances, the immune cell binding moiety binds to MARCO, SELPLG, CD163, MS4A7, CD200R, MGL1, or MGL2. In some instances, the immune cell binding moiety binds to CD33, ABCC3, LILRB4, TMEM37, TMEM119, SIGLEC7, or SIGLEC9. In some instances, the immune cell binding moiety binds to SEMA4A, SEMA4D, or TNFR2. In some instances, the immune cell binding moiety binds to TRAIL-R2, CD33, CD163, or CSF1R. In some instances, the immune cell binding moiety binds to CD33, CD163, or CSF1R. In some cases, the immune cell binding moiety binds to CSF1R. In some cases, the immune cell binding moiety binds to SEMA4A. In some cases, the immune cell binding moiety binds to CD163. In some cases, the immune cell binding moiety binds to TNFR2 (or TNFRSF1B). In some cases, the immune cell binding moiety binds to TRAIL-R2 (or TNFRSF10B or DR5). In some cases, the immune cell binding moiety binds to CD33. In some cases, the immune cell binding moiety binds to SEMA4D. In some cases, the immune cell binding moiety binds to TREM2. In some cases, the immune cell binding moiety binds to C5AR1. In some cases, the immune cell binding moiety binds to LYVE1. In some cases, the immune cell binding moiety binds to ABCC3. In some cases, the immune cell binding moiety binds to LILRB4. In some cases, the immune cell binding moiety binds to MRC1. In some cases, the immune cell binding moiety binds to SIGLEC1. In some cases, the immune cell binding moiety binds to STAB 1. In some cases, the immune cell binding moiety binds to TMEM37. In some cases, the immune cell binding moiety binds to MERTK. In some cases, the immune cell binding moiety binds to TMEM119. In some cases, the immune cell binding moiety binds to SIGLEC7. In some cases, the immune cell binding moiety binds to SIGLEC9. In some cases, the immune cell binding moiety binds to IL4R. In some cases, the immune cell binding moiety binds to MARCO. In some cases, the immune cell binding moiety binds to SELPLG (also referred to as PSLG-1 or CD162). In some cases, the immune cell binding moiety binds to CD163. In some cases, the immune cell binding moiety binds to MS4A7. In some cases, the immune cell binding moiety binds to CD200R. In some cases, the immune cell binding moiety binds to MGL1.

In certain embodiments, a multi-specific binding polypeptide described herein is a multi-specific antibody (e.g., a bispecific antibody) comprising a tumor binding moiety that specifically binds to TROP2, HER2, GPC3, GD2, FOLR1, FLT3, BCMA, MUC16, SLC4A4, STEAP1, CD19, CD20, CD22, CD25, CD33, CD38, CD30, CD47, CD123, mesothelin, MT1-MMP, or PSMA; and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some embodiments, the multi-specific antibody comprises a tumor binding moiety that specifically binds to TROP2, GPC3, HER2, FOLR1, CD33, CD38, FLT3, CD30, CD22, or GD2; and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some embodiments, the multi-specific antibody comprises a tumor binding moiety that specifically binds to TROP2, GPC3, FOLR1, CD33, CD38, or FLT3; and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162).

In some embodiments, the multi-specific antibody comprises a tumor binding moiety that specifically binds to TROP2 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to TROP2 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to TROP2 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to TROP2 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to TROP2 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to TROP2 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to TROP2 and an immune cell binding moiety that specifically binds to MARCO, SELPLG, CD163, MS4A7, CD200R, MGL1, or MGL2. In some instances, the multi-specific antibody comprises a tumor binding moiety that specifically binds to TROP2 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to TROP2 and an immune cell binding moiety that specifically binds to SEMA4A, SEMA4D, or TNFR2. In some instances, the multi-specific antibody comprises a tumor binding moiety that specifically binds to TROP2 and an immune cell binding moiety that specifically binds to TRAIL-R2, CD33, CD163, or CSF1R. In some instances, the multi-specific antibody comprises a tumor binding moiety that specifically binds to TROP2 and an immune cell binding moiety that specifically binds to CD33, CD163, or CSF1R. In some instances, the multi-specific antibody comprises a tumor binding moiety that specifically binds to TROP2 and an immune cell binding moiety that specifically binds to TRAIL-R2. In some instances, the multi-specific antibody comprises a tumor binding moiety that specifically binds to TROP2 and an immune cell binding moiety that specifically binds to CD33. In some instances, the multi-specific antibody comprises a tumor binding moiety that specifically binds to TROP2 and an immune cell binding moiety that specifically binds to CD163. In some instances, the multi-specific antibody comprises a tumor binding moiety that specifically binds to TROP2 and an immune cell binding moiety that specifically binds to CSF1R. In some instances, the multi-specific antibody comprises a tumor binding moiety that specifically binds to TROP2 and an immune cell binding moiety that specifically binds to TREM2. In some instances, the multi-specific antibody comprises a tumor binding moiety that specifically binds to TROP2 and an immune cell binding moiety that specifically binds to C5AR1. In some instances, the multi-specific antibody comprises a tumor binding moiety that specifically binds to TROP2 and an immune cell binding moiety that specifically binds to LYVE1. In some instances, the multi-specific antibody comprises a tumor binding moiety that specifically binds to TROP2 and an immune cell binding moiety that specifically binds to ABCC3. In some instances, the multi-specific antibody comprises a tumor binding moiety that specifically binds to TROP2 and an immune cell binding moiety that specifically binds to LILRB4. In some instances, the multi-specific antibody comprises a tumor binding moiety that specifically binds to TROP2 and an immune cell binding moiety that specifically binds to MRC1. In some instances, the multi-specific antibody comprises a tumor binding moiety that specifically binds to TROP2 and an immune cell binding moiety that specifically binds to SIGLEC1. In some instances, the multi-specific antibody comprises a tumor binding moiety that specifically binds to TROP2 and an immune cell binding moiety that specifically binds to STAB1. In some instances, the multi-specific antibody comprises a tumor binding moiety that specifically binds to TROP2 and an immune cell binding moiety that specifically binds to TMEM37. In some instances, the multi-specific antibody comprises a tumor binding moiety that specifically binds to TROP2 and an immune cell binding moiety that specifically binds to MERTK. In some instances, the multi-specific antibody comprises a tumor binding moiety that specifically binds to TROP2 and an immune cell binding moiety that specifically binds to TMEM119. In some instances, the multi-specific antibody comprises a tumor binding moiety that specifically binds to TROP2 and an immune cell binding moiety that specifically binds to SIGLEC7. In some instances, the multi-specific antibody comprises a tumor binding moiety that specifically binds to TROP2 and an immune cell binding moiety that specifically binds to SIGLEC9. In some instances, the multi-specific antibody comprises a tumor binding moiety that specifically binds to TROP2 and an immune cell binding moiety that specifically binds to IL4. In some instances, the multi-specific antibody comprises a tumor binding moiety that specifically binds to TROP2 and an immune cell binding moiety that specifically binds to MARCO. In some instances, the multi-specific antibody comprises a tumor binding moiety that specifically binds to TROP2 and an immune cell binding moiety that specifically binds to SELPLG. In some instances, the multi-specific antibody comprises a tumor binding moiety that specifically binds to TROP2 and an immune cell binding moiety that specifically binds to MS4A7. In some instances, the multi-specific antibody comprises a tumor binding moiety that specifically binds to TROP2 and an immune cell binding moiety that specifically binds to CD200R. In some instances, the multi-specific antibody comprises a tumor binding moiety that specifically binds to TROP2 and an immune cell binding moiety that specifically binds to MGL1.

In some embodiments, the multi-specific antibody comprises a tumor binding moiety that specifically binds to GPC3 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to GPC3 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to GPC3 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to GPC3 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to GPC3 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to GPC3 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to GPC3 and an immune cell binding moiety that specifically binds to MARCO, SELPLG, CD163, MS4A7, CD200R, MGL1, or MGL2. In some instances, the multi-specific antibody comprises a tumor binding moiety that specifically binds to GPC3 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to GPC3 and an immune cell binding moiety that specifically binds to SEMA4A, SEMA4D, or TNFR2. In some instances, the multi-specific antibody comprises a tumor binding moiety that specifically binds to GPC3 and an immune cell binding moiety that specifically binds to TRAIL-R2, CD33, CD163, or CSF1R. In some instances, the multi-specific antibody comprises a tumor binding moiety that specifically binds to GPC3 and an immune cell binding moiety that specifically binds to CD33, CD163, or CSF1R. In some instances, the multi-specific antibody comprises a tumor binding moiety that specifically binds to GPC3 and an immune cell binding moiety that specifically binds to TRAIL-R2. In some instances, the multi-specific antibody comprises a tumor binding moiety that specifically binds to GPC3 and an immune cell binding moiety that specifically binds to CD33. In some instances, the multi-specific antibody comprises a tumor binding moiety that specifically binds to GPC3 and an immune cell binding moiety that specifically binds to CD163. In some instances, the multi-specific antibody comprises a tumor binding moiety that specifically binds to GPC3 and an immune cell binding moiety that specifically binds to CSF1R. In some instances, the multi-specific antibody comprises a tumor binding moiety that specifically binds to GPC3 and an immune cell binding moiety that specifically binds to TREM2. In some instances, the multi-specific antibody comprises a tumor binding moiety that specifically binds to GPC3 and an immune cell binding moiety that specifically binds to C5AR1. In some instances, the multi-specific antibody comprises a tumor binding moiety that specifically binds to GPC3 and an immune cell binding moiety that specifically binds to LYVE1. In some instances, the multi-specific antibody comprises a tumor binding moiety that specifically binds to GPC3 and an immune cell binding moiety that specifically binds to ABCC3. In some instances, the multi-specific antibody comprises a tumor binding moiety that specifically binds to GPC3 and an immune cell binding moiety that specifically binds to LILRB4. In some instances, the multi-specific antibody comprises a tumor binding moiety that specifically binds to GPC3 and an immune cell binding moiety that specifically binds to MRC1. In some instances, the multi-specific antibody comprises a tumor binding moiety that specifically binds to GPC3 and an immune cell binding moiety that specifically binds to SIGLEC1. In some instances, the multi-specific antibody comprises a tumor binding moiety that specifically binds to GPC3 and an immune cell binding moiety that specifically binds to STAB1. In some instances, the multi-specific antibody comprises a tumor binding moiety that specifically binds to GPC3 and an immune cell binding moiety that specifically binds to TMEM37. In some instances, the multi-specific antibody comprises a tumor binding moiety that specifically binds to GPC3 and an immune cell binding moiety that specifically binds to MERTK. In some instances, the multi-specific antibody comprises a tumor binding moiety that specifically binds to GPC3 and an immune cell binding moiety that specifically binds to TMEM119. In some instances, the multi-specific antibody comprises a tumor binding moiety that specifically binds to GPC3 and an immune cell binding moiety that specifically binds to SIGLEC7. In some instances, the multi-specific antibody comprises a tumor binding moiety that specifically binds to GPC3 and an immune cell binding moiety that specifically binds to SIGLEC9. In some instances, the multi-specific antibody comprises a tumor binding moiety that specifically binds to GPC3 and an immune cell binding moiety that specifically binds to IL4. In some instances, the multi-specific antibody comprises a tumor binding moiety that specifically binds to GPC3 and an immune cell binding moiety that specifically binds to MARCO. In some instances, the multi-specific antibody comprises a tumor binding moiety that specifically binds to GPC3 and an immune cell binding moiety that specifically binds to SELPLG. In some instances, the multi-specific antibody comprises a tumor binding moiety that specifically binds to GPC3 and an immune cell binding moiety that specifically binds to MS4A7. In some instances, the multi-specific antibody comprises a tumor binding moiety that specifically binds to GPC3 and an immune cell binding moiety that specifically binds to CD200R. In some instances, the multi-specific antibody comprises a tumor binding moiety that specifically binds to GPC3 and an immune cell binding moiety that specifically binds to MGL1.

In some embodiments, the multi-specific antibody comprises a tumor binding moiety that specifically binds to FOLR1 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to FOLR1 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to FOLR1 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to FOLR1 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to FOLR1 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to FOLR1 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to FOLR1 and an immune cell binding moiety that specifically binds to MARCO, SELPLG, CD163, MS4A7, CD200R, MGL1, or MGL2. In some instances, the multi-specific antibody comprises a tumor binding moiety that specifically binds to FOLR1 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to FOLR1 and an immune cell binding moiety that specifically binds to SEMA4A, SEMA4D, or TNFR2. In some instances, the multi-specific antibody comprises a tumor binding moiety that specifically binds to FOLR1 and an immune cell binding moiety that specifically binds to TRAIL-R2, CD33, CD163, or CSF1R. In some instances, the multi-specific antibody comprises a tumor binding moiety that specifically binds to FOLR1 and an immune cell binding moiety that specifically binds to CD33, CD163, or CSF1R. In some instances, the multi-specific antibody comprises a tumor binding moiety that specifically binds to FOLR1 and an immune cell binding moiety that specifically binds to TRAIL-R2. In some instances, the multi-specific antibody comprises a tumor binding moiety that specifically binds to FOLR1 and an immune cell binding moiety that specifically binds to CD33. In some instances, the multi-specific antibody comprises a tumor binding moiety that specifically binds to FOLR1 and an immune cell binding moiety that specifically binds to CD163. In some instances, the multi-specific antibody comprises a tumor binding moiety that specifically binds to FOLR1 and an immune cell binding moiety that specifically binds to CSF1R. In some instances, the multi-specific antibody comprises a tumor binding moiety that specifically binds to FOLR1 and an immune cell binding moiety that specifically binds to TREM2. In some instances, the multi-specific antibody comprises a tumor binding moiety that specifically binds to FOLR1 and an immune cell binding moiety that specifically binds to C5AR1. In some instances, the multi-specific antibody comprises a tumor binding moiety that specifically binds to FOLR1 and an immune cell binding moiety that specifically binds to LYVE1. In some instances, the multi-specific antibody comprises a tumor binding moiety that specifically binds to FOLR1 and an immune cell binding moiety that specifically binds to ABCC3. In some instances, the multi-specific antibody comprises a tumor binding moiety that specifically binds to FOLR1 and an immune cell binding moiety that specifically binds to LILRB4. In some instances, the multi-specific antibody comprises a tumor binding moiety that specifically binds to FOLR1 and an immune cell binding moiety that specifically binds to MRC1. In some instances, the multi-specific antibody comprises a tumor binding moiety that specifically binds to FOLR1 and an immune cell binding moiety that specifically binds to SIGLEC1. In some instances, the multi-specific antibody comprises a tumor binding moiety that specifically binds to FOLR1 and an immune cell binding moiety that specifically binds to STAB1. In some instances, the multi-specific antibody comprises a tumor binding moiety that specifically binds to FOLR1 and an immune cell binding moiety that specifically binds to TMEM37. In some instances, the multi-specific antibody comprises a tumor binding moiety that specifically binds to FOLR1 and an immune cell binding moiety that specifically binds to MERTK. In some instances, the multi-specific antibody comprises a tumor binding moiety that specifically binds to FOLR1 and an immune cell binding moiety that specifically binds to TMEM119. In some instances, the multi-specific antibody comprises a tumor binding moiety that specifically binds to FOLR1 and an immune cell binding moiety that specifically binds to SIGLEC7. In some instances, the multi-specific antibody comprises a tumor binding moiety that specifically binds to FOLR1 and an immune cell binding moiety that specifically binds to SIGLEC9. In some instances, the multi-specific antibody comprises a tumor binding moiety that specifically binds to FOLR1 and an immune cell binding moiety that specifically binds to IL4. In some instances, the multi-specific antibody comprises a tumor binding moiety that specifically binds to FOLR1 and an immune cell binding moiety that specifically binds to MARCO. In some instances, the multi-specific antibody comprises a tumor binding moiety that specifically binds to FOLR1 and an immune cell binding moiety that specifically binds to SELPLG. In some instances, the multi-specific antibody comprises a tumor binding moiety that specifically binds to FOLR1 and an immune cell binding moiety that specifically binds to MS4A7. In some instances, the multi-specific antibody comprises a tumor binding moiety that specifically binds to FOLR1 and an immune cell binding moiety that specifically binds to CD200R. In some instances, the multi-specific antibody comprises a tumor binding moiety that specifically binds to FOLR1 and an immune cell binding moiety that specifically binds to MGL1.

In some embodiments, the multi-specific antibody comprises a tumor binding moiety that specifically binds to CD33 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to CD33 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to CD33 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to CD33 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to CD33 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to CD33 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to CD33 and an immune cell binding moiety that specifically binds to MARCO, SELPLG, CD163, MS4A7, CD200R, MGL1, or MGL2. In some instances, the multi-specific antibody comprises a tumor binding moiety that specifically binds to CD33 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to CD33 and an immune cell binding moiety that specifically binds to SEMA4A, SEMA4D, or TNFR2. In some instances, the multi-specific antibody comprises a tumor binding moiety that specifically binds to CD33 and an immune cell binding moiety that specifically binds to TRAIL-R2, CD33, CD163, or CSF1R. In some instances, the multi-specific antibody comprises a tumor binding moiety that specifically binds to CD33 and an immune cell binding moiety that specifically binds to CD33, CD163, or CSF1R. In some instances, the multi-specific antibody comprises a tumor binding moiety that specifically binds to CD33 and an immune cell binding moiety that specifically binds to TRAIL-R2. In some instances, the multi-specific antibody comprises a tumor binding moiety that specifically binds to CD33 and an immune cell binding moiety that specifically binds to CD33. In some instances, the multi-specific antibody comprises a tumor binding moiety that specifically binds to CD33 and an immune cell binding moiety that specifically binds to CD163. In some instances, the multi-specific antibody comprises a tumor binding moiety that specifically binds to CD33 and an immune cell binding moiety that specifically binds to CSF1R. In some instances, the multi-specific antibody comprises a tumor binding moiety that specifically binds to CD33 and an immune cell binding moiety that specifically binds to TREM2. In some instances, the multi-specific antibody comprises a tumor binding moiety that specifically binds to CD33 and an immune cell binding moiety that specifically binds to C5AR1. In some instances, the multi-specific antibody comprises a tumor binding moiety that specifically binds to CD33 and an immune cell binding moiety that specifically binds to LYVE1. In some instances, the multi-specific antibody comprises a tumor binding moiety that specifically binds to CD33 and an immune cell binding moiety that specifically binds to ABCC3. In some instances, the multi-specific antibody comprises a tumor binding moiety that specifically binds to CD33 and an immune cell binding moiety that specifically binds to LILRB4. In some instances, the multi-specific antibody comprises a tumor binding moiety that specifically binds to CD33 and an immune cell binding moiety that specifically binds to MRC1. In some instances, the multi-specific antibody comprises a tumor binding moiety that specifically binds to CD33 and an immune cell binding moiety that specifically binds to SIGLEC1. In some instances, the multi-specific antibody comprises a tumor binding moiety that specifically binds to CD33 and an immune cell binding moiety that specifically binds to STAB1. In some instances, the multi-specific antibody comprises a tumor binding moiety that specifically binds to CD33 and an immune cell binding moiety that specifically binds to TMEM37. In some instances, the multi-specific antibody comprises a tumor binding moiety that specifically binds to CD33 and an immune cell binding moiety that specifically binds to MERTK. In some instances, the multi-specific antibody comprises a tumor binding moiety that specifically binds to CD33 and an immune cell binding moiety that specifically binds to TMEM119. In some instances, the multi-specific antibody comprises a tumor binding moiety that specifically binds to CD33 and an immune cell binding moiety that specifically binds to SIGLEC7. In some instances, the multi-specific antibody comprises a tumor binding moiety that specifically binds to CD33 and an immune cell binding moiety that specifically binds to SIGLEC9. In some instances, the multi-specific antibody comprises a tumor binding moiety that specifically binds to CD33 and an immune cell binding moiety that specifically binds to IL4. In some instances, the multi-specific antibody comprises a tumor binding moiety that specifically binds to CD33 and an immune cell binding moiety that specifically binds to MARCO. In some instances, the multi-specific antibody comprises a tumor binding moiety that specifically binds to CD33 and an immune cell binding moiety that specifically binds to SELPLG. In some instances, the multi-specific antibody comprises a tumor binding moiety that specifically binds to CD33 and an immune cell binding moiety that specifically binds to MS4A7. In some instances, the multi-specific antibody comprises a tumor binding moiety that specifically binds to CD33 and an immune cell binding moiety that specifically binds to CD200R. In some instances, the multi-specific antibody comprises a tumor binding moiety that specifically binds to CD33 and an immune cell binding moiety that specifically binds to MGL1.

In some embodiments, the multi-specific antibody comprises a tumor binding moiety that specifically binds to CD38 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to CD38 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to CD38 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to CD38 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to CD38 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to CD38 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to CD38 and an immune cell binding moiety that specifically binds to MARCO, SELPLG, CD163, MS4A7, CD200R, MGL1, or MGL2. In some instances, the multi-specific antibody comprises a tumor binding moiety that specifically binds to CD38 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to CD38 and an immune cell binding moiety that specifically binds to SEMA4A, SEMA4D, or TNFR2. In some instances, the multi-specific antibody comprises a tumor binding moiety that specifically binds to CD38 and an immune cell binding moiety that specifically binds to TRAIL-R2, CD33, CD163, or CSF1R. In some instances, the multi-specific antibody comprises a tumor binding moiety that specifically binds to CD38 and an immune cell binding moiety that specifically binds to CD33, CD163, or CSF1R. In some instances, the multi-specific antibody comprises a tumor binding moiety that specifically binds to CD38 and an immune cell binding moiety that specifically binds to TRAIL-R2. In some instances, the multi-specific antibody comprises a tumor binding moiety that specifically binds to CD38 and an immune cell binding moiety that specifically binds to CD33. In some instances, the multi-specific antibody comprises a tumor binding moiety that specifically binds to CD38 and an immune cell binding moiety that specifically binds to CD163. In some instances, the multi-specific antibody comprises a tumor binding moiety that specifically binds to CD38 and an immune cell binding moiety that specifically binds to CSF1R. In some instances, the multi-specific antibody comprises a tumor binding moiety that specifically binds to CD38 and an immune cell binding moiety that specifically binds to TREM2. In some instances, the multi-specific antibody comprises a tumor binding moiety that specifically binds to CD38 and an immune cell binding moiety that specifically binds to C5AR1. In some instances, the multi-specific antibody comprises a tumor binding moiety that specifically binds to CD38 and an immune cell binding moiety that specifically binds to LYVE1. In some instances, the multi-specific antibody comprises a tumor binding moiety that specifically binds to CD38 and an immune cell binding moiety that specifically binds to ABCC3. In some instances, the multi-specific antibody comprises a tumor binding moiety that specifically binds to CD38 and an immune cell binding moiety that specifically binds to LILRB4. In some instances, the multi-specific antibody comprises a tumor binding moiety that specifically binds to CD38 and an immune cell binding moiety that specifically binds to MRC1. In some instances, the multi-specific antibody comprises a tumor binding moiety that specifically binds to CD38 and an immune cell binding moiety that specifically binds to SIGLEC1. In some instances, the multi-specific antibody comprises a tumor binding moiety that specifically binds to CD38 and an immune cell binding moiety that specifically binds to STAB1. In some instances, the multi-specific antibody comprises a tumor binding moiety that specifically binds to CD38 and an immune cell binding moiety that specifically binds to TMEM37. In some instances, the multi-specific antibody comprises a tumor binding moiety that specifically binds to CD38 and an immune cell binding moiety that specifically binds to MERTK. In some instances, the multi-specific antibody comprises a tumor binding moiety that specifically binds to CD38 and an immune cell binding moiety that specifically binds to TMEM119. In some instances, the multi-specific antibody comprises a tumor binding moiety that specifically binds to CD38 and an immune cell binding moiety that specifically binds to SIGLEC7. In some instances, the multi-specific antibody comprises a tumor binding moiety that specifically binds to CD38 and an immune cell binding moiety that specifically binds to SIGLEC9. In some instances, the multi-specific antibody comprises a tumor binding moiety that specifically binds to CD38 and an immune cell binding moiety that specifically binds to IL4. In some instances, the multi-specific antibody comprises a tumor binding moiety that specifically binds to CD38 and an immune cell binding moiety that specifically binds to MARCO. In some instances, the multi-specific antibody comprises a tumor binding moiety that specifically binds to CD38 and an immune cell binding moiety that specifically binds to SELPLG. In some instances, the multi-specific antibody comprises a tumor binding moiety that specifically binds to CD38 and an immune cell binding moiety that specifically binds to MS4A7. In some instances, the multi-specific antibody comprises a tumor binding moiety that specifically binds to CD38 and an immune cell binding moiety that specifically binds to CD200R. In some instances, the multi-specific antibody comprises a tumor binding moiety that specifically binds to CD38 and an immune cell binding moiety that specifically binds to MGL1.

In some embodiments, the multi-specific antibody comprises a tumor binding moiety that specifically binds to FLT3 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to FLT3 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to FLT3 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to FLT3 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to FLT3 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to FLT3 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to FLT3 and an immune cell binding moiety that specifically binds to MARCO, SELPLG, CD163, MS4A7, CD200R, MGL1, or MGL2. In some instances, the multi-specific antibody comprises a tumor binding moiety that specifically binds to FLT3 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to FLT3 and an immune cell binding moiety that specifically binds to SEMA4A, SEMA4D, or TNFR2. In some instances, the multi-specific antibody comprises a tumor binding moiety that specifically binds to FLT3 and an immune cell binding moiety that specifically binds to TRAIL-R2, CD33, CD163, or CSF1R. In some instances, the multi-specific antibody comprises a tumor binding moiety that specifically binds to FLT3 and an immune cell binding moiety that specifically binds to CD33, CD163, or CSF1R. In some instances, the multi-specific antibody comprises a tumor binding moiety that specifically binds to FLT3 and an immune cell binding moiety that specifically binds to TRAIL-R2. In some instances, the multi-specific antibody comprises a tumor binding moiety that specifically binds to FLT3 and an immune cell binding moiety that specifically binds to CD33. In some instances, the multi-specific antibody comprises a tumor binding moiety that specifically binds to FLT3 and an immune cell binding moiety that specifically binds to CD163. In some instances, the multi-specific antibody comprises a tumor binding moiety that specifically binds to FLT3 and an immune cell binding moiety that specifically binds to CSF1R. In some instances, the multi-specific antibody comprises a tumor binding moiety that specifically binds to FLT3 and an immune cell binding moiety that specifically binds to TREM2. In some instances, the multi-specific antibody comprises a tumor binding moiety that specifically binds to FLT3 and an immune cell binding moiety that specifically binds to C5AR1. In some instances, the multi-specific antibody comprises a tumor binding moiety that specifically binds to FLT3 and an immune cell binding moiety that specifically binds to LYVE1. In some instances, the multi-specific antibody comprises a tumor binding moiety that specifically binds to FLT3 and an immune cell binding moiety that specifically binds to ABCC3. In some instances, the multi-specific antibody comprises a tumor binding moiety that specifically binds to FLT3 and an immune cell binding moiety that specifically binds to LILRB4. In some instances, the multi-specific antibody comprises a tumor binding moiety that specifically binds to FLT3 and an immune cell binding moiety that specifically binds to MRC1. In some instances, the multi-specific antibody comprises a tumor binding moiety that specifically binds to FLT3 and an immune cell binding moiety that specifically binds to SIGLEC1. In some instances, the multi-specific antibody comprises a tumor binding moiety that specifically binds to FLT3 and an immune cell binding moiety that specifically binds to STAB1. In some instances, the multi-specific antibody comprises a tumor binding moiety that specifically binds to FLT3 and an immune cell binding moiety that specifically binds to TMEM37. In some instances, the multi-specific antibody comprises a tumor binding moiety that specifically binds to FLT3 and an immune cell binding moiety that specifically binds to MERTK. In some instances, the multi-specific antibody comprises a tumor binding moiety that specifically binds to FLT3 and an immune cell binding moiety that specifically binds to TMEM119. In some instances, the multi-specific antibody comprises a tumor binding moiety that specifically binds to FLT3 and an immune cell binding moiety that specifically binds to SIGLEC7. In some instances, the multi-specific antibody comprises a tumor binding moiety that specifically binds to FLT3 and an immune cell binding moiety that specifically binds to SIGLEC9. In some instances, the multi-specific antibody comprises a tumor binding moiety that specifically binds to FLT3 and an immune cell binding moiety that specifically binds to IL4. In some instances, the multi-specific antibody comprises a tumor binding moiety that specifically binds to FLT3 and an immune cell binding moiety that specifically binds to MARCO. In some instances, the multi-specific antibody comprises a tumor binding moiety that specifically binds to FLT3 and an immune cell binding moiety that specifically binds to SELPLG. In some instances, the multi-specific antibody comprises a tumor binding moiety that specifically binds to FLT3 and an immune cell binding moiety that specifically binds to MS4A7. In some instances, the multi-specific antibody comprises a tumor binding moiety that specifically binds to FLT3 and an immune cell binding moiety that specifically binds to CD200R. In some instances, the multi-specific antibody comprises a tumor binding moiety that specifically binds to FLT3 and an immune cell binding moiety that specifically binds to MGL1.

In some embodiments, the multi-specific antibody comprises a tumor binding moiety that specifically binds to HER2 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to HER2 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to HER2 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to HER2 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to HER2 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to HER2 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to HER2 and an immune cell binding moiety that specifically binds to MARCO, SELPLG, CD163, MS4A7, CD200R, MGL1, or MGL2. In some instances, the multi-specific antibody comprises a tumor binding moiety that specifically binds to HER2 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to HER2 and an immune cell binding moiety that specifically binds to SEMA4A, SEMA4D, or TNFR2. In some instances, the multi-specific antibody comprises a tumor binding moiety that specifically binds to HER2 and an immune cell binding moiety that specifically binds to TRAIL-R2, CD33, CD163, or CSF1R. In some instances, the multi-specific antibody comprises a tumor binding moiety that specifically binds to HER2 and an immune cell binding moiety that specifically binds to CD33, CD163, or CSF1R. In some instances, the multi-specific antibody comprises a tumor binding moiety that specifically binds to HER2 and an immune cell binding moiety that specifically binds to TRAIL-R2. In some instances, the multi-specific antibody comprises a tumor binding moiety that specifically binds to HER2 and an immune cell binding moiety that specifically binds to CD33. In some instances, the multi-specific antibody comprises a tumor binding moiety that specifically binds to HER2 and an immune cell binding moiety that specifically binds to CD163. In some instances, the multi-specific antibody comprises a tumor binding moiety that specifically binds to HER2 and an immune cell binding moiety that specifically binds to CSF1R. In some instances, the multi-specific antibody comprises a tumor binding moiety that specifically binds to HER2 and an immune cell binding moiety that specifically binds to TREM2. In some instances, the multi-specific antibody comprises a tumor binding moiety that specifically binds to HER2 and an immune cell binding moiety that specifically binds to C5AR1. In some instances, the multi-specific antibody comprises a tumor binding moiety that specifically binds to HER2 and an immune cell binding moiety that specifically binds to LYVE1. In some instances, the multi-specific antibody comprises a tumor binding moiety that specifically binds to HER2 and an immune cell binding moiety that specifically binds to ABCC3. In some instances, the multi-specific antibody comprises a tumor binding moiety that specifically binds to HER2 and an immune cell binding moiety that specifically binds to LILRB4. In some instances, the multi-specific antibody comprises a tumor binding moiety that specifically binds to HER2 and an immune cell binding moiety that specifically binds to MRC1. In some instances, the multi-specific antibody comprises a tumor binding moiety that specifically binds to HER2 and an immune cell binding moiety that specifically binds to SIGLEC1. In some instances, the multi-specific antibody comprises a tumor binding moiety that specifically binds to HER2 and an immune cell binding moiety that specifically binds to STAB1. In some instances, the multi-specific antibody comprises a tumor binding moiety that specifically binds to HER2 and an immune cell binding moiety that specifically binds to TMEM37. In some instances, the multi-specific antibody comprises a tumor binding moiety that specifically binds to HER2 and an immune cell binding moiety that specifically binds to MERTK. In some instances, the multi-specific antibody comprises a tumor binding moiety that specifically binds to HER2 and an immune cell binding moiety that specifically binds to TMEM119. In some instances, the multi-specific antibody comprises a tumor binding moiety that specifically binds to HER2 and an immune cell binding moiety that specifically binds to SIGLEC7. In some instances, the multi-specific antibody comprises a tumor binding moiety that specifically binds to HER2 and an immune cell binding moiety that specifically binds to SIGLEC9. In some instances, the multi-specific antibody comprises a tumor binding moiety that specifically binds to HER2 and an immune cell binding moiety that specifically binds to IL4. In some instances, the multi-specific antibody comprises a tumor binding moiety that specifically binds to HER2 and an immune cell binding moiety that specifically binds to MARCO. In some instances, the multi-specific antibody comprises a tumor binding moiety that specifically binds to HER2 and an immune cell binding moiety that specifically binds to SELPLG. In some instances, the multi-specific antibody comprises a tumor binding moiety that specifically binds to HER2 and an immune cell binding moiety that specifically binds to MS4A7. In some instances, the multi-specific antibody comprises a tumor binding moiety that specifically binds to HER2 and an immune cell binding moiety that specifically binds to CD200R. In some instances, the multi-specific antibody comprises a tumor binding moiety that specifically binds to HER2 and an immune cell binding moiety that specifically binds to MGL1.

In some embodiments, the multi-specific antibody comprises a tumor binding moiety that specifically binds to CD30 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to CD30 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to CD30 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to CD30 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to CD30 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to CD30 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to CD30 and an immune cell binding moiety that specifically binds to MARCO, SELPLG, CD163, MS4A7, CD200R, MGL1, or MGL2. In some instances, the multi-specific antibody comprises a tumor binding moiety that specifically binds to CD30 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to CD30 and an immune cell binding moiety that specifically binds to SEMA4A, SEMA4D, or TNFR2. In some instances, the multi-specific antibody comprises a tumor binding moiety that specifically binds to CD30 and an immune cell binding moiety that specifically binds to TRAIL-R2, CD33, CD163, or CSF1R. In some instances, the multi-specific antibody comprises a tumor binding moiety that specifically binds to CD30 and an immune cell binding moiety that specifically binds to CD33, CD163, or CSF1R. In some instances, the multi-specific antibody comprises a tumor binding moiety that specifically binds to CD30 and an immune cell binding moiety that specifically binds to TRAIL-R2. In some instances, the multi-specific antibody comprises a tumor binding moiety that specifically binds to CD30 and an immune cell binding moiety that specifically binds to CD33. In some instances, the multi-specific antibody comprises a tumor binding moiety that specifically binds to CD30 and an immune cell binding moiety that specifically binds to CD163. In some instances, the multi-specific antibody comprises a tumor binding moiety that specifically binds to CD30 and an immune cell binding moiety that specifically binds to CSF1R. In some instances, the multi-specific antibody comprises a tumor binding moiety that specifically binds to CD30 and an immune cell binding moiety that specifically binds to TREM2. In some instances, the multi-specific antibody comprises a tumor binding moiety that specifically binds to CD30 and an immune cell binding moiety that specifically binds to C5AR1. In some instances, the multi-specific antibody comprises a tumor binding moiety that specifically binds to CD30 and an immune cell binding moiety that specifically binds to LYVE1. In some instances, the multi-specific antibody comprises a tumor binding moiety that specifically binds to CD30 and an immune cell binding moiety that specifically binds to ABCC3. In some instances, the multi-specific antibody comprises a tumor binding moiety that specifically binds to CD30 and an immune cell binding moiety that specifically binds to LILRB4. In some instances, the multi-specific antibody comprises a tumor binding moiety that specifically binds to CD30 and an immune cell binding moiety that specifically binds to MRC1. In some instances, the multi-specific antibody comprises a tumor binding moiety that specifically binds to CD30 and an immune cell binding moiety that specifically binds to SIGLEC1. In some instances, the multi-specific antibody comprises a tumor binding moiety that specifically binds to CD30 and an immune cell binding moiety that specifically binds to STAB1. In some instances, the multi-specific antibody comprises a tumor binding moiety that specifically binds to CD30 and an immune cell binding moiety that specifically binds to TMEM37. In some instances, the multi-specific antibody comprises a tumor binding moiety that specifically binds to CD30 and an immune cell binding moiety that specifically binds to MERTK. In some instances, the multi-specific antibody comprises a tumor binding moiety that specifically binds to CD30 and an immune cell binding moiety that specifically binds to TMEM119. In some instances, the multi-specific antibody comprises a tumor binding moiety that specifically binds to CD30 and an immune cell binding moiety that specifically binds to SIGLEC7. In some instances, the multi-specific antibody comprises a tumor binding moiety that specifically binds to CD30 and an immune cell binding moiety that specifically binds to SIGLEC9. In some instances, the multi-specific antibody comprises a tumor binding moiety that specifically binds to CD30 and an immune cell binding moiety that specifically binds to IL4. In some instances, the multi-specific antibody comprises a tumor binding moiety that specifically binds to CD30 and an immune cell binding moiety that specifically binds to MARCO. In some instances, the multi-specific antibody comprises a tumor binding moiety that specifically binds to CD30 and an immune cell binding moiety that specifically binds to SELPLG. In some instances, the multi-specific antibody comprises a tumor binding moiety that specifically binds to CD30 and an immune cell binding moiety that specifically binds to MS4A7. In some instances, the multi-specific antibody comprises a tumor binding moiety that specifically binds to CD30 and an immune cell binding moiety that specifically binds to CD200R. In some instances, the multi-specific antibody comprises a tumor binding moiety that specifically binds to CD30 and an immune cell binding moiety that specifically binds to MGL1.

In some embodiments, the multi-specific antibody comprises a tumor binding moiety that specifically binds to CD22 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to CD22 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to CD22 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to CD22 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to CD22 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to CD22 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to CD22 and an immune cell binding moiety that specifically binds to MARCO, SELPLG, CD163, MS4A7, CD200R, MGL1, or MGL2. In some instances, the multi-specific antibody comprises a tumor binding moiety that specifically binds to CD22 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to CD22 and an immune cell binding moiety that specifically binds to SEMA4A, SEMA4D, or TNFR2. In some instances, the multi-specific antibody comprises a tumor binding moiety that specifically binds to CD22 and an immune cell binding moiety that specifically binds to TRAIL-R2, CD33, CD163, or CSF1R. In some instances, the multi-specific antibody comprises a tumor binding moiety that specifically binds to CD22 and an immune cell binding moiety that specifically binds to CD33, CD163, or CSF1R. In some instances, the multi-specific antibody comprises a tumor binding moiety that specifically binds to CD22 and an immune cell binding moiety that specifically binds to TRAIL-R2. In some instances, the multi-specific antibody comprises a tumor binding moiety that specifically binds to CD22 and an immune cell binding moiety that specifically binds to CD33. In some instances, the multi-specific antibody comprises a tumor binding moiety that specifically binds to CD22 and an immune cell binding moiety that specifically binds to CD163. In some instances, the multi-specific antibody comprises a tumor binding moiety that specifically binds to CD22 and an immune cell binding moiety that specifically binds to CSF1R. In some instances, the multi-specific antibody comprises a tumor binding moiety that specifically binds to CD22 and an immune cell binding moiety that specifically binds to TREM2. In some instances, the multi-specific antibody comprises a tumor binding moiety that specifically binds to CD22 and an immune cell binding moiety that specifically binds to C5AR1. In some instances, the multi-specific antibody comprises a tumor binding moiety that specifically binds to CD22 and an immune cell binding moiety that specifically binds to LYVE1. In some instances, the multi-specific antibody comprises a tumor binding moiety that specifically binds to CD22 and an immune cell binding moiety that specifically binds to ABCC3. In some instances, the multi-specific antibody comprises a tumor binding moiety that specifically binds to CD22 and an immune cell binding moiety that specifically binds to LILRB4. In some instances, the multi-specific antibody comprises a tumor binding moiety that specifically binds to CD22 and an immune cell binding moiety that specifically binds to MRC1. In some instances, the multi-specific antibody comprises a tumor binding moiety that specifically binds to CD22 and an immune cell binding moiety that specifically binds to SIGLEC1. In some instances, the multi-specific antibody comprises a tumor binding moiety that specifically binds to CD22 and an immune cell binding moiety that specifically binds to STAB1. In some instances, the multi-specific antibody comprises a tumor binding moiety that specifically binds to CD22 and an immune cell binding moiety that specifically binds to TMEM37. In some instances, the multi-specific antibody comprises a tumor binding moiety that specifically binds to CD22 and an immune cell binding moiety that specifically binds to MERTK. In some instances, the multi-specific antibody comprises a tumor binding moiety that specifically binds to CD22 and an immune cell binding moiety that specifically binds to TMEM119. In some instances, the multi-specific antibody comprises a tumor binding moiety that specifically binds to CD22 and an immune cell binding moiety that specifically binds to SIGLEC7. In some instances, the multi-specific antibody comprises a tumor binding moiety that specifically binds to CD22 and an immune cell binding moiety that specifically binds to SIGLEC9. In some instances, the multi-specific antibody comprises a tumor binding moiety that specifically binds to CD22 and an immune cell binding moiety that specifically binds to IL4. In some instances, the multi-specific antibody comprises a tumor binding moiety that specifically binds to CD22 and an immune cell binding moiety that specifically binds to MARCO. In some instances, the multi-specific antibody comprises a tumor binding moiety that specifically binds to CD22 and an immune cell binding moiety that specifically binds to SELPLG. In some instances, the multi-specific antibody comprises a tumor binding moiety that specifically binds to CD22 and an immune cell binding moiety that specifically binds to MS4A7. In some instances, the multi-specific antibody comprises a tumor binding moiety that specifically binds to CD22 and an immune cell binding moiety that specifically binds to CD200R. In some instances, the multi-specific antibody comprises a tumor binding moiety that specifically binds to CD22 and an immune cell binding moiety that specifically binds to MGL1.

In some embodiments, the multi-specific antibody comprises a tumor binding moiety that specifically binds to GD2 and an immune cell binding moiety that specifically binds to TRAIL-R2 (also referred to as TNFRSF10B, Death Receptor 5, or DR5), CD33, CSF1R, SEMA4A, SEMA4D, CD163, MARCO, TNFR2 (also referred to as TNFRSF1B), TREM2, MS4A7, C5AR1, LYVE1, ABCC3, LILRB4, MRC1 (also referred to as CD206), STAB1, TMEM37, MERTK, TMEM119, SIGLEC1, SIGLEC7, SIGLEC9, IL4R, MGL1 (also referred to as CD301 or CLEC10A), MGL2 (also referred to as CD301B), CD200R, or SELPLG (also referred to as PSLG-1 or CD162). In some instances, the multi-specific antibody comprises a tumor binding moiety that specifically binds to GD2 and an immune cell binding moiety that specifically binds to TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG. In some instances, the multi-specific antibody comprises a tumor binding moiety that specifically binds to GD2 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9. In some instances, the multi-specific antibody comprises a tumor binding moiety that specifically binds to GD2 and an immune cell binding moiety that specifically binds to TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2. In some instances, the multi-specific antibody comprises a tumor binding moiety that specifically binds to GD2 and an immune cell binding moiety that specifically binds to TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R. In some instances, the multi-specific antibody comprises a tumor binding moiety that specifically binds to GD2 and an immune cell binding moiety that specifically binds to TRAIL-R2, CSF1R, TREM2, C5AR1, LYVE1, MRC1, STAB1, MERTK, SIGLEC1, or IL4R. In some instances, the multi-specific antibody comprises a tumor binding moiety that specifically binds to GD2 and an immune cell binding moiety that specifically binds to MARCO, SELPLG, CD163, MS4A7, CD200R, MGL1, or MGL2. In some instances, the multi-specific antibody comprises a tumor binding moiety that specifically binds to GD2 and an immune cell binding moiety that specifically binds to CD33, ABCC3, LILRB4, TMEM37, TMEM119, SIGLEC7, or SIGLEC9. In some instances, the multi-specific antibody comprises a tumor binding moiety that specifically binds to GD2 and an immune cell binding moiety that specifically binds to SEMA4A, SEMA4D, or TNFR2. In some instances, the multi-specific antibody comprises a tumor binding moiety that specifically binds to GD2 and an immune cell binding moiety that specifically binds to TRAIL-R2, CD33, CD163, or CSF1R. In some instances, the multi-specific antibody comprises a tumor binding moiety that specifically binds to GD2 and an immune cell binding moiety that specifically binds to CD33, CD163, or CSF1R. In some instances, the multi-specific antibody comprises a tumor binding moiety that specifically binds to GD2 and an immune cell binding moiety that specifically binds to TRAIL-R2. In some instances, the multi-specific antibody comprises a tumor binding moiety that specifically binds to GD2 and an immune cell binding moiety that specifically binds to CD33. In some instances, the multi-specific antibody comprises a tumor binding moiety that specifically binds to GD2 and an immune cell binding moiety that specifically binds to CD163. In some instances, the multi-specific antibody comprises a tumor binding moiety that specifically binds to GD2 and an immune cell binding moiety that specifically binds to CSF1R. In some instances, the multi-specific antibody comprises a tumor binding moiety that specifically binds to GD2 and an immune cell binding moiety that specifically binds to TREM2. In some instances, the multi-specific antibody comprises a tumor binding moiety that specifically binds to GD2 and an immune cell binding moiety that specifically binds to C5AR1. In some instances, the multi-specific antibody comprises a tumor binding moiety that specifically binds to GD2 and an immune cell binding moiety that specifically binds to LYVE1. In some instances, the multi-specific antibody comprises a tumor binding moiety that specifically binds to GD2 and an immune cell binding moiety that specifically binds to ABCC3. In some instances, the multi-specific antibody comprises a tumor binding moiety that specifically binds to GD2 and an immune cell binding moiety that specifically binds to LILRB4. In some instances, the multi-specific antibody comprises a tumor binding moiety that specifically binds to GD2 and an immune cell binding moiety that specifically binds to MRC1. In some instances, the multi-specific antibody comprises a tumor binding moiety that specifically binds to GD2 and an immune cell binding moiety that specifically binds to SIGLEC1. In some instances, the multi-specific antibody comprises a tumor binding moiety that specifically binds to GD2 and an immune cell binding moiety that specifically binds to STAB1. In some instances, the multi-specific antibody comprises a tumor binding moiety that specifically binds to GD2 and an immune cell binding moiety that specifically binds to TMEM37. In some instances, the multi-specific antibody comprises a tumor binding moiety that specifically binds to GD2 and an immune cell binding moiety that specifically binds to MERTK. In some instances, the multi-specific antibody comprises a tumor binding moiety that specifically binds to GD2 and an immune cell binding moiety that specifically binds to TMEM119. In some instances, the multi-specific antibody comprises a tumor binding moiety that specifically binds to GD2 and an immune cell binding moiety that specifically binds to SIGLEC7. In some instances, the multi-specific antibody comprises a tumor binding moiety that specifically binds to GD2 and an immune cell binding moiety that specifically binds to SIGLEC9. In some instances, the multi-specific antibody comprises a tumor binding moiety that specifically binds to GD2 and an immune cell binding moiety that specifically binds to IL4. In some instances, the multi-specific antibody comprises a tumor binding moiety that specifically binds to GD2 and an immune cell binding moiety that specifically binds to MARCO. In some instances, the multi-specific antibody comprises a tumor binding moiety that specifically binds to GD2 and an immune cell binding moiety that specifically binds to SELPLG. In some instances, the multi-specific antibody comprises a tumor binding moiety that specifically binds to GD2 and an immune cell binding moiety that specifically binds to MS4A7. In some instances, the multi-specific antibody comprises a tumor binding moiety that specifically binds to GD2 and an immune cell binding moiety that specifically binds to CD200R. In some instances, the multi-specific antibody comprises a tumor binding moiety that specifically binds to GD2 and an immune cell binding moiety that specifically binds to MGL1.

In certain embodiments, the multi-specific binding polypeptide (e.g., the multi-specific antibody) comprises a tumor binding moiety that specifically binds to TROP2/TACSTD2. In some embodiments, the multi-specific binding polypeptide (e.g., the multi-specific antibody) comprises an immune cell binding moiety that specifically binds to TRAIL-R2, CSF1R, SEMA4A, TNFR2, TREM2, ABCC3, STAB1, or IL4R; TRAIL-R2, CSF1R, SEMA4A, CD163, TNFR2, TREM2, MS4A7, ABCC3, LILRB4, STAB1, TMEM119, or IL4R; TRAIL-R2, CSF1R, SEMA4A, TNFR2, CD163, TREM2, LILRB4, STAB1, TMEM119, or IL4R; TRAIL-R2, CSF1R, SEMA4A, TNFR2, TREM2, ABCC3, LILRB4, STAB1, TMEM37, or IL4R; TRAIL-R2, CSF1R, TNFR2, TREM2, MS4A7, C5AR1, ABCC3, LILRB4, STAB1, TMEM37, or IL4R; TRAIL-R2, TNFR2, ABCC3, STAB1, or IL4R; TRAIL-R2, CSF1R, SEMA4A, CD163, TNFR2, TREM2, MS4A7, C5AR1, ABCC3, LILRB4, STAB1, TMEM37, or IL4R; TRAIL-R2, CSF1R, SEMA4A, CD163, MARCO, TNFR2, TREM2, MS4A7, C5AR1, ABCC3, LILRB4, MRC1, STAB1, TMEM37, TMEM119, CLEC10A, or IL4R; TRAIL-R2, CSF1R, SEMA4A, TNFR2, TREM2, C5AR1, ABCC3, LILRB4, MRC1, STAB1, MERTK, TMEM37, TMEM119, or IL4R; TRAIL-R2, CSF1R, SEMA4A, CD163, MARCO, TNFR2, TREM2, C5AR1, ABCC3, LILRB4, MRC1, STAB1, MERTK, TMEM37, or IL4R; TRAIL-R2, CSF1R, SEMA4A, CD163, TNFR2, TREM2, MS4A7, C5AR1, ABCC3, LILRB4, MRC1, STAB1, MERTK, TMEM37, TMEM119, or IL4R; or TRAIL-R2, CSF1R, TNFR2, ABCC3, STAB1, TMEM37, TMEM119, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, TNFR2, TREM2, ABCC3, STAB1, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, CD163, TNFR2, TREM2, MS4A7, ABCC3, LILRB4, STAB1, TMEM119, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, TNFR2, CD163, TREM2, LILRB4, STAB1, TMEM119, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, TNFR2, TREM2, ABCC3, LILRB4, STAB1, TMEM37, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, TNFR2, TREM2, MS4A7, C5AR1, ABCC3, LILRB4, STAB1, TMEM37, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, TNFR2, ABCC3, STAB1, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, CD163, TNFR2, TREM2, MS4A7, C5AR1, ABCC3, LILRB4, STAB1, TMEM37, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, CD163, MARCO, TNFR2, TREM2, MS4A7, C5AR1, ABCC3, LILRB4, MRC1, STAB1, TMEM37, TMEM119, CLEC10A, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, TNFR2, TREM2, C5AR1, ABCC3, LILRB4, MRC1, STAB1, MERTK, TMEM37, TMEM119, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, CD163, MARCO, TNFR2, TREM2, C5AR1, ABCC3, LILRB4, MRC1, STAB1, MERTK, TMEM37, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, SEMA4A, CD163, TNFR2, TREM2, MS4A7, C5AR1, ABCC3, LILRB4, MRC1, STAB1, MERTK, TMEM37, TMEM119, or IL4R. In certain embodiments, the multi-specific binding polypeptide (e.g., the multi-specific antibody) comprises a tumor binding moiety that specifically binds to TROP2/TACSTD2 and an immune cell binding moiety that specifically binds to TRAIL-R2, CSF1R, TNFR2, ABCC3, STAB1, TMEM37, TMEM119, or IL4R. In some embodiments, the multi-specific binding polypeptide (e.g., the multi-specific antibody) is a bispecific antibody.

In certain embodiments, the multi-specific binding polypeptide (e.g., the multi-specific antibody) comprises a tumor binding moiety that specifically binds to TROP2/TACSTD2 or FOLR1. In some embodiments, the multi-specific binding polypeptide (e.g., the multi-specific antibody) comprises an immune cell binding moiety that specifically binds to TRAIL-R2, CD33, CSF1R, CD163, MARCO, TNFR2, TREM2, MS4A7, C5AR1, ABCC3, LILRB4, STAB1, TMEM37, or IL4R; TNFRSF10B, TNFRSF1B, CSF1R, SEMA4A, CD163, MARCO, TNFRSF1B, TREM2, MS4A7, C5AR1, ABCC3, LILRB4, MRC1, STAB1, TMEM37, MERTK, TMEM119, SIGLEC1, or CLEC10A; or TRAIL-R2, CSF1R, SEMA4A, CD163, TNFR2, TREM2, STAB1, LILRB4, TMEM37, MERTK, TMEM119, or IL4R. In certain embodiments, the multi-specific binding polypeptide (e.g., the multi-specific antibody) comprises a tumor binding moiety that specifically binds to TROP2/TACSTD2 or FOLR1, and an immune cell binding moiety that specifically binds to TRAIL-R2, CSF1R, CD163, MARCO, TNFR2, TREM2, MS4A7, C5AR1, ABCC3, LILRB4, STAB1, TMEM37, or IL4R. In certain embodiments, the multi-specific binding polypeptide (e.g., the multi-specific antibody) comprises a tumor binding moiety that specifically binds to TROP2/TACSTD2 or FOLR1, and an immune cell binding moiety that specifically binds to TNFRSF10B, TNFRSF1B, CSF1R, SEMA4A, CD163, MARCO, TNFRSF1B, TREM2, MS4A7, C5AR1, ABCC3, LILRB4, MRC1, STAB1, TMEM37, MERTK, TMEM119, SIGLEC1, or CLEC10A. In certain embodiments, the multi-specific binding polypeptide (e.g., the multi-specific antibody) comprises a tumor binding moiety that specifically binds to TROP2/TACSTD2 or FOLR1, and an immune cell binding moiety that specifically binds to TRAIL-R2, CSF1R, SEMA4A, CD163, TNFR2, TREM2, STAB1, LILRB4, TMEM37, MERTK, TMEM119, or IL4R. In some embodiments, the multi-specific binding polypeptide (e.g., the multi-specific antibody) is a bispecific antibody.

In certain embodiments, the multi-specific binding polypeptide (e.g., the multi-specific antibody) comprises a tumor binding moiety that specifically binds to TROP2/TACSTD2 or FOLH1/PSMA. In some embodiments, the multi-specific binding polypeptide (e.g., the multi-specific antibody) comprises an immune cell binding moiety that specifically binds to TRAIL-R2, CSF1R, SEMA4A, TNFR2, TREM2, MS4A7, C5AR1, ABCC3, STAB1, LILRB4, TMEM37, MERTK, TMEM119, or IL4R. In some embodiments, the multi-specific binding polypeptide (e.g., the multi-specific antibody) is a bispecific antibody.

In certain embodiments, the multi-specific binding polypeptide (e.g., the multi-specific antibody) comprises a tumor binding moiety that specifically binds to FOLR1. In some embodiments, the multi-specific binding polypeptide (e.g., the multi-specific antibody) comprises an immune cell binding moiety that specifically binds to TRAIL-R2, CSF1R, SEMA4A, CD163, TNFR2, TREM2, MS4A7, C5AR1, LYVE1, ABCC3, LILRB4, MRC1, STAB1, MERTK, TMEM37, TMEM119, IL4R, or SIGLEC1. In some embodiments, the multi-specific binding polypeptide (e.g., the multi-specific antibody) is a bispecific antibody.

In certain embodiments, the multi-specific binding polypeptide (e.g., the multi-specific antibody) comprises a tumor binding moiety that specifically binds to CD33 or CD123. In some embodiments, the multi-specific binding polypeptide (e.g., the multi-specific antibody) comprises an immune cell binding moiety that specifically binds to TNFRSF10B/TRAIL-R2, TNFRSF1B/TNFR2, CSF1R, SEMA4A, MS4A7, C5AR1, LILRB4, STAB1, MERTK, SIGLEC7, SIGLEC9, CLEC10A, or CD200R1. In some embodiments, the multi-specific binding polypeptide (e.g., the multi-specific antibody) is a bispecific antibody.

In certain embodiments, the multi-specific binding polypeptide (e.g., the multi-specific antibody) comprises a tumor binding moiety that specifically binds to CD123, CD30, CD22, or CD19. In some embodiments, the multi-specific binding polypeptide (e.g., the multi-specific antibody) comprises an immune cell binding moiety that specifically binds to TNFRSF10B, CSF1R, SEMA4A, CD163, MARCO, TNFRSF1B, C5AR1, ABCC3, LILRB4, STAB1, TMEM119, SIGLEC1, SIGLEC7, SIGLEC9, CLEC10A, or IL4R. In some embodiments, the multi-specific binding polypeptide (e.g., the multi-specific antibody) is a bispecific antibody.

### Multi-Specific Antibodies

In certain embodiments, a multi-specific binding polypeptide (e.g., a multi-specific antibody) described herein comprises a tumor binding moiety comprising sequences at least about 80%, 85%, 90%, 95%, 97%, 98%, 99%, or 100% identical to or consist of sequences (e.g., CDRs or VH/VL sequences) selected from Tables 1 and/or 2.

TABLE 1

| NAME* | VH SEQUENCE^{#} | HCDR1 | HCDR2 | HCDR3 | VH |
|---|---|---|---|---|---|
| αTROP2 | QVQLQQSGSELKKPGASVKVSCKASGY | SEQ ID | SEQ ID | SEQ ID | SEQ ID |
| | TFTNYGMNWVKQAPGQGLKWMGWIN | NO: 4 | NO: 5 | NO: 6 | NO: 9 |
| | TYTGEPTYTDDFKGRFAFSLDTSVSTAY | | | | |
| | LQISSLKADDTAVYFCARGGFGSSYWY | | | | |
| | FDVWGQGSLVTVSS | | | | |
| αGPC3 | QVQLVQSGAEVKKPGASVKVSCKASG | SEQ ID | SEQ ID | SEQ ID | SEQ ID |
| | YTFTDYEMHWVRQAPGQGLEWMGAL | NO: 13 | NO: 14 | NO: 15 | NO: 16 |
| | DPKTGDTAYSQKFKGRVTLTADKSTST | | | | |
| | AYMELSSLTSEDTAVYYCTRFYSYTYW | | | | |
| | GQGTLVTVSS | | | | |
| αFOLR1 | QVQLVQSGAEVVKPGASVKISCKASGY | SEQ ID | SEQ ID | SEQ ID | SEQ ID |
| | TFTGYFMNWVKQSPGQSLEWIGRIHPY | NO: 17 | NO: 18 | NO: 19 | NO: 20 |
| | DGDTFYNQKFQGKATLTVDKSSNTAH | | | | |
| | MELLSLTSEDFAVYYCTRYDGSRAMDY | | | | |
| | WGQGTTVTVSS | | | | |
| αCD38 | EVQLLESGGGLVQPGGSLRLSCAVSGFT | SEQ ID | SEQ ID | SEQ ID | SEQ ID |
| | FNSFAMSWVRQAPGKGLEWVSAISGSG | NO: 21 | NO: 22 | NO: 23 | NO: 24 |
| | GGTYYADSVKGRFTISRDNSKNTLYLQ | | | | |
| | MNSLRAEDTAVYFCAKDKILWFGEPVF | | | | |
| | DYWGQGTLVTVSS | | | | |
| αFLT3 | EVQLVQSGAEVKKPGASVKVSCKASG | SEQ ID | SEQ ID | SEQ ID | SEQ ID |
| | YTFTSYYMHWVRQAPGQGLEWMGIIN | NO: 25 | NO: 26 | NO: 27 | NO: 28 |
| | PSGGSTSYAQKFQGRVTMTRDTSTSTV | | | | |
| | YMELSSLRSEDTAVYYCARGVGAHDA | | | | |
| | FDIWGQGTTVTVSS | | | | |

*The notation “α” in front of an exemplary tumor-associated antigen denotes an antibody that specifically binds to the tumor-associated antigen. For example, αTROP2 refers to an antibody that specifically binds to TROP2; αGPC3 refers to an antibody that specifically binds to GPC3; αFOLR1 refers to an antibody that specifically binds to FOLR1; αCD38 refers to an antibody that specifically binds to CD38; and αFLT3 refers to an antibody that specifically binds to FLT3.
^{#}The underlined sequences denote the respective HCDR1, HCDR2, and HCDR3. For example, the first underlined sequence in αTROP2 denotes HCDR1; the second underlined sequence in αTROP2 denotes HCDR2; and the third underlined sequence in αTROP2 denotes HCDR3.

TABLE 2

| NAME* | VL SEQUENCE^{#} | LCDR1 | LCDR2 | LCDR3 | VL |
|---|---|---|---|---|---|
| αTROP2 | DIQLTQSPSSLSASVGDRVSITCKASQD | SEQ ID | SEQ ID | SEQ ID | SEQ ID |
| | VSIAVAWYQQKPGKAPKLLIYSASYRY | NO: 1 | NO: 2 | NO: 3 | NO: 10 |
| | TGVPDRFSGSGSGTDFTLTISSLQPEDFA | | | | |
| | VYYCQQHYITPLTFGAGTKVEIK | | | | |
| αGPC3 | DVVMTQSPLSLPVTPGEPASISCRSSQSL | SEQ ID | SEQ ID | SEQ ID | SEQ ID |
| | VHSNRNTYLHWYLQKPGQSPQLLIYKV | NO: 29 | NO: 30 | NO: 31 | NO: 32 |
| | SNRFSGVPDRFSGSGSGTDFTLKISRVE | | | | |
| | AEDVGVYYCSQNTHVPPTFGQGTKLEI | | | | |
| | K | | | | |
| αFOLR1 | DIVLTQSPLSLAVSLGQPAIISCKASQSV | SEQ ID | SEQ ID | SEQ ID | SEQ ID |
| | SFAGTSLMHWYHQKPGQQPRLLIYRAS | NO: 33 | NO: 34 | NO: 35 | NO: 36 |
| | NLEAGVPDRFSGSGSKTDFTLTISPVEA | | | | |
| | EDAATYYCQQSREYPYTFGGGTKLEIK | | | | |
| αCD38 | EIVLTQSPATLSLSPGERATLSCRASQSV | SEQ ID | SEQ ID | SEQ ID | SEQ ID |
| | SSYLAWYQQKPGQAPRLLIYDASNRAT | NO: 37 | NO: 38 | NO: 39 | NO: 40 |
| | GIPARFSGSGSGTDFTLTISSLEPEDFAV | | | | |
| | YYCQQRSNWPPTFGQGTKVEIK | | | | |
| αFLT3 | DVVMTQSPLSLPVTPGEPASISCRSSQSL | SEQ ID | SEQ ID | SEQ ID | SEQ ID |
| | LHSNGNNYLDWYLQKPGQSPQLLIYLG | NO: 41 | NO: 42 | NO: 43 | NO: 44 |
| | SNRASGVPDRFSGSGSDTDFTLQISRVE | | | | |
| | AEDVGVYYCMQGTHPAISFGQGTRLEI | | | | |
| | K | | | | |

*The notation “α” in front of an exemplary tumor-associated antigen denotes an antibody that specifically binds to the tumor-associated antigen.
^{#}The underlined sequences denote the respective LCDR1, LCDR2, and LCDR3. For example, the first underlined sequence in αTROP2 denotes LCDR1; the second underlined sequence in αTROP2 denotes LCDR2; and the third underlined sequence in αTROP2 denotes LCDR3.

In some embodiments, a multi-specific binding polypeptide (e.g., a multi-specific antibody) described herein comprises a tumor binding moiety comprising an immunoglobulin heavy chain variable region comprising an amino acid sequence at least about 80%, 85%, 90%, 95%, 97%, 98%, 99%, or 100% identical to or consist of the amino acid sequence set forth in SEQ ID NOs: 9, 16, 20, 24, or 28; and an immunoglobulin light chain variable region at least about 80%, 85%, 90%, 95%, 97%, 98%, 99%, or 100% identical to or consist of the amino acid sequence set forth in SEQ ID NOs: 10, 32, 36, 40, or 44. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NOs: 9, 16, 20, 24, or 28. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NOs: 10, 32, 36, 40, or 44. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target tumor antigen. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target tumor antigen.

In some embodiments, a multi-specific binding polypeptide (e.g., a multi-specific antibody) described herein comprises a tumor binding moiety comprising an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 9; and an immunoglobulin light chain variable region at least about 80%, 85%, 90%, 95%, 97%, 98%, 99%, or 100% identical to or consist of the amino acid sequence set forth in SEQ ID NO: 10. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 9. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 10. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target tumor antigen. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target tumor antigen.

In some embodiments, a multi-specific binding polypeptide (e.g., a multi-specific antibody) described herein comprises a tumor binding moiety comprising an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 16; and an immunoglobulin light chain variable region at least about 80%, 85%, 90%, 95%, 97%, 98%, 99%, or 100% identical to or consist of the amino acid sequence set forth in SEQ ID NO: 32. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 16. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 32. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target tumor antigen. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target tumor antigen.

In some embodiments, a multi-specific binding polypeptide (e.g., a multi-specific antibody) described herein comprises a tumor binding moiety comprising an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 20; and an immunoglobulin light chain variable region at least about 80%, 85%, 90%, 95%, 97%, 98%, 99%, or 100% identical to or consist of the amino acid sequence set forth in SEQ ID NO: 36. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 20. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 36. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target tumor antigen. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target tumor antigen.

In some embodiments, a multi-specific binding polypeptide (e.g., a multi-specific antibody) described herein comprises a tumor binding moiety comprising an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 24; and an immunoglobulin light chain variable region at least about 80%, 85%, 90%, 95%, 97%, 98%, 99%, or 100% identical to or consist of the amino acid sequence set forth in SEQ ID NO: 40. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 24. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 40. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target tumor antigen. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target tumor antigen.

In some embodiments, a multi-specific binding polypeptide (e.g., a multi-specific antibody) described herein comprises a tumor binding moiety comprising an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 28; and an immunoglobulin light chain variable region at least about 80%, 85%, 90%, 95%, 97%, 98%, 99%, or 100% identical to or consist of the amino acid sequence set forth in SEQ ID NO: 44. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 28. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 44. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target tumor antigen. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target tumor antigen.

In certain embodiments, a multi-specific binding polypeptide (e.g., a multi-specific antibody) described herein comprises a tumor binding moiety comprising sequences (e.g., CDRs or VH/VL sequences) derived from a known antibody such as RS7, MAAP-9001a, 7E6, 4F6 (TROP2), Codrituzumab/GC33/RO5137382, HN3, YP7, HS20, 4G5, MDX-1414 (GPC3) Trastuzumab (HER2), brentuximab, ramucirumab, iratumumab, olinvacimab, vesencumab (CD30), dinutuximab, Hu3F8, MAb-3F8, MORAb-028/MT228, KM666 (GD2), mirvetuximab/huFR107, farletuzumab, MORAb-003, SP8166, 26B3.F2, HuRA15 (FOLR1), daratumumab, isatuximab, mezagitamab (CD38), or IMC-EB10 (FLT3).

In certain embodiments, a multi-specific binding polypeptide (e.g., a multi-specific antibody) described herein comprises an immune cell binding moiety comprising sequences at least about 80%, 85%, 90%, 95%, 97%, 98%, 99%, or 100% identical to or consist of sequences (e.g., CDRs or VH/VL sequences) selected from Tables 3 and/or 4.

TABLE 3

| NAME* | VH SEQUENCE^{#} | HCDR1 | HCDR2 | HCDR3 | VH |
|---|---|---|---|---|---|
| αCD33 | EVQLVQSGAEVKKPGSSVKVSCKASGY | SEQ ID | SEQ ID | SEQ ID | SEQ ID |
| | TITDSNIHWVRQAPGQSLEWIGYIYPYN | NO: 45 | NO: 46 | NO: 47 | NO: 48 |
| | GGTDYNQKFKNRATLTVDNPTNTAYM | | | | |
| | ELSSLRSEDTAFYYCVNGNPWLAYWG | | | | |
| | QGTLVTVSS | | | | |
| αCD163 | QVQLQESGPGLVKPSETLSLTCTVSGYS | SEQ ID | SEQ ID | SEQ ID | SEQ ID |
| | ITSDYAWNWIRQFPGNKLEWMGYITYS | NO: 49 | NO: 50 | NO: 51 | NO: 52 |
| | GSTYYNPSLKSRVTISVDTSKNQFSLKL | | | | |
| | SSVTAADTATYYCVSGTYYFDYWGQG | | | | |
| | TTLTVSS | | | | |
| αTRAIL- | QVQLQESGPGLVKPSQTLSLTCTVSGGS | SEQ ID | SEQ ID | SEQ ID | SEQ ID |
| R2 | ISSGDYFWSWIRQLPGKGLEWIGHIHNS | NO: 53 | NO: 54 | NO: 55 | NO: 11 |
| (αDR5) | GTTYYNPSLKSRVTISVDTSKKQFSLRL | | | | |
| | SSVTAADTAVYYCARDRGGDYYYGMD | | | | |
| | VWGQGTTVTVSS | | | | |
| αCSF1R | QVQLVQSGAEVKKPGSSVKVSCKASGY | SEQ ID | SEQ ID | SEQ ID | SEQ ID |
| | TFTDNYMIWVRQAPGQGLEWMGDINP | NO: 56 | NO: 57 | NO: 58 | NO: 59 |
| | YNGGTTFNQKFKGRVTITADKSTSTAY | | | | |
| | MELSSLRSEDTAVYYCARESPYFSNLY | | | | |
| | VMDYWGQGTLVTVSS | | | | |

*The notation “α” in front of an exemplary antigen expressed on an immunosuppressive cell denotes an antibody that specifically binds to the antigen expressed on an immunosuppressive cell.
^{#}The underlined sequences denote the respective HCDR1, HCDR2, and HCDR3.

TABLE 4

| NAME* | VL SEQUENCE^{#} | LCDR1 | LCDR2 | LCDR3 | VL |
|---|---|---|---|---|---|
| αCD33 | DIQLTQSPSTLSASVGDRVTITCRASES | SEQ ID | SEQ ID | SEQ ID | SEQ ID |
| | LDNYGIRFLTWFQQKPGKAPKLLMYA | NO: 60 | NO: 61 | NO: 62 | NO: 63 |
| | ASNQGSGVPSRFSGSGSGTEFTLTISSL | | | | |
| | QPDDFATYYCQQTKEVPWSFGQGTKV | | | | |
| | EVK | | | | |
| αCD163 | DIVMTQSPSSLSASVGDRVTITCRASQS | SEQ ID | SEQ ID | SEQ ID | SEQ ID |
| | VSSDVAWFQQKPGKSPKPLIYYASNR | NO: 64 | NO: 65 | NO: 66 | NO: 67 |
| | YSGVPSRFSGSGSGTDFTLTISSLQAED | | | | |
| | FAVYFCGQDYTSPRTFGGGTKLEIK | | | | |
| αTRAIL- | EIVLTQSPGTLSLSPGERATLSCRASQGI | SEQ ID | SEQ ID | SEQ ID | SEQ ID |
| R2 | SRSYLAWYQQKPGQAPSLLIYGASSRA | NO: 68 | NO: 69 | NO: 70 | NO: 12 |
| (αDR5) | TGIPDRFSGSGSGTDFTLTISRLEPEDFA | | | | |
| | VYYCQQFGSSPWTFGQGTKVEI | | | | |
| αCSF1R | EIVLTQSPATLSLSPGERATLSCKASQS | SEQ ID | SEQ ID | SEQ ID | SEQ ID |
| | VDYDGDNYMNWYQQKPGQAPRLLIY | NO: 71 | NO: 72 | NO: 73 | NO: 74 |
| | AASNLESGIPARFSGSGSGTDFTLTISSL | | | | |
| | EPEDFAVYYCHLSNEDLSTFGGGTKVE | | | | |
| | IK | | | | |

*The notation “α” in front of an exemplary antigen expressed on an immunosuppressive cell denotes an antibody that specifically binds to the antigen expressed on an immunosuppressive cell.
^{#}The underlined sequences denote the respective LCDR1, LCDR2, and LCDR3.

In some embodiments, a multi-specific binding polypeptide (e.g., a multi-specific antibody) described herein comprises an immune cell binding moiety comprising an immunoglobulin heavy chain variable region comprising an amino acid sequence at least about 80%, 85%, 90%, 95%, 97%, 98%, 99%, or 100% identical to the amino acid sequence set forth in SEQ ID NOs: 11, 48, 52, or 59; and an immunoglobulin light chain variable region at least about 80%, 85%, 90%, 95%, 97%, 98%, 99%, or 100% identical to the amino acid sequence set forth in SEQ ID NOs: 12, 63, 67, or 74. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NOs: 11, 48, 52, or 59. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NOs: 12, 63, 67, or 74. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target antigen expressed on an immunosuppressive cell.

In some embodiments, a multi-specific binding polypeptide (e.g., a multi-specific antibody) described herein comprises an immune cell binding moiety comprising an immunoglobulin heavy chain variable region comprising an amino acid sequence at least about 80%, 85%, 90%, 95%, 97%, 98%, 99%, or 100% identical to the amino acid sequence set forth in SEQ ID NO: 11; and an immunoglobulin light chain variable region at least about 80%, 85%, 90%, 95%, 97%, 98%, 99%, or 100% identical to the amino acid sequence set forth in SEQ ID NO: 12. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 11. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 12. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target antigen expressed on an immunosuppressive cell.

In some embodiments, a multi-specific binding polypeptide (e.g., a multi-specific antibody) described herein comprises an immune cell binding moiety comprising an immunoglobulin heavy chain variable region comprising an amino acid sequence at least about 80%, 85%, 90%, 95%, 97%, 98%, 99%, or 100% identical to the amino acid sequence set forth in SEQ ID NO: 48; and an immunoglobulin light chain variable region at least about 80%, 85%, 90%, 95%, 97%, 98%, 99%, or 100% identical to the amino acid sequence set forth in SEQ ID NO: 63. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 48. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 63. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target antigen expressed on an immunosuppressive cell.

In some embodiments, a multi-specific binding polypeptide (e.g., a multi-specific antibody) described herein comprises an immune cell binding moiety comprising an immunoglobulin heavy chain variable region comprising an amino acid sequence at least about 80%, 85%, 90%, 95%, 97%, 98%, 99%, or 100% identical to the amino acid sequence set forth in SEQ ID NO: 52; and an immunoglobulin light chain variable region at least about 80%, 85%, 90%, 95%, 97%, 98%, 99%, or 100% identical to the amino acid sequence set forth in SEQ ID NO: 67. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 52. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 67. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target antigen expressed on an immunosuppressive cell.

In some embodiments, a multi-specific binding polypeptide (e.g., a multi-specific antibody) described herein comprises an immune cell binding moiety comprising an immunoglobulin heavy chain variable region comprising an amino acid sequence at least about 80%, 85%, 90%, 95%, 97%, 98%, 99%, or 100% identical to the amino acid sequence set forth in SEQ ID NO: 59; and an immunoglobulin light chain variable region at least about 80%, 85%, 90%, 95%, 97%, 98%, 99%, or 100% identical to the amino acid sequence set forth in SEQ ID NO: 74. In some instances, the amino acid differences contributing to the at least about 80%, 85%, 90%, 95%, 97%, 98%, or 99% sequence identity resides in a framework region of the immunoglobulin heavy chain variable region and the CDRs remain unchanged relative to the CDRs set forth in SEQ ID NO: 59. In some instances, the amino acid differences contributing to the at least about 80%, 85%, 90%, 95%, 97%, 98%, or 99% sequence identity resides in a framework region of the immunoglobulin light chain variable region and the CDRs remain unchanged relative to the CDRs set forth in SEQ ID NO: 74. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain variable region but the multi-specific binding polypeptide (e.g., the multi-specific antibody) retains binding to a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain variable region but the multi-specific binding polypeptide (e.g., the multi-specific antibody) retains binding to a target antigen expressed on an immunosuppressive cell.

In certain embodiments, the immune cell binding moiety of the multi-specific binding polypeptide (e.g., multi-specific antibody) comprises sequences (e.g., CDRs or VH/VL sequences) derived from a known antibody such as Conatumumab, Tigatuzumab, drozitumab, Lexatumumab, Benufutamab, Zaptuzumab (TRAIL-R2), Cabiralizumab, Emactuzumab, LY3022855, AMG820 (CSF1R), Gemtuzumab, Vadastuximab, Lintuzumab (CD33), or TBI 304H (CD163).

In certain embodiments, a multi-specific binding polypeptide (e.g., a multi-specific antibody) described herein comprises an immunoglobulin heavy chain comprising an amino acid sequence at least about 80%, 85%, 90%, 95%, 97%, 98%, 99%, or 100% identical to or consist of the amino acid sequence set forth under the heavy chain (HC) sequences of Table 5; and an immunoglobulin light chain comprising an amino acid sequence at least about 80%, 85%, 90%, 95%, 97%, 98%, 99%, or 100% identical to or consist of the amino acid sequence set forth under the light chain (LC) sequences of Table 5. In some instances, the amino acid differences contributing to the at least about 80%, 85%, 90%, 95%, 97%, 98%, or 99% sequence identity resides in a framework region of the immunoglobulin heavy chain and the CDRs remain unchanged relative to the CDRs set forth in the respective HC sequence in Table 5. In some instances, the amino acid differences contributing to the at least about 80%, 85%, 90%, 95%, 97%, 98%, or 99% sequence identity resides in a framework region of the immunoglobulin light chain and the CDRs remain unchanged relative to the CDRs set forth in the respective LC sequence in Table 5. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain but the multi-specific binding polypeptide (e.g., the multi-specific antibody) retains binding to a target tumor antigen and/or a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain but the multi-specific binding polypeptide (e.g., the multi-specific antibody) retains binding to a target tumor antigen and/or a target antigen expressed on an immunosuppressive cell.

As shown below in Table 5, the underlined portion in the HC sequences indicate the scFv portion of the multi-specific antibody. The respective linker sequences in the heavy chain are shown in lower cases.

TABLE 5

| | | SEQ | | SEQ |
|---|---|---|---|---|
| | | ID | | ID |
| NAME | HC | NO: | LC | NO: |
| DABA_1 | QVQLQQSGSELKKPGASVKVSCKASGYT | 7 | DIQLTQSPSSLS | 8 |
| (αTROP2 × | FTNYGMNWVKQAPGQGLKWMGWINTY | | ASVGDRVSITC | |
| αTRAIL-R2 | TGEPTYTDDFKGRFAFSLDTSVSTAYLQIS | | KASQDVSIAVA | |
| in IgG1 | SLKADDTAVYFCARGGFGSSYWYFDVW | | WYQQKPGKAP | |
| backbone) | GQGSLVTVSSASTKGPSVFPLAPSSKSTSG | | KLLIYSASYRYT | |
| | GTAALGCLVKDYFPEPVTVSWNSGALTS | | GVPDRFSGSGS | |
| | GVHTFPAVLQSSGLYSLSSVVTVPSSSLGT | | GTDFTLTISSLQ | |
| | QTYICNVNHKPSNTKVDKKVEPKSCDKT | | PEDFAVYYCQQ | |
| | HTCPPCPAPELLGGPSVFLFPPKPKDTLMI | | HYITPLTFGAGT | |
| | SRTPEVTCVVVDVEHEDPEVKFNWYVDG | | KVEIKRTVAAP | |
| | VEVHNAKTKPREEQYNSTYRVVSVLTVL | | SVFIFPPSDEQL | |
| | HQDWLNGKEYKCKVSNKALPAPIEKTIS | | KSGTASVVCLL | |
| | KAKGQPREPQVYTLPPSREEMTKNQVSL | | NNFYPREAKVQ | |
| | TCLVKGFYPSDIAVEWESNGQPENNYKT | | WKVDNALQSG | |
| | TPPVLDSDGSFFLYSKLTVDKSRWQQGN | | NSQESVTEQDS | |
| | VFSCSVMHEALHNHYTQKSLSLSLggggsg | | KDSTYSLSSTLT | |
| | gggsEIVLTQSPGTLSLSPGERATLSCRASQGI | | LSKADYEKHKV | |
| | SRSYLAWYQQKPGQAPSLLIYGASSRATGIP | | YACEVTHQGLS | |
| | DRFSGSGSGTDFTLTISRLEPEDFAVYYCQQ | | SPVTKSFNRGE | |
| | FGSSPWTFGQGTKVEIKggggsggggsggggsgg | | C | |
| | ggsQVQLQESGPGLVKPSQTLSLTCTVSGGSI | | | |
| | SSGDYFWSWIRQLPGKGLEWIGHIHNSGTT | | | |
| | YYNPSLKSRVTISVDTSKKQFSLRLSSVTAAD | | | |
| | TAVYYCARDRGGDYYYGMDVWGQGTTVTV | | | |
| | SS | | | |
| DABA_2 | QVQLQQSGSELKKPGASVKVSCKASGYT | 75 | DIQLTQSPSSLS | 8 |
| (αTROP2 × | FTNYGMNWVKQAPGQGLKWMGWINTY | | ASVGDRVSITC | |
| αCD33 in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone) | GQGSLVTVSSASTKGPSVFPLAPSSKSTSG | | KLLIYSASYRYT | |
| | GTAALGCLVKDYFPEPVTVSWNSGALTS | | GVPDRFSGSGS | |
| | GVHTFPAVLQSSGLYSLSSVVTVPSSSLGT | | GTDFTLTISSLQ | |
| | QTYICNVNHKPSNTKVDKRVEPKSCDKT | | PEDFAVYYCQQ | |
| | HTCPPCPAPELLGGPSVFLFPPKPKDTLMI | | HYITPLTFGAGT | |
| | SRTPEVTCVVVDVSHEDPEVKFNWYVDG | | KVEIKRTVAAP | |
| | VEVHNAKTKPREEQYNSTYRVVSVLTVL | | SVFIFPPSDEQL | |
| | HQDWLNGKEYKCKVSNKALPAPIEKTIS | | KSGTASVVCLL | |
| | KAKGQPREPQVYTLPPSREEMTKNQVSL | | NNFYPREAKVQ | |
| | TCLVKGFYPSDIAVEWESNGQPENNYKT | | WKVDNALQSG | |
| | TPPVLDSDGSFFLYSKLTVDKSRWQQGN | | NSQESVTEQDS | |
| | VFSCSVMHEALHNHYTQKSLSLSLggggsg | | KDSTYSLSSTLT | |
| | gggsEVQLVQSGAEVKKPGSSVKVSCKASGY | | LSKADYEKHKV | |
| | TITDSNIHWVRQAPGQSLEWIGYIYPYNGGT | | YACEVTHQGLS | |
| | DYNQKFKNRATLTVDNPTNTAYMELSSLRSE | | SPVTKSFNRGE | |
| | DTAFYYCVNGNPWLAYWGQGTLVTVSSgggg | | C | |
| | sggggsggggsggggsDIQLTQSPSTLSASVGDRV | | | |
| | TITCRASESLDNYGIRFLTWFQQKPGKAPKL | | | |
| | LMYAASNQGSGVPSRFSGSGSGTEFTLTISSL | | | |
| | QPDDFATYYCQQTKEVPWSFGQGTKVEVK | | | |
| DABA_3 | QVQLQQSGSELKKPGASVKVSCKASGYT | 76 | DIQLTQSPSSLS | 8 |
| (αTROP2 × | FTNYGMNWVKQAPGQGLKWMGWINTY | | ASVGDRVSITC | |
| αCSF1R in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone) | GQGSLVTVSSASTKGPSVFPLAPSSKSTSG | | KLLIYSASYRYT | |
| | GTAALGCLVKDYFPEPVTVSWNSGALTS | | GVPDRFSGSGS | |
| | GVHTFPAVLQSSGLYSLSSVVTVPSSSLGT | | GTDFTLTISSLQ | |
| | QTYICNVNHKPSNTKVDKRVEPKSCDKT | | PEDFAVYYCQQ | |
| | HTCPPCPAPELLGGPSVFLFPPKPKDTLMI | | HYITPLTFGAGT | |
| | SRTPEVTCVVVDVSHEDPEVKFNWYVDG | | KVEIKRTVAAP | |
| | VEVHNAKTKPREEQYNSTYRVVSVLTVL | | SVFIFPPSDEQL | |
| | HQDWLNGKEYKCKVSNKALPAPIEKTIS | | KSGTASVVCLL | |
| | KAKGQPREPQVYTLPPSREEMTKNQVSL | | NNFYPREAKVQ | |
| | TCLVKGFYPSDIAVEWESNGQPENNYKT | | WKVDNALQSG | |
| | TPPVLDSDGSFFLYSKLTVDKSRWQQGN | | NSQESVTEQDS | |
| | VFSCSVMHEALHNHYTQKSLSLSLggggsg | | KDSTYSLSSTLT | |
| | gggsQVQLVQSGAEVKKPGSSVKVSCKASGY | | LSKADYEKHKV | |
| | TFTDNYMIWVRQAPGQGLEWMGDINPYNG | | YACEVTHQGLS | |
| | GTTFNQKFKGRVTITADKSTSTAYMELSSLR | | SPVTKSFNRGE | |
| | SEDTAVYYCARESPYFSNLYVMDYWGQGTL | | C | |
| | VTVSSggggsggggsggggsggggsEIVLTQSPATLS | | | |
| | LSPGERATLSCKASQSVDYDGDNYMNWYQ | | | |
| | QKPGQAPRLLIYAASNLESGIPARFSGSGSGT | | | |
| | DFTLTISSLEPEDFAVYYCHLSNEDLSTFGG | | | |
| | GTKVEIK | | | |
| DABA_4 | QVQLQQSGSELKKPGASVKVSCKASGYT | 77 | DIQLTQSPSSLS | 8 |
| (αTROP2 × | FTNYGMNWVKQAPGQGLKWMGWINTY | | ASVGDRVSITC | |
| αCD163 in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone) | GQGSLVTVSSASTKGPSVFPLAPSSKSTSG | | KLLIYSASYRYT | |
| | GTAALGCLVKDYFPEPVTVSWNSGALTS | | GVPDRFSGSGS | |
| | GVHTFPAVLQSSGLYSLSSVVTVPSSSLGT | | GTDFTLTISSLQ | |
| | QTYICNVNHKPSNTKVDKRVEPKSCDKT | | PEDFAVYYCQQ | |
| | HTCPPCPAPELLGGPSVFLFPPKPKDTLMI | | HYITPLTFGAGT | |
| | SRTPEVTCVVVDVSHEDPEVKFNWYVDG | | KVEIKRTVAAP | |
| | VEVHNAKTKPREEQYNSTYRVVSVLTVL | | SVFIFPPSDEQL | |
| | HQDWLNGKEYKCKVSNKALPAPIEKTIS | | KSGTASVVCLL | |
| | KAKGQPREPQVYTLPPSREEMTKNQVSL | | NNFYPREAKVQ | |
| | TCLVKGFYPSDIAVEWESNGQPENNYKT | | WKVDNALQSG | |
| | TPPVLDSDGSFFLYSKLTVDKSRWQQGN | | NSQESVTEQDS | |
| | VFSCSVMHEALHNHYTQKSLSLSLggggsg | | KDSTYSLSSTLT | |
| | gggsQVQLQESGPGLVKPSETLSLTCTVSGYS | | LSKADYEKHKV | |
| | ITSDYAWNWIRQFPGNKLEWMGYITYSGSTY | | YACEVTHQGLS | |
| | YNPSLKSRVTISVDTSKIVQFSLKLSSVTAADT | | SPVTKSFNRGE | |
| | ATYYCVSGTYYFDYWGQGTTLTVSSggggsggg | | C | |
| | gsggggsggggsDIVMTQSPSSLSASVGDRVTIT | | | |
| | CRASQSVSSDVAWFQQKPGKSPKPLIYYASN | | | |
| | RYSGVPSRFSGSGSGTDFTLTISSLQAEDFAV | | | |
| | YFCGQDYTSPRTFGGGTKLEIK | | | |
| DABA_5 | QVQLVQSGAEVKKPGASVKVSCKASGYT | 79 | DVVMTQSPLSL | 80 |
| (αGPC3 × | FTDYEMHWVRQAPGQGLEWMGALDPKT | | PVTPGEPASISC | |
| αCD33 in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone) | TVSSASTKGPSVFPLAPSSKSTSGGTAALG | | PGQSPQLLIYKV | |
| | CLVKDYFPEPVTVSWNSGALTSGVHTFP | | SNRFSGVPDRFS | |
| | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPELLGGPSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPAPIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsEVQLVQ | | TEQDSKDSTYS | |
| | SGAEVKKPGSSVKVSCKASGYTITDSNIHWV | | LSSTLTLSKAD | |
| | RQAPGQSLEWIGYIYPYNGGTDYNQKFKNR | | YEKHKVYACE | |
| | ATLTVDNPTNTAYMELSSLRSEDTAFYYCVN | | VTHQGLSSPVT | |
| | GNPWLAYWGQGTLVTVSSggggsggggsggggs | | KSFNRGEC | |
| | ggggsDIQLTQSPSTLSASVGDRVTITCRASES | | | |
| | LDNYGIRFLTWFQQKPGKAPKLLMYAASNQ | | | |
| | GSGVPSRFSGSGSGTEFTLTISSLQPDDFATY | | | |
| | YCQQTKEVPWSFGQGTKVEVK | | | |
| DABA_6 | QVQLVQSGAEVKKPGASVKVSCKASGYT | 81 | DVVMTQSPLSL | 80 |
| (αGPC3 × | FTDYEMHWVRQAPGQGLEWMGALDPKT | | PVTPGEPASISC | |
| αCSF1R in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone) | TVSSASTKGPSVFPLAPSSKSTSGGTAALG | | PGQSPQLLIYKV | |
| | CLVKDYFPEPVTVSWNSGALTSGVHTFP | | SNRFSGVPDRFS | |
| | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPELLGGPSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPAPIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsQVQLV | | TEQDSKDSTYS | |
| | QSGAEVKKPGSSVKVSCKASGYTFTDNYMI | | LSSTLTLSKAD | |
| | WVRQAPGQGLEWMGDINPYNGGTTFNQK | | YEKHKVYACE | |
| | FKGRVTITADKSTSTAYMELSSLRSEDTAVYY | | VTHQGLSSPVT | |
| | CARESPYFSNLYVMDYWGQGTLVTVSSggggs | | KSFNRGEC | |
| | ggggsggggsggggsEIVLTQSPATLSLSPGERAT | | | |
| | LSCKASQSVDYDGDNYMNWYQQKPGQAPR | | | |
| | LLIYAASNLESGIPARFSGSGSGTDFTLTISSL | | | |
| | EPEDFAVYYCHLSNEDLSTFGGGTKVEIK | | | |
| DABA_7 | QVQLVQSGAEVKKPGASVKVSCKASGYT | 82 | DVVMTQSPLSL | 80 |
| (αGPC3 × | FTDYEMHWVRQAPGQGLEWMGALDPKT | | PVTPGEPASISC | |
| αCD163 in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone) | TVSSASTKGPSVFPLAPSSKSTSGGTAALG | | PGQSPQLLIYKV | |
| | CLVKDYFPEPVTVSWNSGALTSGVHTFP | | SNRFSGVPDRFS | |
| | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPELLGGPSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPAPIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsQVQLQ | | TEQDSKDSTYS | |
| | ESGPGLVKPSETLSLTCTVSGYSITSDYAWN | | LSSTLTLSKAD | |
| | WIRQFPGNKLEWMGYITYSGSTYYNPSLKSR | | YEKHKVYACE | |
| | VTISVDTSKNQFSLKLSSVTAADTATYYCVSG | | | |
| | TYYFDYWGQGTTLTVSSggggsggggsggggsggg | | VTHQGLSSPVT | |
| | gsDIVMTQSPSSLSASVGDRVTITCRASQSVSS | | KSFNRGEC | |
| | DVAWFQQKPGKSPKPLIYYASNRYSGVPSRF | | | |
| | SGSGSGTDFTLTISSLQAEDFAVYFCGQDYT | | | |
| | SPRTFGGGTKLEIK | | | |
| DABA_8 | QVQLVQSGAEVKKPGASVKVSCKASGYT | 83 | DVVMTQSPLSL | 80 |
| (αGPC3 × | FTDYEMHWVRQAPGQGLEWMGALDPKT | | PVTPGEPASISC | |
| αTRAIL-R2 | GDTAYSQKFKGRVTLTADKSTSTAYMEL | | RSSQSLVHSNR | |
| in IgG1 | SSLTSEDTAVYYCTRFYSYTYWGQGTLV | | NTYLHWYLQK | |
| Backbone) | TVSSASTKGPSVFPLAPSSKSTSGGTAALG | | PGQSPQLLIYKV | |
| | CLVKDYFPEPVTVSWNSGALTSGVHTFP | | SNRFSGVPDRFS | |
| | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPELLGGPSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPAPIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsEIVLTQS | | TEQDSKDSTYS | |
| | PGTLSLSPGERATLSCRASQGISRSYLAWYQ | | LSSTLTLSKAD | |
| | QKPGQAPSLLIYGASSRATGIPDRFSGSGSG | | YEKHKVYACE | |
| | TDFTLTISRLEPEDFAVYYCQQFGSSPWTFG | | VTHQGLSSPVT | |
| | QGTKVEIKggggsggggsggggsggggsQVQLQES | | KSFNRGEC | |
| | GPGLVKPSQTLSLTCTVSGGSISSGDYFWSW | | | |
| | IRQLPGKGLEWIGHIHNSGTTYYNPSLKSRV | | | |
| | TISVDTSKKQFSLRLSSVTAADTAVYYCARDR | | | |
| | GGDYYYGMDVWGQGTTVTVSS | | | |
| DABA_9 | QVQLVQSGAEVVKPGASVKISCKASGYT | 84 | DIVLTQSPLSLA | 85 |
| (αFOLR1 × | FTGYFMNWVKQSPGQSLEWIGRIHPYDG | | VSLGQPAIISCK | |
| αCD33 in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone) | TTVTVSSASTKGPSVFPLAPSSKSTSGGTA | | QQPRLLIYRAS | |
| | ALGCLVKDYFPEPVTVSWNSGALTSGVH | | NLEAGVPDRFS | |
| | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPAPIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsEV | | TEQDSKDSTYS | |
| | QLVQSGAEVKKPGSSVKVSCKASGYTITDSN | | LSSTLTLSKAD | |
| | IHWVRQAPGQSLEWIGYIYPYNGGTDYNQK | | YEKHKVYACE | |
| | FKNRATLTVDNPTNTAYMELSSLRSEDTAFY | | VTHQGLSSPVT | |
| | YCVNGNPWLAYWGQGTLVTVSSggggsggggs | | KSFNRGEC | |
| | ggggsggggsDIQLTQSPSTLSASVGDRVTITCR | | | |
| | ASESLDNYGIRFLTWFQQKPGKAPKLLMYA | | | |
| | ASNQGSGVPSRFSGSGSGTEFTLTISSLQPD | | | |
| | DFATYYCQQTKEVPWSFGQGTKVEVK | | | |
| DABA_10 | QVQLVQSGAEVVKPGASVKISCKASGYT | 86 | DIVLTQSPLSLA | 85 |
| (αFOLR1 × | FTGYFMNWVKQSPGQSLEWIGRIHPYDG | | VSLGQPAIISCK | |
| αCSF1R in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone) | TTVTVSSASTKGPSVFPLAPSSKSTSGGTA | | QQPRLLIYRAS | |
| | ALGCLVKDYFPEPVTVSWNSGALTSGVH | | NLEAGVPDRFS | |
| | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPAPIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsQV | | TEQDSKDSTYS | |
| | QLVQSGAEVKKPGSSVKVSCKASGYTFTDN | | LSSTLTLSKAD | |
| | YMIWVRQAPGQGLEWMGDINPYNGGTTFN | | YEKHKVYACE | |
| | QKFKGRVTITADKSTSTAYMELSSLRSEDTA | | VTHQGLSSPVT | |
| | VYYCARESPYFSNLYVMDYWGQGTLVTVSSg | | KSFNRGEC | |
| | gggsggggsggggsggggsEIVLTQSPATLSLSPGE | | | |
| | RATLSCKASQSVDYDGDNYMNWYQQKPGQ | | | |
| | APRLLIYAASNLESGIPARFSGSGSGTDFTLTI | | | |
| | SSLEPEDFAVYYCHLSNEDLSTFGGGTKVEI | | | |
| | K | | | |
| DABA_11 | QVQLVQSGAEVVKPGASVKISCKASGYT | 87 | DIVLTQSPLSLA | 85 |
| (αFOLR1 × | FTGYFMNWVKQSPGQSLEWIGRIHPYDG | | VSLGQPAIISCK | |
| αCD163 in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone) | TTVTVSSASTKGPSVFPLAPSSKSTSGGTA | | QQPRLLIYRAS | |
| | ALGCLVKDYFPEPVTVSWNSGALTSGVH | | NLEAGVPDRFS | |
| | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPAPIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsQV | | TEQDSKDSTYS | |
| | QLQESGPGLVKPSETLSLTCTVSGYSITSDYA | | LSSTLTLSKAD | |
| | WNWIRQFPGNKLEWMGYITYSGSTYYNPSL | | YEKHKVYACE | |
| | KSRVTISVDTSKIVQFSLKLSSVTAADTATYYC | | VTHQGLSSPVT | |
| | VSGTYYFDYWGQGTTLTVSSggggsggggsgggg | | KSFNRGEC | |
| | sggggsDIVMTQSPSSLSASVGDRVTITCRASQ | | | |
| | SVSSDVAWFQQKPGKSPKPLIYYASNRYSGV | | | |
| | PSRFSGSGSGTDFTLTISSLQAEDFAVYFCG | | | |
| | QDYTSPRTFGGGTKLEIK | | | |
| DABA_12 | QVQLVQSGAEVVKPGASVKISCKASGYT | 88 | DIVLTQSPLSLA | 85 |
| (αFOLR1 × | FTGYFMNWVKQSPGQSLEWIGRIHPYDG | | VSLGQPAIISCK | |
| αTRAIL-R2 | DTFYNQKFQGKATLTVDKSSNTAHMELL | | ASQSVSFAGTS | |
| in IgG1 | SLTSEDFAVYYCTRYDGSRAMDYWGQG | | LMHWYHQKPG | |
| Backbone) | TTVTVSSASTKGPSVFPLAPSSKSTSGGTA | | QQPRLLIYRAS | |
| | ALGCLVKDYFPEPVTVSWNSGALTSGVH | | NLEAGVPDRFS | |
| | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPAPIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsEIV | | TEQDSKDSTYS | |
| | LTQSPGTLSLSPGERATLSCRASQGISRSYLA | | LSSTLTLSKAD | |
| | WYQQKPGQAPSLLIYGASSRATGIPDRFSGS | | YEKHKVYACE | |
| | GSGTDFTLTISRLEPEDFAVYYCQQFGSSPW | | VTHQGLSSPVT | |
| | TFGQGTKVEIKggggsggggsggggsggggsQVQL | | KSFNRGEC | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_13 | EVQLVQSGAEVKKPGSSVKVSCKASGYTI | 89 | DIQLTQSPSTLS | 90 |
| (αCD33 × | TDSNIHWVRQAPGQSLEWIGYIYPYNGGT | | ASVGDRVTITC | |
| αTRAIL-R2 | DYNQKFKNRATLTVDNPTNTAYMELSSL | | RASESLDNYGI | |
| in IgG1 | RSEDTAFYYCVNGNPWLAYWGQGTLVT | | RFLTWFQQKPG | |
| Backbone) | VSSASTKGPSVFPLAPSSKSTSGGTAALGC | | KAPKLLMYAAS | |
| | LVKDYFPEPVTVSWNSGALTSGVHTFPA | | NQGSGVPSRFS | |
| | VLQSSGLYSLSSVVTVPSSSLGTQTYICNV | | GSGSGTEFTLTI | |
| | NHKPSNTKVDKKVEPKSCDKTHTCPPCP | | SSLQPDDFATY | |
| | APELLGGPSVFLFPPKPKDTLMISRTPEVT | | YCQQTKEVPWS | |
| | CVVVDVEHEDPEVKFNWYVDGVEVHNA | | FGQGTKVEVKR | |
| | KTKPREEQYNSTYRVVSVLTVLHQDWLN | | TVAAPSVFIFPP | |
| | GKEYKCKVSNKALPAPIEKTISKAKGQPR | | SDEQLKSGTAS | |
| | EPQVYTLPPSREEMTKNQVSLTCLVKGFY | | VVCLLNNFYPR | |
| | PSDIAVEWESNGQPENNYKTTPPVLDSDG | | EAKVQWKVDN | |
| | SFFLYSKLTVDKSRWQQGNVFSCSVMHE | | ALQSGNSQESV | |
| | ALHNHYTQKSLSLSLggggsggggsEIVLTQSP | | TEQDSKDSTYS | |
| | GTLSLSPGERATLSCRASQGISRSYLAWYQQ | | LSSTLTLSKAD | |
| | KPGQAPSLLIYGASSRATGIPDRFSGSGSGT | | YEKHKVYACE | |
| | DFTLTISRLEPEDFAVYYCQQFGSSPWTFGQ | | VTHQGLSSPVT | |
| | GTKVEIKggggsggggsggggsggggsQVQLQESG | | KSFNRGEC | |
| | PGLVKPSQTLSLTCTVSGGSISSGDYFWSWIR | | | |
| | QLPGKGLEWIGHIHNSGTTYYNPSLKSRVTIS | | | |
| | VDTSKKQFSLRLSSVTAADTAVYYCARDRGG | | | |
| | DYYYGMDVWGQGTTVTVSS | | | |
| DABA_14 | EVQLLESGGGLVQPGGSLRLSCAVSGFTF | 91 | EIVLTQSPATLS | 92 |
| (αCD38 × | NSFAMSWVRQAPGKGLEWVSAISGSGGG | | LSPGERATLSCR | |
| αTRAIL-R2 | TYYADSVKGRFTISRDNSKNTLYLQMNS | | ASQSVSSYLAW | |
| in IgG1 | LRAEDTAVYFCAKDKILWFGEPVFDYWG | | YQQKPGQAPRL | |
| Backbone) | QGTLVTVSSASTKGPSVFPLAPSSKSTSGG | | LIYDASNRATGI | |
| | TAALGCLVKDYFPEPVTVSWNSGALTSG | | PARFSGSGSGT | |
| | VHTFPAVLQSSGLYSLSSVVTVPSSSLGTQ | | DFTLTISSLEPE | |
| | TYICNVNHKPSNTKVDKRVEPKSCDKTH | | DFAVYYCQQRS | |
| | TCPPCPAPELLGGPSVFLFPPKPKDTLMIS | | NWPPTFGQGTK | |
| | RTPEVTCVVVDVSHEDPEVKFNWYVDG | | VEIKRTVAAPS | |
| | VEVHNAKTKPREEQYNSTYRVVSVLTVL | | VFIFPPSDEQLK | |
| | HQDWLNGKEYKCKVSNKALPAPIEKTIS | | SGTASVVCLLN | |
| | KAKGQPREPQVYTLPPSREEMTKNQVSL | | NFYPREAKVQ | |
| | TCLVKGFYPSDIAVEWESNGQPENNYKT | | WKVDNALQSG | |
| | TPPVLDSDGSFFLYSKLTVDKSRWQQGN | | NSQESVTEQDS | |
| | VFSCSVMHEALHNHYTQKSLSLSLggggsg | | KDSTYSLSSTLT | |
| | gggsEIVLTQSPGTLSLSPGERATLSCRASQGI | | LSKADYEKHKV | |
| | SRSYLAWYQQKPGQAPSLLIYGASSRATGIP | | YACEVTHQGLS | |
| | DRFSGSGSGTDFTLTISRLEPEDFAVYYCQQ | | SPVTKSFNRGE | |
| | FGSSPWTFGQGTKVEIKggggsggggsggggsgg | | C | |
| | ggsQVQLQESGPGLVKPSQTLSLTCTVSGGSI | | | |
| | SSGDYFWSWIRQLPGKGLEWIGHIHNSGTT | | | |
| | YYNPSLKSRVTISVDTSKKQFSLRLSSVTAAD | | | |
| | TAVYYCARDRGGDYYYGMDVWGQGTTVTV | | | |
| | SS | | | |
| DABA_15 | EVQLVQSGAEVKKPGASVKVSCKASGYT | 93 | DVVMTQSPLSL | 94 |
| (αFLT3 × | FTSYYMHWVRQAPGQGLEWMGIINPSGG | | PVTPGEPASISC | |
| αTRAIL-R2 | STSYAQKFQGRVTMTRDTSTSTVYMELS | | RSSQSLLHSNG | |
| in IgG1 | SLRSEDTAVYYCARGVGAHDAFDIWGQG | | NNYLDWYLQK | |
| Backbone) | TTVTVSSASTKGPSVFPLAPSSKSTSGGTA | | PGQSPQLLIYLG | |
| | ALGCLVKDYFPEPVTVSWNSGALTSGVH | | SNRASGVPDRF | |
| | TFPAVLQSSGLYSLSSVVTVPSSSLGTQTY | | SGSGSDTDFTL | |
| | ICNVNHKPSNTKVDKRVEPKSCDKTHTCP | | QISRVEAEDVG | |
| | PCPAPELLGGPSVFLFPPKPKDTLMISRTP | | VYYCMQGTHP | |
| | EVTCVVVDVSHEDPEVKFNWYVDGVEV | | AISFGQGTRLEI | |
| | HNAKTKPREEQYNSTYRVVSVLTVLHQD | | KRTVAAPSVFIF | |
| | WLNGKEYKCKVSNKALPAPIEKTISKAK | | PPSDEQLKSGT | |
| | GQPREPQVYTLPPSREEMTKNQVSLTCLV | | ASVVCLLNNFY | |
| | KGFYPSDIAVEWESNGQPENNYKTTPPVL | | PREAKVQWKV | |
| | DSDGSFFLYSKLTVDKSRWQQGNVFSCS | | DNALQSGNSQE | |
| | VMHEALHNHYTQKSLSLSLggggsggggsEIV | | SVTEQDSKDST | |
| | LTQSPGTLSLSPGERATLSCRASQGISRSYLA | | YSLSSTLTLSKA | |
| | WYQQKPGQAPSLLIYGASSRATGIPDRFSGS | | DYEKHKVYAC | |
| | GSGTDFTLTISRLEPEDFAVYYCQQFGSSPW | | EVTHQGLSSPV | |
| | TFGQGTKVEIKggggsggggsggggsggggsQVQL | | TKSFNRGEC | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_16 | QVQLQQSGSELKKPGASVKVSCKASGYT | 95 | DIQLTQSPSSLS | 8 |
| (αTROP 2 × | FTNYGMNWVKQAPGQGLKWMGWINTY | | ASVGDRVSITC | |
| αCD33 in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with Impaired | STAALGCLVKDYFPEPVTVSWNSGALTS | | GVPDRFSGSGS | |
| Fc) | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPEAAGGSSVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPASIEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggs | | KDSTYSLSTLT | |
| | ggggsEVQLVQSGAEVKKPGSSVKVSCKASGY | | LSKADYEKHKV | |
| | TITDSNIHWVRQAPGQSLEWIGYIYPYNGGT | | YACEVTHQGLS | |
| | DYNQKFKNRATLTVDNPTNTAYMELSSLRSE | | SPVTKSFNRGE | |
| | DTAFYYCVNGNPWLAYWGQGTLVTVSSgggg | | C | |
| | sggggsggggsggggsDIQLTQSPSTLSASVGDRV | | | |
| | TITCRASESLDNYGIRFLTWFQQKPGKAPKL | | | |
| | LMYAASNQGSGVPSRFSGSGSGTEFTLTISSL | | | |
| | QPDDFATYYCQQTKEVPWSFGQGTKVEVK | | | |
| DABA_17 | QVQLQQSGSELKKPGASVKVSCKASGYT | 96 | DIQLTQSPSSLS | 8 |
| (αTROP2 × | FTNYGMNWVKQAPGQGLKWMGWINTY | | ASVGDRVSITC | |
| αCSF1R in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with impaired | STAALGCLVKDYFPEPVTVSWNSGALTS | | GVPDRFSGSGS | |
| Fc) | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPEAAGGSSVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPASIEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggs | | KDSTYSLSSTLT | |
| | ggggsQVQLVQSGAEVKKPGSSVKVSCKASG | | LSKADYEKHKV | |
| | YTFTDNYMIWVRQAPGQGLEWMGDINPYN | | YACEVTHQGLS | |
| | GGTTFNQKFKGRVTITADKSTSTAYMELSSL | | SPVTKSFNRGE | |
| | RSEDTAVYYCARESPYFSNLYVMDYWGQGT | | C | |
| | LVTVSSggggsggggsggggsggggsEIVLTQSPAT | | | |
| | LSLSPGERATLSCKASQSVDYDGDNYMNWY | | | |
| | QQKPGQAPRLLIYAASNLESGIPARFSGSGS | | | |
| | GTDFTLTISSLEPEDFAVYYCHLSNEDLSTFG | | | |
| | GGTKVEIK | | | |
| DABA_18 | QVQLQQSGSELKKPGASVKVSCKASGYT | 97 | DIQLTQSPSSLS | 8 |
| (αTROP2 × | FTNYGMNWVKQAPGQGLKWMGWINTY | | ASVGDRVSITC | |
| αCD163 in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with impaired | STAALGCLVKDYFPEPVTVSWNSGALTS | | GVPDRFSGSGS | |
| Fc) | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPEAAGGSSVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPASIEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggs | | KDSTYSLSSTLT | |
| | ggggsQVQLQESGPGLVKPSETLSLTCTVSGY | | LSKADYEKHKV | |
| | SITSDYAWNWIRQFPGNKLEWMGYITYSGST | | YACEVTHQGLS | |
| | YYNPSLKSRVTISVDTSKNQFSLKLSSVTAAD | | SPVTKSFNRGE | |
| | TATYYCVSGTYYFDYWGQGTTLTVSSggggsg | | C | |
| | gggsggggsggggsDIVMTQSPSSLSASVGDRVTI | | | |
| | TCRASQSVSSDVAWFQQKPGKSPKPLIYYAS | | | |
| | NRYSGVPSRFSGSGSGTDFTLTISSLQAEDFA | | | |
| | VYFCGQDYTSPRTFGGGTKLEIK | | | |
| DABA_19 | QVQLQQSGSELKKPGASVKVSCKASGYT | 98 | DIQLTQSPSSLS | 8 |
| (αTROP2 × | FTNYGMNWVKQAPGQGLKWMGWINTY | | ASVGDRVSITC | |
| αDR5 in IgG1 | TGEPTYTDDFKGRFAFSLDTSVSTAYLQIS | | KASQDVSIAVA | |
| Backbone | SLKADDTAVYFCARGGFGSSYWYFDVW | | WYQQKPGKAP | |
| with impaired | GQGSLVTVSSASTKGPSVFPLAPCSRSTSE | | KLLIYSASYRYT | |
| Fc) | STAALGCLVKDYFPEPVTVSWNSGALTS | | GVPDRFSGSGS | |
| | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPEAAGGSSVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPASIEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggs | | KDSTYSLSSTLT | |
| | ggggsEIVLTQSPGTLSLSPGERATLSCRASQG | | LSKADYEKHKV | |
| | ISRSYLAWYQQKPGQAPSLLIYGASSRATGIP | | YACEVTHQGLS | |
| | DRFSGSGSGTDFTLTISRLEPEDFAVYYCQQ | | SPVTKSFNRGE | |
| | FGSSPWTFGQGTKVEIKggggsggggsggggsgg | | C | |
| | ggsQVQLQESGPGLVKPSQTLSLTCTVSGGSI | | | |
| | SSGDYFWSWIRQLPGKGLEWIGHIHNSGTT | | | |
| | YYNPSLKSRVTISVDTSKKQFSLRLSSVTAAD | | | |
| | TAVYYCARDRGGDYYYGMDVWGQGTTVTV | | | |
| | SS | | | |
| DABA_20 | QVQLVQSGAEVKKPGASVKVSCKASGYT | 99 | DVVMTQSPLSL | 80 |
| (αGPC3 × | FTDYEMHWVRQAPGQGLEWMGALDPKT | | PVTPGEPASISC | |
| αCD33 in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone | TVSSASTKGPSVFPLAPSSKSTSGGTAALG | | PGQSPQLLIYKV | |
| with impaired | CLVKDYFPEPVTVSWNSGALTSGVHTFP | | SNRFSGVPDRFS | |
| Fc) | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPEAAGGSSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPASIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsEVQLVQ | | TEQDSKDSTYS | |
| | SGAEVKKPGSSVKVSCKASGYTITDSNIHWV | | LS STLTLSKAD | |
| | RQAPGQSLEWIGYIYPYNGGTDYNQKFKNR | | YEKHKVYACE | |
| | ATLTVDNPTNTAYMELSSLRSEDTAFYYCVN | | VTHQGLSSPVT | |
| | GNPWLAYWGQGTLVTVSSggggsggggsggggs | | KSFNRGEC | |
| | ggggsDIQLTQSPSTLSASVGDRVTITCRASES | | | |
| | LDNYGIRFLTWFQQKPGKAPKLLMYAASNQ | | | |
| | GSGVPSRFSGSGSGTEFTLTISSLQPDDFATY | | | |
| | YCQQTKEVPWSFGQGTKVEVK | | | |
| DABA_21 | QVQLVQSGAEVKKPGASVKVSCKASGYT | 100 | DVVMTQSPLSL | 80 |
| (αGPC3 × | FTDYEMHWVRQAPGQGLEWMGALDPKT | | PVTPGEPASISC | |
| αCSF1R in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone | TVSSASTKGPSVFPLAPSSKSTSGGTAALG | | PGQSPQLLIYKV | |
| with impaired | CLVKDYFPEPVTVSWNSGALTSGVHTFP | | SNRFSGVPDRFS | |
| Fc) | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPEAAGGSSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPASIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsQVQLV | | TEQDSKDSTYS | |
| | QSGAEVKKPGSSVKVSCKASGYTFTDNYMI | | LSSTLTLSKAD | |
| | WVRQAPGQGLEWMGDINPYNGGTTFNQK | | YEKHKVYACE | |
| | FKGRVTITADKSTSTAYMELSSLRSEDTAVYY | | VTHQGLSSPVT | |
| | CARESPYFSNLYVMDYWGQGTLVTVSSggggs | | KSFNRGEC | |
| | ggggsggggsggggsEIVLTQSPATLSLSPGERAT | | | |
| | LSCKASQSVDYDGDNYMNWYQQKPGQAPR | | | |
| | LLIYAASNLESGIPARFSGSGSGTDFTLTISSL | | | |
| | EPEDFAVYYCHLSNEDLSTFGGGTKVEIK | | | |
| DABA_22 | QVQLVQSGAEVKKPGASVKVSCKASGYT | 101 | DVVMTQSPLSL | 80 |
| (αGPC3 × | FTDYEMHWVRQAPGQGLEWMGALDPKT | | PVTPGEPASISC | |
| αCD163 in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone | TVSSASTKGPSVFPLAPSSKSTSGGTAALG | | PGQSPQLLIYKV | |
| with impaired | CLVKDYFPEPVTVSWNSGALTSGVHTFP | | SNRFSGVPDRFS | |
| Fc) | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPEAAGGSSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPASIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsQVQLQ | | TEQDSKDSTYS | |
| | ESGPGLVKPSETLSLTCTVSGYSITSDYAWN | | LSSTLTLSKAD | |
| | WIRQFPGNKLEWMGYITYSGSTYYNPSLKSR | | YEKHKVYACE | |
| | VTISVDTSKNQFSLKLSSVTAADTATYYCVSG | | VTHQGLSSPVT | |
| | TYYFDYWGQGTTLTVSSggggsggggsggggsggg | | KSFNRGEC | |
| | gsDIVMTQSPSSLSASVGDRVTITCRASQSVSS | | | |
| | DVAWFQQKPGKSPKPLIYYASNRYSGVPSRF | | | |
| | SGSGSGTDFTLTISSLQAEDFAVYFCGQDYT | | | |
| | SPRTFGGGTKLEIK | | | |
| DABA_23 | QVQLVQSGAEVKKPGASVKVSCKASGYT | 102 | DVVMTQSPLSL | 80 |
| (αGPC3 × | FTDYEMHWVRQAPGQGLEWMGALDPKT | | PVTPGEPASISC | |
| αDR5 in IgG1 | GDTAYSQKFKGRVTLTADKSTSTAYMEL | | RSSQSLVHSNR | |
| Backbone | SSLTSEDTAVYYCTRFYSYTYWGQGTLV | | NTYLHWYLQK | |
| with impaired | TVSSASTKGPSVFPLAPSSKSTSGGTAALG | | PGQSPQLLIYKV | |
| Fc) | CLVKDYFPEPVTVSWNSGALTSGVHTFP | | SNRFSGVPDRFS | |
| | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPEAAGGSSVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPASIEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsEIVLTQS | | TEQDSKDSTYS | |
| | PGTLSLSPGERATLSCRASQGISRSYLAWYQ | | LSSTLTLSKAD | |
| | QKPGQAPSLLIYGASSRATGIPDRFSGSGSG | | YEKHKVYACE | |
| | TDFTLTISRLEPEDFAVYYCQQFGSSPWTFG | | VTHQGLSSPVT | |
| | QGTKVEIKggggsggggsggggsggggsQVQLQES | | KSFNRGEC | |
| | GPGLVKPSQTLSLTCTVSGGSISSGDYFWSW | | | |
| | IRQLPGKGLEWIGHIHNSGTTYYNPSLKSRV | | | |
| | TISVDTSKKQFSLRLSSVTAADTAVYYCARDR | | | |
| | GGDYYYGMDVWGQGTTVTVSS | | | |
| DABA_24 | QVQLVQSGAEVVKPGASVKISCKASGYT | 103 | DIVLTQSPLSLA | 85 |
| (αFOLR1 × | FTGYFMNWVKQSPGQSLEWIGRIHPYDG | | VSLGQPAIISCK | |
| αCD33 in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone | TTVTVSSASTKGPSVFPLAPSSKSTSGGTA | | QQPRLLIYRAS | |
| with impaired | ALGCLVKDYFPEPVTVSWNSGALTSGVH | | NLEAGVPDRFS | |
| Fc) | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPEAAGGSSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPASIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsEV | | TEQDSKDSTYS | |
| | QLVQSGAEVKKPGSSVKVSCKASGYTITDSN | | LSSTLTLSKAD | |
| | IHWVRQAPGQSLEWIGYIYPYNGGTDYNQK | | YEKHKVYACE | |
| | FKNRATLTVDNPTNTAYMELSSLRSEDTAFY | | VTHQGLSSPVT | |
| | YCVNGNPWLAYWGQGTLVTVSSggggsggggs | | KSFNRGEC | |
| | ggggsggggsDIQLTQSPSTLSASVGDRVTITCR | | | |
| | ASESLDNYGIRFLTWFQQKPGKAPKLLMYA | | | |
| | ASNQGSGVPSRFSGSGSGTEFTLTISSLQPD | | | |
| | DFATYYCQQTKEVPWSFGQGTKVEVK | | | |
| DABA_25 | QVQLVQSGAEVVKPGASVKISCKASGYT | 104 | DIVLTQSPLSLA | 85 |
| (αFOLR1 × | FTGYFMNWVKQSPGQSLEWIGRIHPYDG | | VSLGQPAIISCK | |
| αCSF1R in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone | TTVTVSSASTKGPSVFPLAPSSKSTSGGTA | | QQPRLLIYRAS | |
| with impaired | ALGCLVKDYFPEPVTVSWNSGALTSGVH | | NLEAGVPDRFS | |
| Fc) | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPEAAGGSSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPASIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsQV | | TEQDSKDSTYS | |
| | QLVQSGAEVKKPGSSVKVSCKASGYTFTDN | | LSSTLTLSKAD | |
| | YMIWVRQAPGQGLEWMGDINPYNGGTTFN | | YEKHKVYACE | |
| | QKFKGRVTITADKSTSTAYMELSSLRSEDTA | | VTHQGLSSPVT | |
| | VYYCARESPYFSNLYVMDYWGQGTLVTVSSg | | KSFNRGEC | |
| | gggsggggsggggsggggsEIVLTQSPATLSLSPGE | | | |
| | RATLSCKASQSVDYDGDNYMNWYQQKPGQ | | | |
| | APRLLIYAASNLESGIPARFSGSGSGTDFTLTI | | | |
| | SSLEPEDFAVYYCHLSNEDLSTFGGGTKVEI | | | |
| | K | | | |
| DABA_26 | QVQLVQSGAEVVKPGASVKISCKASGYT | 105 | DIVLTQSPLSLA | 85 |
| (αFOLR1 × | FTGYFMNWVKQSPGQSLEWIGRIHPYDG | | VSLGQPAIISCK | |
| αCD163 in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone | TTVTVSSASTKGPSVFPLAPSSKSTSGGTA | | QQPRLLIYRAS | |
| with impaired | ALGCLVKDYFPEPVTVSWNSGALTSGVH | | NLEAGVPDRFS | |
| Fc) | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPEAAGGSSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPASIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsQV | | TEQDSKDSTYS | |
| | QLQESGPGLVKPSETLSLTCTVSGYSITSDYA | | LSSTLTLSKAD | |
| | WNWIRQFPGNKLEWMGYITYSGSTYYNPSL | | YEKHKVYACE | |
| | KSRVTISVDTSKIVQFSLKLSSVTAADTATYYC | | VTHQGLSSPVT | |
| | VSGTYYFDYWGQGTTLTVSSggggsggggsgggg | | KSFNRGEC | |
| | sggggsDIVMTQSPSSLSASVGDRVTITCRASQ | | | |
| | SVSSDVAWFQQKPGKSPKPLIYYASNRYSGV | | | |
| | PSRFSGSGSGTDFTLTISSLQAEDFAVYFCG | | | |
| | QDYTSPRTFGGGTKLEIK | | | |
| DABA_27 | QVQLVQSGAEVVKPGASVKISCKASGYT | 106 | DIVLTQSPLSLA | 85 |
| (αFOLR1 × | FTGYFMNWVKQSPGQSLEWIGRIHPYDG | | VSLGQPAIISCK | |
| αDR5 in IgG1 | DTFYNQKFQGKATLTVDKSSNTAHMELL | | ASQSVSFAGTS | |
| Backbone | SLTSEDFAVYYCTRYDGSRAMDYWGQG | | LMHWYHQKPG | |
| with impaired | TTVTVSSASTKGPSVFPLAPSSKSTSGGTA | | QQPRLLIYRAS | |
| Fc) | ALGCLVKDYFPEPVTVSWNSGALTSGVH | | NLEAGVPDRFS | |
| | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPEAAGGSSVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPASIEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsEIV | | TEQDSKDSTYS | |
| | LTQSPGTLSLSPGERATLSCRASQGISRSYLA | | LSSTLTLSKAD | |
| | WYQQKPGQAPSLLIYGASSRATGIPDRFSGS | | YEKHKVYACE | |
| | GSGTDFTLTISRLEPEDFAVYYCQQFGSSPW | | VTHQGLSSPVT | |
| | TFGQGTKVEIKggggsggggsggggsggggsQVQL | | KSFNRGEC | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_28 | EVQLVQSGAEVKKPGSSVKVSCKASGYTI | 107 | DIQLTQSPSTLS | 90 |
| (αCD33 × | TDSNIHWVRQAPGQSLEWIGYIYPYNGGT | | ASVGDRVTITC | |
| αDR5 in IgG1 | DYNQKFKNRATLTVDNPTNTAYMELSSL | | RASESLDNYGI | |
| Backbone | RSEDTAFYYCVNGNPWLAYWGQGTLVT | | RFLTWFQQKPG | |
| with impaired | VSSASTKGPSVFPLAPSSKSTSGGTAALGC | | KAPKLLMYAAS | |
| Fc) | LVKDYFPEPVTVSWNSGALTSGVHTFPA | | NQGSGVPSRFS | |
| | VLQSSGLYSLSSVVTVPSSSLGTQTYICNV | | GSGSGTEFTLTI | |
| | NHKPSNTKVDKRVEPKSCDKTHTCPPCP | | SSLQPDDFATY | |
| | APEAAGGSSVFLFPPKPKDTLMISRTPEVT | | YCQQTKEVPWS | |
| | CVVVDVSHEDPEVKFNWYVDGVEVHNA | | FGQGTKVEVKR | |
| | KTKPREEQYNSTYRVVSVLTVLHQDWLN | | TVAAP SVFIFPP | |
| | GKEYKCKVSNKALPASIEKTISKAKGQPR | | SDEQLKSGTAS | |
| | EPQVYTLPPSREEMTKNQVSLTCLVKGFY | | VVCLLNNFYPR | |
| | PSDIAVEWESNGQPENNYKTTPPVLDSDG | | EAKVQWKVDN | |
| | SFFLYSKLTVDKSRWQQGNVFSCSVMHE | | ALQSGNSQESV | |
| | ALHNHYTQKSLSLSLggggsggggsEIVLTQSP | | TEQDSKDSTYS | |
| | GTLSLSPGERATLSCRASQGISRSYLAWYQQ | | LSSTLTLSKAD | |
| | KPGQAPSLLIYGASSRATGIPDRFSGSGSGT | | YEKHKVYACE | |
| | DFTLTISRLEPEDFAVYYCQQFGSSPWTFGQ | | VTHQGLS SPVT | |
| | GTKVEIKggggsggggsggggsggggsQVQLQESG | | KSFNRGEC | |
| | PGLVKPSQTLSLTCTVSGGSISSGDYFWSWIR | | | |
| | QLPGKGLEWIGHIHNSGTTYYNPSLKSRVTIS | | | |
| | VDTSKKQFSLRLSSVTAADTAVYYCARDRGG | | | |
| | DYYYGMDVWGQGTTVTVSS | | | |
| DABA_29 | EVQLLESGGGLVQPGGSLRLSCAVSGFTF | 108 | EIVLTQSPATLS | 92 |
| (αCD38 × | NSFAMSWVRQAPGKGLEWVSAISGSGGG | | LSPGERATLSCR | |
| αDR5 in IgG1 | TYYADSVKGRFTISRDNSKNTLYLQMNS | | ASQSVSSYLAW | |
| Backbone | LRAEDTAVYFCAKDKILWFGEPVFDYWG | | YQQKPGQAPRL | |
| with impaired | QGTLVTVSSASTKGPSVFPLAPSSKSTSGG | | LIYDASNRATGI | |
| Fc) | TAALGCLVKDYFPEPVTVSWNSGALTSG | | PARFSGSGSGT | |
| | VHTFPAVLQSSGLYSLSSVVTVPSSSLGTQ | | DFTLTISSLEPE | |
| | TYICNVNHKPSNTKVDKRVEPKSCDKTH | | DFAVYYCQQRS | |
| | TCPPCPAPEAAGGSSVFLFPPKPKDTLMIS | | NWPPTFGQGTK | |
| | RTPEVTCVVVDVSHEDPEVKFNWYVDG | | VEIKRTVAAPS | |
| | VEVHNAKTKPREEQYNSTYRVVSVLTVL | | VFIFPPSDEQLK | |
| | HQDWLNGKEYKCKVSNKALPASIEKTIS | | SGTASVVCLLN | |
| | KAKGQPREPQVYTLPPSREEMTKNQVSL | | NFYPREAKVQ | |
| | TCLVKGFYPSDIAVEWESNGQPENNYKT | | WKVDNALQSG | |
| | TPPVLDSDGSFFLYSKLTVDKSRWQQGN | | NSQESVTEQDS | |
| | VFSCSVMHEALHNHYTQKSLSLSLggggsg | | KDSTYSLSSTLT | |
| | gggsEIVLTQSPGTLSLSPGERATLSCRASQGI | | LSKADYEKHKV | |
| | SRSYLAWYQQKPGQAPSLLIYGASSRATGIP | | YACEVTHQGLS | |
| | DRFSGSGSGTDFTLTISRLEPEDFAVYYCQQ | | SPVTKSFNRGE | |
| | FGSSPWTFGQGTKVEIKggggsggggsggggsgg | | C | |
| | ggsQVQLQESGPGLVKPSQTLSLTCTVSGGSI | | | |
| | SSGDYFWSWIRQLPGKGLEWIGHIHNSGTT | | | |
| | YYNPSLKSRVTISVDTSKKQFSLRLSSVTAAD | | | |
| | TAVYYCARDRGGDYYYGMDVWGQGTTVTV | | | |
| | SS | | | |
| DABA_30 | EVQLVQSGAEVKKPGASVKVSCKASGYT | 109 | DVVMTQSPLSL | 94 |
| (αFLT3 × | FTSYYMHWVRQAPGQGLEWMGIINPSGG | | PVTPGEPASISC | |
| αDR5 in IgG1 | STSYAQKFQGRVTMTRDTSTSTVYMELS | | RSSQSLLHSNG | |
| Backbone | SLRSEDTAVYYCARGVGAHDAFDIWGQG | | NNYLDWYLQK | |
| with impaired | TTVTVSSASTKGPSVFPLAPSSKSTSGGTA | | PGQSPQLLIYLG | |
| Fc) | ALGCLVKDYFPEPVTVSWNSGALTSGVH | | SNRASGVPDRF | |
| | TFPAVLQSSGLYSLSSVVTVPSSSLGTQTY | | SGSGSDTDFTL | |
| | ICNVNHKPSNTKVDKRVEPKSCDKTHTCP | | QISRVEAEDVG | |
| | PCPAPEAAGGSSVFLFPPKPKDTLMISRTP | | VYYCMQGTHP | |
| | EVTCVVVDVSHEDPEVKFNWYVDGVEV | | AISFGQGTRLEI | |
| | HNAKTKPREEQYNSTYRVVSVLTVLHQD | | KRTVAAPSVFIF | |
| | WLNGKEYKCKVSNKALPASIEKTISKAK | | PPSDEQLKSGT | |
| | GQPREPQVYTLPPSREEMTKNQVSLTCLV | | ASVVCLLNNFY | |
| | KGFYPSDIAVEWESNGQPENNYKTTPPVL | | PREAKVQWKV | |
| | DSDGSFFLYSKLTVDKSRWQQGNVFSCS | | DNALQSGNSQE | |
| | VMHEALHNHYTQKSLSLSLggggsggggsEIV | | SVTEQDSKDST | |
| | LTQSPGTLSLSPGERATLSCRASQGISRSYLA | | YSLSSTLTLSKA | |
| | WYQQKPGQAPSLLIYGASSRATGIPDRFSGS | | DYEKHKVYAC | |
| | GSGTDFTLTISRLEPEDFAVYYCQQFGSSPW | | EVTHQGLSSPV | |
| | TFGQGTKVEIKggggsggggsggggsggggsQVQL | | TKSFNRGEC | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_31 | QVQLQQSGSELKKPGASVKVSCKASGYT | 110 | DIQLTQSPSSLS | 8 |
| (αTROP2 × | FTNYGMNWVKQAPGQGLKWMGWINTY | | ASVGDRVSITC | |
| αCD33 in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with ADCC | STAALGCLVKDYFPEPVTVSWNSGALTS | | GVPDRFSGSGS | |
| Enhanced) | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPELLGGPDVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPLPEEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggg | | KDSTYSLSSTLT | |
| | sggggsEVQLVQSGAEVKKPGSSVKVSCKASG | | LSKADYEKHKV | |
| | YTITDSNIHWVRQAPGQSLEWIGYIYPYNGG | | YACEVTHQGLS | |
| | TDYNQKFKNRATLTVDNPTNTAYMELSSLRS | | SPVTKSFNRGE | |
| | EDTAFYYCVNGNPWLAYWGQGTLVTVSSgg | | C | |
| | ggsggggsggggsggggsDIQLTQSPSTLSASVGD | | | |
| | RVTITCRASESLDNYGIRFLTWFQQKPGKAP | | | |
| | KLLMYAASNQGSGVPSRFSGSGSGTEFTLTI | | | |
| | SSLQPDDFATYYCQQTKEVPWSFGQGTKVE | | | |
| | VK | | | |
| DABA_32 | QVQLQQSGSELKKPGASVKVSCKASGYT | 111 | DIQLTQSPSSLS | 8 |
| (αTROP2 × | FTNYGMNWVKQAPGQGLKWMGWINTY | | ASVGDRVSITC | |
| αCSF1R in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with ADCC | STAALGCLVKDYFPEPVTVSWNSGALTS | | GVPDRFSGSGS | |
| Enhanced) | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPELLGGPDVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPLPEEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggs | | KDSTYSLSSTLT | |
| | ggggsQVQLVQSGAEVKKPGSSVKVSCKASG | | LSKADYEKHKV | |
| | YTFTDNYMIWVRQAPGQGLEWMGDINPYN | | YACEVTHQGLS | |
| | GGTTFNQKFKGRVTITADKSTSTAYMELSSL | | SPVTKSFNRGE | |
| | RSEDTAVYYCARESPYFSNLYVMDYWGQGT | | C | |
| | LVTVSSggggsggggsggggsggggsEIVLTQSPAT | | | |
| | LSLSPGERATLSCKASQSVDYDGDNYMNWY | | | |
| | QQKPGQAPRLLIYAASNLESGIPARFSGSGS | | | |
| | GTDFTLTISSLEPEDFAVYYCHLSNEDLSTFG | | | |
| | GGTKVEIK | | | |
| DABA_33 | QVQLQQSGSELKKPGASVKVSCKASGYT | 112 | DIQLTQSPSSLS | 8 |
| (αTROP2 × | FTNYGMNWVKQAPGQGLKWMGWINTY | | ASVGDRVSITC | |
| αCD163 in | TGEPTYTDDFKGRFAFSLDTSVSTAYLQIS | | KASQDVSIAVA | |
| IgG1 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with ADCC | STAALGCLVKDYFPEPVTVSWNSGALTS | | GVPDRFSGSGS | |
| Enhanced) | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPELLGGPDVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPLPEEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggs | | KDSTYSLSSTLT | |
| | ggggsQVQLQESGPGLVKPSETLSLTCTVSGY | | LSKADYEKHKV | |
| | SITSDYAWNWIRQFPGNKLEWMGYITYSGST | | YACEVTHQGLS | |
| | YYNPSLKSRVTISVDTSKNQFSLKLSSVTAAD | | SPVTKSFNRGE | |
| | TATYYCVSGTYYFDYWGQGTTLTVSSggggsg | | C | |
| | gggsggggsggggsDIVMTQSPSSLSASVGDRVTI | | | |
| | TCRASQSVSSDVAWFQQKPGKSPKPLIYYAS | | | |
| | NRYSGVPSRFSGSGSGTDFTLTISSLQAEDFA | | | |
| | VYFCGQDYTSPRTFGGGTKLEIK | | | |
| DABA_34 | QVQLQQSGSELKKPGASVKVSCKASGYT | 113 | DIQLTQSPSSLS | 8 |
| (αTROP2 × | FTNYGMNWVKQAPGQGLKWMGWINTY | | ASVGDRVSITC | |
| αDR5 in IgG1 | TGEPTYTDDFKGRFAFSLDTSVSTAYLQIS | | KASQDVSIAVA | |
| Backbone | SLKADDTAVYFCARGGFGSSYWYFDVW | | WYQQKPGKAP | |
| with ADCC | GQGSLVTVSSASTKGPSVFPLAPCSRSTSE | | KLLIYSASYRYT | |
| Enhanced) | STAALGCLVKDYFPEPVTVSWNSGALTS | | GVPDRFSGSGS | |
| | GVHTFPAVLQSSGLYSLSSVVTVPSSNFG | | GTDFTLTISSLQ | |
| | TQTYICNVNHKPSNTKVDKRVEPKSCDK | | PEDFAVYYCQQ | |
| | THTCPPCPAPELLGGPDVFLFPPKPKDTL | | HYITPLTFGAGT | |
| | MISRTPEVTCVVVDVSHEDPEVKFNWYV | | KVEIKRTVAAP | |
| | DGVEVHNAKTKPREEQYNSTYRVVSVLT | | SVFIFPPSDEQL | |
| | VLHQDWLNGKEYKCKVSNKALPLPEEKT | | KSGTASVVCLL | |
| | ISKAKGQPREPQVYTLPPSREEMTKNQVS | | NNFYPREAKVQ | |
| | LTCLVKGFYPSDIAVEWESNGQPENNYK | | WKVDNALQSG | |
| | TTPPVLDSDGSFFLYSKLTVDKSRWQQG | | NSQESVTEQDS | |
| | NVFSCSVMHEALHNHYTQKSLSLSLggggs | | KDSTYSLSSTLT | |
| | ggggsEIVLTQSPGTLSLSPGERATLSCRASQG | | LSKADYEKHKV | |
| | ISRSYLAWYQQKPGQAPSLLIYGASSRATGIP | | YACEVTHQGLS | |
| | DRFSGSGSGTDFTLTISRLEPEDFAVYYCQQ | | SPVTKSFNRGE | |
| | FGSSPWTFGQGTKVEIKggggsggggsggggsgg | | C | |
| | ggsQVQLQESGPGLVKPSQTLSLTCTVSGGSI | | | |
| | SSGDYFWSWIRQLPGKGLEWIGHIHNSGTT | | | |
| | YYNPSLKSRVTISVDTSKKQFSLRLSSVTAAD | | | |
| | TAVYYCARDRGGDYYYGMDVWGQGTTVTV | | | |
| | SS | | | |
| DABA_35 | QVQLVQSGAEVKKPGASVKVSCKASGYT | 114 | DVVMTQSPLSL | 80 |
| (αGPC3 × | FTDYEMHWVRQAPGQGLEWMGALDPKT | | PVTPGEPASISC | |
| αCD33 in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone | TVSSASTKGPSVFPLAPSSKSTSGGTAALG | | PGQSPQLLIYKV | |
| with ADCC | CLVKDYFPEPVTVSWNSGALTSGVHTFP | | SNRFSGVPDRFS | |
| Enhanced) | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPELLGGPDVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPLPEEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsEVQLVQ | | TEQDSKDSTYS | |
| | SGAEVKKPGSSVKVSCKASGYTITDSNIHWV | | LSSTLTLSKAD | |
| | RQAPGQSLEWIGYIYPYNGGTDYNQKFKNR | | YEKHKVYACE | |
| | ATLTVDNPTNTAYMELSSLRSEDTAFYYCVN | | VTHQGLSSPVT | |
| | GNPWLAYWGQGTLVTVSSggggsggggsggggs | | KSFNRGEC | |
| | ggggsDIQLTQSPSTLSASVGDRVTITCRASES | | | |
| | LDNYGIRFLTWFQQKPGKAPKLLMYAASNQ | | | |
| | GSGVPSRFSGSGSGTEFTLTISSLQPDDFATY | | | |
| | YCQQTKEVPWSFGQGTKVEVK | | | |
| DABA_36 | QVQLVQSGAEVKKPGASVKVSCKASGYT | 115 | DVVMTQSPLSL | 80 |
| (αGPC3 × | FTDYEMHWVRQAPGQGLEWMGALDPKT | | PVTPGEPASISC | |
| αCSF1R in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone | TVSSASTKGPSVFPLAPSSKSTSGGTAALG | | PGQSPQLLIYKV | |
| with ADCC | CLVKDYFPEPVTVSWNSGALTSGVHTFP | | SNRFSGVPDRFS | |
| Enhanced) | AVLQSSGLYSLSSVVTVPSSSLGTQTYICN | | GSGSGTDFTLKI | |
| | VNHKPSNTKVDKRVEPKSCDKTHTCPPC | | SRVEAEDVGVY | |
| | PAPELLGGPDVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPLPEEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsQVQLV | | TEQDSKDSTYS | |
| | QSGAEVKKPGSSVKVSCKASGYTFTDNYMI | | LSSTLTLSKAD | |
| | WVRQAPGQGLEWMGDINPYNGGTTFNQK | | YEKHKVYACE | |
| | FKGRVTITADKSTSTAYMELSSLRSEDTAVYY | | VTHQGLSSPVT | |
| | CARESPYFSNLYVMDYWGQGTLVTVSSggggs | | KSFNRGEC | |
| | ggggsggggsggggsEIVLTQSPATLSLSPGERAT | | | |
| | LSCKASQSVDYDGDNYMNWYQQKPGQAPR | | | |
| | LLIYAASNLESGIPARFSGSGSGTDFTLTISSL | | | |
| | EPEDFAVYYCHLSNEDLSTFGGGTKVEIK | | | |
| DABA_37 | QVQLVQSGAEVKKPGASVKVSCKASGYT | 116 | DVVMTQSPLSL | 80 |
| (αGPC3 × | FTDYEMHWVRQAPGQGLEWMGALDPKT | | PVTPGEPASISC | |
| αCD163 in | GDTAYSQKFKGRVTLTADKSTSTAYMEL | | RSSQSLVHSNR | |
| IgG1 | SSLTSEDTAVYYCTRFYSYTYWGQGTLV | | NTYLHWYLQK | |
| Backbone | TVSSASTKGPSVFPLAPSSKSTSGGTAALG | | PGQSPQLLIYKV | |
| with ADCC | CLVKDYFPEPVTVSWNSGALTSGVHTFP | | SNRFSGVPDRFS | |
| Enhanced) | AVLQSSGLYSLSSVVTVPSSSLGTQTYICN | | GSGSGTDFTLKI | |
| | VNFIKPSNTKVDKRVEPKSCDKTHTCPPC | | SRVEAEDVGVY | |
| | PAPELLGGPDVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPLPEEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsQVQLQ | | TEQDSKDSTYS | |
| | ESGPGLVKPSETLSLTCTVSGYSITSDYAWN | | LSSTLTLSKAD | |
| | WIRQFPGNKLEWMGYITYSGSTYYNPSLKSR | | YEKHKVYACE | |
| | VTISVDTSKNQFSLKLSSVTAADTATYYCVSG | | VTHQGLSSPVT | |
| | TYYFDYWGQGTTLTVSSggggsggggsggggsggg | | KSFNRGEC | |
| | gsDIVMTQSPSSLSASVGDRVTITCRASQSVSS | | | |
| | DVAWFQQKPGKSPKPLIYYASNRYSGVPSRF | | | |
| | SGSGSGTDFTLTISSLQAEDFAVYFCGQDYT | | | |
| | SPRTFGGGTKLEIK | | | |
| DABA_38 | QVQLVQSGAEVKKPGASVKVSCKASGYT | 117 | DVVMTQSPLSL | 80 |
| (αGPC3 × | FTDYEMHWVRQAPGQGLEWMGALDPKT | | PVTPGEPASISC | |
| αDR5 in IgG1 | GDTAYSQKFKGRVTLTADKSTSTAYMEL | | RS QSLVHSNR | |
| Backbone | SSLTSEDTAVYYCTRFYSYTYWGQGTLV | | NTYLHWYLQK | |
| with ADCC | TVSSASTKGPSVFPLAPSSKSTSGGTAALG | | PGQSPQLLIYKV | |
| Enhanced) | CLVKDYFPEPVTVSWNSGALTSGVHTFP | | SNRFSGVPDRFS | |
| | AVLQSSGLYSLSSVVTVPSSSLGTQTYICN | | GSGSGTDFTLKI | |
| | VNFIKPSNTKVDKRVEPKSCDKTHTCPPC | | SRVEAEDVGVY | |
| | PAPELLGGPDVFLFPPKPKDTLMISRTPEV | | YCSQNTHVPPT | |
| | TCVVVDVSHEDPEVKFNWYVDGVEVHN | | FGQGTKLEIKR | |
| | AKTKPREEQYNSTYRVVSVLTVLHQDWL | | TVAAPSVFIFPP | |
| | NGKEYKCKVSNKALPLPEEKTISKAKGQP | | SDEQLKSGTAS | |
| | REPQVYTLPPSREEMTKNQVSLTCLVKGF | | VVCLLNNFYPR | |
| | YPSDIAVEWESNGQPENNYKTTPPVLDSD | | EAKVQWKVDN | |
| | GSFFLYSKLTVDKSRWQQGNVFSCSVMH | | ALQSGNSQESV | |
| | EALHNHYTQKSLSLSLggggsggggsEIVLTQS | | TEQDSKDSTYS | |
| | PGTLSLSPGERATLSCRASQGISRSYLAWYQ | | LSSTLTLSKAD | |
| | QKPGQAPSLLIYGASSRATGIPDRFSGSGSG | | YEKHKVYACE | |
| | TDFTLTISRLEPEDFAVYYCQQFGSSPWTFG | | VTHQGLSSPVT | |
| | QGTKVEIKggggsggggsggggsggggsQVQLQES | | KSFNRGEC | |
| | GPGLVKPSQTLSLTCTVSGGSISSGDYFWSW | | | |
| | IRQLPGKGLEWIGHIHNSGTTYYNPSLKSRV | | | |
| | TISVDTSKKQFSLRLSSVTAADTAVYYCARDR | | | |
| | GGDYYYGMDVWGQGTTVTVSS | | | |
| DABA_39 | QVQLVQSGAEVVKPGASVKISCKASGYT | 118 | DIVLTQSPLSLA | 85 |
| (αFOLR1 × | FTGYFMNWVKQSPGQSLEWIGRIHPYDG | | VSLGQPAIISCK | |
| αCD33 in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone | TTVTVSSASTKGPSVFPLAPSSKSTSGGTA | | QQPRLLIYRAS | |
| with ADCC | ALGCLVKDYFPEPVTVSWNSGALTSGVH | | NLEAGVPDRFS | |
| Enhanced) | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPDVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPLPEEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsEV | | TEQDSKDSTYS | |
| | QLVQSGAEVKKPGSSVKVSCKASGYTITDSN | | LSSTLTLSKAD | |
| | IHWVRQAPGQSLEWIGYIYPYNGGTDYNQK | | YEKHKVYACE | |
| | FKNRATLTVDNPTNTAYMELSSLRSEDTAFY | | VTHQGLSSPVT | |
| | YCVNGNPWLAYWGQGTLVTVSSggggsggggs | | KSFNRGEC | |
| | ggggsggggsDIQLTQSPSTLSASVGDRVTITCR | | | |
| | ASESLDNYGIRFLTWFQQKPGKAPKLLMYA | | | |
| | ASNQGSGVPSRFSGSGSGTEFTLTISSLQPD | | | |
| | DFATYYCQQTKEVPWSFGQGTKVEVK | | | |
| DABA_40 | QVQLVQSGAEVVKPGASVKISCKASGYT | 119 | DIVLTQSPLSLA | 85 |
| (αFOLR1 × | FTGYFMNWVKQSPGQSLEWIGRIHPYDG | | VSLGQPAIISCK | |
| αCSF1R in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone | TTVTVSSASTKGPSVFPLAPSSKSTSGGTA | | QQPRLLIYRAS | |
| with ADCC | ALGCLVKDYFPEPVTVSWNSGALTSGVH | | NLEAGVPDRFS | |
| Enhanced) | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPDVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPLPEEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsQV | | TEQDSKDSTYS | |
| | QLVQSGAEVKKPGSSVKVSCKASGYTFTDN | | LSSTLTLSKAD | |
| | YMIWVRQAPGQGLEWMGDINPYNGGTTFN | | YEKHKVYACE | |
| | QKFKGRVTITADKSTSTAYMELSSLRSEDTA | | VTHQGLSSPVT | |
| | VYYCARESPYFSNLYVMDYWGQGTLVTVSSg | | KSFNRGEC | |
| | gggsggggsggggsggggsEIVLTQSPATLSLSPGE | | | |
| | RATLSCKASQSVDYDGDNYMNWYQQKPGQ | | | |
| | APRLLIYAASNLESGIPARFSGSGSGTDFTLTI | | | |
| | SSLEPEDFAVYYCHLSNEDLSTFGGGTKVEI | | | |
| | K | | | |
| DABA_41 | QVQLVQSGAEVVKPGASVKISCKASGYT | 120 | DIVLTQSPLSLA | 85 |
| (αFOLR1 × | FTGYFMNWVKQSPGQSLEWIGRIHPYDG | | VSLGQPAIISCK | |
| αCD163 in | DTFYNQKFQGKATLTVDKSSNTAHMELL | | ASQSVSFAGTS | |
| IgG1 | SLTSEDFAVYYCTRYDGSRAMDYWGQG | | LMHWYHQKPG | |
| Backbone | TTVTVSSASTKGPSVFPLAPSSKSTSGGTA | | QQPRLLIYRAS | |
| with ADCC | ALGCLVKDYFPEPVTVSWNSGALTSGVH | | NLEAGVPDRFS | |
| Enhanced) | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPDVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPLPEEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsQV | | TEQDSKDSTYS | |
| | QLQESGPGLVKPSETLSLTCTVSGYSITSDYA | | LSSTLTLSKAD | |
| | WNWIRQFPGNKLEWMGYITYSGSTYYNPSL | | YEKHKVYACE | |
| | KSRVTISVDTSKNQFSLKLSSVTAADTATYYC | | VTHQGLSSPVT | |
| | VSGTYYFDYWGQGTTLTVSSggggsggggsgggg | | KSFNRGEC | |
| | sggggsDIVMTQSPSSLSASVGDRVTITCRASQ | | | |
| | SVSSDVAWFQQKPGKSPKPLIYYASNRYSGV | | | |
| | PSRFSGSGSGTDFTLTISSLQAEDFAVYFCG | | | |
| | QDYTSPRTFGGGTKLEIK | | | |
| DABA_42 | QVQLVQSGAEVVKPGASVKISCKASGYT | 121 | DIVLTQSPLSLA | 85 |
| (αFOLR1 × | FTGYFMNWVKQSPGQSLEWIGRIHPYDG | | VSLGQPAIISCK | |
| αDR5 in IgG1 | DTFYNQKFQGKATLTVDKSSNTAHMELL | | ASQSVSFAGTS | |
| Backbone | SLTSEDFAVYYCTRYDGSRAMDYWGQG | | LMHWYHQKPG | |
| with ADCC | TTVTVSSASTKGPSVFPLAPSSKSTSGGTA | | QQPRLLIYRAS | |
| Enhanced) | ALGCLVKDYFPEPVTVSWNSGALTSGVH | | NLEAGVPDRFS | |
| | TFPAVLQSSGLYSLSSVVTVPSSSLGTQTY | | GSGSKTDFTLTI | |
| | ICNVNHKPSNTKVDKRVEPKSCDKTHTCP | | SPVEAEDAATY | |
| | PCPAPELLGGPDVFLFPPKPKDTLMISRTP | | YCQQSREYPYT | |
| | EVTCVVVDVSHEDPEVKFNWYVDGVEV | | FGGGTKLEIKR | |
| | HNAKTKPREEQYNSTYRVVSVLTVLHQD | | TVAAPSVFIFPP | |
| | WLNGKEYKCKVSNKALPLPEEKTISKAK | | SDEQLKSGTAS | |
| | GQPREPQVYTLPPSREEMTKNQVSLTCLV | | VVCLLNNFYPR | |
| | KGFYPSDIAVEWESNGQPENNYKTTPPVL | | EAKVQWKVDN | |
| | DSDGSFFLYSKLTVDKSRWQQGNVFSCS | | ALQSGNSQESV | |
| | VMHEALHNHYTQKSLSLSLggggsggggsEIV | | TEQDSKDSTYS | |
| | LTQSPGTLSLSPGERATLSCRASQGISRSYLA | | LSSTLTLSKAD | |
| | WYQQKPGQAPSLLIYGASSRATGIPDRFSGS | | YEKHKVYACE | |
| | GSGTDFTLTISRLEPEDFAVYYCQQFGSSPW | | VTHQGLSSPVT | |
| | TFGQGTKVEIKggggsggggsggggsggggsQVQL | | KSFNRGEC | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_43 | EVQLVQSGAEVKKPGSSVKVSCKASGYTI | 122 | DIQLTQSPSTLS | 90 |
| (αCD33 × | TDSNIHWVRQAPGQSLEWIGYIYPYNGGT | | ASVGDRVTITC | |
| αDR5 in IgG1 | DYNQKFKNRATLTVDNPTNTAYMELSSL | | RASESLDNYGI | |
| Backbone | RSEDTAFYYCVNGNPWLAYWGQGTLVT | | RFLTWFQQKPG | |
| with ADCC | VSSASTKGPSVFPLAPCSRSTSESTAALGC | | KAPKLLMYAAS | |
| Enhanced) | LVKDYFPEPVTVSWNSGALTSGVHTFPA | | NQGSGVPSRFS | |
| | VLQSSGLYSLSSVVTVPSSNFGTQTYICNV | | GSGSGTEFTLTI | |
| | NHKPSNTKVDKRVEPKSCDKTHTCPPCP | | SSLQPDDFATY | |
| | APELLGGPDVFLFPPKPKDTLMISRTPEVT | | YCQQTKEVPWS | |
| | CVVVDVSHEDPEVKFNWYVDGVEVHNA | | FGQGTKVEVKR | |
| | KTKPREEQYNSTYRVVSVLTVLHQDWLN | | TVAAPSVFIFPP | |
| | GKEYKCKVSNKALPLPEEKTISKAKGQPR | | SDEQLKSGTAS | |
| | EPQVYTLPPSREEMTKNQVSLTCLVKGFY | | VVCLLNNFYPR | |
| | PSDIAVEWESNGQPENNYKTTPPVLDSDG | | EAKVQWKVDN | |
| | SFFLYSKLTVDKSRWQQGNVFSCSVMHE | | ALQSGNSQESV | |
| | ALHNHYTQKSLSLSLggggsggggsEIVLTQSP | | TEQDSKDSTYS | |
| | GTLSLSPGERATLSCRASQGISRSYLAWYQQ | | LSSTLTLSKAD | |
| | KPGQAPSLLIYGASSRATGIPDRFSGSGSGT | | YEKHKVYACE | |
| | DFTLTISRLEPEDFAVYYCQQFGSSPWTFGQ | | VTHQGLSSPVT | |
| | GTKVEIKggggsggggsggggsggggsQVQLQESG | | KSFNRGEC | |
| | PGLVKPSQTLSLTCTVSGGSISSGDYFWSWIR | | | |
| | QLPGKGLEWIGHIHNSGTTYYNPSLKSRVTIS | | | |
| | VDTSKKQFSLRLSSVTAADTAVYYCARDRGG | | | |
| | DYYYGMDVWGQGTTVTVSS | | | |
| DABA_44 | EVQLLESGGGLVQPGGSLRLSCAVSGFTF | 123 | EIVLTQSPATLS | 92 |
| (αCD38 × | NSFAMSWVRQAPGKGLEWVSAISGSGGG | | LSPGERATLSCR | |
| αDR5 in IgG1 | TYYADSVKGRFTISRDNSKNTLYLQMNS | | ASQSVSSYLAW | |
| Backbone | LRAEDTAVYFCAKDKILWFGEPVFDYWG | | YQQKPGQAPRL | |
| with ADCC | QGTLVTVSSASTKGPSVFPLAPSSKSTSGG | | LIYDASNRATGI | |
| Enhanced) | TAALGCLVKDYFPEPVTVSWNSGALTSG | | PARFSGSGSGT | |
| | VHTFPAVLQSSGLYSLSSVVTVPSSSLGTQ | | DFTLTISSLEPE | |
| | TYICNVNHKPSNTKVDKRVEPKSCDKTH | | DFAVYYCQQRS | |
| | TCPPCPAPELLGGPDVFLFPPKPKDTLMIS | | NWPPTFGQGTK | |
| | RTPEVTCVVVDVSHEDPEVKFNWYVDG | | VEIKRTVAAPS | |
| | VEVHNAKTKPREEQYNSTYRVVSVLTVL | | VFIFPPSDEQLK | |
| | HQDWLNGKEYKCKVSNKALPLPEEKTIS | | SGTASVVCLLN | |
| | KAKGQPREPQVYTLPPSREEMTKNQVSL | | NFYPREAKVQ | |
| | TCLVKGFYPSDIAVEWESNGQPENNYKT | | WKVDNALQSG | |
| | TPPVLDSDGSFFLYSKLTVDKSRWQQGN | | NSQESVTEQDS | |
| | VFSCSVMHEALHNHYTQKSLSLSLggggsg | | KDSTYSLSSTLT | |
| | gggsEIVLTQSPGTLSLSPGERATLSCRASQGI | | LSKADYEKHKV | |
| | SRSYLAWYQQKPGQAPSLLIYGASSRATGIP | | YACEVTHQGLS | |
| | DRFSGSGSGTDFTLTISRLEPEDFAVYYCQQ | | SPVTKSFNRGE | |
| | FGSSPWTFGQGTKVEIKggggsggggsggggsgg | | C | |
| | ggsQVQLQESGPGLVKPSQTLSLTCTVSGGSI | | | |
| | SSGDYFWSWIRQLPGKGLEWIGHIHNSGTT | | | |
| | YYNPSLKSRVTISVDTSKKQFSLRLSSVTAAD | | | |
| | TAVYYCARDRGGDYYYGMDVWGQGTTVTV | | | |
| | SS | | | |
| DABA_45 | EV Q LV Q SGAEVKKPGASVKVSCKASGYT | 124 | DVVMTQ SPLSL | 94 |
| (αFLT3 × | FTSYYMHWVRQAPGQGLEWMGIINPSGG | | PVTPGEPASISC | |
| αDR5 in IgG1 | STSYAQKFQGRVTMTRDTSTSTVYMELS | | RSSQSLLHSNG | |
| Backbone | SLRSEDTAVYYCARGVGAHDAFDIWGQG | | NNYLDWYLQK | |
| with ADCC | TTVTVSSASTKGPSVFPLAPSSKSTSGGTA | | PGQSPQLLIYLG | |
| Enhanced) | ALGCLVKDYFPEPVTVSWNSGALTSGVH | | SNRASGVPDRF | |
| | TFPAVLQSSGLYSLSSVVTVPSSSLGTQTY | | SGSGSDTDFTL | |
| | ICNVNHKPSNTKVDKRVEPKSCDKTHTCP | | QISRVEAEDVG | |
| | PCPAPELLGGPDVFLFPPKPKDTLMISRTP | | VYYCMQGTHP | |
| | EVTCVVVDVSHEDPEVKFNWYVDGVEV | | AISFGQGTRLEI | |
| | HNAKTKPREEQYNSTYRVVSVLTVLHQD | | KRTVAAPSVFIF | |
| | WLNGKEYKCKVSNKALPLPEEKTISKAK | | PPSDEQLKSGT | |
| | GQPREPQVYTLPPSREEMTKNQVSLTCLV | | ASVVCLLNNFY | |
| | KGFYPSDIAVEWESNGQPENNYKTTPPVL | | PREAKVQWKV | |
| | DSDGSFFLYSKLTVDKSRWQQGNVFSCS | | DNALQSGNSQE | |
| | VMHEALHNHYTQKSLSLSLggggsggggsEIV | | SVTEQDSKDST | |
| | LTQSPGTLSLSPGERATLSCRASQGISRSYLA | | YSLSSTLTLSKA | |
| | WYQQKPGQAPSLLIYGASSRATGIPDRFSGS | | DYEKHKVYAC | |
| | GSGTDFTLTISRLEPEDFAVYYCQQFGSSPW | | EVTHQGLSSPV | |
| | TFGQGTKVEIKggggsggggsggggsggggsQVQL | | TKSFNRGEC | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_46 | QVQLQQSGSELKKPGASVKVSCKASGYT | 125 | DIQLTQSPSSLS | 8 |
| (αTROP2 × | FTNYGMNWVKQAPGQGLKWMGWINTY | | ASVGDRVSITC | |
| αCD33 in | TGEPTYTDDFKGRFAFSLDTSVSTAYLQIS | | KASQDVSIAVA | |
| IgG4 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with S228P | STAALGCLVKDYFPEPVTVSWNSGALTS | | GVPDRFSGSGS | |
| Mutation) | GVHTFPAVLQSSGLYSLSSVVTVPSSSLGT | | GTDFTLTISSLQ | |
| | KTYTCNVDHKPSNTKVDKRVESKYGPPC | | PEDFAVYYCQQ | |
| | PPCPAPEFLGGPSVFLFPPKPKDTLMISRTP | | HYITPLTFGAGT | |
| | EVTCVVVDVSQEDPEVQFNWYVDGVEV | | KVEIKRTVAAP | |
| | HNAKTKPREEQFNSTYRVVSVLTVLHQD | | SVFIFPPSDEQL | |
| | WLNGKEYKCKVSNKGLPSSIEKTISKAKG | | KSGTASVVCLL | |
| | QPREPQVYTLPPSQEEMTKNQVSLTCLVK | | NNFYPREAKVQ | |
| | GFYPSDIAVEWESNGQPENNYKTTPPVLD | | WKVDNALQSG | |
| | SDGSFFLYSRLTVDKSRWQEGNVFSCSV | | NSQESVTEQDS | |
| | MHEALHNHYTQKSLSLSLggggsggggsEVQ | | KDSTYSLSSTLT | |
| | LVQSGAEVKKPGSSVKVSCKASGYTITDSNI | | LSKADYEKHKV | |
| | HWVRQAPGQSLEWIGYIYPYNGGTDYNQKF | | YACEVTHQGLS | |
| | KNRATLTVDNPTNTAYMELSSLRSEDTAFYY | | SPVTKSFNRGE | |
| | CVNGNPWLAYWGQGTLVTVSSggggsggggsg | | C | |
| | gggsggggsDIQLTQSPSTLSASVGDRVTITCRA | | | |
| | SESLDNYGIRFLTWFQQKPGKAPKLLMYAA | | | |
| | SNQGSGVPSRFSGSGSGTEFTLTISSLQPDD | | | |
| | FATYYCQQTKEVPWSFGQGTKVEVK | | | |
| DABA_47 | QVQLQQSGSELKKPGASVKVSCKASGYT | 126 | DIQLTQSPSSLS | 8 |
| (αTROP2 × | FTNYGMNWVKQAPGQGLKWMGWINTY | | ASVGDRVSITC | |
| αCSF1R in | TGEPTYTDDFKGRFAFSLDTSVSTAYLQIS | | KASQDVSIAVA | |
| IgG4 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with S228P | STAALGCLVKDYFPEPVTVSWNSGALTS | | GVPDRFSGSGS | |
| Mutation) | GVHTFPAVLQSSGLYSLSSVVTVPSSSLGT | | GTDFTLTISSLQ | |
| | KTYTCNVDHKPSNTKVDKRVESKYGPPC | | PEDFAVYYCQQ | |
| | PPCPAPEFLGGPSVFLFPPKPKDTLMISRTP | | HYITPLTFGAGT | |
| | EVTCVVVDVSQEDPEVQFNWYVDGVEV | | KVEIKRTVAAP | |
| | HNAKTKPREEQFNSTYRVVSVLTVLHQD | | SVFIFPPSDEQL | |
| | WLNGKEYKCKVSNKGLPSSIEKTISKAKG | | KSGTASVVCLL | |
| | QPREPQVYTLPPSQEEMTKNQVSLTCLVK | | NNFYPREAKVQ | |
| | GFYPSDIAVEWESNGQPENNYKTTPPVLD | | WKVDNALQSG | |
| | SDGSFFLYSRLTVDKSRWQEGNVFSCSV | | NSQESVTEQDS | |
| | MHEALHNHYTQKSLSLSLggggsggggsQVQ | | KDSTYSLSSTLT | |
| | LVQSGAEVKKPGSSVKVSCKASGYTFTDNY | | LSKADYEKHKV | |
| | MIWVRQAPGQGLEWMGDINPYNGGTTFNQ | | YACEVTHQGLS | |
| | KFKGRVTITADKSTSTAYMELSSLRSEDTAVY | | SPVTKSFNRGE | |
| | YCARESPYFSNLYVMDYWGQGTLVTVSSggg | | C | |
| | gsggggsggggsggggsEIVLTQSPATLSTSPGERA | | | |
| | TLSCKASQSVDYDGDNYMNWYQQKPGQAP | | | |
| | RLLIYAASNLESGIPARFSGSGSGTDFTLTISS | | | |
| | LEPEDFAVYYCHLSNEDLSTFGGGTKVEIK | | | |
| DABA_48 | QVQLQQSGSELKKPGASVKVSCKASGYT | 127 | DIQLTQSPSSLS | 8 |
| (αTROP2 × | FTNYGMNWVKQAPGQGLKWMGWINTY | | ASVGDRVSITC | |
| αCD163 in | TGEPTYTDDFKGRFAFSLDTSVSTAYLQIS | | KASQDVSIAVA | |
| IgG4 | SLKADDTAVYFCARGGFGSSYWYFDVW | | WYQQKPGKAP | |
| Backbone | GQGSLVTVSSASTKGPSVFPLAPCSRSTSE | | KLLIYSASYRYT | |
| with S228P | STAALGCLVKDYFPEPVTVSWNSGALTS | | GVPDRFSGSGS | |
| Mutation) | GVHTFPAVLQSSGLYSLSSVVTVPSSSLGT | | GTDFTLTISSLQ | |
| | KTYTCNVDHKPSNTKVDKRVESKYGPPC | | PEDFAVYYCQQ | |
| | PPCPAPEFLGGPSVFLFPPKPKDTLMISRTP | | HYITPLTFGAGT | |
| | EVTCVVVDVSQEDPEVQFNWYVDGVEV | | KVEIKRTVAAP | |
| | HNAKTKPREEQFNSTYRVVSVLTVLHQD | | SVFIFPPSDEQL | |
| | WLNGKEYKCKVSNKGLPSSIEKTISKAKG | | KSGTASVVCLL | |
| | QPREPQVYTLPPSQEEMTKNQVSLTCLVK | | NNFYPREAKVQ | |
| | GFYPSDIAVEWESNGQPENNYKTTPPVLD | | WKVDNALQSG | |
| | SDGSFFLYSRLTVDKSRWQEGNVFSCSV | | NSQESVTEQDS | |
| | MHEALHNHYTQKSLSLSLggggsggggsQVQ | | KDSTYSLSSTLT | |
| | LQESGPGLVKPSETLSLTCTVSGYSITSDYAW | | LSKADYEKHKV | |
| | NWIRQFPGNKLEWMGYITYSGSTYYNPSLKS | | YACEVTHQGLS | |
| | RVTISVDTSKIVQFSLKLSSVTAADTATYYCVS | | SPVTKSFNRGE | |
| | GTYYFDYWGQGTTLTVSSggggsggggsggggsg | | C | |
| | gggsDIVMTQSPSSLSASVGDRVTITCRASQSV | | | |
| | SSDVAWFQQKPGKSPKPLIYYASNRYSGVPS | | | |
| | RFSGSGSGTDFTLTISSLQAEDFAVYFCGQD | | | |
| | YTSPRTFGGGTKLEIK | | | |
| DABA_49 | QVQLQQSGSELKKPGASVKVSCKASGYT | 128 | DIQLTQSPSSLS | 8 |
| (αTROP2 × | FTNYGMNWVKQAPGQGLKWMGWINTY | | ASVGDRVSITC | |
| αDR5 in IgG4 | TGEPTYTDDFKGRFAFSLDTSVSTAYLQIS | | KASQDVSIAVA | |
| Backbone | SLKADDTAVYFCARGGFGSSYWYFDVW | | WYQQKPGKAP | |
| with S228P | GQGSLVTVSSASTKGPSVFPLAPCSRSTSE | | KLLIYSASYRYT | |
| Mutation) | STAALGCLVKDYFPEPVTVSWNSGALTS | | GVPDRFSGSGS | |
| | GVHTFPAVLQSSGLYSLSSVVTVPSSSLGT | | GTDFTLTISSLQ | |
| | KTYTCNVDHKPSNTKVDKRVESKYGPPC | | PEDFAVYYCQQ | |
| | PPCPAPEFLGGPSVFLFPPKPKDTLMISRTP | | HYITPLTFGAGT | |
| | EVTCVVVDVSQEDPEVQFNWYVDGVEV | | KVEIKRTVAAP | |
| | HNAKTKPREEQFNSTYRVVSVLTVLHQD | | SVFIFPPSDEQL | |
| | WLNGKEYKCKVSNKGLPSSIEKTISKAKG | | KSGTASVVCLL | |
| | QPREPQVYTLPPSQEEMTKNQVSLTCLVK | | NNFYPREAKVQ | |
| | GFYPSDIAVEWESNGQPENNYKTTPPVLD | | WKVDNALQSG | |
| | SDGSFFLYSRLTVDKSRWQEGNVFSCSV | | NSQESVTEQDS | |
| | MHEALHNHYTQKSLSLSLggggsggggsEIVL | | KDSTYSLSSTLT | |
| | TQSPGTLSLSPGERATLSCRASQGISRSYLAW | | LSKADYEKHKV | |
| | YQQKPGQAPSLLIYGASSRATGIPDRFSGSG | | YACEVTHQGLS | |
| | SGTDFTLTISRLEPEDFAVYYCQQFGSSPWT | | SPVTKSFNRGE | |
| | FGQGTKVEIKggggsggggsggggsggggsQVQL | | C | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_50 | QVQLVQSGAEVKKPGASVKVSCKASGYT | 129 | DVVMTQSPLSL | 80 |
| (αGPC3 × | FTDYEMHWVRQAPGQGLEWMGALDPKT | | PVTPGEPASISC | |
| αCD33 in | GDTAYSQKFKGRVTLTADKSTSTAYMEL | | RSSQSLVHSNR | |
| IgG4 | SSLTSEDTAVYYCTRFYSYTYWGQGTLV | | NTYLHWYLQK | |
| Backbone | TVSSASTKGPSVFPLAPCSRSTSESTAALG | | PGQSPQLLIYKV | |
| with S228P | CLVKDYFPEPVTVSWNSGALTSGVHTFP | | SNRFSGVPDRFS | |
| Mutation) | AVLQSSGLYSLSSVVTVPSSSLGTKTYTC | | GSGSGTDFTLKI | |
| | NVDHKPSNTKVDKRVESKYGPPCPPCPAP | | SRVEAEDVGVY | |
| | EFLGGPSVFLFPPKPKDTLMISRTPEVTCV | | YCSQNTHVPPT | |
| | VVDVSQEDPEVQFNWYVDGVEVHNAKT | | FGQGTKLEIKR | |
| | KPREEQFNSTYRVVSVLTVLHQDWLNGK | | TVAAPSVFIFPP | |
| | EYKCKVSNKGLPSSIEKTISKAKGQPREPQ | | SDEQLKSGTAS | |
| | VYTLPPSQEEMTKNQVSLTCLVKGFYPSD | | VVCLLNNFYPR | |
| | IAVEWESNGQPENNYKTTPPVLDSDGSFF | | EAKVQWKVDN | |
| | LYSRLTVDKSRWQEGNVFSCSVMHEALH | | ALQSGNSQESV | |
| | NHYTQKSLSLSLggggsggggsEVQLVQSGAE | | TEQDSKDSTYS | |
| | VKKPGSSVKVSCKASGYTITDSNIHWVRQAP | | LSSTLTLSKAD | |
| | GQSLEWIGYIYPYNGGTDYNQKFKNRATLTV | | YEKHKVYACE | |
| | DNPTNTAYMELSSLRSEDTAFYYCVNGNPW | | VTHQGLSSPVT | |
| | LAYWGQGTLVTVSSggggsggggsggggsggggsD | | KSFNRGEC | |
| | IQLTQSPSTLSASVGDRVTITCRASESLDNYGI | | | |
| | RFLTWFQQKPGKAPKLLMYAASNQGSGVPS | | | |
| | RFSGSGSGTEFTLTISSLQPDDFATYYCQQT | | | |
| | KEVPWSFGQGTKVEVK | | | |
| DABA_51 | QVQLVQSGAEVKKPGASVKVSCKASGYT | 130 | DVVMTQSPLSL | 80 |
| (αGPC3 × | FTDYEMHWVRQAPGQGLEWMGALDPKT | | PVTPGEPASISC | |
| αCSF1R in | GDTAYSQKFKGRVTLTADKSTSTAYMEL | | RSSQSLVHSNR | |
| IgG4 | SSLTSEDTAVYYCTRFYSYTYWGQGTLV | | NTYLHWYLQK | |
| Backbone | TVSSASTKGPSVFPLAPCSRSTSESTAALG | | PGQSPQLLIYKV | |
| with S228P | CLVKDYFPEPVTVSWNSGALTSGVHTFP | | SNRFSGVPDRFS | |
| Mutation) | AVLQSSGLYSLSSVVTVPSSSLGTKTYTC | | GSGSGTDFTLKI | |
| | NVDHKPSNTKVDKRVESKYGPPCPPCPAP | | SRVEAEDVGVY | |
| | EFLGGPSVFLFPPKPKDTLMISRTPEVTCV | | YCSQNTHVPPT | |
| | VVDVSQEDPEVQFNWYVDGVEVHNAKT | | FGQGTKLEIKR | |
| | KPREEQFNSTYRVVSVLTVLHQDWLNGK | | TVAAPSVFIFPP | |
| | EYKCKVSNKGLPSSIEKTISKAKGQPREPQ | | SDEQLKSGTAS | |
| | VYTLPPSQEEMTKNQVSLTCLVKGFYPSD | | VVCLLNNFYPR | |
| | IAVEWESNGQPENNYKTTPPVLDSDGSFF | | EAKVQWKVDN | |
| | LYSRLTVDKSRWQEGNVFSCSVMHEALH | | ALQSGNSQESV | |
| | NHYTQKSLSLSLggggsggggsQVQLVQSGAE | | TEQDSKDSTYS | |
| | VKKPGSSVKVSCKASGYTFTDNYMIWVRQA | | LSSTLTLSKAD | |
| | PGQGLEWMGDINPYNGGTTFNQKFKGRVT | | YEKHKVYACE | |
| | ITADKSTSTAYMELSSLRSEDTAVYYCARESP | | VTHQGLSSPVT | |
| | YFSNLYVMDYWGQGTLVTVSSggggsggggsgg | | KSFNRGEC | |
| | ggsggggsEIVLTQSPATLSLSPGERATLSCKAS | | | |
| | QSVDYDGDNYMNWYQQKPGQAPRLLIYAA | | | |
| | SNLESGIPARFSGSGSGTDFTLTISSLEPEDF | | | |
| | AVYYCHLSNEDLSTFGGGTKVEIK | | | |
| DABA_52 | QVQLVQSGAEVKKPGASVKVSCKASGYT | 131 | DVVMTQSPLSL | 80 |
| (αGPC3 × | FTDYEMHWVRQAPGQGLEWMGALDPKT | | PVTPGEPASISC | |
| αCD163 in | GDTAYSQKFKGRVTLTADKSTSTAYMEL | | RSSQSLVHSNR | |
| IgG4 | SSLTSEDTAVYYCTRFYSYTYWGQGTLV | | NTYLHWYLQK | |
| Backbone | TVSSASTKGPSVFPLAPCSRSTSESTAALG | | PGQSPQLLIYKV | |
| with S228P | CLVKDYFPEPVTVSWNSGALTSGVHTFP | | SNRFSGVPDRFS | |
| Mutation) | AVLQSSGLYSLSSVVTVPSSSLGTKTYTC | | GSGSGTDFTLKI | |
| | NVDHKPSNTKVDKRVESKYGPPCPPCPAP | | SRVEAEDVGVY | |
| | EFLGGPSVFLFPPKPKDTLMISRTPEVTCV | | YCSQNTHVPPT | |
| | VVDVSQEDPEVQFNWYVDGVEVHNAKT | | FGQGTKLEIKR | |
| | KPREEQFNSTYRVVSVLTVLHQDWLNGK | | TVAAPSVFIFPP | |
| | EYKCKVSNKGLPSSIEKTISKAKGQPREPQ | | SDEQLKSGTAS | |
| | VYTLPPSQEEMTKNQVSLTCLVKGFYPSD | | VVCLLNNFYPR | |
| | IAVEWESNGQPENNYKTTPPVLDSDGSFF | | EAKVQWKVDN | |
| | LYSRLTVDKSRWQEGNVFSCSVMHEALH | | ALQSGNSQESV | |
| | NHYTQKSLSLSLggggsggggsQVQLQESGPG | | TEQDSKDSTYS | |
| | LVKPSETLSLTCTVSGYSITSDYAWNWIRQFP | | LSSTLTLSKAD | |
| | GNKLEWMGYITYSGSTYYNPSLKSRVTISVDT | | YEKHKVYACE | |
| | SKNQFSLKLSSVTAADTATYYCVSGTYYFDY | | VTHQGLSSPVT | |
| | WGQGTTLTVSSggggsggggsggggsggggsDIVM | | KSFNRGEC | |
| | TQSPSSLSASVGDRVTITCRASQSVSSDVAWF | | | |
| | QQKPGKSPKPLIYYASNRYSGVPSRFSGSGS | | | |
| | GTDFTLTISSLQAEDFAVYFCGQDYTSPRTF | | | |
| | GGGTKLEIK | | | |
| DABA_53 | QV QLVQ SGAEVKKPGASVKVSCKASGYT | 132 | DVVMTQSPLSL | 80 |
| (αGPC3 × | FTDYEMHWVRQAPGQGLEWMGALDPKT | | PVTPGEPASISC | |
| αDR5 in IgG4 | GDTAYSQKFKGRVTLTADKSTSTAYMEL | | RSSQSLVHSNR | |
| Backbone | SSLTSEDTAVYYCTRFYSYTYWGQGTLV | | NTYLHWYLQK | |
| with S228P | TVSSASTKGPSVFPLAPCSRSTSESTAALG | | PGQSPQLLIYKV | |
| Mutation) | CLVKDYFPEPVTVSWNSGALTSGVHTFP | | SNRFSGVPDRFS | |
| | AVLQSSGLYSLSSVVTVPSSSLGTKTYTC | | GSGSGTDFTLKI | |
| | NVDHKPSNTKVDKRVESKYGPPCPPCPAP | | SRVEAEDVGVY | |
| | EFLGGPSVFLFPPKPKDTLMISRTPEVTCV | | YCSQNTHVPPT | |
| | VVDVSQEDPEVQFNWYVDGVEVHNAKT | | FGQGTKLEIKR | |
| | KPREEQFNSTYRVVSVLTVLHQDWLNGK | | TVAAPSVFIFPP | |
| | EYKCKVSNKGLPSSIEKTISKAKGQPREPQ | | SDEQLKSGTAS | |
| | VYTLPPSQEEMTKNQVSLTCLVKGFYPSD | | VVCLLNNFYPR | |
| | IAVEWESNGQPENNYKTTPPVLDSDGSFF | | EAKVQWKVDN | |
| | LYSRLTVDKSRWQEGNVFSCSVMHEALH | | ALQSGNSQESV | |
| | NHYTQKSLSLSLggggsggggsEIVLTQSPGTL | | TEQDSKDSTYS | |
| | SLSPGERATLSCRASQGISRSYLAWYQQKPG | | LSSTLTLSKAD | |
| | QAPSLLIYGASSRATGIPDRFSGSGSGTDFTL | | YEKHKVYACE | |
| | TISRLEPEDFAVYYCQQFGSSPWTFGQGTK | | VTHQGLSSPVT | |
| | VEIKggggsggggsggggsggggsQVQLQESGPGL | | KSFNRGEC | |
| | VKPSQTLSLTCTVSGGSISSGDYFWSWIRQLP | | | |
| | GKGLEWIGHIHNSGTTYYNPSLKSRVTISVDT | | | |
| | SKKQFSLRLSSVTAADTAVYYCARDRGGDYY | | | |
| | YGMDVWGQGTTVTVSS | | | |
| DABA_54 | QVQLVQSGAEVVKPGASVKISCKASGYT | 133 | DIVLTQSPLSLA | 85 |
| (αFOLR1 × | FTGYFMNWVKQSPGQSLEWIGRIHPYDG | | VSLGQPAIISCK | |
| αCD33 in | DTFYNQKFQGKATLTVDKSSNTAHMELL | | ASQSVSFAGTS | |
| IgG4 | SLTSEDFAVYYCTRYDGSRAMDYWGQG | | LMHWYHQKPG | |
| Backbone | TTVTVSSASTKGPSVFPLAPCSRSTSESTA | | QQPRLLIYRAS | |
| with S228P | ALGCLVKDYFPEPVTVSWNSGALTSGVH | | NLEAGVPDRFS | |
| Mutation) | TFPAVLQSSGLYSLSSVVTVPSSSLGTKTY | | GSGSKTDFTLTI | |
| | TCNVDHKPSNTKVDKRVESKYGPPCPPCP | | SPVEAEDAATY | |
| | APEFLGGPSVFLFPPKPKDTLMISRTPEVT | | YCQQSREYPYT | |
| | CVVVDVSQEDPEVQFNWYVDGVEVHNA | | FGGGTKLEIKR | |
| | KTKPREEQFNSTYRVVSVLTVLHQDWLN | | TVAAPSVFIFPP | |
| | GKEYKCKVSNKGLPSSIEKTISKAKGQPR | | SDEQLKSGTAS | |
| | EPQVYTLPPSQEEMTKNQVSLTCLVKGFY | | VVCLLNNFYPR | |
| | PSDIAVEWESNGQPENNYKTTPPVLDSDG | | EAKVQWKVDN | |
| | SFFLYSRLTVDKSRWQEGNVFSCSVMHE | | ALQSGNSQESV | |
| | ALHNHYTQKSLSLSLggggsggggsEVQLVQS | | TEQDSKDSTYS | |
| | GAEVKKPGSSVKVSCKASGYTITDSNIHWVR | | LSSTLTLSKAD | |
| | QAPGQSLEWIGYIYPYNGGTDYNQKFKNRA | | YEKHKVYACE | |
| | TLTVDNPTNTAYMELSSLRSEDTAFYYCVNG | | VTHQGLSSPVT | |
| | NPWLAYWGQGTLVTVSSggggsggggsggggsgg | | KSFNRGEC | |
| | ggsDIQLTQSPSTLSASVGDRVTITCRASESLD | | | |
| | NYGIRFLTWFQQKPGKAPKLLMYAASNQGS | | | |
| | GVPSRFSGSGSGTEFTLTISSLQPDDFATYYC | | | |
| | QQTKEVPWSFGQGTKVEVK | | | |
| DABA_55 | QVQLVQSGAEVVKPGASVKISCKASGYT | 134 | DIVLTQSPLSLA | 85 |
| (αFOLR1 × | FTGYFMNWVKQSPGQSLEWIGRIHPYDG | | VSLGQPAIISCK | |
| αCSF1R in | DTFYNQKFQGKATLTVDKSSNTAHMELL | | ASQSVSFAGTS | |
| IgG4 | SLTSEDFAVYYCTRYDGSRAMDYWGQG | | LMHWYHQKPG | |
| Backbone | TTVTVSSASTKGPSVFPLAPCSRSTSESTA | | QQPRLLIYRAS | |
| with S228P | ALGCLVKDYFPEPVTVSWNSGALTSGVH | | NLEAGVPDRFS | |
| Mutation) | TFPAVLQSSGLYSLSSVVTVPSSSLGTKTY | | GSGSKTDFTLTI | |
| | TCNVDHKPSNTKVDKRVESKYGPPCPPCP | | SPVEAEDAATY | |
| | APEFLGGPSVFLFPPKPKDTLMISRTPEVT | | YCQQSREYPYT | |
| | CVVVDVSQEDPEVQFNWYVDGVEVHNA | | FGGGTKLEIKR | |
| | KTKPREEQFNSTYRVVSVLTVLHQDWLN | | TVAAPSVFIFPP | |
| | GKEYKCKVSNKGLPSSIEKTISKAKGQPR | | SDEQLKSGTAS | |
| | EPQVYTLPPSQEEMTKNQVSLTCLVKGFY | | VVCLLNNFYPR | |
| | PSDIAVEWESNGQPENNYKTTPPVLDSDG | | EAKVQWKVDN | |
| | SFFLYSRLTVDKSRWQEGNVFSCSVMHE | | ALQSGNSQESV | |
| | ALHNHYTQKSLSLSLggggsggggsQVQLVQS | | TEQDSKDSTYS | |
| | GAEVKKPGSSVKVSCKASGYTFTDNYMIWV | | LSSTLTLSKAD | |
| | RQAPGQGLEWMGDINPYNGGTTFNQKFKG | | YEKHKVYACE | |
| | RVTITADKSTSTAYMELSSLRSEDTAVYYCAR | | VTHQGLSSPVT | |
| | ESPYFSNLYVMDYWGQGTLVTVSSggggsggg | | KSFNRGEC | |
| | gsggggsggggsEIVLTQSPATLSLSPGERATLSC | | | |
| | KASQSVDYDGDNYMNWYQQKPGQAPRLLI | | | |
| | YAASNLESGIPARFSGSGSGTDFTLTISSLEPE | | | |
| | DFAVYYCHLSNEDLSTFGGGTKVEIK | | | |
| DABA_56 | QVQLVQSGAEVVKPGASVKISCKASGYT | 135 | DIVLTQSPLSLA | 85 |
| (αFOLR1 × | FTGYFMNWVKQSPGQSLEWIGRIHPYDG | | VSLGQPAIISCK | |
| αCD163 in | DTFYNQKFQGKATLTVDKSSNTAHMELL | | ASQSVSFAGTS | |
| IgG4 | SLTSEDFAVYYCTRYDGSRAMDYWGQG | | LMHWYHQKPG | |
| Backbone | TTVTVSSASTKGPSVFPLAPCSRSTSESTA | | QQPRLLIYRAS | |
| with S228P | ALGCLVKDYFPEPVTVSWNSGALTSGVH | | NLEAGVPDRFS | |
| Mutation) | TFPAVLQSSGLYSLSSVVTVPSSSLGTKTY | | GSGSKTDFTLTI | |
| | TCNVDHKPSNTKVDKRVESKYGPPCPPCP | | SPVEAEDAATY | |
| | APEFLGGPSVFLFPPKPKDTLMISRTPEVT | | YCQQSREYPYT | |
| | CVVVDVSQEDPEVQFNWYVDGVEVHNA | | FGGGTKLEIKR | |
| | KTKPREEQFNSTYRVVSVLTVLHQDWLN | | TVAAPSVFIFPP | |
| | GKEYKCKVSNKGLPSSIEKTISKAKGQPR | | SDEQLKSGTAS | |
| | EPQVYTLPPSQEEMTKNQVSLTCLVKGFY | | VVCLLNNFYPR | |
| | PSDIAVEWESNGQPENNYKTTPPVLDSDG | | EAKVQWKVDN | |
| | SFFLYSRLTVDKSRWQEGNVFSCSVMHE | | ALQSGNSQESV | |
| | ALHNHYTQKSLSLSLggggsggggsQVQLQES | | TEQDSKDSTYS | |
| | GPGLVKPSETLSLTCTVSGYSITSDYAWNWIR | | LSSTLTLSKAD | |
| | QFPGNKLEWMGYITYSGSTYYNPSLKSRVTIS | | YEKHKVYACE | |
| | VDTSKNQFSLKLSSVTAADTATYYCVSGTYYF | | VTHQGLSSPVT | |
| | DYWGQGTTLTVSSggggsggggsggggsggggsDI | | KSFNRGEC | |
| | VMTQSPSSLSASVGDRVTITCRASQSVSSDVA | | | |
| | WFQQKPGKSPKPLIYYASNRYSGVPSRFSGS | | | |
| | GSGTDFTLTISSLQAEDFAVYFCGQDYTSPR | | | |
| | TFGGGTKLEIK | | | |
| DABA_57 | QVQLVQSGAEVVKPGASVKISCKASGYT | 136 | DIVLTQSPLSLA | 85 |
| (αFOLR1 × | FTGYFMNWVKQSPGQSLEWIGRIHPYDG | | VSLGQPAIISCK | |
| αDR5 in IgG4 | DTFYNQKFQGKATLTVDKSSNTAHMELL | | ASQSVSFAGTS | |
| Backbone | SLTSEDFAVYYCTRYDGSRAMDYWGQG | | LMHWYHQKPG | |
| with S228P | TTVTVSSASTKGPSVFPLAPCSRSTSESTA | | QQPRLLIYRAS | |
| Mutation) | ALGCLVKDYFPEPVTVSWNSGALTSGVH | | NLEAGVPDRFS | |
| | TFPAVLQSSGLYSLSSVVTVPSSSLGTKTY | | GSGSKTDFTLTI | |
| | TCNVDHKPSNTKVDKRVESKYGPPCPPCP | | SPVEAEDAATY | |
| | APEFLGGPSVFLFPPKPKDTLMISRTPEVT | | YCQQSREYPYT | |
| | CVVVDVSQEDPEVQFNWYVDGVEVHNA | | FGGGTKLEIKR | |
| | KTKPREEQFNSTYRVVSVLTVLHQDWLN | | TVAAPSVFIFPP | |
| | GKEYKCKVSNKGLPSSIEKTISKAKGQPR | | SDEQLKSGTAS | |
| | EPQVYTLPPSQEEMTKNQVSLTCLVKGFY | | VVCLLNNFYPR | |
| | PSDIAVEWESNGQPENNYKTTPPVLDSDG | | EAKVQWKVDN | |
| | SFFLYSRLTVDKSRWQEGNVFSCSVMHE | | ALQSGNSQESV | |
| | ALHNHYTQKSLSLSLggggsggggsEIVLTQSP | | TEQDSKDSTYS | |
| | GTLSLSPGERATLSCRASQGISRSYLAWYQQ | | LSSTLTLSKAD | |
| | KPGQAPSLLIYGASSRATGIPDRFSGSGSGT | | YEKHKVYACE | |
| | DFTLTISRLEPEDFAVYYCQQFGSSPWTFGQ | | VTHQGLSSPVT | |
| | GTKVEIKggggsggggsggggsggggsQVQLQESG | | KSFNRGEC | |
| | PGLVKPSQTLSLTCTVSGGSISSGDYFWSWIR | | | |
| | QLPGKGLEWIGHIHNSGTTYYNPSLKSRVTIS | | | |
| | VDTSKKQFSLRLSSVTAADTAVYYCARDRGG | | | |
| | DYYYGMDVWGQGTTVTVSS | | | |
| DABA_58 | EV QLVQ SGAEVKKPGSSVKVSCKASGYTI | 137 | DIQLTQSPSTLS | 90 |
| (αCD33 × | TDSNIHWVRQAPGQSLEWIGYIYPYNGGT | | ASVGDRVTITC | |
| αDR5 in IgG4 | DYNQKFKNRATLTVDNPTNTAYMELSSL | | RASESLDNYGI | |
| Backbone | RSEDTAFYYCVNGNPWLAYWGQGTLVT | | RFLTWFQQKPG | |
| with S228P | VSSASTKGPSVFPLAPCSRSTSESTAALGC | | KAPKLLMYAAS | |
| Mutation) | LVKDYFPEPVTVSWNSGALTSGVHTFPA | | NQGSGVPSRFS | |
| | VLQSSGLYSLSSVVTVPSSSLGTKTYTCN | | GSGSGTEFTLTI | |
| | VDFIKPSNTKVDKRVESKYGPPCPPCPAPE | | SSLQPDDFATY | |
| | FLGGPSVFLFPPKPKDTLMISRTPEVTCVV | | YCQQTKEVPWS | |
| | VDVSQEDPEVQFNWYVDGVEVHNAKTK | | FGQGTKVEVKR | |
| | PREEQFNSTYRVVSVLTVLHQDWLNGKE | | TVAAP SVFIFPP | |
| | YKCKVSNKGLPSSIEKTISKAKGQPREPQ | | SDEQLKSGTAS | |
| | VYTLPPSQEEMTKNQVSLTCLVKGFYPSD | | VVCLLNNFYPR | |
| | IAVEWESNGQPENNYKTTPPVLDSDGSFF | | EAKVQWKVDN | |
| | LYSRLTVDKSRWQEGNVFSCSVMHEALH | | ALQSGNSQESV | |
| | NHYTQKSLSLSLggggsggggsEIVLTQSPGTL | | TEQDSKDSTYS | |
| | SLSPGERATLSCRASQGISRSYLAWYQQKPG | | LSSTLTLSKAD | |
| | QAPSLLIYGASSRATGIPDRFSGSGSGTDFTL | | YEKHKVYACE | |
| | TISRLEPEDFAVYYCQQFGSSPWTFGQGTK | | VTHQGLS SPVT | |
| | VEIKggggsggggsggggsggggsQVQLQESGPGL | | KSFNRGEC | |
| | VKPSQTLSLTCTVSGGSISSGDYFWSWIRQLP | | | |
| | GKGLEWIGHIHNSGTTYYNPSLKSRVTISVDT | | | |
| | SKKQFSLRLSSVTAADTAVYYCARDRGGDYY | | | |
| | YGMDVWGQGTTVTVSS | | | |
| DABA_59 | EVQLLESGGGLVQPGGSLRLSCAVSGFTF | 138 | EIVLTQSPATLS | 92 |
| (αCD38 × | NSFAMSWVRQAPGKGLEWVSAISGSGGG | | LSPGERATLSCR | |
| αDR5 in IgG4 | TYYADSVKGRFTISRDNSKNTLYLQMNS | | ASQSVSSYLAW | |
| Backbone | LRAEDTAVYFCAKDKILWFGEPVFDYWG | | YQQKPGQAPRL | |
| with S228P | QGTLVTVSSASTKGPSVFPLAPCSRSTSES | | LIYDASNRATGI | |
| Mutation) | TAALGCLVKDYFPEPVTVSWNSGALTSG | | PARFSGSGSGT | |
| | VHTFPAVLQ S SGLYSLSSVVTVPS SSLGTK | | DFTLTISSLEPE | |
| | TYTCNVDHKPSNTKVDKRVESKYGPPCP | | DFAVYYCQQRS | |
| | PCPAPEFLGGPSVFLFPPKPKDTLMISRTP | | NWPPTFGQGTK | |
| | EVTCVVVDVSQEDPEVQFNWYVDGVEV | | VEIKRTVAAP S | |
| | HNAKTKPREEQFNSTYRVVSVLTVLHQD | | VFIFPPSDEQLK | |
| | WLNGKEYKCKVSNKGLPSSIEKTISKAKG | | SGTASVVCLLN | |
| | QPREPQVYTLPPSQEEMTKNQVSLTCLVK | | NFYPREAKVQ | |
| | GFYPSDIAVEWESNGQPENNYKTTPPVLD | | WKVDNALQ | SG |
| | SDGSFFLYSRLTVDKSRWQEGNVFSCSV | | NSQESVTEQDS | |
| | MHEALHNHYTQKSLSLSLggggsggggsEIVL | | KDSTYSLSSTLT | |
| | TQSPGTLSLSPGERATLSCRASQGISRSYLAW | | LSKADYEKHKV | |
| | YQQKPGQAPSLLIYGASSRATGIPDRFSGSG | | YACEVTHQGLS | |
| | SGTDFTLTISRLEPEDFAVYYCQQFGSSPWT | | SPVTKSFNRGE | |
| | FGQGTKVEIKggggsggggsggggsggggsQVQL | | C | |
| | QESGPGLVKPSQTLSLTCTVSGGSISSGDYF | | | |
| | WSWIRQLPGKGLEWIGHIHNSGTTYYNPSLK | | | |
| | SRVTISVDTSKKQFSLRLSSVTAADTAVYYCA | | | |
| | RDRGGDYYYGMDVWGQGTTVTVSS | | | |
| DABA_60 | EVQLVQSGAEVKKPGASVKVSCKASGYT | 139 | DVVMTQSPLSL | 94 |
| (αFLT3 × | FTSYYMEIWVRQAPGQGLEWMGIINPSGG | | PVTPGEPASISC | |
| αDR5 in IgG4 | STSYAQKFQGRVTMTRDTSTSTVYMELS | | RSSQSLLHSNG | |
| Backbone | SLRSEDTAVYYCARGVGAHDAFDIWGQG | | NNYLDWYLQK | |
| with S228P | TTVTVSSASTKGPSVFPLAPCSRSTSESTA | | PGQSPQLLIYLG | |
| Mutation) | ALGCLVKDYFPEPVTVSWNSGALTSGVH | | SNRASGVPDRF | |
| | TFPAVLQSSGLYSLSSVVTVPSSSLGTKTY | | SGSGSDTDFTL | |
| | TCNVDHKPSNTKVDKRVESKYGPPCPPCP | | QISRVEAEDVG | |
| | APEFLGGPSVFLFPPKPKDTLMISRTPEVT | | VYYCMQGTHP | |
| | CVVVDVSQEDPEVQFNWYVDGVEVHNA | | AISFGQGTRLEI | |
| | KTKPREEQFNSTYRVVSVLTVLHQDWLN | | KRTVAAPSVFIF | |
| | GKEYKCKVSNKGLPSSIEKTISKAKGQPR | | PPSDEQLKSGT | |
| | EPQVYTLPPSQEEMTKNQVSLTCLVKGFY | | ASVVCLLNNFY | |
| | PSDIAVEWESNGQPENNYKTTPPVLDSDG | | PREAKVQWKV | |
| | SFFLYSRLTVDKSRWQEGNVFSCSVMHE | | DNALQSGNSQE | |
| | ALHNHYTQKSLSLSLggggsggggsEIVLTQSP | | SVTEQDSKDST | |
| | GTLSLSPGERATLSCRASQGISRSYLAWYQQ | | YSLSSTLTLSKA | |
| | KPGQAPSLLIYGASSRATGIPDRFSGSGSGT | | DYEKHKVYAC | |
| | DFTLTISRLEPEDFAVYYCQQFGSSPWTFGQ | | EVTHQGLSSPV | |
| | GTKVEIKggggsggggsggggsggggsQVQLQESG | | TKSFNRGEC | |
| | PGLVKPSQTLSLTCTVSGGSISSGDYFWSWIR | | | |
| | QLPGKGLEWIGHIHNSGTTYYNPSLKSRVTIS | | | |
| | VDTSKKQFSLRLSSVTAADTAVYYCARDRGG | | | |
| | DYYYGMDVWGQGTTVTVSS | | | |

In certain embodiments, a multi-specific binding polypeptide (e.g., a multi-specific antibody) described herein comprises an immunoglobulin heavy chain comprising an amino acid sequence at least about 80%, 85%, 90%, 95%, 97%, 98%, 99%, or 100% identical to or consist of the amino acid sequence set forth in SEQ ID NOs: 7 or 75-77; and an immunoglobulin light chain at least about 80%, 85%, 90%, 95%, 97%, 98%, 99%, or 100% identical to or consist of the amino acid sequence set forth in SEQ ID NO: 8. In some instances, the amino acid differences contributing to the at least about 80%, 85%, 90%, 95%, 97%, 98%, or 99% sequence identity resides in a framework region of the immunoglobulin heavy chain and the CDRs remain unchanged relative to the CDRs set forth in SEQ ID NOs: 7 or 75-77. In some instances, the amino acid differences contributing to the at least about 80%, 85%, 90%, 95%, 97%, 98%, or 99% sequence identity resides in a framework region of the immunoglobulin light chain and the CDRs remain unchanged relative to the CDRs set forth in SEQ ID NO: 8. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain but the multi-specific binding polypeptide (e.g., the multi-specific antibody) retains binding to a target tumor antigen and/or a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain but the multi-specific binding polypeptide (e.g., the multi-specific antibody) retains binding to a target tumor antigen and/or a target antigen expressed on an immunosuppressive cell.

In certain embodiments, a multi-specific binding polypeptide (e.g., a multi-specific antibody) described herein comprises an immunoglobulin heavy chain comprising an amino acid sequence at least about 80%, 85%, 90%, 95%, 97%, 98%, 99%, or 100% identical to or consist of the amino acid sequence set forth in SEQ ID NOs: 79-83; and an immunoglobulin light chain at least about 80%, 85%, 90%, 95%, 97%, 98%, 99%, or 100% identical to or consist of the amino acid sequence set forth in SEQ ID NO: 80. In some instances, the amino acid differences contributing to the at least about 80%, 85%, 90%, 95%, 97%, 98%, or 99% sequence identity resides in a framework region of the immunoglobulin heavy chain and the CDRs remain unchanged relative to the CDRs set forth in SEQ ID NOs: 79-83. In some instances, the amino acid differences contributing to the at least about 80%, 85%, 90%, 95%, 97%, 98%, or 99% sequence identity resides in a framework region of the immunoglobulin light chain and the CDRs remain unchanged relative to the CDRs set forth in SEQ ID NO: 80. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain but the multi-specific binding polypeptide (e.g., the multi-specific antibody) retains binding to a target tumor antigen and/or a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain but the multi-specific binding polypeptide (e.g., the multi-specific antibody) retains binding to a target tumor antigen and/or a target antigen expressed on an immunosuppressive cell.

In certain embodiments, a multi-specific binding polypeptide (e.g., a multi-specific antibody) described herein comprises an immunoglobulin heavy chain comprising an amino acid sequence at least about 80%, 85%, 90%, 95%, 97%, 98%, 99%, or 100% identical to or consist of the amino acid sequence set forth in SEQ ID NOs: 84-88; and an immunoglobulin light chain at least about 80%, 85%, 90%, 95%, 97%, 98%, 99%, or 100% identical to or consist of the amino acid sequence set forth in SEQ ID NO: 85. In some instances, the amino acid differences contributing to the at least about 80%, 85%, 90%, 95%, 97%, 98%, or 99% sequence identity resides in a framework region of the immunoglobulin heavy chain and the CDRs remain unchanged relative to the CDRs set forth in SEQ ID NOs: 84-88. In some instances, the amino acid differences contributing to the at least about 80%, 85%, 90%, 95%, 97%, 98%, or 99% sequence identity resides in a framework region of the immunoglobulin light chain and the CDRs remain unchanged relative to the CDRs set forth in SEQ ID NO: 85. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain but the multi-specific binding polypeptide (e.g., the multi-specific antibody) retains binding to a target tumor antigen and/or a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain but the multi-specific binding polypeptide (e.g., the multi-specific antibody) retains binding to a target tumor antigen and/or a target antigen expressed on an immunosuppressive cell.

In certain embodiments, a multi-specific binding polypeptide (e.g., a multi-specific antibody) described herein comprises an immunoglobulin heavy chain comprising an amino acid sequence at least about 80%, 85%, 90%, 95%, 97%, 98%, 99%, or 100% identical to or consist of the amino acid sequence set forth in SEQ ID NOs: 89, 91, or 93; and an immunoglobulin light chain at least about 80%, 85%, 90%, 95%, 97%, 98%, 99%, or 100% identical to or consist of the amino acid sequence set forth in SEQ ID NO: 90, 92, or 94. In some instances, the amino acid differences contributing to the at least about 80%, 85%, 90%, 95%, 97%, 98%, or 99% sequence identity resides in a framework region of the immunoglobulin heavy chain and the CDRs remain unchanged relative to the CDRs set forth in SEQ ID NO: 89, 91, or 93. In some instances, the amino acid differences contributing to the at least about 80%, 85%, 90%, 95%, 97%, 98%, or 99% sequence identity resides in a framework region of the immunoglobulin light chain and the CDRs remain unchanged relative to the CDRs set forth in SEQ ID NO: 90, 92, or 94. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin heavy chain but the multi-specific binding polypeptide (e.g., the multi-specific antibody) retains binding to a target tumor antigen and/or a target antigen expressed on an immunosuppressive cell. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin light chain but the multi-specific binding polypeptide (e.g., the multi-specific antibody) retains binding to a target tumor antigen and/or a target antigen expressed on an immunosuppressive cell.

In certain embodiments, the multi-specific binding polypeptide (e.g., multi-specific antibody) comprises a tumor binding moiety that specifically binds to TROP2/TACSTD2 and an immune cell binding moiety that specifically binds to TRAIL-R2. In certain embodiments the moiety that binds to TROP2/TACSTD2 comprises a heavy chain variable region that comprises an amino acid sequence at least about 80%, 85%, 90%, 95%, 97%, 98%, 99% or 100% identical to that set forth in SEQ ID NO: 9; and a light chain variable region that comprises an amino acid sequence at least about 80%, 85%, 90%, 95%, 97%, 98%, 99% or 100% identical to that set forth in SEQ ID NO: 10. In certain embodiments the moiety that binds to TRAIL-R2 comprises a heavy chain variable region that comprises an amino acid sequence at least about 80%, 85%, 90%, 95%, 97%, 98%, 99% or 100% identical to that set forth in SEQ ID NO: 11; and a light chain variable region that comprises an amino acid sequence at least about 80%, 85%, 90%, 95%, 97%, 98%, 99% or 100% identical to that set forth in SEQ ID NO: 12. In certain embodiments, the heavy chain of the multi-specific binding polypeptide comprises an amino acid sequence at least about 80%, 85%, 90%, 95%, 97%, 98%, 99% or 100% identical to that set forth in SEQ ID NO: 7; and the light chain comprises an amino acid sequence at least about 80%, 85%, 90%, 95%, 97%, 98%, 99% or 100% identical to that set forth in SEQ ID NO: 8. SEQ ID NO: 7 and 8 form a bispecific molecule depicted as in FIG. 1A that can be conjugated to a cytotoxic moiety as in FIG. 1B. In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In certain embodiments, the multi-specific binding polypeptide comprises an IgG that specifically binds TROP2 comprising a light chain variable region and a heavy chain variable region, wherein the light chain variable region comprises a CDR1 with the amino acid sequence set forth as KASQDVSIAVA (SEQ ID NO: 1), a CDR2 with the amino acid sequence set forth as SASYRYT (SEQ ID NO: 2), and a CDR3 with the amino acid sequence set forth as QQHYITPLT (SEQ ID NO: 3); and the heavy chain variable region comprises a CDR1 with the amino acid sequence set forth as NYGMN (SEQ ID NO: 4), a CDR2 with the amino acid sequence set forth as WINTYTGEPTYTDDFKG (SEQ ID NO: 5), and a CDR3 with the amino acid sequence set forth as GGFGSSYWYFDV (SEQ ID NO: 6). In certain embodiments, the multi-specific binding polypeptide comprises an scFv that specifically binds TROP2 comprising a light chain variable region and a heavy chain variable region, wherein the light chain variable region comprises a CDR1 with the amino acid sequence set forth as KASQDVSIAVA (SEQ ID NO: 1), a CDR2 with the amino acid sequence set forth as SASYRYT (SEQ ID NO: 2), and a CDR3 with the amino acid sequence set forth as QQHYITPLT (SEQ ID NO: 3); and the heavy chain variable region comprises a CDR1 with the amino acid sequence set forth as NYGMN (SEQ ID NO: 4), a CDR2 with the amino acid sequence set forth as WINTYTGEPTYTDDFKG (SEQ ID NO: 5), and a CDR3 with the amino acid sequence set forth as GGFGSSYWYFDV (SEQ ID NO: 6).

In some embodiments, a multi-specific binding polypeptide (e.g., a multi-specific antibody) described herein comprises a tumor binding moiety that specifically binds to TROP2 and an immune cell binding moiety that specifically binds to CD33. In some instances, the tumor biding moiety that specifically binds to TROP2 comprises an immunoglobulin heavy chain variable region comprising CDR1 (HCDR1), CDR2 (HCDR2), and CDR3 (HCDR3), in which HCDR1 comprises SEQ ID NO: 4, HCDR2 comprises SEQ ID NO: 5, and HCDR3 comprises SEQ ID NO: 6. In some instances, the tumor biding moiety that specifically binds to TROP2 comprises an immunoglobulin light chain variable region comprising CDR1 (LCDR1), CDR2 (LCDR2), and CDR3 (LCDR3), in which LCDR1 comprises SEQ ID NO: 1, LCDR2 comprises SEQ ID NO: 2, and LCDR3 comprises SEQ ID NO: 3. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 9; and an immunoglobulin light chain variable region at least about 80%, 85%, 90%, 95%, 97%, 98%, 99%, or 100% identical to or consist of the amino acid sequence set forth in SEQ ID NO: 10. In some instances, the immune cell binding moiety that specifically binds to CD33 comprises an immunoglobulin heavy chain variable region comprising CDR1 (HCDR1), CDR2 (HCDR2), and CDR3 (HCDR3), in which HCDR1 comprises SEQ ID NO: 45, HCDR2 comprises SEQ ID NO: 46, and HCDR3 comprises SEQ ID NO: 47. In some instances, the immune cell binding moiety that specifically binds to CD33 comprises an immunoglobulin light chain variable region comprising CDR1 (LCDR1), CDR2 (LCDR2), and CDR3 (LCDR3), in which LCDR1 comprises SEQ ID NO: 60, LCDR2 comprises SEQ ID NO: 61, and LCDR3 comprises SEQ ID NO: 62. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 48; and an immunoglobulin light chain variable region at least about 80%, 85%, 90%, 95%, 97%, 98%, 99%, or 100% identical to or consist of the amino acid sequence set forth in SEQ ID NO: 63. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 75; and an immunoglobulin light chain at least about 80%, 85%, 90%, 95%, 97%, 98%, 99%, or 100% identical to or consist of the amino acid sequence set forth in SEQ ID NO: 8. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to TROP2 and an immune cell binding moiety that specifically binds to CD163. In some instances, the tumor biding moiety that specifically binds to TROP2 comprises an immunoglobulin heavy chain variable region comprising CDR1 (HCDR1), CDR2 (HCDR2), and CDR3 (HCDR3), in which HCDR1 comprises SEQ ID NO: 4, HCDR2 comprises SEQ ID NO: 5, and HCDR3 comprises SEQ ID NO: 6. In some instances, the tumor biding moiety that specifically binds to TROP2 comprises an immunoglobulin light chain variable region comprising CDR1 (LCDR1), CDR2 (LCDR2), and CDR3 (LCDR3), in which LCDR1 comprises SEQ ID NO: 1, LCDR2 comprises SEQ ID NO: 2, and LCDR3 comprises SEQ ID NO: 3. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 9; and an immunoglobulin light chain variable region at least about 80%, 85%, 90%, 95%, 97%, 98%, 99%, or 100% identical to or consist of the amino acid sequence set forth in SEQ ID NO: 10. In some instances, the immune cell binding moiety that specifically binds to CD163 comprises an immunoglobulin heavy chain variable region comprising CDR1 (HCDR1), CDR2 (HCDR2), and CDR3 (HCDR3), in which HCDR1 comprises SEQ ID NO: 49, HCDR2 comprises SEQ ID NO: 50, and HCDR3 comprises SEQ ID NO: 51. In some instances, the immune cell binding moiety that specifically binds to CD163 comprises an immunoglobulin light chain variable region comprising CDR1 (LCDR1), CDR2 (LCDR2), and CDR3 (LCDR3), in which LCDR1 comprises SEQ ID NO: 64, LCDR2 comprises SEQ ID NO: 65, and LCDR3 comprises SEQ ID NO: 66. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 52; and an immunoglobulin light chain variable region at least about 80%, 85%, 90%, 95%, 97%, 98%, 99%, or 100% identical to or consist of the amino acid sequence set forth in SEQ ID NO: 67. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 77; and an immunoglobulin light chain at least about 80%, 85%, 90%, 95%, 97%, 98%, 99%, or 100% identical to or consist of the amino acid sequence set forth in SEQ ID NO: 8. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to TROP2 and an immune cell binding moiety that specifically binds to CSF1R. In some instances, the tumor biding moiety that specifically binds to TROP2 comprises an immunoglobulin heavy chain variable region comprising CDR1 (HCDR1), CDR2 (HCDR2), and CDR3 (HCDR3), in which HCDR1 comprises SEQ ID NO: 4, HCDR2 comprises SEQ ID NO: 5, and HCDR3 comprises SEQ ID NO: 6. In some instances, the tumor biding moiety that specifically binds to TROP2 comprises an immunoglobulin light chain variable region comprising CDR1 (LCDR1), CDR2 (LCDR2), and CDR3 (LCDR3), in which LCDR1 comprises SEQ ID NO: 1, LCDR2 comprises SEQ ID NO: 2, and LCDR3 comprises SEQ ID NO: 3. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 9; and an immunoglobulin light chain variable region at least about 80%, 85%, 90%, 95%, 97%, 98%, 99%, or 100% identical to or consist of the amino acid sequence set forth in SEQ ID NO: 10. In some instances, the immune cell binding moiety that specifically binds to CSF1R comprises an immunoglobulin heavy chain variable region comprising CDR1 (HCDR1), CDR2 (HCDR2), and CDR3 (HCDR3), in which HCDR1 comprises SEQ ID NO: 56, HCDR2 comprises SEQ ID NO: 57, and HCDR3 comprises SEQ ID NO: 58. In some instances, the immune cell binding moiety that specifically binds to CSF1R comprises an immunoglobulin light chain variable region comprising CDR1 (LCDR1), CDR2 (LCDR2), and CDR3 (LCDR3), in which LCDR1 comprises SEQ ID NO: 71, LCDR2 comprises SEQ ID NO: 72, and LCDR3 comprises SEQ ID NO: 73. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 59; and an immunoglobulin light chain variable region at least about 80%, 85%, 90%, 95%, 97%, 98%, 99%, or 100% identical to or consist of the amino acid sequence set forth in SEQ ID NO: 74. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 76; and an immunoglobulin light chain at least about 80%, 85%, 90%, 95%, 97%, 98%, 99%, or 100% identical to or consist of the amino acid sequence set forth in SEQ ID NO: 8. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to TROP2 and an immune cell binding moiety that specifically binds to TRAIL-R2. In some instances, the tumor biding moiety that specifically binds to TROP2 comprises an immunoglobulin heavy chain variable region comprising CDR1 (HCDR1), CDR2 (HCDR2), and CDR3 (HCDR3), in which HCDR1 comprises SEQ ID NO: 4, HCDR2 comprises SEQ ID NO: 5, and HCDR3 comprises SEQ ID NO: 6. In some instances, the tumor biding moiety that specifically binds to TROP2 comprises an immunoglobulin light chain variable region comprising CDR1 (LCDR1), CDR2 (LCDR2), and CDR3 (LCDR3), in which LCDR1 comprises SEQ ID NO: 1, LCDR2 comprises SEQ ID NO: 2, and LCDR3 comprises SEQ ID NO: 3. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 9; and an immunoglobulin light chain variable region at least about 80%, 85%, 90%, 95%, 97%, 98%, 99%, or 100% identical to or consist of the amino acid sequence set forth in SEQ ID NO: 10. In some instances, the immune cell binding moiety that specifically binds to TRAIL-R2 comprises an immunoglobulin heavy chain variable region comprising CDR1 (HCDR1), CDR2 (HCDR2), and CDR3 (HCDR3), in which HCDR1 comprises SEQ ID NO: 53, HCDR2 comprises SEQ ID NO: 54, and HCDR3 comprises SEQ ID NO: 55. In some instances, the immune cell binding moiety that specifically binds to TRAIL-R2 comprises an immunoglobulin light chain variable region comprising CDR1 (LCDR1), CDR2 (LCDR2), and CDR3 (LCDR3), in which LCDR1 comprises SEQ ID NO: 68, LCDR2 comprises SEQ ID NO: 69, and LCDR3 comprises SEQ ID NO: 70. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 11; and an immunoglobulin light chain variable region at least about 80%, 85%, 90%, 95%, 97%, 98%, 99%, or 100% identical to or consist of the amino acid sequence set forth in SEQ ID NO: 12. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 78; and an immunoglobulin light chain at least about 80%, 85%, 90%, 95%, 97%, 98%, 99%, or 100% identical to or consist of the amino acid sequence set forth in SEQ ID NO: 8. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to GPC3 and an immune cell binding moiety that specifically binds to CD33. In some instances, the tumor biding moiety that specifically binds to GPC3 comprises an immunoglobulin heavy chain variable region comprising CDR1 (HCDR1), CDR2 (HCDR2), and CDR3 (HCDR3), in which HCDR1 comprises SEQ ID NO: 13, HCDR2 comprises SEQ ID NO: 14, and HCDR3 comprises SEQ ID NO: 15. In some instances, the tumor biding moiety that specifically binds to GPC3 comprises an immunoglobulin light chain variable region comprising CDR1 (LCDR1), CDR2 (LCDR2), and CDR3 (LCDR3), in which LCDR1 comprises SEQ ID NO: 29, LCDR2 comprises SEQ ID NO: 30, and LCDR3 comprises SEQ ID NO: 31. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 16; and an immunoglobulin light chain variable region at least about 80%, 85%, 90%, 95%, 97%, 98%, 99%, or 100% identical to or consist of the amino acid sequence set forth in SEQ ID NO: 32. In some instances, the immune cell binding moiety that specifically binds to CD33 comprises an immunoglobulin heavy chain variable region comprising CDR1 (HCDR1), CDR2 (HCDR2), and CDR3 (HCDR3), in which HCDR1 comprises SEQ ID NO: 45, HCDR2 comprises SEQ ID NO: 46, and HCDR3 comprises SEQ ID NO: 47. In some instances, the immune cell binding moiety that specifically binds to CD33 comprises an immunoglobulin light chain variable region comprising CDR1 (LCDR1), CDR2 (LCDR2), and CDR3 (LCDR3), in which LCDR1 comprises SEQ ID NO: 60, LCDR2 comprises SEQ ID NO: 61, and LCDR3 comprises SEQ ID NO: 62. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 48; and an immunoglobulin light chain variable region at least about 80%, 85%, 90%, 95%, 97%, 98%, 99%, or 100% identical to or consist of the amino acid sequence set forth in SEQ ID NO: 63. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 79; and an immunoglobulin light chain at least about 80%, 85%, 90%, 95%, 97%, 98%, 99%, or 100% identical to or consist of the amino acid sequence set forth in SEQ ID NO: 80. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to GPC3 and an immune cell binding moiety that specifically binds to CD163. In some instances, the tumor biding moiety that specifically binds to GPC3 comprises an immunoglobulin heavy chain variable region comprising CDR1 (HCDR1), CDR2 (HCDR2), and CDR3 (HCDR3), in which HCDR1 comprises SEQ ID NO: 13, HCDR2 comprises SEQ ID NO: 14, and HCDR3 comprises SEQ ID NO: 15. In some instances, the tumor biding moiety that specifically binds to GPC3 comprises an immunoglobulin light chain variable region comprising CDR1 (LCDR1), CDR2 (LCDR2), and CDR3 (LCDR3), in which LCDR1 comprises SEQ ID NO: 29, LCDR2 comprises SEQ ID NO: 30, and LCDR3 comprises SEQ ID NO: 31. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 16; and an immunoglobulin light chain variable region at least about 80%, 85%, 90%, 95%, 97%, 98%, 99%, or 100% identical to or consist of the amino acid sequence set forth in SEQ ID NO: 32. In some instances, the immune cell binding moiety that specifically binds to CD163 comprises an immunoglobulin heavy chain variable region comprising CDR1 (HCDR1), CDR2 (HCDR2), and CDR3 (HCDR3), in which HCDR1 comprises SEQ ID NO: 49, HCDR2 comprises SEQ ID NO: 50, and HCDR3 comprises SEQ ID NO: 51. In some instances, the immune cell binding moiety that specifically binds to CD163 comprises an immunoglobulin light chain variable region comprising CDR1 (LCDR1), CDR2 (LCDR2), and CDR3 (LCDR3), in which LCDR1 comprises SEQ ID NO: 64, LCDR2 comprises SEQ ID NO: 65, and LCDR3 comprises SEQ ID NO: 66. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 52; and an immunoglobulin light chain variable region at least about 80%, 85%, 90%, 95%, 97%, 98%, 99%, or 100% identical to or consist of the amino acid sequence set forth in SEQ ID NO: 67. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 82; and an immunoglobulin light chain at least about 80%, 85%, 90%, 95%, 97%, 98%, 99%, or 100% identical to or consist of the amino acid sequence set forth in SEQ ID NO: 80. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to GPC3 and an immune cell binding moiety that specifically binds to CSF1R. In some instances, the tumor biding moiety that specifically binds to GPC3 comprises an immunoglobulin heavy chain variable region comprising CDR1 (HCDR1), CDR2 (HCDR2), and CDR3 (HCDR3), in which HCDR1 comprises SEQ ID NO: 13, HCDR2 comprises SEQ ID NO: 14, and HCDR3 comprises SEQ ID NO: 15. In some instances, the tumor biding moiety that specifically binds to GPC3 comprises an immunoglobulin light chain variable region comprising CDR1 (LCDR1), CDR2 (LCDR2), and CDR3 (LCDR3), in which LCDR1 comprises SEQ ID NO: 29, LCDR2 comprises SEQ ID NO: 30, and LCDR3 comprises SEQ ID NO: 31. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 16; and an immunoglobulin light chain variable region at least about 80%, 85%, 90%, 95%, 97%, 98%, 99%, or 100% identical to or consist of the amino acid sequence set forth in SEQ ID NO: 32. In some instances, the immune cell binding moiety that specifically binds to CSF1R comprises an immunoglobulin heavy chain variable region comprising CDR1 (HCDR1), CDR2 (HCDR2), and CDR3 (HCDR3), in which HCDR1 comprises SEQ ID NO: 56, HCDR2 comprises SEQ ID NO: 57, and HCDR3 comprises SEQ ID NO: 58. In some instances, the immune cell binding moiety that specifically binds to CSF1R comprises an immunoglobulin light chain variable region comprising CDR1 (LCDR1), CDR2 (LCDR2), and CDR3 (LCDR3), in which LCDR1 comprises SEQ ID NO: 71, LCDR2 comprises SEQ ID NO: 72, and LCDR3 comprises SEQ ID NO: 73. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 59; and an immunoglobulin light chain variable region at least about 80%, 85%, 90%, 95%, 97%, 98%, 99%, or 100% identical to or consist of the amino acid sequence set forth in SEQ ID NO: 74. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 81; and an immunoglobulin light chain at least about 80%, 85%, 90%, 95%, 97%, 98%, 99%, or 100% identical to or consist of the amino acid sequence set forth in SEQ ID NO: 80. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to FOLR1 and an immune cell binding moiety that specifically binds to CD33. In some instances, the tumor biding moiety that specifically binds to FOLR1 comprises an immunoglobulin heavy chain variable region comprising CDR1 (HCDR1), CDR2 (HCDR2), and CDR3 (HCDR3), in which HCDR1 comprises SEQ ID NO: 17, HCDR2 comprises SEQ ID NO: 18, and HCDR3 comprises SEQ ID NO: 19. In some instances, the tumor biding moiety that specifically binds to FOLR1 comprises an immunoglobulin light chain variable region comprising CDR1 (LCDR1), CDR2 (LCDR2), and CDR3 (LCDR3), in which LCDR1 comprises SEQ ID NO: 33, LCDR2 comprises SEQ ID NO: 34, and LCDR3 comprises SEQ ID NO: 35. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 20; and an immunoglobulin light chain variable region at least about 80%, 85%, 90%, 95%, 97%, 98%, 99%, or 100% identical to or consist of the amino acid sequence set forth in SEQ ID NO: 36. In some instances, the immune cell binding moiety that specifically binds to CD33 comprises an immunoglobulin heavy chain variable region comprising CDR1 (HCDR1), CDR2 (HCDR2), and CDR3 (HCDR3), in which HCDR1 comprises SEQ ID NO: 45, HCDR2 comprises SEQ ID NO: 46, and HCDR3 comprises SEQ ID NO: 47. In some instances, the immune cell binding moiety that specifically binds to CD33 comprises an immunoglobulin light chain variable region comprising CDR1 (LCDR1), CDR2 (LCDR2), and CDR3 (LCDR3), in which LCDR1 comprises SEQ ID NO: 60, LCDR2 comprises SEQ ID NO: 61, and LCDR3 comprises SEQ ID NO: 62. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 48; and an immunoglobulin light chain variable region at least about 80%, 85%, 90%, 95%, 97%, 98%, 99%, or 100% identical to or consist of the amino acid sequence set forth in SEQ ID NO: 63. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 84; and an immunoglobulin light chain at least about 80%, 85%, 90%, 95%, 97%, 98%, 99%, or 100% identical to or consist of the amino acid sequence set forth in SEQ ID NO: 85. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to FOLR1 and an immune cell binding moiety that specifically binds to CD163. In some instances, the tumor biding moiety that specifically binds to FOLR1 comprises an immunoglobulin heavy chain variable region comprising CDR1 (HCDR1), CDR2 (HCDR2), and CDR3 (HCDR3), in which HCDR1 comprises SEQ ID NO: 17, HCDR2 comprises SEQ ID NO: 18, and HCDR3 comprises SEQ ID NO: 19. In some instances, the tumor biding moiety that specifically binds to FOLR1 comprises an immunoglobulin light chain variable region comprising CDR1 (LCDR1), CDR2 (LCDR2), and CDR3 (LCDR3), in which LCDR1 comprises SEQ ID NO: 33, LCDR2 comprises SEQ ID NO: 34, and LCDR3 comprises SEQ ID NO: 35. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 20; and an immunoglobulin light chain variable region at least about 80%, 85%, 90%, 95%, 97%, 98%, 99%, or 100% identical to or consist of the amino acid sequence set forth in SEQ ID NO: 36. In some instances, the immune cell binding moiety that specifically binds to CD163 comprises an immunoglobulin heavy chain variable region comprising CDR1 (HCDR1), CDR2 (HCDR2), and CDR3 (HCDR3), in which HCDR1 comprises SEQ ID NO: 49, HCDR2 comprises SEQ ID NO: 50, and HCDR3 comprises SEQ ID NO: 51. In some instances, the immune cell binding moiety that specifically binds to CD163 comprises an immunoglobulin light chain variable region comprising CDR1 (LCDR1), CDR2 (LCDR2), and CDR3 (LCDR3), in which LCDR1 comprises SEQ ID NO: 64, LCDR2 comprises SEQ ID NO: 65, and LCDR3 comprises SEQ ID NO: 66. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 52; and an immunoglobulin light chain variable region at least about 80%, 85%, 90%, 95%, 97%, 98%, 99%, or 100% identical to or consist of the amino acid sequence set forth in SEQ ID NO: 67. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 87; and an immunoglobulin light chain at least about 80%, 85%, 90%, 95%, 97%, 98%, 99%, or 100% identical to or consist of the amino acid sequence set forth in SEQ ID NO: 85. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to FOLR1 and an immune cell binding moiety that specifically binds to CSF1R. In some instances, the tumor biding moiety that specifically binds to FOLR1 comprises an immunoglobulin heavy chain variable region comprising CDR1 (HCDR1), CDR2 (HCDR2), and CDR3 (HCDR3), in which HCDR1 comprises SEQ ID NO: 17, HCDR2 comprises SEQ ID NO: 18, and HCDR3 comprises SEQ ID NO: 19. In some instances, the tumor biding moiety that specifically binds to FOLR1 comprises an immunoglobulin light chain variable region comprising CDR1 (LCDR1), CDR2 (LCDR2), and CDR3 (LCDR3), in which LCDR1 comprises SEQ ID NO: 33, LCDR2 comprises SEQ ID NO: 34, and LCDR3 comprises SEQ ID NO: 35. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 20; and an immunoglobulin light chain variable region at least about 80%, 85%, 90%, 95%, 97%, 98%, 99%, or 100% identical to or consist of the amino acid sequence set forth in SEQ ID NO: 36. In some instances, the immune cell binding moiety that specifically binds to CSF1R comprises an immunoglobulin heavy chain variable region comprising CDR1 (HCDR1), CDR2 (HCDR2), and CDR3 (HCDR3), in which HCDR1 comprises SEQ ID NO: 56, HCDR2 comprises SEQ ID NO: 57, and HCDR3 comprises SEQ ID NO: 58. In some instances, the immune cell binding moiety that specifically binds to CSF1R comprises an immunoglobulin light chain variable region comprising CDR1 (LCDR1), CDR2 (LCDR2), and CDR3 (LCDR3), in which LCDR1 comprises SEQ ID NO: 71, LCDR2 comprises SEQ ID NO: 72, and LCDR3 comprises SEQ ID NO: 73. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 59; and an immunoglobulin light chain variable region at least about 80%, 85%, 90%, 95%, 97%, 98%, 99%, or 100% identical to or consist of the amino acid sequence set forth in SEQ ID NO: 74. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 86; and an immunoglobulin light chain at least about 80%, 85%, 90%, 95%, 97%, 98%, 99%, or 100% identical to or consist of the amino acid sequence set forth in SEQ ID NO: 85. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to GPC3 and an immune cell binding moiety that specifically binds to TRAIL-R2. In some instances, the tumor biding moiety that specifically binds to GPC3 comprises an immunoglobulin heavy chain variable region comprising CDR1 (HCDR1), CDR2 (HCDR2), and CDR3 (HCDR3), in which HCDR1 comprises SEQ ID NO: 13, HCDR2 comprises SEQ ID NO: 14, and HCDR3 comprises SEQ ID NO: 15. In some instances, the tumor biding moiety that specifically binds to GPC3 comprises an immunoglobulin light chain variable region comprising CDR1 (LCDR1), CDR2 (LCDR2), and CDR3 (LCDR3), in which LCDR1 comprises SEQ ID NO: 29, LCDR2 comprises SEQ ID NO: 30, and LCDR3 comprises SEQ ID NO: 31. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 16; and an immunoglobulin light chain variable region at least about 80%, 85%, 90%, 95%, 97%, 98%, 99%, or 100% identical to or consist of the amino acid sequence set forth in SEQ ID NO: 32. In some instances, the immune cell binding moiety that specifically binds to TRAIL-R2 comprises an immunoglobulin heavy chain variable region comprising CDR1 (HCDR1), CDR2 (HCDR2), and CDR3 (HCDR3), in which HCDR1 comprises SEQ ID NO: 53, HCDR2 comprises SEQ ID NO: 54, and HCDR3 comprises SEQ ID NO: 55. In some instances, the immune cell binding moiety that specifically binds to TRAIL-R2 comprises an immunoglobulin light chain variable region comprising CDR1 (LCDR1), CDR2 (LCDR2), and CDR3 (LCDR3), in which LCDR1 comprises SEQ ID NO: 68, LCDR2 comprises SEQ ID NO: 69, and LCDR3 comprises SEQ ID NO: 70. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 11; and an immunoglobulin light chain variable region at least about 80%, 85%, 90%, 95%, 97%, 98%, 99%, or 100% identical to or consist of the amino acid sequence set forth in SEQ ID NO: 12. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 83; and an immunoglobulin light chain at least about 80%, 85%, 90%, 95%, 97%, 98%, 99%, or 100% identical to or consist of the amino acid sequence set forth in SEQ ID NO: 80. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to FOLR1 and an immune cell binding moiety that specifically binds to TRAIL-R2. In some instances, the tumor biding moiety that specifically binds to FOLR1 comprises an immunoglobulin heavy chain variable region comprising CDR1 (HCDR1), CDR2 (HCDR2), and CDR3 (HCDR3), in which HCDR1 comprises SEQ ID NO: 17, HCDR2 comprises SEQ ID NO: 18, and HCDR3 comprises SEQ ID NO: 19. In some instances, the tumor biding moiety that specifically binds to FOLR1 comprises an immunoglobulin light chain variable region comprising CDR1 (LCDR1), CDR2 (LCDR2), and CDR3 (LCDR3), in which LCDR1 comprises SEQ ID NO: 33, LCDR2 comprises SEQ ID NO: 34, and LCDR3 comprises SEQ ID NO: 35. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 20; and an immunoglobulin light chain variable region at least about 80%, 85%, 90%, 95%, 97%, 98%, 99%, or 100% identical to or consist of the amino acid sequence set forth in SEQ ID NO: 36. In some instances, the immune cell binding moiety that specifically binds to TRAIL-R2 comprises an immunoglobulin heavy chain variable region comprising CDR1 (HCDR1), CDR2 (HCDR2), and CDR3 (HCDR3), in which HCDR1 comprises SEQ ID NO: 53, HCDR2 comprises SEQ ID NO: 54, and HCDR3 comprises SEQ ID NO: 55. In some instances, the immune cell binding moiety that specifically binds to TRAIL-R2 comprises an immunoglobulin light chain variable region comprising CDR1 (LCDR1), CDR2 (LCDR2), and CDR3 (LCDR3), in which LCDR1 comprises SEQ ID NO: 68, LCDR2 comprises SEQ ID NO: 69, and LCDR3 comprises SEQ ID NO: 70. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 11; and an immunoglobulin light chain variable region at least about 80%, 85%, 90%, 95%, 97%, 98%, 99%, or 100% identical to or consist of the amino acid sequence set forth in SEQ ID NO: 12. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 88; and an immunoglobulin light chain at least about 80%, 85%, 90%, 95%, 97%, 98%, 99%, or 100% identical to or consist of the amino acid sequence set forth in SEQ ID NO: 85. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to CD33 and an immune cell binding moiety that specifically binds to TRAIL-R2. In some instances, the tumor biding moiety that specifically binds to CD33 comprises an immunoglobulin heavy chain variable region comprising CDR1 (HCDR1), CDR2 (HCDR2), and CDR3 (HCDR3), in which HCDR1 comprises SEQ ID NO: 45, HCDR2 comprises SEQ ID NO: 46, and HCDR3 comprises SEQ ID NO: 47. In some instances, the tumor biding moiety that specifically binds to CD33 comprises an immunoglobulin light chain variable region comprising CDR1 (LCDR1), CDR2 (LCDR2), and CDR3 (LCDR3), in which LCDR1 comprises SEQ ID NO: 60, LCDR2 comprises SEQ ID NO: 61, and LCDR3 comprises SEQ ID NO: 62. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 48; and an immunoglobulin light chain variable region at least about 80%, 85%, 90%, 95%, 97%, 98%, 99%, or 100% identical to or consist of the amino acid sequence set forth in SEQ ID NO: 63. In some instances, the immune cell binding moiety that specifically binds to TRAIL-R2 comprises an immunoglobulin heavy chain variable region comprising CDR1 (HCDR1), CDR2 (HCDR2), and CDR3 (HCDR3), in which HCDR1 comprises SEQ ID NO: 53, HCDR2 comprises SEQ ID NO: 54, and HCDR3 comprises SEQ ID NO: 55. In some instances, the immune cell binding moiety that specifically binds to TRAIL-R2 comprises an immunoglobulin light chain variable region comprising CDR1 (LCDR1), CDR2 (LCDR2), and CDR3 (LCDR3), in which LCDR1 comprises SEQ ID NO: 68, LCDR2 comprises SEQ ID NO: 69, and LCDR3 comprises SEQ ID NO: 70. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 11; and an immunoglobulin light chain variable region at least about 80%, 85%, 90%, 95%, 97%, 98%, 99%, or 100% identical to or consist of the amino acid sequence set forth in SEQ ID NO: 12. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 89; and an immunoglobulin light chain at least about 80%, 85%, 90%, 95%, 97%, 98%, 99%, or 100% identical to or consist of the amino acid sequence set forth in SEQ ID NO: 90. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to CD38 and an immune cell binding moiety that specifically binds to TRAIL-R2. In some instances, the tumor biding moiety that specifically binds to CD38 comprises an immunoglobulin heavy chain variable region comprising CDR1 (HCDR1), CDR2 (HCDR2), and CDR3 (HCDR3), in which HCDR1 comprises SEQ ID NO: 21, HCDR2 comprises SEQ ID NO: 22, and HCDR3 comprises SEQ ID NO: 23. In some instances, the tumor biding moiety that specifically binds to CD38 comprises an immunoglobulin light chain variable region comprising CDR1 (LCDR1), CDR2 (LCDR2), and CDR3 (LCDR3), in which LCDR1 comprises SEQ ID NO: 37, LCDR2 comprises SEQ ID NO: 38, and LCDR3 comprises SEQ ID NO: 39. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 24; and an immunoglobulin light chain variable region at least about 80%, 85%, 90%, 95%, 97%, 98%, 99%, or 100% identical to or consist of the amino acid sequence set forth in SEQ ID NO: 40. In some instances, the immune cell binding moiety that specifically binds to TRAIL-R2 comprises an immunoglobulin heavy chain variable region comprising CDR1 (HCDR1), CDR2 (HCDR2), and CDR3 (HCDR3), in which HCDR1 comprises SEQ ID NO: 53, HCDR2 comprises SEQ ID NO: 54, and HCDR3 comprises SEQ ID NO: 55. In some instances, the immune cell binding moiety that specifically binds to TRAIL-R2 comprises an immunoglobulin light chain variable region comprising CDR1 (LCDR1), CDR2 (LCDR2), and CDR3 (LCDR3), in which LCDR1 comprises SEQ ID NO: 68, LCDR2 comprises SEQ ID NO: 69, and LCDR3 comprises SEQ ID NO: 70. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 11; and an immunoglobulin light chain variable region at least about 80%, 85%, 90%, 95%, 97%, 98%, 99%, or 100% identical to or consist of the amino acid sequence set forth in SEQ ID NO: 12. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 91; and an immunoglobulin light chain at least about 80%, 85%, 90%, 95%, 97%, 98%, 99%, or 100% identical to or consist of the amino acid sequence set forth in SEQ ID NO: 92. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

In some embodiments, a multi-specific binding polypeptide (e.g., a multi-specific antibody) described herein comprises a tumor binding moiety that specifically binds to FLT3 and an immune cell binding moiety that specifically binds to TRAIL-R2. In some instances, the tumor biding moiety that specifically binds to FLT3 comprises an immunoglobulin heavy chain variable region comprising CDR1 (HCDR1), CDR2 (HCDR2), and CDR3 (HCDR3), in which HCDR1 comprises SEQ ID NO: 25, HCDR2 comprises SEQ ID NO: 26, and HCDR3 comprises SEQ ID NO: 27. In some instances, the tumor biding moiety that specifically binds to FLT3 comprises an immunoglobulin light chain variable region comprising CDR1 (LCDR1), CDR2 (LCDR2), and CDR3 (LCDR3), in which LCDR1 comprises SEQ ID NO: 41, LCDR2 comprises SEQ ID NO: 42, and LCDR3 comprises SEQ ID NO: 43. In some instances, the tumor biding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 28; and an immunoglobulin light chain variable region at least about 80%, 85%, 90%, 95%, 97%, 98%, 99%, or 100% identical to or consist of the amino acid sequence set forth in SEQ ID NO: 44. In some instances, the immune cell binding moiety that specifically binds to TRAIL-R2 comprises an immunoglobulin heavy chain variable region comprising CDR1 (HCDR1), CDR2 (HCDR2), and CDR3 (HCDR3), in which HCDR1 comprises SEQ ID NO: 53, HCDR2 comprises SEQ ID NO: 54, and HCDR3 comprises SEQ ID NO: 55. In some instances, the immune cell binding moiety that specifically binds to TRAIL-R2 comprises an immunoglobulin light chain variable region comprising CDR1 (LCDR1), CDR2 (LCDR2), and CDR3 (LCDR3), in which LCDR1 comprises SEQ ID NO: 68, LCDR2 comprises SEQ ID NO: 69, and LCDR3 comprises SEQ ID NO: 70. In some instances, the immune cell binding moiety comprises an immunoglobulin heavy chain variable region comprising an amino acid sequence at least about 80%, 85%, 90%, 95%, 97%, 98%, 99%, or 100% identical to or consist of the amino acid sequence set forth in SEQ ID NO: 11; and an immunoglobulin light chain variable region at least about 80%, 85%, 90%, 95%, 97%, 98%, 99%, or 100% identical to or consist of the amino acid sequence set forth in SEQ ID NO: 12. In some cases, the multi-specific binding polypeptide (e.g., the multi-specific antibody) comprises an immunoglobulin heavy chain comprising an amino acid sequence at least about 80%, 85%, 90%, 95%, 97%, 98%, 99%, or 100% identical to or consist of the amino acid sequence set forth in SEQ ID NO: 93; and an immunoglobulin light chain at least about 80%, 85%, 90%, 95%, 97%, 98%, 99%, or 100% identical to or consist of the amino acid sequence set forth in SEQ ID NO: 94. In some cases, the multi-specific binding polypeptide (e.g., the multi-specific antibody) further comprises a payload (e.g., a cytotoxic moiety). In some instances, the multi-specific binding polypeptide (e.g., the multi-specific antibody) further comprises one or more Fc modifications (e.g., substitutions) to reduce the affinity for human neonatal Fc receptor (FcRn), to reduce ADCC functionality (e.g., a modification at L234, L235, P238, or P331, or a combination thereof), to reduce neutropenia (e.g., a modification at L234, S239, S442, or a combination thereof), to enhance ADCC (e.g., a modification at S239, A330, I332, or a combination thereof), and/or to modulate hinge region rigidity (e.g., a modification at S228).

The affinities of the tumor binding moiety and the immune cell binding moiety can be tuned to reduce off-target cytotoxic effects. In certain embodiments, the affinity of the tumor binding moiety for its target is greater than the affinity of the immune cell binding moiety for its target. In certain embodiments, the affinity of the tumor binding moiety for its target is about 1.25-fold, about 1.5-fold, about 2-fold, about 3-fold, about 4-fold, about 5-fold or about 10-fold greater than the affinity of the immune cell binding moiety for its target. In certain embodiments, the affinity of the tumor binding moiety is less than about 50 nanomolar, about 40 nanomolar, about 30 nanomolar, about 20 nanomolar, about 10 nanomolar, about 5 nanomolar, about 4 nanomolar, about 3 nanomolar, about 2 nanomolar, or about 1 nanomolar. In certain embodiments, the affinity of the immune cell binding moiety is greater than about 1 nanomolar, about 2 nanomolar, about 3 nanomolar, about 4 nanomolar, about 5 nanomolar, about 10 nanomolar, about 20 nanomolar, about 30 nanomolar, about 40 nanomolar, about 50 nanomolar, or about 100 nanomolar. In some cases, the affinity of the immune cell binding moiety to the antigen expressed on the immunosuppressive cell is less than the affinity of the tumor binding moiety to the tumor-associated antigen by about 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, or higher.

In certain embodiments, when the multi-specific binding polypeptides (e.g., multi-specific antibodies) of the current disclosure are conjugated to cytotoxic payloads they have an EC₅₀ of killing (e.g., at least about 75%) of a tumor cell of less than about 100 nanomolar, less than about 75 nanomolar, less than about 50 nanomolar, less than about 25 nanomolar, less than about 10 nanomolar, less than about 5 nanomolar. In certain embodiments, when the multi-specific binding polypeptides (e.g., multi-specific antibodies) of the current disclosure are conjugated to cytotoxic payloads they have an EC₅₀ of killing (e.g., at least about 75%) of an immunosuppressive cell of less than about 100 nanomolar, less than about 75 nanomolar, less than about 50 nanomolar, less than about 25 nanomolar, less than about 10 nanomolar, less than about 5 nanomolar.

In some embodiments, the multi-specific binding polypeptides (e.g., multi-specific antibodies) are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn297 of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (See e.g., WO 08/077546). Asn297 refers to the asparagine residue located at about position 297 in the Fc region (EU numbering of Fc region residues; See e.g., Edelman et al. Proc Natl Acad Sci USA. 1969 May; 63(1):78-85). However, Asn297 may also be located about ±3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87: 614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng., 94(4):680-688 (2006)). Other antibody glycosylation variants are also included (See e.g., U.S. Pat. No. 6,602,684).

In some embodiments, one or more amino acid modifications may be introduced into the Fc region of the multi-specific binding polypeptides provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the multi-specific binding polypeptides (e.g., multi-specific antibodies) of this disclosure comprise Fc variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies and multi-specific binding polypeptides can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, a multi-specific binding polypeptide (e.g., a multi-specific antibody) comprises an Fc region that has been modified to reduce the affinity for human neonatal Fc receptor (FcRn).

In some embodiments, a multi-specific binding polypeptide (e.g., a multi-specific antibody) comprises an Fc region that has been modified to reduce antibody-dependent cellular cytotoxicity (ADCC). In some instances, the Fc region comprises a modification at L234, L235, P238, or P331, or a combination thereof, wherein L234, L235, P238, and P331 correspond to positions 234, 235, 238, and 331 of a wild-type IgG1, according to the EU numbering convention. In some instances, the Fc region comprises a modification at L234, L235, P238, and P331, wherein L234, L235, P238, and P331 correspond to positions 234, 235, 238, and 331 of a wild-type IgG1, according to the EU numbering convention. In some instances, the Fc region comprises L234A, L235A, P238S, P331S, or a combination thereof. In some cases, the Fc region comprises L234A, L235A, P238S, and P331S.

In some embodiments, a multi-specific binding polypeptide (e.g., a multi-specific antibody) comprises an Fc region that has been modified to reduce neutropenia. In some instances, the Fc region comprises a modification at L234, S239, S442, or a combination thereof, wherein L234, S239, and S442 correspond to positions 234, 239, 442 of a wild-type IgG1, according to the EU numbering convention. In some instances, the Fc region comprises L234F, S239C, S442C, or a combination thereof.

In some embodiments, a multi-specific binding polypeptide (e.g., a multi-specific antibody) comprises an Fc region that has been modified to enhance antibody-dependent cellular cytotoxicity (ADCC). In some instances, the Fc region comprises a modification at S239, A330, I332, or a combination thereof, wherein S239, A330, and 1332 correspond to positions 239, 330, and 332 of a wild-type IgG1, according to the EU numbering convention. In some instances, the Fc region comprises S239D, A330L, 1332E, or a combination thereof. In some cases, the Fc region comprises S239D, A330L, and 1332E.

In some embodiments, a multi-specific binding polypeptide (e.g., a multi-specific antibody) comprises a modification to a hinge region. In some instances, the hinge region comprises a modification at S228, wherein S228 correspond to position 228 of a wild-type IgG4, according to the EU numbering convention. In some instances, the hinge region comprises S228P.

In some embodiments, it may be desirable to create cysteine variant multi-specific binding polypeptides, in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A114 (Kabat numbering) of the heavy chain; and S400 (EU numbering) of the heavy chain Fc region.

In some embodiments, multi-specific binding polypeptides (e.g., multi-specific antibodies) provided herein may be further modified to contain additional non-proteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

In some embodiments, the multi-specific binding polypeptides (e.g., multi-specific antibodies) provided herein further comprise a polyethylene glycol molecule, comprising, e.g., an average molecule weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some embodiments, a discrete PEG (dPEG) comprises from 2 to 60, from 2 to 50, or from 2 to 48 repeating ethylene oxide units. In some embodiments, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In such embodiments, the multi-specific binding polypeptide (e.g., multi-specific antibodies) provided herein further comprises a discrete PEG comprising, e.g., from 2 to 60, from 2 to 50, or from 2 to 48 repeating ethylene oxide units.

In some embodiments, also disclosed herein is a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in Table 5; and an immunoglobulin light chain at least about 90%, 95%, 97%, 98%, 99%, or 100% identical to or consist of the amino acid sequence set forth in Table 5.

In some embodiments, disclosed herein is a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 7 or 75-77; and optionally an immunoglobulin light chain at least about 90%, 95%, 97%, 98%, 99%, or 100% identical to or consist of the amino acid sequence set forth in SEQ ID NO: 8.

In some embodiments, disclosed herein is a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 79-83; and optionally an immunoglobulin light chain at least about 90%, 95%, 97%, 98%, 99%, or 100% identical to or consist of the amino acid sequence set forth in SEQ ID NO: 80.

In some embodiments, disclosed herein is a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 84-88; and optionally an immunoglobulin light chain at least about 90%, 95%, 97%, 98%, 99%, or 100% identical to or consist of the amino acid sequence set forth in SEQ ID NO: 85.

In some embodiments, disclosed herein is a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 89, 91, or 93; and optionally an immunoglobulin light chain at least about 90%, 95%, 97%, 98%, 99%, or 100% identical to or consist of the amino acid sequence set forth in SEQ ID NO: 90, 92, or 94.

In some embodiments, disclosed herein is a nucleic acid encoding a multi-specific antibody comprising a VH and a VL disclosed in Tables 1 and 2 and optionally in combination with a VH and a VL of Tables 3 and 4.

In some embodiments, additionally disclosed herein is a vector comprising a nucleic acid of encoding a multi-specific antibody comprising an immunoglobulin heavy chain comprising an amino acid sequence at least about 90%, 95%, 97%, 98%, 99%, or 100% identical to or consist of the amino acid sequence set forth in Table 5; and an immunoglobulin light chain at least about 90%, 95%, 97%, 98%, 99%, or 100% identical to or consist of the amino acid sequence set forth in Table 5. In some cases, the vector comprises a viral vector (e.g., a retroviral vector, a lentiviral vector, an adenoviral vector, an adeno-associated viral vector, a herpes simplex virus vector, or a chimeric viral vector). In additional cases, the vector comprises a non-viral vector.

### Methods of Target Selection

In certain embodiments, described herein is a method of selecting a first target receptor and a second target receptor for generating a multi-specific polypeptide molecule (e.g., a multi-specific antibody) for the treatment of a cancer. In some embodiments, the method comprises the steps:
a. selecting antigens/receptors expressed in cancer cells that are detected in tumors with intensity score of +2 to +3 determined by IHC, and/or selecting antigens/receptors expressed in cancer cells that are detected in tumors with expression level score of RNASeqV2 (Log)>5;
b. selecting antigens/receptors expressed in immunosuppressive cells that are detected in tumors with intensity score of +2 to +3 determined by IHC, and/or selecting antigens/receptors expressed in immunosuppressive cells that are detected in tumors with expression level score of RNASeqV2 (Log)>5; and
c. selecting antigens/receptors in respective cell types that are detected in the same tumor with expression level score of RNASeqV2 (Log)>5, and/or selecting antigens/receptors in respective cell types that are detected in the same tumor with intensity score of +2 to +3 determined by IHC;
   wherein the selected targets in respective cell types are expressed in non-tumor tissues with intensity score 0 to +1 as determined by IHC, and/or the selected targets in respective cell types are expressed in non-tumor tissues with expression level score of RNASeqV2 (Log)<4. In certain embodiments, the receptors are selected by analyzing cancer genome databases that reports the expression level of receptors by estimating RNA level, protein level and staining intensities by IHC. In certain embodiments, the receptors are selected by analyzing cancer genome databases using data analytics software that executes steps a-c.

### Production of Multi-Specific Binding Polypeptides

Included herein are methods to manufacture a multi-specific binding polypeptide (e.g., a multi-specific antibody). The multi-specific binding polypeptides (e.g., multi-specific antibodies) can be produced by several methods known in the art. For example, the multi-specific binding polypeptides can be encoded by nucleic acid(s). This nucleic acid can be, for example, a plasmid or viral vector that is then transferred to a suitable cell line such as, for example, a eukaryotic host cell or a prokaryotic host cell.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HCC-38, HCC-1806, HepaRG™ cells, MCF-7, MDA-MB-468, MDA-MB-231, SK-BR-3, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris yeast strains such as GS115, KM71H, SMD1168, SMD1168H, and X-33, and Saccharomyces cerevisiae yeast strain such as INVSc1.

In some embodiments, a eukaryotic host cell is a plant host cell. In some embodiments, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Machl™, DH10B™, TOP10, DH5α, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some embodiments, the plasmid vector includes a vector from bacteria (e.g., E. coli), insects, yeast (e.g., Pichia pastoris), algae, or mammalian source. Bacterial vectors include, for example, pACYC177, pASK75, pBAD vector series, pBADM vector series, pET vector series, pE™ vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

A cell line comprising the nucleic acid encoding the multi-specific binding polypeptide can then be cultured under suitable conditions such that the multi-specific binding polypeptide is expressed and secreted into the cell supernatant. The cell supernatant can be subjected to one or more steps that include filtration, centrifugation, precipitation, purification by ion exchange, protein A/G affinity, dialysis, desalting, or buffer exchange.

In some embodiments, an expression vector comprising the nucleotide sequence of a multi-specific binding polypeptide is transferred to a host cell by conventional techniques (e.g., viral transduction, electroporation, liposomal transfection, calcium phosphate precipitation), and the transfected cells are then cultured to produce the multi-specific binding polypeptide. In specific embodiments, the expression of the multi-specific binding polypeptide is regulated by a constitutive, an inducible, or a tissue specific promoter.

In some embodiments, a variety of host-expression vector systems are utilized to express a multi-specific binding polypeptide described herein. Such host-expression systems represent vehicles, by which the coding sequences of the multi-specific binding polypeptide (e.g., multi-specific antibody) is produced and subsequently purified, but also represent cells that are, when transformed or transfected with the appropriate nucleotide coding sequences, express an multi-specific binding polypeptide in situ. These include, but are not limited to, microorganisms such as bacteria (e.g., E. coli and B. subtilis) transformed with recombinant bacteriophage DNA, plasmid DNA or cosmid DNA expression vectors containing multi-specific binding polypeptide coding sequences; yeast (e.g., Saccharomyces Pichia) transformed with recombinant yeast expression vectors containing multi-specific binding polypeptide coding sequences; insect cell systems infected with recombinant virus expression vectors (e.g., baculovirus) containing multi-specific binding polypeptide coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing multi-specific binding polypeptide coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some embodiments, cell lines that stably express a multi-specific binding polypeptide (e.g., a multi-specific antibody) are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the multi-specific binding polypeptide.

In some embodiments, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some embodiments, the expression levels of a multi-specific binding polypeptide (e.g., a multi-specific antibody) are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing a multi-specific binding polypeptide is amplifiable, an increase in the level of inhibitor present in culture of host cell will increase the number of copies of the marker gene. Since the amplified region is associated with the nucleotide sequence of the multi-specific binding polypeptide, production of the multi-specific binding polypeptide will also increase (Crouse et al., 1983, Mol. Cell Biol. 3:257).

In some embodiments, the multi-specific binding polypeptide (e.g., multi-specific antibody) is produced under a cell-free system. In some embodiments, a cell-free system comprises a mixture of cytoplasmic and/or nuclear components from a cell and is suitable for in vitro nucleic acid synthesis. In some embodiments, a cell-free system utilizes prokaryotic cell components. In other embodiments, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

### Cytotoxic Payloads

In certain embodiments, the multi-specific binding polypeptides (e.g., multi-specific antibodies) of the current disclosure can be conjugated to payloads (e.g., cytotoxic moieties) as shown in FIG. 1B. These cytotoxic moieties are payloads specifically targeted to kill both tumors and immunosuppressive cells. In certain embodiments, the multi-specific polypeptide molecule comprises at least one cytotoxic moiety. In other embodiments, the multi-specific polypeptide molecule comprises two or more cytotoxic moieties.

In some embodiments, the cytotoxic payload comprises a microtubule disrupting agent. Exemplary microtubule disrupting agents include, but are not limited to, 2-methoxyestradiol, chalcones, colchicine, combretastatin, dictyostatin, discodermolide, eleutherobin, epothilone, laulimalide, peloruside, podophyllotoxin, taxane, cryptophycin, halichondrin, maytansine, phomopsin, rhizoxin, spongistatin, tubulysin, vinca alkaloid, noscapinoid, auristatin, dolastain, or derivatives or analogs thereof. In some embodiments, the cytotoxic payload is combretastatin or a derivative or analog thereof. In some embodiments, an analog of combretastatin is ombrabulin. In some embodiments, the epothilone is epothilone B, patupilone, ixabepilone, sagopilone, BMS-310705, or BMS-247550. In some embodiments, the tubulysin is a tubulysin analog or derivative such as described in U.S. Pat. Nos. 8,580,820 and 8,980,833 and in U.S. Publication Nos. 20130217638, 20130224228, and 201400363454. In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268. In some embodiments, the taxane is paclitaxel or docetaxel. In some embodiments, the vica alkaloid is vinblastine, vincristine, vindesine, vinorelbine, desoxyvincaminol, vincaminol, vincamajine, vineridine, vinburnine, vinpocetine, or vincamine.

In some embodiments, the cytotoxic payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714 and 8,871,720. In some embodiments, the dolastatin 15 analog is cemadotin or tasidotin.

In some embodiments, the cytotoxic payload comprises a DNA modifying agent. In some embodiments, the DNA modifying agent comprises amsacrine, anthracycline, camptothecin, doxorubicin, duocarmycin, enediyne, etoposide, indolinobenzodiazepine, netropsin, teniposide, pyrrolobenzodiazepine, or derivatives or analogs thereof. In some embodiments, the anthracycline is doxorubicin, daunorubicin, epirubicin, idarubicin, mitomycin-C, dactinomycin, mithramycin, nemorubicin, pixantrone, sabarubicin, or valrubicin. In some embodiments, the analog of camptothecin is topotecan, irinotecan, silatecan, cositecan, exatecan, lurtotecan, gimatecan, belotecan, rubitecan, or SN-38. In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the cytotoxic payload is pyrrolobenzodiazepine. In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In certain embodiments, the at least one cytotoxic moiety comprises an auristatin derivative, maytansine, a maytansinoid, a taxane, a calicheamicin, cemadotin, a duocarmycin, a pyrrolobenzodiazepine (PBD), or a tubulysin. the auristatin derivative is monomethyl auristatin E (MMAE) or monomethyl auristatin F (MMAF). In certain embodiments, the maytansinoid is DM1 (emtansine). In certain embodiments, the maytansinoid is DM2 (mertansine) or DM4 (ravtansine/soravtansine). In certain embodiments, the pyrrolobenzodiazepine is a pyrrolobenzodiazepine dimer.

The cytotoxic moiety can be conjugated to the multi-specific binding polypeptide at a suitable stoichiometry. In certain embodiments, the cytotoxic moiety is conjugated at a ratio of about 1:1; about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, or about 20:1 cytotoxic moiety:multi-specific binding polypeptide.

### Linkers

In certain embodiments, the cytotoxic moiety is conjugated to lysine residues, cysteine residues, or lysine and cysteine residues by a suitable linker molecule. In certain embodiments, the linker is a cleavable linker. In certain embodiments, the linker is a pH-cleavable linker. In certain embodiments, the linker is an enzyme-cleavable linker. In certain embodiments, the linker is a protease-sensitive linker. In certain embodiments, the enzyme cleavage linker is a valine-citruline linker. In certain embodiments, the linker is a self-immolative linker. In certain embodiments, the linker is a non-cleavable linker. In certain embodiments, the linker comprises a zero-length linker, a homobifunctional linker, a heterobifunctional linker, a di-peptide linker, a spacer, a maleimide-based conjugating moiety, or a combination thereof. In certain embodiments, the linker comprises a polymer. In certain embodiments, the polymer comprises a linear or branched polyethylene glycol. In certain embodiments, the linker is a peptide. In certain embodiments, the linker is a peptidomimetic linker. In certain embodiments, the linker is a thioether linker.

Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino)hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (ρNPDP), p-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a reactive functional group. In some embodiments, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and acylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (mc). In some embodiments, the linker comprises maleimidocaproyl (mc). In some embodiments, the linker is maleimidocaproyl (mc). In other embodiments, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some embodiments, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some embodiments, the linker comprises a self-stabilizing maleimide. In some embodiments, the linker is a self-stabilizing maleimide.

In some embodiments, the linker comprises a peptide moiety. In some embodiments, the peptide moiety comprises at least 2, 3, 4, 5, 6, 7, 8, or more amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly (SEQ ID NO: 142), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 143), or Gly-Phe-Leu-Gly (SEQ ID NO: 144). In some embodiments, the linker comprises a peptide moiety such as: Val-Cit (valine-citrulline), Gly-Gly-Phe-Gly (SEQ ID NO: 142), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 143), or Gly-Phe-Leu-Gly (SEQ ID NO: 144). In some embodiments, the linker comprises Val-Cit. In some embodiments, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (mc). In some embodiments, the peptide group is val-cit. In some embodiments, the benzoic acid group is PABA. In some embodiments, the linker comprises a mc-val-cit group. In some embodiments, the linker comprises a val-cit-PABA group. In additional embodiments, the linker comprises a mc-val-cit-PABA group.

In some embodiments, the linker is a self-immolative linker or a self-elimination linker. In some embodiments, the linker is a self-immolative linker. In other embodiments, the linker is a self-elimination linker (e.g., a cyclization self-elimination linker). In some embodiments, the linker comprises a linker described in U.S. Pat. No. 9,089,614 or PCT Publication No. WO2015038426.

In some embodiments, the linker is a traceless linker or a linker in which after cleavage does not leave behind a linker moiety (e.g., an atom or a linker group) to a payload or to a multi-specific binding polypeptide described herein. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some embodiments, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., “Traceless solid-phase organic synthesis,” Chem. Rev. 102: 2607-2024 (2002). In some embodiments, a linker is a traceless linker as described in U.S. Pat. No. 6,821,783.

In some embodiments, the linker is a dendritic type linker. In some embodiments, the dendritic type linker comprises a branching, multifunctional linker moiety. In some embodiments, the dendritic type linker comprises PAMAM dendrimers.

In some embodiments, the linker is an acid cleavable linker. In some embodiments, the acid cleavable linker comprises a hydrazone linkage, which is susceptible to hydrolytic cleavage. In some embodiments, the acid cleavable linker comprises a thiomaleamic acid linker. In some embodiments, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some embodiments, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

### Conjugation Chemistry

Various conjugation reactions are described herein and contemplated for reacting a multi-specific binding polypeptide described herein (e.g., a multi-specific antibody, optionally a bispecific antibody) with a payload (e.g., a cytotoxic payload). In some embodiments, the reaction occurs at a natural (“canonical”) amino acid in the multi-specific binding polypeptide described herein (e.g., the multi-specific antibody, optionally the bispecific antibody). In some embodiments, the amino acid for conjugation is a natural amino acid found in a wild type sequence, or alternatively the amino acid has been mutated. In some embodiments, a conjugation reaction comprises formation of a disulfide bond at a cysteine residue. In some embodiments, a conjugation reaction comprises a 1,4 Michael addition reaction of a cysteine or lysine. In some embodiments, a conjugation reaction comprises a cyanobenzothiazole ligation of a cysteine. In some embodiments, a conjugation reaction comprises crosslinking with an acetone moiety, such as 1,3-dichloro-2-propionone. In some embodiments, a conjugation reaction comprises a 1,4 Michael addition to a dehydroalanine, formed by reaction of cysteine with O-mesitylenesulfonylhydroxylamine. In some embodiments a conjugation reaction comprises reaction of a tyrosine with a triazolinedione (TAD), or TAD derivative. In some embodiments a conjugation reaction comprises reaction of a tryptophan with a rhodium carbenoid.

In some embodiments, a multi-specific binding polypeptide described herein (e.g., a multi-specific antibody, optionally a bispecific antibody) is conjugated to a payload described supra by a chemical ligation process. In some instances, the multi-specific binding polypeptide described herein (e.g., the multi-specific antibody, optionally the bispecific antibody) is conjugated to the payload by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some embodiments, the multi-specific binding polypeptide described herein (e.g., the multi-specific antibody, optionally the bispecific antibody) is conjugated to the payload either site-specifically or non-specifically via native ligation chemistry.

In some instances, a multi-specific binding polypeptide described herein (e.g., a multi-specific antibody, optionally a bispecific antibody) is conjugated to a payload described supra by a “Click” chemistry. In some instances, the conjugation reaction comprises a 1,3-dipolar cycloaddition reaction. In some embodiments, the 1,3-dipolar cycloaddition reaction comprises reaction of an azide and a phosphine (“Click” reaction). In some embodiments, the conjugation reaction is catalyzed by copper. In some embodiments, the conjugation reaction comprises reaction of an azide with a strained olefin. In some embodiments, a conjugation reaction comprises reaction of an azide with a strained alkyne. In some embodiments, a conjugation reaction comprises reaction of an azide with a cycloalkyne, for example, OCT, DIFO, DIFBO, DIBO, BARAC, TMTH, or other strained cycloalkyne, the structures of which are shown in Gong, Y., Pan, L. Tett. Lett. 2015, 56, 2123. In some embodiments, a 1,3-dipolar cycloaddition reaction is catalyzed by light (“photoclick”). In some embodiments, a conjugation reaction comprises reaction of a terminal allyl group with a tetrazole and light. In some embodiments, a conjugation reaction comprises reaction of a terminal alkynyl group with a tetrazole and light. In some embodiments, a conjugation reaction comprises reaction of an O-allyl amino acid with a tetrazine and light. In some embodiments, a conjugation reaction comprises reaction of O-allyl tyrosine with a tetrazine and light.

In some instances, a multi-specific binding polypeptide described herein (e.g., a multi-specific antibody, optionally a bispecific antibody) is conjugated to a payload described supra by a GlyCLICK® site-specific conjugation technology (Life Technologies Corporation). In some instances, the GlyCLICK® site-specific conjugation chemistry comprises a Fc-glycan remodeling which comprises a deglycosylation of the antibody to allow site-specific conjugation using click-chemistry.

In some instances, a multi-specific binding polypeptide described herein (e.g., a multi-specific antibody, optionally a bispecific antibody) is conjugated to a payload described supra by a GlycoConnect™ conjugation technology (Synaffix BV). In some instances, the GlycoConnect™ conjugation technology utilizes enzymatic modification of two naturally occurring glycan anchor points to engage in site-specific conjugation.

In some instances, a multi-specific binding polypeptide described herein (e.g., a multi-specific antibody, optionally a bispecific antibody) is conjugated to a payload described supra by an immunoglobulin binding peptide. In some instances, the immunoglobulin binding peptide utilizes IgG Fc-affinity reagents to directly conjugate cytotoxic payloads to the multi-specific binding polypeptide (e.g., the multi-specific antibody).

In some instances, a multi-specific binding polypeptide described herein (e.g., a multi-specific antibody, optionally a bispecific antibody) is conjugated to a payload described supra by an AJICAP™ conjugation technology (Ajinomoto Co. Inc.). In some instances, the AJICAP™ conjugation technology utilizes a class of IgG Fc-affinity reagents to directly conjugate one or more cytotoxic payloads to the multi-specific binding polypeptide (e.g., the multi-specific antibody).

In some instances, a multi-specific binding polypeptide described herein (e.g., a multi-specific antibody, optionally a bispecific antibody) is conjugated to a payload described supra by a conjugation reaction comprising an inverse-electron demand cycloaddition reaction that comprises a diene and a dienophile. In some embodiments, the diene comprises a tetrazine. In some embodiments, the dienophile comprises an alkene. In some embodiments, the dienophile comprises an alkyne. In some embodiments, the alkyne is a strained alkyne. In some embodiments, the alkene is a strained diene. In some embodiments, the alkyne is a trans-cyclooctyne. In some embodiments, the alkyne is a cyclooctene. In some embodiments, the alkene is a cyclopropene. In some embodiments, the alkene is a fluorocyclopropene. In some embodiments, a conjugation reaction results in the formation of a multi-specific binding polypeptide (e.g., a multi-specific antibody) attached to a linker or payload via a 6-membered ring heterocycle comprising two nitrogen atoms in the ring.

In some instances, a multi-specific binding polypeptide described herein (e.g., a multi-specific antibody, optionally a bispecific antibody) is conjugated to a payload described supra by a conjugation reaction comprising an olefin metathesis reaction. In some embodiments, a conjugation reaction comprises reaction of an alkene and an alkyne with a ruthenium catalyst. In some embodiments, a conjugation reaction comprises reaction of two alkenes with a ruthenium catalyst. In some embodiments, a conjugation reaction comprises reaction of two alkynes with a ruthenium catalyst. In some embodiments, a conjugation reaction comprises reaction of an alkene or alkyne with a ruthenium catalyst and an amino acid comprising an allyl group. In some embodiments, a conjugation reaction comprises reaction of an alkene or alkyne with a ruthenium catalyst and an amino acid comprising an allyl sulfide or selenide. In some embodiments, a ruthenium catalyst is Hoveda-Grubbs 2nd generation catalyst. In some embodiments, an olefin metathesis reaction comprises reaction of one or more strained alkenes or alkynes.

In some instances, a multi-specific binding polypeptide described herein (e.g., a multi-specific antibody, optionally a bispecific antibody) is conjugated to a payload described supra by a conjugation reaction comprising a cross-coupling reaction. In some embodiments, cross-coupling reactions comprise transition metal catalysts, such as iridium, gold, ruthenium, rhodium, palladium, nickel, platinum, or other transition metal catalyst and one or more ligands. In some embodiments, transition metal catalysts are water-soluble. In some embodiments, a conjugation reaction comprises a Suzuki-Miyaura cross-coupling reaction. In some embodiments, a conjugation reaction comprises reaction of an aryl halide (or triflate, or tosylate), an aryl or alkenyl boronic acid, and a palladium catalyst. In some embodiments, a conjugation reaction comprises a Sonogashira cross-coupling reaction. In some embodiments, a conjugation reaction comprises reaction of an aryl halide (or triflate, or tosylate), an alkyne, and a palladium catalyst. In some embodiments, cross-coupling reactions result in attachment of a linker or payload to a multi-specific binding polypeptide (e.g., a multi-specific antibody) via a carbon-carbon bond.

In some instances, a multi-specific binding polypeptide described herein (e.g., a multi-specific antibody, optionally a bispecific antibody) is conjugated to a payload described supra by a site-directed method utilizing a “traceless” coupling technology (Philochem). In some instances, the “traceless” coupling technology utilizes an N-terminal 1,2-aminothiol group on the multi-specific binding polypeptide which is then conjugate with a payload containing an aldehyde group. (see Casi et al., “Site-specific traceless coupling of potent cytotoxic drugs to recombinant antibodies for pharmacodelivery,” JACS 134(13): 5887-5892 (2012)).

In some instances, a multi-specific binding polypeptide described herein (e.g., a multi-specific antibody, optionally a bispecific antibody) is conjugated to a payload described supra by a site-directed method utilizing an unnatural amino acid incorporated into the multi-specific binding polypeptide (e.g., the multi-specific antibody, optionally the bispecific antibody). In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, a multi-specific binding polypeptide described herein (e.g., a multi-specific antibody, optionally a bispecific antibody) is conjugated to a payload described supra by a site-directed method utilizing an enzyme-catalyzed process. In some instances, the site-directed method utilizes SMARTag™ technology (Catalent Biologics). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized multi-specific binding polypeptide (e.g., functionalized multi-specific antibody, optionally functionalized bispecific antibody) via hydrazino-Pictet-Spengler (HIPS) ligation. In some instances, a 6-amino acid consensus sequence is incorporated into the heavy chain, light chain, or both chains of the multi-specific antibody (optionally the bispecific antibody) for recognition by the FGE to generate a functional aldehyde group for site-specific conjugation to the payload. In some instances, the 6-amino acid consensus sequence is LCXPXR, wherein X is any amino acid. In some cases, the 6-amino acid consensus sequence is incorporated into the N-terminus, C-terminus, or within a Fc region of the multi-specific antibody (optionally the bispecific antibody). (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, a multi-specific binding polypeptide described herein (e.g., a multi-specific antibody, optionally a bispecific antibody) is conjugated to a payload described supra utilizing a microbial transglutaminze catalyzed process. In some instances, mTG catalyzes the formation of a covalent bond between the amide side chain of a glutamine within the recognition sequence and a primary amine of a functionalized multi-specific binding polypeptide (e.g., a functionalized multi-specific antibody, optionally a functionalized bispecific antibody). In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, a multi-specific binding polypeptide described herein (e.g., a multi-specific antibody, optionally a bispecific antibody) is conjugated to a payload described supra by a method as described in PCT Publication No. WO2014/140317, which utilizes a sequence-specific transpeptidase.

In some instances, a multi-specific binding polypeptide described herein (e.g., a multi-specific antibody, optionally a bispecific antibody) is conjugated to a payload described supra by a method as described in U.S. Patent Publication Nos. 2015/0105539 and 2015/0105540.

### Methods of Use

The multi-specific binding polypeptides (e.g., multi-specific antibodies) of the current disclosure are useful in the treatment of a disease or condition. In some instances, the disease or condition is a cancer (e.g., a carcinoma, sarcoma, leukemia, or lymphoma). In certain embodiments, the multi-specific binding polypeptides (e.g., multi-specific antibodies) are for use in a method to treat a cancer. In certain embodiments, the multi-specific binding polypeptides (e.g., multi-specific antibodies) are for use in the manufacture of a medicament for treating a cancer. Many cancers can be treated with the multi-specific binding polypeptides (e.g., multi-specific antibodies) described herein including solid tumors/cancers and hematological malignancies. In certain embodiments, the solid cancer is bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer. In certain embodiments, the hematological malignancy comprises a Hodgkin's lymphoma or a non-Hodgkin's lymphoma. Exemplary hematological malignancies include, but are not limited to, chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, or acute myeloid leukemia. In some embodiments, the hematological malignancy comprises chronic lymphocytic leukemia (CLL), acute myeloid leukemia (AML), diffuse large B-cell lymphoma (DLBCL), chronic myeloid leukemia, or acute myeloid leukemia.

In some embodiments, the cancer is a metastatic cancer. In some embodiments, the metastatic cancer is a metastatic solid tumor/cancer. In other embodiments, the metastatic cancer is a metastatic hematologic malignancy. In some embodiments, the metastatic solid tumor comprises metastatic bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer. In some embodiments, the metastatic hematologic malignancy comprises metastatic chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, or acute myeloid leukemia.

In some embodiments, the cancer is a relapsed or refractory cancer. In some embodiments, the relapsed or refractory cancer is a solid cancer, e.g., a relapsed or refractory bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer. In some embodiments, the relapsed or refractory cancer is a hematologic malignancy, e.g., a relapsed or refractory chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, or acute myeloid leukemia.

In certain embodiments, the cancer is a breast cancer. In some embodiments, the breast cancer is luminal A breast cancer, luminal B breast cancer, triple-negative breast cancer, HER2-enriched breast cancer, or normal-like breast cancer. In some embodiments, the breast cancer is ductal carcinoma in situ (DCIS), invasive ductal carcinoma (IDC), invasive lobular carcinoma (ILC), inflammatory breast cancer, lubular carcinoma in situ (LCIS), male breast cancer, Paget's disease of the Nipple, or phyllodes tumors of the breast. In some embodiments, the IDC comprises tubular carcinoma of the breast, medullary carcinoma of the breast, papillary carcinoma of the breast, or cribriform carcinoma of the breast. In some embodiments, the breast cancer is triple negative breast cancer. In some embodiments, the breast cancer is a metastatic breast cancer. In additional embodiments, the breast cancer is a relapsed or refractory breast cancer.

In certain embodiments, the cancer is an ovarian cancer. In some embodiments, the ovarian cancer is epithelial carcinoma, serous carcinoma, small-cell carcinoma, primary peritoneal carcinoma, clear-cell carcinoma, clear-cell adenocarcinoma, endometrioid, malignant mixed mullerian tumor, mucinous, mucinous adenocarcinoma, pseudomyxoma peritonel, undifferentiated epithelial, malignant Brener tumor, transitional cell carcinoma, sex cord-stromal tumor, granulosa cell tumor, adult granulosa cell tumor, juvenile granulosa cell tumor, Sertoli-Leydig cell tumor, sclerosing stromal tumors, germ cell tumor, dysgerminoma, choriocarcinoma, immature (solid) teratoma, mature teratoma (dermoid cyst), yolk sac tumor (endodermal sinus tumor), embryonal carcinoma, polyembryoma, squamous cell carcinoma, mixed tumors, or low malignant potential tumors. In some embodiments, the ovarian cancer is a metastatic ovarian cancer. In additional embodiments, the ovarian cancer is a relapsed or refractory ovarian cancer.

In certain embodiments, the cancer is a lung cancer. In some embodiments, the lung cancer is non-small cell lung carcinoma (NSCLC) or small cell lung cancer (SCLC). In some embodiments, the lung cancer is a metastatic lung cancer. In additional embodiments, the lung cancer is a relapsed or refractory lung cancer.

In certain embodiments, the cancer is a liver cancer. In some embodiments, the liver cancer is hepatocellular carcinoma (HCC), cholangiocarcinoma, liver angiosarcoma, or hepatoblastoma. In some instances, the liver cancer is a metastatic liver cancer. In some cases, the liver cancer is a relapsed or refractory liver cancer.

In certain embodiments, the cancer is a prostate cancer. In some embodiments, the prostate cancer is acinar adenocarcinoma, ductal adenocarcinoma, transitional cell (or urothelial) cancer, squamous cell cancer, small cell prostate cancer, carcinoid in the prostate, or sarcoma in the prostate. In some embodiments, the prostate cancer is a metastatic prostate cancer. In additional embodiments, the prostate cancer is a relapsed or refractory prostate cancer.

In certain embodiments, the cancer is a brain cancer. In some instances, the brain cancer is glioblastoma (e.g., glioblastoma multiforme or GBM). In some instances, the brain cancer is neuroblastoma. In some cases, the brain cancer is a metastatic brain cancer.

In certain embodiments, the cancer is a carcinoma.

In certain embodiments, the cancer is a sarcoma.

In certain embodiments, the cancer is acute myeloid leukemia, diffuse large B cell lymphoma (DLBCL), bladder urothelial carcinoma, breast carcinoma, triple negative breast carcinoma, liver hepatocellular carcinoma, cervical squamous cell carcinoma, cholangiocarcinoma, colon adenocarcinoma, esophageal carcinoma, head and neck squamous cell carcinoma, kidney renal papillary cell carcinoma, lung adenocarcinoma, lung squamous cell carcinoma, NSCLC, SCLC, ovarian serous cystadenocarcinoma, pancreatic adenocarcinoma, prostate adenocarcinoma, kidney renal clear cell carcinoma, uterine corpus endometrial carcinoma, thyroid carcinoma, stomach adenocarcinoma, rectal adenocarcinoma, sarcoma, testis and germ cell tumors, or uterine carcinosarcoma.

In some embodiments, the cancer is a pediatric cancer. Exemplary pediatric cancers include, but not limited to, bone cancer, brain cancer, leukemia, hepatoblastoma, lymphoma (e.g., Hodgkin's and non-Hodgkin's lymphoma), neuroblastoma, rhabdomyosarcoma, retinoblastoma, rhabdoid tumor, sarcoma, spinal cord tumor, and Wilms tumor. In some instances, a pediatric cancer occurs in a child less than 18 years age.

In certain embodiments, the disease or condition is a liver disease or condition. In some cases, the liver disease or condition is non-alcoholic fatty liver disease (NASH) or alcoholic steatohepatitis.

The multi-specific binding polypeptides (e.g., multi-specific antibodies) described herein can be used to treat cancers and/or tumors that are refractory to treatment with immune checkpoint inhibitor therapies, fail treatment with immune checkpoint inhibitor therapies, or respond sub-optimally to treatment with immune checkpoint inhibitor therapies. In certain embodiments, checkpoint inhibitor therapies include therapies that target PD-1, PDL-1, PDL-2, CTLA4, LAG-3, TIM-3, KIR, VISTA or other immune checkpoint receptors. Current checkpoint inhibitor therapies include, for example, nivolumab (Opdivo®), pembrolizumab (Keytruda®), pidilizumab (CT-011), BMS-936559, atezolizumab (MPDL3280A), avelumab, ipilimumab (Yervoy®), or tremelimumab. In certain embodiments, a subject is selected for treatment with the multi-specific binding polypeptides (e.g., multi-specific antibodies) described herein after receiving a checkpoint inhibitor therapy. In certain embodiments, a subject is selected for treatment with the multi-specific binding polypeptides (e.g., multi-specific antibodies) described herein after failing a checkpoint inhibitor therapy. In certain embodiments, failing a checkpoint inhibitor therapy is a lack of a complete response (e.g., complete reduction of tumor or signs of cancer). In certain embodiments, failing a checkpoint inhibitor therapy is a lack of an objective response (e.g., lack of measurable tumor shrinkage or reduction in signs of cancer). In certain embodiments, the multi-specific binding polypeptides (e.g., multi-specific antibodies) are for use in a method to treat a subject that has been administered one or more previous immune checkpoint inhibitor treatments. In certain embodiments, subjects are selected for treatment with the multi-specific binding polypeptides (e.g., multi-specific antibodies) provided herein based upon low expression or an absence of expression of an immune checkpoint inhibitor. In certain embodiments, subjects are selected for treatment with the multi-specific binding polypeptides (e.g., multi-specific antibodies) provided herein based upon low expression or an absence of expression of PD-1, PDL-1, PDL-2, CTLA4, LAG-3, TIM-3, KIR, VISTA or other immune checkpoint receptors. Low expression or absent expression can be suitably determined using methods such as immunohistochemistry, flow-cytometry, RT-PCR, RNA-Seq, ELISA or western blot. Exemplary assays for PDL-1 diagnostic purposes are known, and, for example, can be found in Udall et al. Diagn Pathol. 2018; 13: 12. In some embodiments, the subject is insensitive to treatment with an immune checkpoint inhibitor, has failed to respond to treatment with an immune checkpoint inhibitor, or who expresses low level of or does not express an immune checkpoint protein. In some instances, the multi-specific binding polypeptides (e.g., multi-specific antibodies) described herein are administered to the subject in conjugation with an immune checkpoint modulator, e.g., simultaneously with an immune checkpoint modulator or sequentially with an immune checkpoint modulator.

The choice of tumor associated antigen (TAA) and immune cell binding moiety of the multi-specific binding polypeptide can be dependent upon the type of cancer treated. Table 6 lists some specific cancers and antigen choices. In certain embodiments, a multi-specific binding polypeptide to treat the indicated cancer comprises the binding specificities listed in Table 6.

TABLE 6

| | | Antigen expressed on an |
|---|---|---|
| Cancer | TAA | immunosuppressive cell |
| Acute Myeloid | CD33; | TRAIL-R2, TNFR2, CSF1R, |
| Leukemia | CD123; | SEMA4A, SEMA4D, |
| | CD38; or | SEMA4D, CD163, |
| | FLT3 | MS4A7, C5AR1, |
| | | LILRB4, MRC1, STAB1, |
| | | MERTK, SIGLEC7, |
| | | SIGLEC9, IL4R, CLEC10A, |
| | | CD200R1, SELPLG |
| Diffuse Large B Cell | CD123; | TRAIL-R2, CD33, CSF1R, |
| Lymphoma | CD30; | SEMA4A, SEMA4D, |
| | CD33; or | CD163, MARCO, TNFR2, |
| | CD19 | MS4A7, C5AR1, ABCC3, |
| | | LILRB4, STAB1, TMEM119, |
| | | SIGLEC1, SIGLEC7, |
| | | SIGLEC9, CLEC10A, |
| | | IL4R, SELPLG |
| Bladder Urothelial | TROP2/TACSTD2 | TRAIL-R2, CSF1R, |
| Carcinoma | | SEMA4A, SEMA4D, |
| | | CD163, TNFR2, TREM2, |
| | | MS4A7, TMEM119, ABCC3, |
| | | STAB1, IL4R, SELPLG |
| Breast Carcinoma | TROP2/TACSTD2; | TRAIL-R2, CD33, CSF1R, |
| | or | SEMA4A, SEMA4D, |
| | HER2/ERBB2 | CD163, TNFR2, TREM2, |
| | | MS4A7, C5AR1, |
| | | ABCC3, LILRB4, |
| | | STAB1, TMEM119, |
| | | TMEM37, IL4R, |
| | | CLEC10A, SELPLG |
| Triple negative | TROP2/TACSTD2; | TRAIL-R2, CD33, |
| breast carcinoma | or | CSF1R, SEMA4A, |
| | FOLR1 | SEMA4D, CD163, MARCO, |
| | | TNFR2, TREM2, MS4A7, |
| | | C5AR1, ABCC3, |
| | | LILRB4, MRC1, |
| | | STAB1, TMEM119, |
| | | TMEM37, SIGLEC1, |
| | | CLEC10A, SELPLG |
| Liver Hepatocellular | GPC3 | ABCC3, TMEM37, STAB1, |
| Carcinoma | | IL4R, TNFRSF10B, |
| | | TNFRSF1B, CSF1R, |
| | | TREM2, MS4A7, CD163 |
| Cervical Squamous | TROP2/TACSTD2 | TRAIL-R2, CSF1R, SEMA4A, |
| cell carcinoma | | SEMA4D, TNFR2, |
| | | TREM2, ABCC3, |
| | | LILRB4, STAB1, |
| | | TMEM37, IL4R |
| Cholangiocarcinoma | TROP2/TACSTD2 | TRAIL-R2, CSF1R, TNFR2, |
| | | TREM2, MS4A7, CSAR1, |
| | | ABCC3, LILRB4, STAB1, |
| | | TMEM37, IL4R |
| Colon | TROP2/TACSTD2; | TRAIL-R2, TNFR2, ABCC3, |
| Adenocarcinoma | or HER2/ERBB2 | STAB1, IL4R |
| Esophageal | TROP2/TACSTD2; | TRAIL-R2, CSF1R, SEMA4A, |
| Carcinoma | GPC3; or | SEMA4D, CD163, |
| | HER2/ERBB2 | TNFR2, TREM2, MS4A7, |
| | | C5AR1, ABCC3, STAB1, |
| | | TMEM37, IL4R, MGL1/ |
| | | CD301/CLEC10A, CD200R |
| | | SELPLG/PSLG-1/CD162 |
| Head and Neck | TROP2/TACSTD2 | TRAIL-R2, CSF1R, SEMA4A, |
| Squamous Cell | | SEMA4D, CD163, TNFR2, |
| Carcinoma | | TREM2, MS4A7, C5AR1, |
| | | ABCC3, LILRB4, STAB1, |
| | | TMEM37, IL4R |
| Kidney Renal | TROP2/TACSTD2; | TRAIL-R2, CSF1R, |
| Papillary Cell | or FOLR1 | CD163, MARCO, |
| Carcinoma | | TNFR2, TREM2, MS4A7, |
| | | C5AR1, ABCC3, LILRB4, |
| | | STAB1, TMEM37, IL4R |
| Lung | TROP2/TACSTD2; | TRAIL-R2, CD33, CSF1R, |
| Adenocarcinoma | FOLR1; or | SEMA4A, SEMA4D, |
| (Non-Small Cell | GPC3 | CD163, MARCO, TNFR2, |
| Lung Carcinoma) | | TREM2, MS4A7, C5AR1, |
| | | ABCC3, LILRB4, MRC1, |
| | | STAB1, TMEM37, MERTK, |
| | | TMEM119, SIGLEC1, IL4R, |
| | | CLEC10A, SELPLG |
| Lung Squamous | TROP2/TACSTD2; | TRAIL-R2, CSF1R, SEMA4A, |
| Cell Carcinoma | GPC3; or | SEMA4D, CD163, MARCO, |
| (Non Small Cell | FOLR1 | TNFR2, TREM2, MS4A7, |
| Lung Carcinoma) | | C5AR1, ABCC3, LILRB4, |
| | | MRC1, STAB1, TMEM37, |
| | | MERTK, TMEM119, IL4R, |
| | | CLEC10A, SELPLG |
| Ovarian Serous | TROP2/TACSTD2; | TRAIL-R2, CSF1R, SEMA4A, |
| Cystadenocarcinoma | FOLR1 | SEMA4D, CD163, |
| | | TNFR2, TREM2, MS4A7, |
| | | C5AR1, LILRB4, STAB1, |
| | | SIGLEC1, IL4R, SELPLG |
| Pancreatic | TROP2/TACSTD2; | TRAIL-R2, CD33, CSF1R, |
| Adenocarcinoma | or | SEMA4A, SEMA4D, |
| | FOLR1 | CD163, MARCO, |
| | | TNFR2, TREM2, MS4A7, |
| | | C5AR1, ABCC3, LILRB4, |
| | | MRC1, STAB1, TMEM37, |
| | | TMEM119, IL4R, SELPLG |
| Prostate | TROP2/TACSTD2; | TRAIL-R2, CSF1R, SEMA4A, |
| Adenocarcinoma | or | SEMA4D, TNFR2, TREM2, |
| | FOLH1/PSMA | MS4A7, C5AR1, ABCC3, |
| | | STAB1, LILRB4, MERTK, |
| | | TMEM119, IL4R, SEMA4D, |
| | | CLEC10A, SELPLG |
| Kidney Renal Clear | FOLR1 | TRAIL-R2, CD33, CSF1R, |
| Cell Carcinoma | | SEMA4A, SEMA4D, CD163, |
| | | TNFR2, TREM2, MS4A7, |
| | | C5AR1, LYVE1, ABCC3, |
| | | LILRB4, STAB1, TMEM37, |
| | | IL4R, SIGLEC1, SELPLG |
| Uterine Corpus | TROP2/TACSTD2 | TRAIL-R2, CSF1R, SEMA4A, |
| Endometrial | | SEMA4D, TNFR2, TREM2, |
| Carcinoma | | C5AR1, ABCC3, LILRB4, |
| | | MRC1, STAB1, MERTK, |
| | | TMEM37, TMEM119, IL4R |
| Thyroid Carcinoma | TROP2/TACSTD2 | TRAIL-R2, CSF1R, |
| | | SEMA4A, SEMA4D, |
| | | CD163, MARCO, TNFR2, |
| | | TREM2, C5AR1, ABCC3, |
| | | LILRB4, MRC1, STAB1, |
| | | MERTK, TMEM37, IL4R |
| Stomach | TROP2/TACSTD2 | TRAIL-R2, CSF1R, SEMA4A, |
| Adenocarcinoma | | SEMA4D, CD163, TNFR2, |
| | | TREM2, MS4A7, C5AR1, |
| | | ABCC3, LILRB4, MRC1, |
| | | STAB1, MERTK, TMEM37, |
| | | TMEM119, IL4R |
| Rectal | TROP2/TACSTD2 | TRAIL-R2, CSF1R, TNFR2, |
| Adenocarcinoma | | ABCC3, STAB1, TMEM37, |
| | | TMEM119, IL4R |
| Sarcoma | GPC3 | TRAIL-R2, CD33, CSF1R, |
| | | SEMA4D, CD163, MARCO, |
| | | TNFR2, TREM2, MS4A7, |
| | | C5AR1, LYVE1, LILRB4, |
| | | MRC1, STAB1, TMEM119, |
| | | SIGLEC1, IL4R, CLEC10A, |
| | | SELPLG |
| Testis and Germ | GPC3 | TRAIL-R2, CD33, CSF1R, |
| Cell Tumors | | SEMA4A, SEMA4D, |
| | | SEMA4D, CD163, |
| | | TNFR2, TREM2, |
| | | ABCC3, MRC1, STAB1, |
| | | TMEM37, TMEM119, IL4R, |
| | | CLEC10A, SELPLG |
| Uterine | GPC3 | TRAIL-R2, CSF1R, |
| Carcinosarcoma | | SEMA4D, STAB1, |
| | | TMEM37, TMEM119, IL4R, |

In some embodiments, the method further comprises administering an additional therapeutic agent. In some embodiments, the additional therapeutic agent is a chemotherapeutic agent, radiation, or a combination thereof. Exemplary additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; corticosteroids such as prednisone, methylprednisolone, or dexamethasone; or SRC-family protein-tyrosine kinase inhibitors such as dasatinib.

In some cases, the additional therapeutic agent comprises an immune checkpoint modulator. Exemplary immune checkpoint modulators include, but are not limited to, nivolumab (Opdivo®), pembrolizumab (Keytruda®), pidilizumab (CT-011), BMS-936559, atezolizumab (MPDL3280A), avelumab, ipilimumab (Yervoy®), or tremelimumab. In some instances, the additional therapeutic agent includes an immune chckpoint modulator that specifically binds to and/or modulate an activity of PD-L1, PD-L2, PD1, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprises a first-line therapy. As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers.

In some cases, the additional therapeutic agent comprises a second-line therapy, a third-line therapy, a fourth-line therapy, or a fifth line therapy. In some instances, a second-line therapy encompasses treatments that are utilized after the primary or first-line treatment stops. In some instances, a third-line therapy, a fourth-line therapy, or a fifth-line therapy encompass subsequent treatments. As indicated by the naming convention, a third-line therapy encompasses a treatment course upon which a primary and a second-line therapy have stopped.

In some instances, the additional therapeutic agent comprises an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprises use of allogeneic T lymphocytes with, e.g., anti-tumor activity, expansion of the T lymphocytes in vitro, and subseqent infusion of the expanded allogeneic T lymphocytes into a subject in need thereof.

In some instances, the additional therapeutic agent comprises a vaccine, optionally an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the additional therapeutic agent comprises a salvage therapy.

In some cases, the additional therapeutic agent comprises a palliative therapy.

In some embodiments, the multi-specific binding polypeptide (e.g., the multi-specific antibody) and the additional therapeutic agent are administered simultaneously. In other embodiments, the multi-specific binding polypeptide (e.g., the multi-specific antibody) and the additional therapeutic agent are administered sequentially. In some embodiments, the multi-specific binding polypeptide (e.g., the multi-specific antibody) is administered to the subject prior to administering the additional therapeutic agent. In some embodiments, the additional therapeutic agent is administered to the subject prior to administering the multi-specific binding polypeptide (e.g., the multi-specific antibody). In some embodiments, the multi-specific binding polypeptide (e.g., the multi-specific antibody) and the additional therapeutic agent are administered as a combination. In some embodiments, the multi-specific binding polypeptide (e.g., the multi-specific antibody) and the additional therapeutic agent are administered as separate dosage forms.

In some embodiments, the subject has undergone surgery.

### Tumor and Immunosuppressive Cell Killing

In some embodiments, disclosed herein is a method of inducing a tumor and immunosuppressive cell killing effect in a target cell population. In some embodiments, the method comprises contacting the target cell population comprising at least one tumor cell and at least one immunosuppressive cell with a multi-specific binding polypeptide (e.g., a multi-specific antibody) described above or a pharmaceutical composition described infra for a time sufficient to induce a cell killing effect, thereby killing the at least one tumor cell and the at least one immunosuppressive cell in the target cell population.

In some embodiments, the time sufficient to induce a cell killing effect is about 5 minutes, 10 minutes, 20 minutes, 30 minutes, 1 hour, 2 hours, 3 hours, 4 hours, 5 hours, 6 hours, 7 hours, 8 hours, 9 hours, 10 hours, 12 hours, 18 hours, 24 hours, or more.

In some embodiments, the tumor cell is a cell from a solid tumor. In some embodiments, the tumor cell is a cell from a hematological malignancy. In some embodiments, the tumor cell is from a bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer. In some embodiments, the tumor cell is from chronic lymphocytic leukemia (CLL), small lymphocytic lymphoma (SLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, acute myeloid leukemia.

In some embodiments, the immunosuppressive cell is MDSC, a tumor-associated macrophage, or a Treg cell. In some embodiments, the immunosuppressive cell is MDSC. In some embodiments, the immunosuppressive cell is a tumor-associated macrophage (TAM). In some embodiments, the immunosuppressive cell is a Treg cell.

In some instances, the multi-specific binding polypeptide (e.g., the multi-specific antibody) or the pharmaceutical composition comprising the multi-specific binding polypeptide (e.g., the multi-specific antibody) modulates T-cell proliferation. In some instances, the multi-specific binding polypeptide (e.g., the multi-specific antibody) or the pharmaceutical composition comprising the multi-specific binding polypeptide (e.g., the multi-specific antibody) modulates tumor-infiltrating lymphocyte (TIL) proliferation.

In some instances, the multi-specific binding polypeptide (e.g., the multi-specific antibody) or the pharmaceutical composition comprising the multi-specific binding polypeptide (e.g., the multi-specific antibody) enhances T-cell proliferation. In some instances, the multi-specific binding polypeptide (e.g., the multi-specific antibody) or the pharmaceutical composition comprising the multi-specific binding polypeptide (e.g., the multi-specific antibody) enhances tumor-infiltrating lymphocyte (TIL) proliferation. In some cases, the T-cell proliferation (optionally TIL proliferation) is enhanced by about 1-fold, 2-fold, 3-fold, 4-fold, 5-fold, 6-fold, 7-fold, 8-fold, 9-fold, 10-fold, 20-fold, 30-fold, 40-fold, 50-fold, or more. In some cases, the T-cell proliferation (optionally TIL proliferation) is enhanced by about 5%, 10%, 20%, 30%, 40%, 50%, 60%, 70%, 80%, 90%, 100%, or more.

In some instances, the multi-specific binding polypeptide (e.g., the multi-specific antibody) or the pharmaceutical composition comprising the multi-specific binding polypeptide (e.g., the multi-specific antibody) decreases tumor cells in the target cell population by about 20%, 30%, 40%, 50%, 60%, 70%, 80%, 90%, 100%, or more.

In some instances, the multi-specific binding polypeptide (e.g., the multi-specific antibody) or the pharmaceutical composition comprising the multi-specific binding polypeptide (e.g., the multi-specific antibody) decreases tumor cells in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

In some instances, the multi-specific binding polypeptide (e.g., the multi-specific antibody) or the pharmaceutical composition comprising the multi-specific binding polypeptide (e.g., the multi-specific antibody) decreases tumor cell proliferation in the target cell population by about 20%, 30%, 40%, 50%, 60%, 70%, 80%, 90%, 100%, or more.

In some instances, the multi-specific binding polypeptide (e.g., the multi-specific antibody) or the pharmaceutical composition comprising the multi-specific binding polypeptide (e.g., the multi-specific antibody) decreases tumor cell proliferation in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

In some instances, the multi-specific binding polypeptide (e.g., the multi-specific antibody) or the pharmaceutical composition comprising the multi-specific binding polypeptide (e.g., the multi-specific antibody) decreases immunosuppressive cells in the target cell population by about 20%, 30%, 40%, 50%, 60%, 70%, 80%, 90%, 100%, or more.

In some instances, the multi-specific binding polypeptide (e.g., the multi-specific antibody) or the pharmaceutical composition comprising the multi-specific binding polypeptide (e.g., the multi-specific antibody) decreases immunosuppressive cells in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

In some instances, the multi-specific binding polypeptide (e.g., the multi-specific antibody) or the pharmaceutical composition comprising the multi-specific binding polypeptide (e.g., the multi-specific antibody) decreases immunosuppressive cell proliferation in the target cell population by about 20%, 30%, 40%, 50%, 60%, 70%, 80%, 90%, 100%, or more.

In some instances, the multi-specific binding polypeptide (e.g., the multi-specific antibody) or the pharmaceutical composition comprising the multi-specific binding polypeptide (e.g., the multi-specific antibody) decreases immunosuppressive cell proliferation in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

In some embodiments, the target cell population is an in vivo target cell population.

In other embodiments, the target cell population is an in vitro cell population.

In some embodiments, the subject described supra is a human.

### Tumor Cell Killing by TRAIL-R2-Mediated Apoptosis

TRAIL receptor 2 (also referred to as TRAIL-R2, death receptor 5, DR5, tumor necrosis factor receptor superfamily member 10B, or TNFRSF10B), is a cell surface receptor of the Tumor Necrosis Factor (TNF)-receptor superfamily that binds to TRAIL and mediates apoptosis via an intracellular Death Domain (DD). In some instances, oligomerization of TRAIL-R2 occurs prior to activation of the apoptotic signaling pathway. As utilized herein, oligomerization of TRAIL-R2 encompasses two or more TRAIL-R2 monomers, optionally two, three, four, five, six, or more TRAIL-R2 monomers. In some cases, TRAIL-R2 dimerization occurs prior to activation of the apoptotic signaling pathway. In such cases, oligomerization (e.g., dimerization) is facilitated by a disulfide bond between, e.g., Cys209 of each TRAIL-R2 monomer.

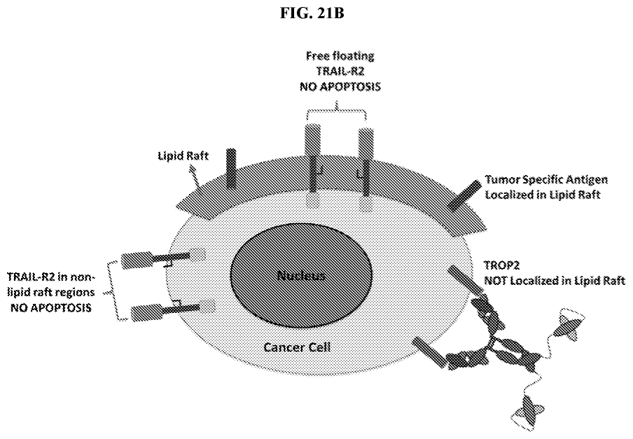

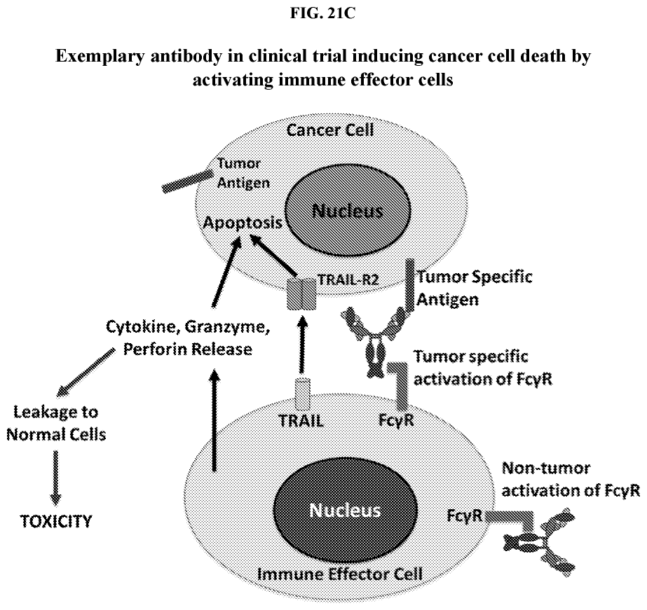

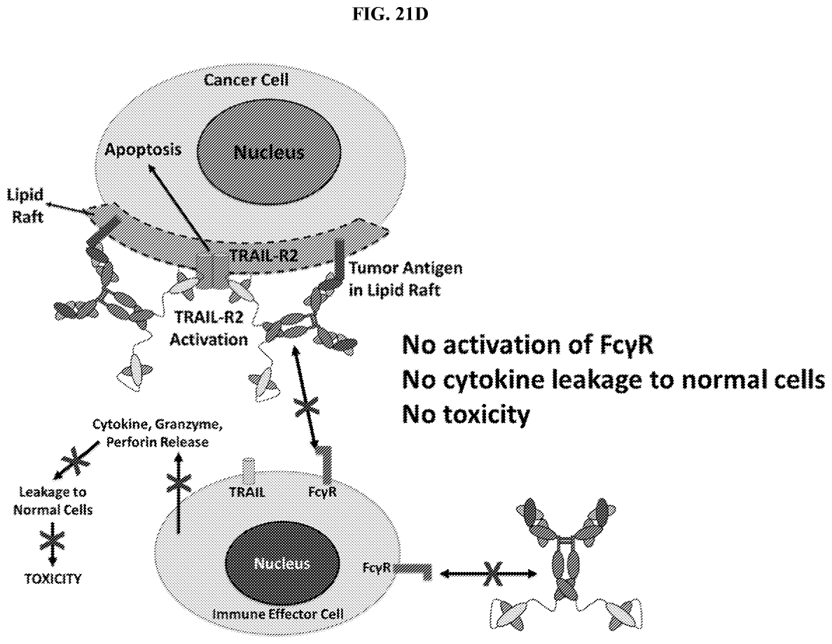

In some embodiments, oligomerization of TRAIL-R2 occurs in a lipid raft of a target cell. In some instances, two or more TRAIL-R2 monomers, optionally two, three, four, five, six, or more TRAIL-R2 monomers oligomerizes in the lipid raft. In such instances and upon oligomerization, the apoptotic signaling pathway is activated within the target cell. In some cases, dimerization of the TRAIL-R2 occurs in a lipid raft of the target cell, optionally facilitated by a disulfide bond between, e.g., Cys209 of each TRAIL-R2 monomer. In some cases, the apoptotic signaling pathway is activated after dimerization of the TRAIL-R2 in the lipid raft. In some cases, the target cell is a cancer cell. Also see FIG. 21A and FIG. 21B. FIG. 21A illustrates an exemplary multi-specific antibody that interacts with a tumor-associated antigen located within a lipid raft of a cancer cell which facilitates recruitment and oligomerization (e.g., dimerization) of the TRAIL-R2 within the lipid raft for subsequent activation of the apoptotic signaling pathway. FIG. 21B illustrates an exemplary multi-specific antibody that interacts with a tumor-associated antigen located outside the lipid raft of a cancer cell, which upon binding by the antibody does not activate the TRAIL-R2 mediated apoptotic signaling pathway.

In some embodiments, a multi-specific binding polypeptide (e.g., a multi-specific antibody) described herein comprises a first binding moiety that binds to a receptor on a target cell and a second binding moiety that binds to TRAIL-R2 expressed on the same target cell. In some instances, the multi-specific binding polypeptide (e.g., the multi-specific antibody) does not activate immune cells (e.g., immune effector cells). In such instances, the activation of the immune cells decreases or prevents toxicity associated from leakage of cytokines and death inducing enzymes produced from the activated immune cells to normal cells. Also see FIG. 21C and FIG. 21D, which show that conventional antibody therapy can eliminate cancer cells by activating immune effector cells upon binding to FcγRs leading to non-tumor toxicity due to leakage of cytokines, performs, and/or granzymes to neighboring normal cells (FIG. 21C); but activation of TRAIL-R2 mediated apoptotic signaling pathway reduces the need to activate immune effector cells, thereby eliminating immune cell-related toxicity leakage to normal cells (FIG. 21D).

In some embodiments, a multi-specific binding polypeptide (e.g., a multi-specific antibody) described herein comprises a first binding moiety that binds to a receptor on a cancer cell and a second binding moiety that binds to TRAIL-R2 expressed on the same cancer cell. In some instances, the first binding moiety binds to a tumor-associated antigen expressed on the cancer cell. In other instances, the first binding moiety binds to a protein expressed on the surface of the cancer cell. In additional instances, the first binding moiety comprises a ganglioside. In further instances, the first binding moiety comprises a small molecule. In some cases, the first binding moiety has a higher binding affinity toward the receptor (e.g., by 1, 2, 3, 4, 5, 10, 20, 50, or more fold) than the second binding moiety toward TRAIL-R2. In some cases, the first binding moiety facilitates oligomerization (e.g., dimerization) of the TRAIL-R2. In some cases, the first binding moiety facilitates oligomerization (e.g., dimerization) of the TRAIL-R2 in the lipid raft of the cancer cell. In some cases, oligomerization (e.g., dimerization) of the TRAIL-R2 by the multi-specific binding polypeptide (e.g., the multi-specific antibody) modulates activation of the apoptotic signaling pathway of the cancer cell. In some cases, the multi-specific binding polypeptide (e.g., the multi-specific antibody) is used for the treatment of a cancer by activation of the TRAIL-R2 mediated apoptosis.

In some embodiments, the first binding moiety binds to a receptor on a target cell (e.g., a cancer cell). In some instances, the first binding moiety binds to a tumor-associated antigen. In some instances, the tumor-associated antigen comprises FOLR1, CD33, CD38, FLT3, or GPC3. In some cases, the multi-specific binding polypeptide (e.g., the multi-specific antibody) comprises a first binding moiety that binds to FOLR1, CD33, CD38, FLT3, or GPC3 expressed on the target cell (e.g., the cancer cell) and a second binding moiety that binds to TRAIL-R2 expressed on the same target cell (e.g., the cancer cell). In some cases, the multi-specific binding polypeptide (e.g., the multi-specific antibody) comprises a first binding moiety that binds to FOLR1 expressed on the target cell (e.g., the cancer cell) and a second binding moiety that binds to TRAIL-R2 expressed on the same target cell (e.g., the cancer cell). In some cases, the multi-specific binding polypeptide (e.g., the multi-specific antibody) comprises a first binding moiety that binds to CD33 expressed on the target cell (e.g., the cancer cell) and a second binding moiety that binds to TRAIL-R2 expressed on the same target cell (e.g., the cancer cell). In some cases, the multi-specific binding polypeptide (e.g., the multi-specific antibody) comprises a first binding moiety that binds to CD38 expressed on the target cell (e.g., the cancer cell) and a second binding moiety that binds to TRAIL-R2 expressed on the same target cell (e.g., the cancer cell). In some cases, the multi-specific binding polypeptide (e.g., the multi-specific antibody) comprises a first binding moiety that binds to FLT3 expressed on the target cell (e.g., the cancer cell) and a second binding moiety that binds to TRAIL-R2 expressed on the same target cell (e.g., the cancer cell). In some cases, the multi-specific binding polypeptide (e.g., the multi-specific antibody) comprises a first binding moiety that binds to GPC3 expressed on the target cell (e.g., the cancer cell) and a second binding moiety that binds to TRAIL-R2 expressed on the same target cell (e.g., the cancer cell). In some cases, the first binding moiety has a higher binding affinity toward FOLR1, CD33, CD38, FLT3, or GPC3 (e.g., by 1, 2, 3, 4, 5, 10, 20, 50, or more fold) than the second binding moiety toward TRAIL-R2. In some cases, the first binding moiety facilitates oligomerization (e.g., dimerization) of the TRAIL-R2. In some cases, the first binding moiety facilitates oligomerization (e.g., dimerization) of the TRAIL-R2 in the lipid raft of the target cell (e.g., the cancer cell). In some cases, oligomerization (e.g., dimerization) of the TRAIL-R2 by the multi-specific binding polypeptide (e.g., the multi-specific antibody) modulates activation of the apoptotic signaling pathway of the target cell (e.g., the cancer cell). In some cases, the multi-specific binding polypeptide (e.g., the multi-specific antibody) is used for the treatment of a disease or indication (e.g., a cancer) by activation of the TRAIL-R2 mediated apoptosis.

In some embodiments, the first binding moiety binds to CD32a (also referred to as FcγRIIa). In some cases, the multi-specific binding polypeptide (e.g., the multi-specific antibody) comprises a first binding moiety that binds to CD32a expressed on the target cell (e.g., the cancer cell) and a second binding moiety that binds to TRAIL-R2 expressed on the same target cell (e.g., the cancer cell). In some cases, the first binding moiety has a higher binding affinity toward CD32a (e.g., by 1, 2, 3, 4, 5, 10, 20, 50, or more fold) than the second binding moiety toward TRAIL-R2. In some cases, the first binding moiety facilitates oligomerization (e.g., dimerization) of the TRAIL-R2. In some cases, the first binding moiety facilitates oligomerization (e.g., dimerization) of the TRAIL-R2 in the lipid raft of the target cell (e.g., the cancer cell). In some cases, oligomerization (e.g., dimerization) of the TRAIL-R2 by the multi-specific binding polypeptide (e.g., the multi-specific antibody) modulates activation of the apoptotic signaling pathway of the target cell (e.g., the cancer cell). In some cases, the multi-specific binding polypeptide (e.g., the multi-specific antibody) has an impaired binding or no binding to CD16 and/or CD64. In some cases, the multi-specific binding polypeptide (e.g., the multi-specific antibody) is used for the treatment of a disease or indication (e.g., a cancer) by activation of the TRAIL-R2 mediated apoptosis.

In some embodiments, the first binding moiety binds to a protein on a target cell (e.g., a cancer cell). In some instances, the protein is a Glycosylphosphatidylinositol (GPI)-anchored protein. In some instances, the multi-specific binding polypeptide (e.g., the multi-specific antibody) comprises a first binding moiety that binds to GPI-anchored protein expressed on the target cell (e.g., the cancer cell) and a second binding moiety that binds to TRAIL-R2 expressed on the same target cell (e.g., the cancer cell). In some cases, the first binding moiety has a higher binding affinity toward GPI-anchored protein (e.g., by 1, 2, 3, 4, 5, 10, 20, 50, or more fold) than the second binding moiety toward TRAIL-R2. In some cases, the first binding moiety facilitates oligomerization (e.g., dimerization) of the TRAIL-R2. In some cases, the first binding moiety facilitates oligomerization (e.g., dimerization) of the TRAIL-R2 in the lipid raft of the target cell (e.g., the cancer cell). In some cases, oligomerization (e.g., dimerization) of the TRAIL-R2 by the multi-specific binding polypeptide (e.g., the multi-specific antibody) modulates activation of the apoptotic signaling pathway of the target cell (e.g., the cancer cell). In some cases, the multi-specific binding polypeptide (e.g., the multi-specific antibody) is used for the treatment of a disease or indication (e.g., a cancer) by activation of the TRAIL-R2 mediated apoptosis.

In some embodiments, the first binding moiety binds to a ganglioside (GD) on a target cell (e.g., a cancer cell). In some instances, the multi-specific binding polypeptide (e.g., the multi-specific antibody) comprises a first binding moiety that binds to the ganglioside (GD) expressed on the target cell (e.g., the cancer cell) and a second binding moiety that binds to TRAIL-R2 expressed on the same target cell (e.g., the cancer cell). In some cases, the first binding moiety has a higher binding affinity toward the ganglioside (e.g., by 1, 2, 3, 4, 5, 10, 20, 50, or more fold) than the second binding moiety toward TRAIL-R2. In some cases, the first binding moiety facilitates oligomerization (e.g., dimerization) of the TRAIL-R2. In some cases, the first binding moiety facilitates oligomerization (e.g., dimerization) of the TRAIL-R2 in the lipid raft of the target cell (e.g., the cancer cell). In some cases, oligomerization (e.g., dimerization) of the TRAIL-R2 by the multi-specific binding polypeptide (e.g., the multi-specific antibody) modulates activation of the apoptotic signaling pathway of the target cell (e.g., the cancer cell). In some cases, the multi-specific binding polypeptide (e.g., the multi-specific antibody) is used for the treatment of a disease or indication (e.g., a cancer) by activation of the TRAIL-R2 mediated apoptosis.

In some embodiments, the first binding moiety comprises a small molecule that binds to a receptor on a target cell (e.g., a cancer cell). In some instances, the small molecule is a folic acid or its salt, derivative, or analog thereof that binds to FOLR1. Exemplary folic acid salts, derivatives, and analogs include, but are not limited to, tetrahydrofolic acid, leucovorin, levoleucovorin, levomefolic, acid, triglu-5-formyl-tetrahydrofolate, (6S)-5,6,7,8,-tetrahydrofolic acid, 5-methyltetrahydrofolic acid, and (6R)-folinic acid. In some instances, the multi-specific binding polypeptide (e.g., the multi-specific antibody) comprises a first binding moiety comprising a folic acid or its salt, derivative, or analog thereof that binds to FOLR1 expressed on the target cell (e.g., the cancer cell) and a second binding moiety that binds to TRAIL-R2 expressed on the same target cell (e.g., the cancer cell). In some cases, the first binding moiety has a higher binding affinity toward FOLR1 (e.g., by 1, 2, 3, 4, 5, 10, 20, 50, or more fold) than the second binding moiety toward TRAIL-R2. In some cases, the first binding moiety facilitates oligomerization (e.g., dimerization) of the TRAIL-R2. In some cases, the first binding moiety facilitates oligomerization (e.g., dimerization) of the TRAIL-R2 in the lipid raft of the target cell (e.g., the cancer cell). In some cases, oligomerization (e.g., dimerization) of the TRAIL-R2 by the multi-specific binding polypeptide (e.g., the multi-specific antibody) modulates activation of the apoptotic signaling pathway of the target cell (e.g., the cancer cell). In some cases, the multi-specific binding polypeptide (e.g., the multi-specific antibody) is used for the treatment of a disease or indication (e.g., a cancer) by activation of the TRAIL-R2 mediated apoptosis.

In some embodiments, a multi-specific binding polypeptide (e.g., a multi-specific antibody) comprising the second binding moiety that binds to TRAIL-R2 further comprises a Fc modification to impair or inhibit FcγR interaction. In some instances, the modification comprises a mutation at N297, L234, P238, P331, S239, S442, or a combination thereof, wherein the residue position corresponds to positions 297, 234, 238, 331, 239, and 442 of IgG1, according to EU numbering convention. In some cases, the modification comprises N297A, L234A, L235A, P238S, P331S, L234F, S239C, S442C, or a combination thereof.

### Pharmaceutical Composition and Formulation

In some embodiments, the pharmaceutical composition and formulations described herein are administered to a subject by multiple administration routes, including but not limited to, parenteral, oral, buccal, rectal, sublingual, or transdermal administration routes. In some embodiments, parenteral administration comprises intravenous, subcutaneous, intramuscular, intracerebral, intranasal, intra-arterial, intra-articular, intradermal, intravitreal, intraosseous infusion, intraperitoneal, or intratechal administration. In some embodiments, the pharmaceutical composition is formulated for local administration. In other embodiments, the pharmaceutical composition is formulated for systemic administration.

In certain embodiments, the multi-specific binding polypeptides (e.g., multi-specific antibodies) of the current disclosure are included in a pharmaceutical composition comprising one or more pharmaceutically acceptable excipients, carriers, or diluents. In certain embodiments, the multi-specific binding polypeptides (e.g., multi-specific antibodies) of the current disclosure are administered suspended in a sterile solution. In certain embodiments, the solution comprises 0.9% NaCl. In certain embodiments, the solution comprises about 5% glucose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EGTA or EGTA. In certain embodiments, the multi-specific binding polypeptides (e.g., multi-specific antibodies) of the current disclosure are shipped/stored lyophilized and reconstituted before administration. In certain embodiments, lyophilized multi-specific binding polypeptide formulations comprise a bulking agent such as, mannitol, sorbitol, sucrose, trehalose, or dextran-40. The lyophilized formulation can be contained in a vial comprised of glass. The multi-specific binding polypeptides when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

In one embodiment the pharmaceutical composition of the present invention can be formulated in a Tris-Cl buffer, the concentration of Tris-Cl being at least about 5 mM, about 10 mM, about 15 mM, about 20 mM, about 25 mM, about 30 mM, about 35 mM, about 40 mM, or about 50 mM. In another embodiment, the concentration of Tris-Cl is about 20 mM. In other embodiments, the composition is formulated in a citrate buffer, the concentration of citrate being at least about 5 mM, about 10 mM, about 15 mM, about 20 mM, about 25 mM, about 30 mM, about 35 mM, about 40 mM, or about 50 mM. In a particular embodiment, the citrate concentration is about 10 mM or about 20 mM. In some embodiments, the composition is formulated in a histidine buffer, the concentration of histidine being at least about 5 mM, about 10 mM, about 15 mM, about 20 mM, about 25 mM, about 30 mM, about 35 mM, about 40 mM, or about 50 mM. In some embodiments, the histidine concentration is about 20 mM. In other embodiments, the composition is formulated in a Tris-citrate buffer, the concentration of Tris-Cl being at least about 5 mM, about 10 mM, about 15 mM, about 20 mM, about 25 mM, about 30 mM, about 35 mM, about 40 mM, or about 50 mM, and the concentration of citrate being at least about 2 mM, about 5 mM, about 10 mM, about 15 mM, about 20 mM, about 25 mM, about 30 mM, about 35 mM, about 40 mM, or about 50 mM. In certain embodiments, the concentration of Tris-Cl is about 13.3 mM and the concentration of citrate is about 6.7 mM.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins1999).

In some embodiments, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some embodiments, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some embodiments, one or more pharmaceutical compositions described herein comprising the provided multi-specific binding polypeptides (e.g., multi-specific antibodies) are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some embodiments, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it is expressed as the ratio between LD50 and ED₅₀. Compounds exhibiting high therapeutic indices are preferred. The data obtained from cell culture assays and animal studies are used in formulating a range of dosage for use in human. The dosage of such compounds lies preferably within a range of circulating concentrations that include the ED₅₀ with minimal toxicity. The dosage varies within this range depending upon the dosage form employed and the route of administration utilized.

### Kits/Article of Manufacture

Disclosed herein are kits and articles of manufacture suitable for carrying out the methods disclosed herein. In some embodiments, the kit comprises two or more components required for performing a therapeutic method described herein. In some embodiments, kit components include, but are not limited to, one or more multi-specific binding polypeptides (e.g., multi-specific antibodies), or one or more multi-specific binding polypeptide conjugates (e.g., multi-specific antibody-payload conjugates) described herein, appropriate reagents, and/or equipment. In some embodiments, the kit is packaged in a vial, pouch, ampoule, and/or any container suitable for a therapeutic method. Additional examples of pharmaceutical packaging materials include, but are not limited to, blister packs, bottles, tubes, bags, containers, bottles, and any packaging material suitable for a selected formulation and optionally intended mode of administration and treatment. In some embodiments, kit components are provided as concentrates (including lyophilized compositions), which are further diluted prior to use or provided at the concentration of use. In some embodiments, when the one or more multi-specific polypeptide conjugates (e.g., multi-specific antibody-payload conjugates) is provided for use in vivo, a single dosage is provided in a sterilized container having the desired amount and concentration of antibody-payload conjugate.

In some embodiments, a kit includes labels listing contents and/or instructions for use, and package inserts with instructions for use. A set of instructions will also typically be included.

In some embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein, the terms “individual(s),” “subject(s),” and “patient(s)” are interchangeable and refer to any mammal diagnosed with, suspected of being afflicted with, or at risk of being afflicted with a disease or condition, e.g., a cancer, tumor, or neoplasm marked by uncontrolled cell-growth. In certain embodiments, the mammal is a human person. In some embodiments, the mammal is a non-human such as a cat, dog, mouse, rat, non-human primate, cow, pig, sheep, goat, llama, alpaca, or horse.

As used herein “binding” or “specific binding” refers to the binding of an affinity molecule like a receptor or an antibody to a distinct portion of a molecule, or epitopes and variants thereof. Specific binding is generally mediated by the CDR residues of an antibody, but framework residues can sometimes also be involved in conjunction with a CDR residue. In certain embodiments, specific binding does not encompass interactions of the Fc region of an antibody with another molecule such as protein A, G, or A/G.

The terms “polypeptide” and “protein” are used interchangeably to refer to a polymer of amino acid residues and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “antibody” herein is used in the broadest sense and includes monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multi-specific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

Antibodies bind to target antigens or epitopes based on their complementarity determining regions. The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, and CDR-H3 or HCDR1, HCDR2, and HCDR3) and three CDRs in each light chain variable region (CDR-L1, CDR-L2, and CDR-L3 or LCDR1, LCDR2, and LCDR3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al.,“IMGT unique numbering for immunoglobulin and T cell receptor variable domains and Ig superfamily V-like domains,” Dev Comp Immunol, 2003 January;27(1):55-77 (“IMGT” numbering scheme); Honegger A and Plückthun A, “Yet another numbering scheme for immunoglobulin variable domains: an automatic modeling and analysis tool,” J Mol Biol, 2001 Jun 8;309(3):657-70, (“Aho” numbering scheme); and Whitelegg NR and Rees AR, “WAM: an improved algorithm for modelling antibodies on the WEB,” Protein Eng.2000 December;13(12):819-24 (“AbM” numbering scheme.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In certain embodiments, the CDRs of the provided antibodies and antigen binding fragments are defined by the Kabat, IMGT, Chothia, Contact, Aho, AbM scheme or any combination thereof. In certain embodiments, the CDRs of the provided antibodies and antigen binding fragments are defined by the Kabat, IMGT, Chothia scheme, or any combination thereof. In certain embodiments, the CDR boundaries are defined by the longest contiguous overlapping subset of the Kabat, IMGT, and Chothia scheme, or any combination thereof.

CDRs are endogenously associated with a variable region of an antibody. The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g. scFv or sFv); and multi-specific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody or multi-specific binding polypeptide is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

As used herein, a “multi-specific” antibody refers to an antibody that binds to more than one antigen. For instances, a “multi-specific” antibody can bind to two, three, four, or more antigens. In some cases, a “multi-specific” antibody binds to at least one tumor-associated antigen, at least one antigen expressed on an immunosuppressive cell, or a combination thereof. In some cases, the “multi-specific” antibody further comprises a modification in the Fc region. In some cases, the “multi-specific” antibody binds to at least one tumor-associated antigen and/or at least one antigen expressed on an immunosuppressive cell and further binds to a receptor that interacts with the antibody's Fc region.

In some embodiments, a multi-specific antibody is a bispecific antibody. As used in such instances, bispecific antibody refers to an antibody that can bind to at least one tumor-associated antigen, at least one antigen expressed on an immunosuppressive cell, or a combination thereof. In some cases, the bispecific antibody further comprises a modification in the Fc region. In some cases, the bispecific antibody binds to at least one tumor-associated antigen and/or at least one antigen expressed on an immunosuppressive cell and further binds to a receptor that interacts with the antibody's Fc region.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved ADCC (antibody-dependent cell cytotoxicity), ADCP (antibody-dependent cellular phagocytosis), or CDC (complement-dependent cytotoxicity). In some embodiments, the multispecific binding polypeptides, provided herein, can induce direct cell death of both cancer cells and immunosuppressive cells by Fc-based effector function, such as ADCC, ADCP, and CDC. In some embodiments, the multispecific binding polypeptides comprise mutations in the Fc-region to enhance Fc-based effector functions, such as ADCC, ADCP and CDC.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments of the variant V_{H} and V_{L} sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

As used herein, the term “conservative substitution” denotes the replacement of an amino acid residue by another, chemically or biologically similar residue. Biologically similar means that the substitution does not destroy a biological activity or function.

Structurally similar means that the amino acids have side chains with similar length, such as alanine, glycine, and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such multi-specific binding polypeptides (e.g., multi-specific antibodies) can be encoded by a nucleic acid.

Modified multi-specific binding polypeptides (e.g., multi-specific antibodies) also include one or more D-amino acids substituted for L-amino acids (and mixtures thereof), structural and functional analogues, for example, peptidomimetics having synthetic or non-natural amino acids or amino acid analogues and derivatizes forms. Modifications include cyclic structures such as an end-to-end amide bond between the amino and carboxy-terminus of the multi-specific binding polypeptides (e.g., multi-specific antibodies).

Modified multi-specific binding polypeptides (e.g., multi-specific antibodies) further include “chemical derivatives,” in which one or more amino acids has a side chain chemically altered or derivatived. Such derivatized multi-specific binding polypeptides (e.g., multi-specific antibodies) include, for example, amino acids in which free amino groups from amine hydrochlorides, p-toluene sulfonyl groups, carobenzoxy groups, the free carboxy groups that form salts, methyl and ethyl esters; free hydroxl groups that form O-acyl or O-alkyl derivatives as well as naturally occurring amino acid derivatives, for example, 4-hdroxyproline for proline, 5-hydroxylysine for lysine, homoserine for serine, ornithine for lysine. Also included are amino acid derivatives that can alter covalent bonding, for example, the disulfide linkage that forms between two cysteine residues.

GPC3, also referred to herein as glypican 3, DGSX, GTR2-2, MXR7, OCI-5, SDYS, SGB, SGBS, or SGBS1, is a tumor-associated antigen and has been implicated in cell proliferation. As used herein, GPC3 encompasses a full-length wild-type GPC3 protein, a functional fragment thereof, a variant (including naturally occurring and non-native modifications), or an isoform. Exemplary GPC3 proteins contemplated and encompass herein include, but are not limited to, GPC3s illustrated in the following NCBI accession numbers: AAH35972.1 (full-length), NP_001158089.1 (Isoform 1 precursor), NP_004475.1 (Isoform 2 precursor), NP_001158090.1 (Isoform 3 precursor), NP_001158091.1 (Isoform 4 precursor), AQN67628.1 (variant), ABC72126.1 (Splice variant A), ABC72125.1 (Splice variant B), and ABC72127.1 (Splice variant C).

In certain embodiments, a multi-specific binding polypeptide (e.g., a multi-specific antibody) described herein comprises a tumor binding moiety that recognizes an epitope on the extracellular portion of the GPC3 protein. In some embodiments, the multi-specific binding polypeptide (e.g., the multi-specific antibody) comprises a tumor binding moiety that recognizes an epitope on the extracellular portion of a full-length wild-type GPC3 protein, a functional fragment thereof, a variant (including naturally occurring and non-native modifications), or an isoform, and optionally one or more of GPC3 proteins defined by the NCBI accession number disclosed above.

TROP2, also referred to herein as TACSTD2, EGP-1, EGP1, GA733-1, GA7331, GP50, M151, TROP2, tumor-associated calcium signal transducer 2, or tumor associated calcium signal transducer 2, is a tumor-associated antigen. As used herein, TROP2 encompasses a full-length wild-type TROP2 protein, a functional fragment thereof, a variant (including naturally occurring and non-native modifications), or an isoform. Exemplary TROP2 proteins contemplated and encompass herein include, but are not limited to, TROP2s illustrated in the following NCBI accession numbers: CAG47056.1 (full length) or NP_002344.2 (precursor).

In certain embodiments, a multi-specific binding polypeptide (e.g., a multi-specific antibody) described herein comprises a tumor binding moiety that recognizes an epitope on the extracellular portion of the TROP2 protein. In some embodiments, the multi-specific binding polypeptide (e.g., the multi-specific antibody) comprises a tumor binding moiety that recognizes an epitope on the extracellular portion of a full-length wild-type TROP2 protein, a functional fragment thereof, a variant (including naturally occurring and non-native modifications), or an isoform, and optionally one or more of TROP2 proteins defined by the NCBI accession number disclosed above.

FOLR1, also referred to herein as FBP, FOLR, folate receptor 1, folate receptor alpha, or FRalpha, is a tumor-associated antigen. As used herein, FOLR1 encompasses a full-length wild-type FOLR1 protein, a functional fragment thereof, a variant (including naturally occurring and non-native modifications), or an isoform. Exemplary FOLR1 proteins contemplated and encompass herein include, but are not limited to, full-length FOLR1 illustrated in NCBI accession number AAH02947.1.

In certain embodiments, a multi-specific binding polypeptide (e.g., a multi-specific antibody) described herein comprises a tumor binding moiety that recognizes an epitope on the extracellular portion of the FOLR1 protein. In some embodiments, the multi-specific binding polypeptide (e.g., the multi-specific antibody) comprises a tumor binding moiety that recognizes an epitope on the extracellular portion of a full-length wild-type FOLR1 protein, a functional fragment thereof, a variant (including naturally occurring and non-native modifications), or an isoform, and optionally one or more of FOLR1 proteins defined by the NCBI accession number disclosed above.

CD38, also referred to herein as ADPRC1, is a tumor-associated antigen. As used herein, CD38 encompasses a full-length wild-type CD38 protein, a functional fragment thereof, a variant (including naturally occurring and non-native modifications), or an isoform. Exemplary CD38 proteins contemplated and encompass herein include, but are not limited to, CD38s illustrated in the following NCBI accession numbers: BAA18966.1 (full length), EAW92745.1 (isoform CRA_a), and EAW92746.1 (isoform CRA_b).

In certain embodiments, a multi-specific binding polypeptide (e.g., a multi-specific antibody) described herein comprises a tumor binding moiety that recognizes an epitope on the extracellular portion of the CD38 protein. In some embodiments, the multi-specific binding polypeptide (e.g., the multi-specific antibody) comprises a tumor binding moiety that recognizes an epitope on the extracellular portion of a full-length wild-type CD38 protein, a functional fragment thereof, a variant (including naturally occurring and non-native modifications), or an isoform, and optionally one or more of CD38 proteins defined by the NCBI accession number disclosed above.

FLT3, also referred to herein as CD135, FLK-2, FLK2, STK1, Fetal Liver Kinase 2, FLK2, or fms related tyrosine kinase 3, is a tumor-associated antigen. As used herein, FLT3 encompasses a full-length wild-type FLT3 protein, a functional fragment thereof, a variant (including naturally occurring and non-native modifications), or an isoform. Exemplary FLT3 proteins contemplated and encompass herein include, but are not limited to, FLT3s illustrated in the following NCBI accession numbers: NP_004110.2 (full length), XP_011533317.1 (Isoform X1), XP_016875975.1 (Isoform X2), XP_016875976.1 (Isoform X3), XP_016875977.1 (Isoform X4), XP_016875978.1 (Isoform X5), CAA81393.1 (precursor), and AAI44040.1 (variant).

In certain embodiments, a multi-specific binding polypeptide (e.g., a multi-specific antibody) described herein comprises a tumor binding moiety that recognizes an epitope on the extracellular portion of the FLT3 protein. In some embodiments, the multi-specific binding polypeptide (e.g., the multi-specific antibody) comprises a tumor binding moiety that recognizes an epitope on the extracellular portion of a full-length wild-type FLT3 protein, a functional fragment thereof, a variant (including naturally occurring and non-native modifications), or an isoform, and optionally one or more of FLT3 proteins defined by the NCBI accession number disclosed above.

CD33, also referred to herein as Siglec-3, sialic acid binding Ig-like lectin 3, SIGLEC3, SIGLEC-3, gp67, or p67, is expressed on cancer cells and immunosuppressive cells. As used herein, CD33 encompasses a full-length wild-type CD33, a functional fragment thereof, a variant (including naturally occurring and non-native modifications), or an isoform. Exemplary CD33 proteins contemplated and encompass herein include, but are not limited to, CD33s illustrated in the following NCBI accession numbers: AAH28152.1 (full length), NP_001763.3 (Isoform 1 precursor), NP_001076087.1 (Isoform 2), NP_001171079.1 (Isoform 3 precursor), XP_016882997.1 (Isoform X1), XP_016882998.1 (Isoform X2), XP_011525834.1 (Isoform X3), and XP_016882999.1 (Isoform X4).

In certain embodiments, a multi-specific binding polypeptide (e.g., a multi-specific antibody) described herein comprises a tumor binding moiety that recognizes an epitope on the extracellular portion of the CD33 protein. In some embodiments, the multi-specific binding polypeptide (e.g., the multi-specific antibody) comprises a tumor binding moiety that recognizes an epitope on the extracellular portion of a full-length wild-type CD33 protein, a functional fragment thereof, a variant (including naturally occurring and non-native modifications), or an isoform, and optionally one or more of CD33 proteins defined by the NCBI accession number disclosed above.

CD163, also referred to herein as M130, MM130, or SCARI1, is an antigen expressed on an immunosuppressive cell. As used herein, CD163 encompasses a full-length wild-type CD163, a functional fragment thereof, a variant (including naturally occurring and non-native modifications), or an isoform. Exemplary CD163 proteins contemplated and encompass herein include, but are not limited to, CD163s illustrated in the following NCBI accession numbers: AAH51281.1 (full length), NP_004235.4 (M130 isoform a precursor), NP_981961.2 (M130 isoform b precursor), NP_001357075.1 (M130 isoform c precursor), CAB45233.1 (variant), AAY99762.1 (variant), EAW88665.1 (Isoform CRA_a), and EAW88666.1 (Isoform CRA_b).

In certain embodiments, a multi-specific binding polypeptide (e.g., a multi-specific antibody) described herein comprises a tumor binding moiety that recognizes an epitope on the extracellular portion of the CD163 protein. In some embodiments, the multi-specific binding polypeptide (e.g., the multi-specific antibody) comprises a tumor binding moiety that recognizes an epitope on the extracellular portion of a full-length wild-type CD163 protein, a functional fragment thereof, a variant (including naturally occurring and non-native modifications), or an isoform, and optionally one or more of CD163 proteins defined by the NCBI accession number disclosed above.

CSF1R, also referred to herein as C-FMS, CD115, CSF-1R, CSFR, FIM2, FMS, HDLS, macrophage colony-stimulating factor receptor, M-CSF-R, colony stimulating factor 1 receptor, or BANDDOS, is an antigen expressed on an immunosuppressive cell. As used herein, CSF1R encompasses a full-length wild-type CSF1R, a functional fragment thereof, a variant (including naturally occurring and non-native modifications), or an isoform. Exemplary CSF1R proteins contemplated and encompass herein include, but are not limited to, CSF1Rs illustrated in the following NCBI accession numbers: AAH47521.1 (full length), NP_001275634.1 (Isoform a precursor), NP_001362250.1 (Isoform b precursor), AAI29940.1 (variant), and ACF47629.1 (soluble variant 1).

In certain embodiments, a multi-specific binding polypeptide (e.g., a multi-specific antibody) described herein comprises a tumor binding moiety that recognizes an epitope on the extracellular portion of the CSF1R protein. In some embodiments, the multi-specific binding polypeptide (e.g., the multi-specific antibody) comprises a tumor binding moiety that recognizes an epitope on the extracellular portion of a full-length wild-type CSF1R protein, a functional fragment thereof, a variant (including naturally occurring and non-native modifications), or an isoform, and optionally one or more of CSF1R proteins defined by the NCBI accession number disclosed above.

TRAIL-R2, also referred to herein as CD262, DR5, KILLER, KILLER/DR5, TRAILR2, TRICK2, TRICK2A, TRICK2B, TRICKB, or ZTNFR9, is an antigen expressed on an immunosuppressive cell. As used herein, TRAIL-R2 encompasses a full-length wild-type TRAIL-R2, a functional fragment thereof, a variant (including naturally occurring and non-native modifications), or an isoform. Exemplary TRAIL-R2 proteins contemplated and encompass herein include, but are not limited to, TRAIL-R2s illustrated in the following NCBI accession numbers: AAH01281.1 (full length), NP_003833.4 (Isoform 1 precursor), NP_671716.2 (isoform 2 precursor), and AAC51778.1 (variant).

In certain embodiments, a multi-specific binding polypeptide (e.g., a multi-specific antibody) described herein comprises a tumor binding moiety that recognizes an epitope on the extracellular portion of the TRAIL-R2 protein. In some embodiments, the multi-specific binding polypeptide (e.g., the multi-specific antibody) comprises a tumor binding moiety that recognizes an epitope on the extracellular portion of a full-length wild-type TRAIL-R2 protein, a functional fragment thereof, a variant (including naturally occurring and non-native modifications), or an isoform, and optionally one or more of TRAIL-R2 proteins defined by the NCBI accession number disclosed above.

As used herein and under the context of a multi-specific antibody, a 80%, 85%, 90%, 95%, 97%, 98%, or 99% sequence identity in reference to a parent sequence that an antibody is compared to can refer to amino acid differences in the framework region of the antibody, and/or conservative substitutions in a CDR region. For example, an amino acid difference contributing to at least about 80%, 85%, 90%, 95%, 97%, 98%, or 99% sequence identity resides in a framework region of an immunoglobulin VH, VL, HC, and/or LC, and the CDRs remain unchanged relative to the CDRs of the parent antibody. In some cases, the amino acid differences contributing to the at least about 80%, 85%, 90%, 95%, 97%, 98%, or 99% sequence identity comprises conservative substitutions in the framework region, CDRs region, or a combination thereof, of the immunoglobulin VH, VL, HC, and/or LC but the multi-specific antibody retains binding to a target tumor antigen and/or a target antigen expressed on an immunosuppressor cell.

As used herein, the terms “immunosuppressive cell(s)” and “immunosuppressor cell(s)” are used interchangeably and refer to a cell that exerts an immunosuppressive effect in a tumor microenvironment. In some instances, the immunosuppressive cells or immunosuppressor cells comprise CD4+ T regulatory (Treg) cells, MDSCs, TAMs, or a combination thereof.

As utilized herein, the term “single action” in reference to a single action bispecific antibody refers to reducing or inhibiting cancer cell proliferation or tumor growth and/or killing of cancer cells. In some instances, the “single action” does not comprise reducing or inhibiting of other cell types such as immunosuppressor cells.

As utilized herein, the term “multi-action” in reference to a multi-action multi-specific binding polypeptide (e.g., a multi-specific antibody) refers to reducing or inhibiting cancer cell proliferation or tumor growth, killing of cancer cells, reducing or inhibiting immunosuppressor cell proliferation, and killing of the immunosuppressor cells. In such instances, the term “multi-action” encompasses inhibiting and killing of both cancer cells and immunosuppressor cells.

### EXAMPLES

These examples are provided for illustrative purposes only and not to limit the scope of the claims provided herein.

### Example 1—Generation of Exemplary Multi-Specific Antibodies

The heavy chain of an exemplary bispecific antibody can comprise the following format: N—VH-CH1-CH2-CH3-peptide-scFv-C, where N and C denote the N-terminal and C-terminal ends of the construct. The heavy chain is an IgG1, IgG2, IgG3, or IgG4 isotype and can further be cloned into a mammalian expression vector. The 5′-end of the heavy chain is cloned into the 3′-end of the leader sequence of the vector to enable secretion from the mammalian expression cells. The light chain is constructed in a separate expression vector where VL is cloned either with Kappa or Lamda CL domain. Similarly, the 5′-end of the DNA sequence of the light chain is cloned into the 3′-end of the leader sequence of the vector.

The plasmids containing the heavy and light chains are transiently co-expressed in HEK293 or CHO cells.

The cells are grown in flasks on an orbital shaker platform rotating at 140 rpm at 37° C., 5% CO₂ and sub-cultured following the manufacturer's protocol. Co-transfection is performed with polyethyleneimine (PEI) as the transfection reagent. Briefly, HEK293/CHO cells are sub-cultured to a cell density of 0.5-0.7×10⁶ cells/ml for 24 hours before transfection. Immediately before transfection, cell density is adjusted to 1×10⁶ cells/ml. Five hundred micrograms of each purified plasmid (1 mg/ml) is added to 19 ml Optipro (Invitrogen). Two milliliters of 1 mg/ml PEI, pH 7.0 (molecular weight (MW) of 25 000) dissolved in water is added to 18 ml Optipro. Both the solutions are incubated at room temperature for 5 min. The DNA/Optipro solution is added to the PEI/Optipro solution and incubated for 10 min at room temperature and added drop wise to 1 L HEK293/CHO culture. The supernatant is collected 6-8 days after transfection. Antibodies are purified and checked for expression, purity and quality, as described in the following examples.

### Example 2—Target Selection

The receptors/antigens expressed on the cancer cells and the immunosuppressive cells were selected based on expression level measured by log₂(TPM+1) or TPM or by RNASeqV2 or by staining intensity by IHC. The expression level was analyzed from genomics databases, such as The Cancer Genome Atlas (TCGA), Human Protein Atlas, FireBrowse, Tumor Portal, etc. Following were performed to select specific pairing combinations of targets on cancer cell and immunosuppressive cell:

(1) Antigens/receptors expressed in cancer cells and immunosuppressive cells that were detected in same tumor samples with score of TPM>5 or log₂(TPM+1)≥2.3 were selected;
(2) Antigens/receptors expressed in cancer cells and immunosuppressive cells that were detected in same tumor samples with median expression level score of RNASeqV2 (Log)>5 were selected;
(3) Antigens/receptors expressed in cancer cells and immunosuppressive cells that were detected in same tumor samples with staining intensity score of +2 to +3 as determined by IHC;
(4) Immunosuppressive cells such as MDSC was detected in the tumor samples by detecting the presence of following antigens: CD33, CD14, FUT4/CD15, ITGAM/CD11b;
(5) Immunosuppressive cells such as M2-Tumor associated macrophage (M2-TAM) was detected in the tumor samples by detecting the presence of the following antigens: MRC1, CD163, CD204/MSR1, CD301/CLEC10A, CD209, CD206/MRC1, CLEC7A, ITGAM/CD1 lb, CD200R;
(6) The specific receptors mentioned in 4 and 5 were detected in the tumor samples with expression level score of TPM>5 or log₂(TPM+1)≥2.3;
(7) The specific receptors mentioned in 4 and 5 were detected in the tumor samples with expression level score of RNASeqV2 (Log)>5;
(8) The specific receptors mentioned in 4 and 5 were detected in the tumor samples with staining intensity score of +2 to +3 as determined by IHC;
(9) The selected targets were expressed in non-tumor tissues with intensity score 0 to +1 as determined by IHC;
(10) The selected targets were expressed in non-tumor tissues with median expression level score of RNASeqV2 (Log)<4;
(11) The selected targets in respective cell types were expressed in non-tumor tissues with score of TPM<4 or log₂(TPM+1)≤2;
(12) The receptors were selected by analyzing cancer genome databases that reports the expression level of receptors by estimating RNA level, protein level and staining intensities by IHC;
(13) The receptors were selected by analyzing genomic databases using data analytics software that executes steps 1-12.

### Example 3—Expression of TROP2 and Immunosuppressive Cell Surface Markers in Different Cancers

The following in silico experiments established clinical relevance of an approach using exemplary multi-specific binding polypeptides that bind to tumor associated antigens and immunosuppressive cells in triple-negative breast cancer (TNBC), Lung Adenocarcinoma (LUAD), Lung Squamous Cell Carcinoma (LUSC), and Prostate Adenocarcinoma by detecting expression levels of oncogenic receptor (TROP2) and several receptors expressed on the surface of immunosuppressive cells in the same biopsies.

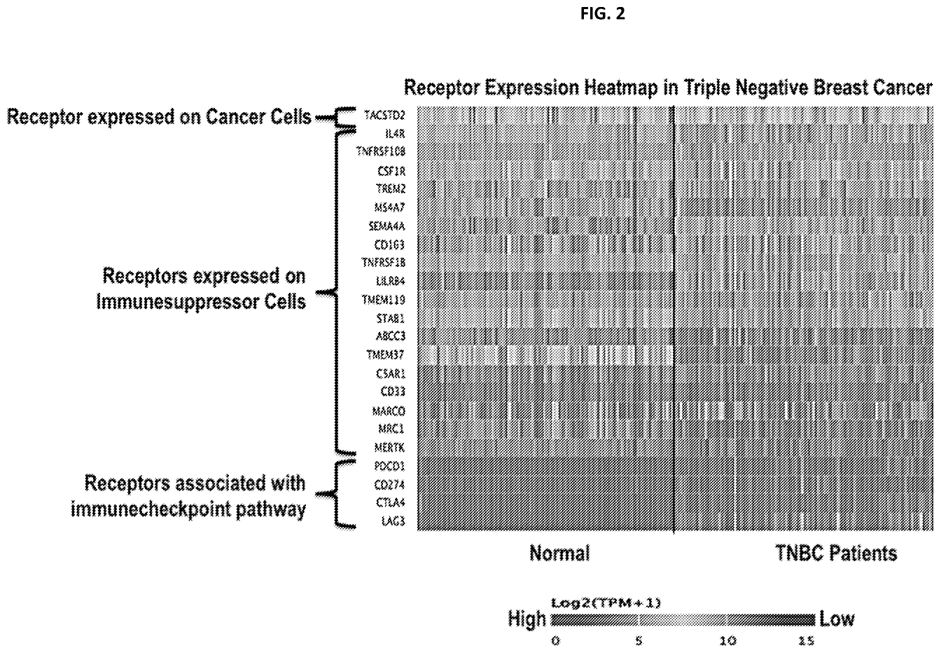

In Triple Negative Breast Cancer (TNBC) biopsies, as shown in FIG. 2, it was noted that >80% of TNBC tumor patients in The Cancer Genome Atlas (TCGA) pool (n=116) exhibited high expression level of TROP2 with TRAIL-R2, CSF1R, SEMA4A, SEMA4D, TNFR2, CD163, TREM2, LILRB4, STAB1, TMEM119, MS4A7, IL4R, and SELPLG with individual gene expression level measured by Log2(TPM+1)≥2.3 in the same biopsy sample. In that pool about 10% of TNBC patients exhibited TROP2 and CD33 with individual gene expression level, Log2(TPM+1)≥2.3 in the same biopsy sample. Analysis of TNBC tumor biopsies in the TCGA pool suggests low expression level of immune checkpoint receptors, PD-1, PD-L1, CTLA-4 and LAG-3; >80% patient tumor biopsies exhibited a gene expression level, Log2 (TPM+1)≤2.3.

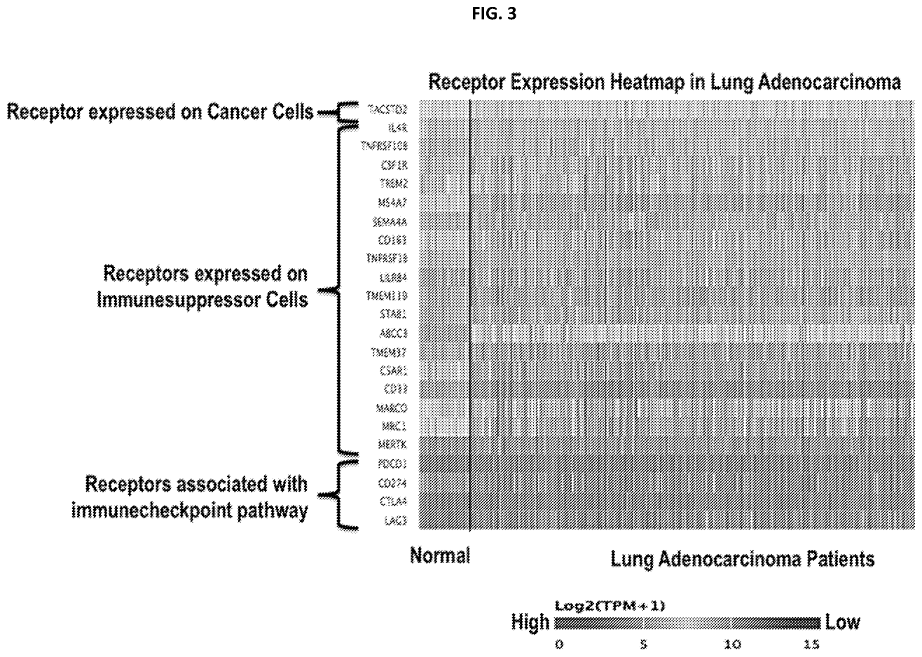

In Lung Adenocarcinoma (LUAD) biopsies, as shown in FIG. 3, it was noted that >80% of TNBC tumor patients in The Cancer Genome Atlas (TCGA) pool (n=515) exhibited high expression level of TROP2 with TRAIL-R2, CSF1R, IL4R, SEMA4A, SEMA4D, CD163, TREM2, TNFR2, MARCO, TREM2, MS4A7, C5AR1, ABCC3, LILRB4, MRC1, STAB1, TMEM37, MERTK, TMEM119, SIGLEC1, CLEC10A, and SELPLG with individual gene expression level measured by Log₂ (TPM+1)≥2.3 in the same biopsy sample. In that pool about 20% of TNBC patients exhibited TROP2 and CD33 with individual gene expression level, Log₂ (TPM+1)≥2.3 in the same biopsy sample. Analysis of LUAD tumor biopsies in the TCGA pool suggests low expression level of immune checkpoint receptors, PD-1, PD-L1, CTLA-4 and LAG-3; >50% patient tumor biopsies exhibited a gene expression level, Log₂ (TPM+1)≤2.3.

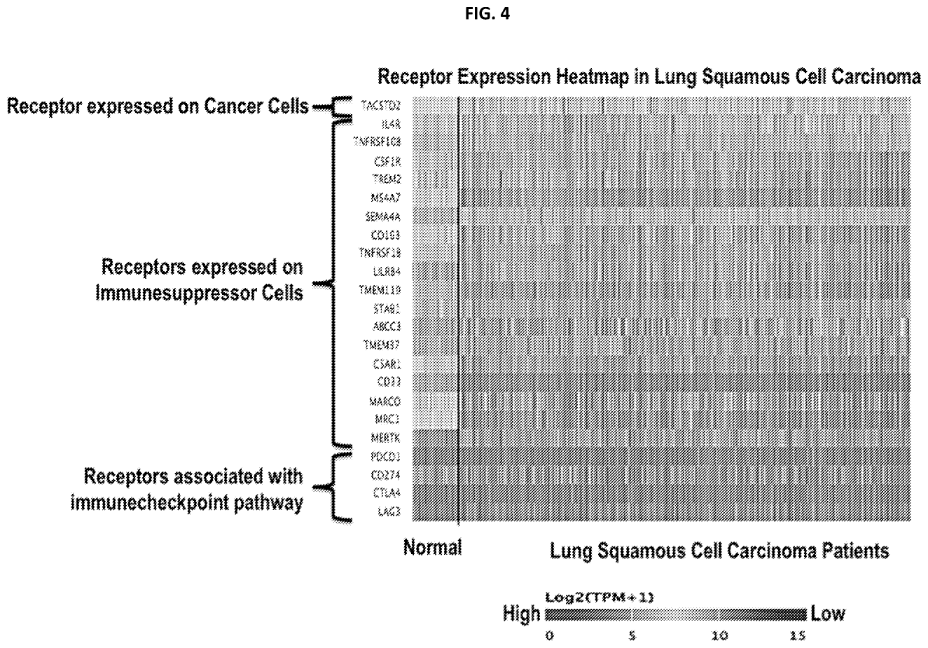

In Lung Squamous Cell Carcinoma (LUSC) biopsies, as shown in FIG. 4, it was noted that >80% of LUSC tumor patients in The Cancer Genome Atlas (TCGA) pool (n=503) exhibited high expression level of TROP2 with TRAIL-R2, CSF1R, IL4R, SEMA4A, SEMA4D, TREM2, TNFR2, MS4A7, C5AR1, ABCC3, LILRB4, MRC1, STAB1, TMEM37, MERTK, TMEM119, SIGLEC1, CLEC10A with individual gene expression level measured by Log2 (TPM+1)≥2.3 in the same biopsy sample. In that pool about 10-15% of LUSC patients exhibited TROP2 and CD33 with individual gene expression level, Log2 (TPM+1)≥2.3 in the same biopsy sample. Analysis of LUSC tumor biopsies in the TCGA pool suggests low expression level of immune checkpoint receptors, PD-1, PD-L1, CTLA-4 and LAG-3; >50% patient tumor biopsies exhibited a gene expression level, Log2 (TPM+1)≤2.3.

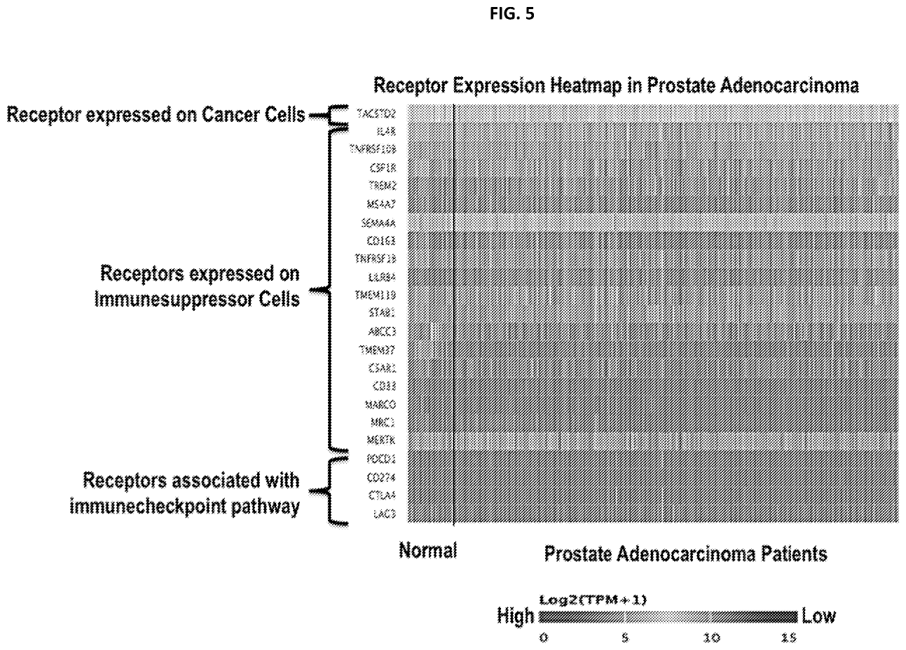

In Prostate Adenocarcinoma (PRAD) tumor biopsies, as shown in FIG. 5, it was noted that >70% of TNBC tumor patients in The Cancer Genome Atlas (TCGA) pool (n=497) exhibited high expression level of TROP2 with TRAIL-R2, CSF1R, SEMA4A, SEMA4D, TNFR2, TREM2, MS4A7, C5AR1, ABCC3, STAB1, LILRB4, MERTK, TMEM119, IL4R, SEMA4D, CLEC10A and SELPLG with individual gene expression level measured by Log2(TPM+1)≥2.3 in the same biopsy sample. In that pool <10% of PRAD patients exhibited TROP2 and CD33 with individual gene expression level, Log₂(TPM+1)≥2.3 in the same biopsy sample. Analysis of PRAD tumor biopsies in the TCGA pool suggests low expression level of immune checkpoint receptors, PD-1, PD-L1, CTLA-4 and LAG-3; >80% patient tumor biopsies exhibited a gene expression level, Log2 (TPM+1)≤2.3.

### Example 4—Expression of GPC3 and Immunosuppressive Cell Surface Markers in Different Cancers

The following in silico experiments established clinical relevance of an approach using relevant multi-specific binding polypeptides that bind to tumor associated antigens and immunosuppressive cells in Liver Hepatocellular Carcinoma (LIHC) and Lung Adenocarcinoma (LUAD) by detecting expression levels of oncogenic receptor (GPC3) and several receptors expressed on the surface of immunosuppressive cells in the same biopsies.

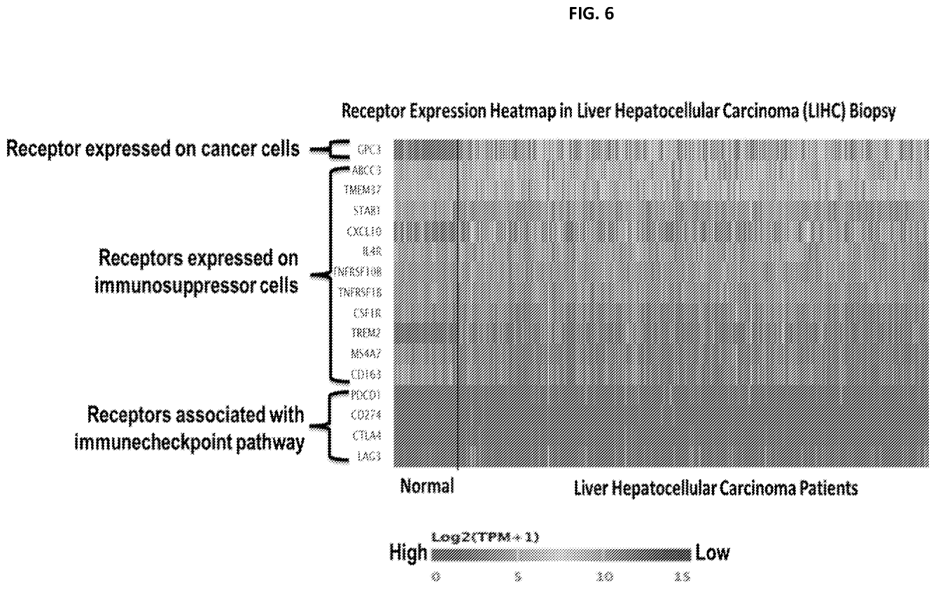

In Liver Hepatocellular Carcinoma (LIHC) biopsies, as shown in FIG. 6, it was noted that >80% of LIHC tumor patients in The Cancer Genome Atlas (TCGA) pool (n=371) exhibited high expression level of GPC3 with ABCC3, TMEM37, STAB1, IL4R, TNFRSF10B, TNFRSF1B, CSF1R, TREM2, MS4A7 and CD163, with individual gene expression level measured by Log₂(TPM+1)≥2.3 in the same biopsy sample. Analysis of LIHC tumor biopsies in the TCGA pool suggests >80% patient tumor biopsies exhibit low expression level of immune checkpoint receptors, PD-1, PD-L1, CTLA-4 and LAG-3(gene expression level, Log₂(TPM+1)≤2.3).

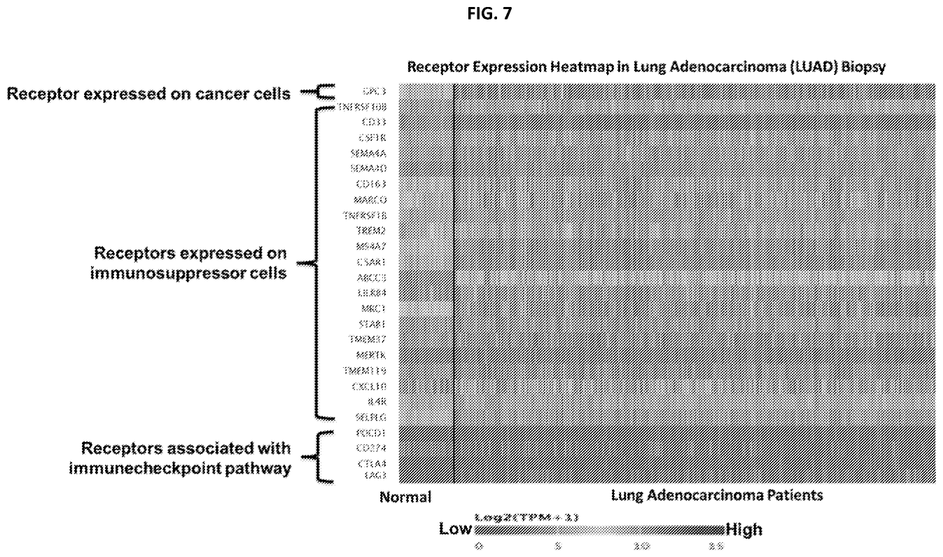

In LUAD biopsies, as shown in FIG. 7, it was noted that >50% of LUAD tumor patients in The Cancer Genome Atlas (TCGA) pool (n=515) exhibited high expression level of GPC3 with TRAIL-R2, CSF1R, IL4R, SEMA4A, SEMA4D, CD163, TREM2, TNFR2, MARCO, TREM2, MS4A7, C5AR1, ABCC3, LILRB4, MRC1, STAB1, TMEM37, MERTK, TMEM119, SIGLEC1 and CLEC10A, with individual gene expression level measured by Log₂(TPM+1)≥2.3 in the same biopsy sample. Analysis of LUAD tumor biopsies in the TCGA pool suggests >80% patient tumor biopsies exhibit low expression level of immune checkpoint receptors, PD-1, PD-L1, CTLA-4 and LAG-3(gene expression level, Log₂(TPM+1)≤2.3).

### Example 5—Expression of FOLR1 and Immunosuppressive Cell Surface Markers in Different Cancers

The following in silico experiments established clinical relevance of an approach using relevant multi-specific binding polypeptides that bind to tumor associated antigens and immunosuppressive cells in Lung Adenocarcinoma (LUAD), Lung Squamous Cell Carcinoma (LUSC) and Ovarian Serous Cystadenocarcinoma (OV) by detecting expression levels of oncogenic receptor (FOLR1) and several receptors expressed on the surface of immunosuppressive cells in the same biopsies.

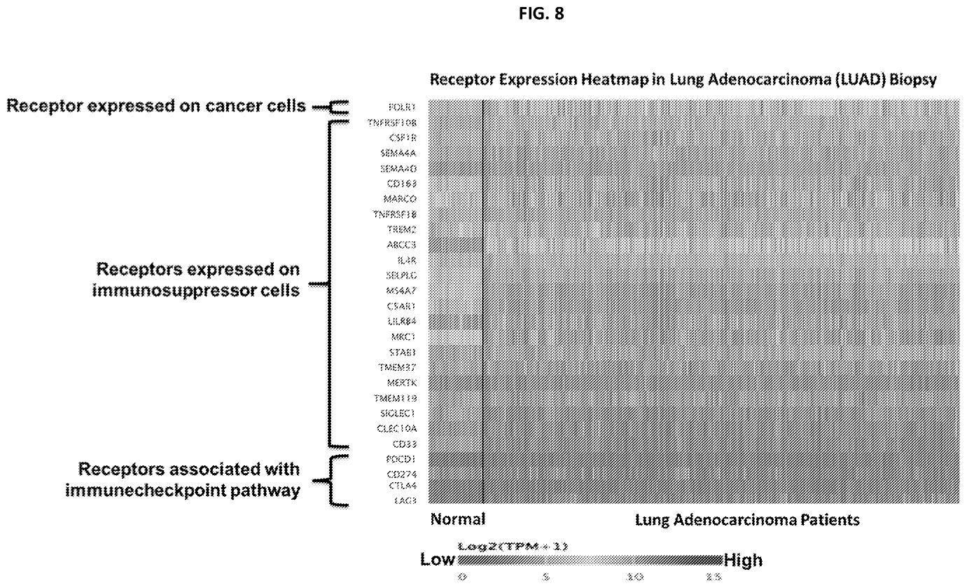

In LUAD tumor biopsies, as shown in FIG. 8, it was noted that >80% of LUAD tumor patients in The Cancer Genome Atlas (TCGA) pool (n=515) exhibited high expression level of FOLR1 with TRAIL-R2, CD33, CSF1R, SEMA4A, SEMA4D, SEMA4D, CD163, MARCO, TNFR2, TREM2, MS4A7, C5AR1, ABCC3, LILRB4, MRC1, STAB1, TMEM37, MERTK, TMEM119, SIGLEC1, IL4R, CLEC10A and SELPLG with individual gene expression level measured by Log₂(TPM+1)≥2.3 in the same biopsy sample. Analysis of LUAD tumor biopsies in the TCGA pool suggests >80% patient tumor biopsies exhibit low expression level of immune checkpoint receptors, PD-1, PD-L1, CTLA-4 and LAG-3(gene expression level, Log₂(TPM+1)≤2.3).

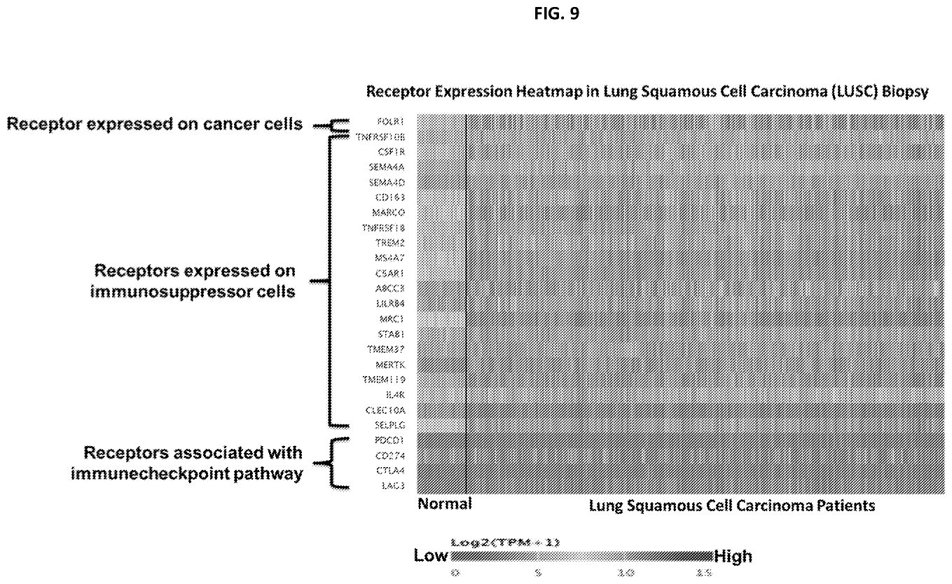

In LUSC tumor biopsies, as shown in FIG. 9, it was noted that >50% of LUSC tumor patients in The Cancer Genome Atlas (TCGA) pool (n=503) exhibited high expression level of FOLR1 with TRAIL-R2, CSF1R, SEMA4A, SEMA4D, SEMA4D, CD163, MARCO, TNFR2, TREM2, MS4A7, C5AR1, ABCC3, LILRB4, MRC1, STAB1, TMEM37, MERTK, TMEM119, IL4R, CLEC10A and SELPLG with individual gene expression level measured by Log₂(TPM+1)≥2.3 in the same biopsy sample. Analysis of LUSC tumor biopsies in the TCGA pool suggests >80% patient tumor biopsies exhibit low expression level of immune checkpoint receptors, PD-1, PD-L1, CTLA-4 and LAG-3 (gene expression level, Log₂(TPM+1)≤2.3).

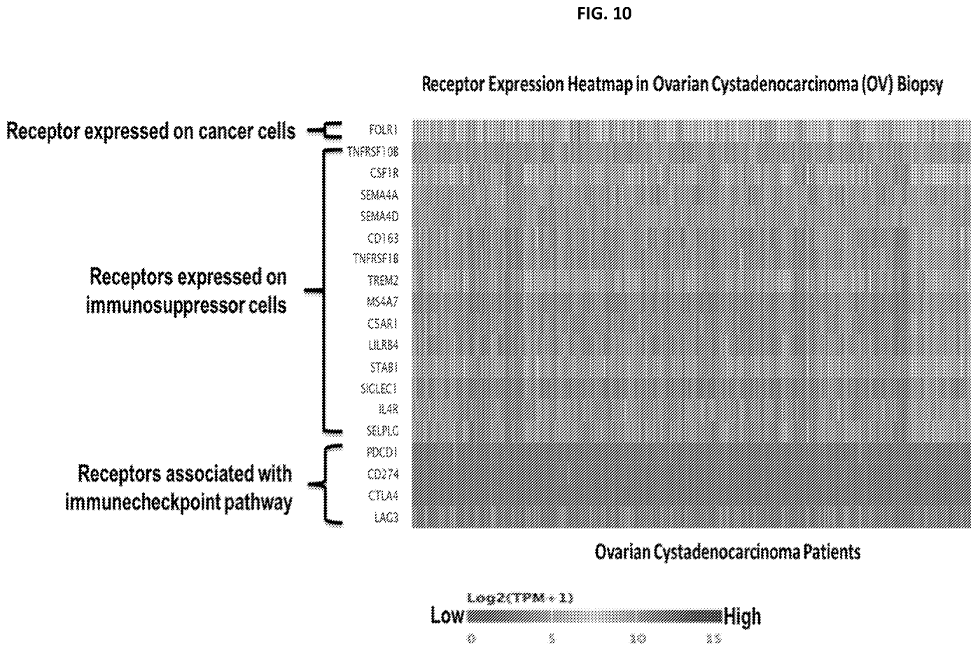

In Ovarian Cystadenocarcinoma (OV) biopsies, as shown in FIG. 10, it was noted that >50% of OV tumor patients in The Cancer Genome Atlas (TCGA) pool (n=291) exhibited high expression level of FOLR1 with TRAIL-R2, CSF1R, SEMA4A, SEMA4D, SEMA4D, CD163, TNFR2, TREM2, MS4A7, C5AR1, LILRB4, STAB1, SIGLEC1, IL4R and SELPLG with individual gene expression level measured by Log₂(TPM+1)≥2.3 in the same biopsy sample. Analysis of OV tumor biopsies in the TCGA pool suggests >80% patient tumor biopsies exhibit low expression level of immune checkpoint receptors, PD-1, PD-L1, CTLA-4 and LAG-3(gene expression level, Log₂(TPM+1)≤2.3).

### Example 6—Expression of FOLH1 and Immunosuppressive Cell Surface Markers in Different Cancers

The following in silico experiments established clinical relevance of an approach using relevant multi-specific binding polypeptides that bind to tumor associated antigens and immunosuppressive cells in Prostate Adenocarcinoma (PRAD) by detecting expression levels of oncogenic receptor (FOLH1) and several receptors expressed on the surface of immunosuppressive cells in the same biopsies.

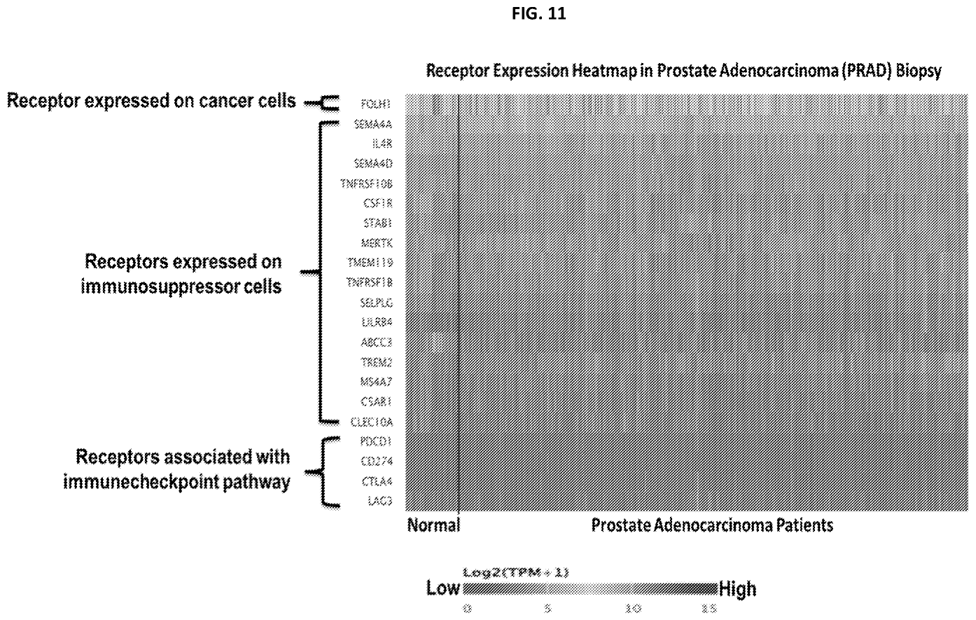

In PRAD tumor biopsies, as shown in FIG. 11, it was noted that >50% of PRAD tumor patients in The Cancer Genome Atlas (TCGA) pool (n=497) exhibited high expression level of FOLH1 with TRAIL-R2, CSF1R, SEMA4A, SEMA4D, TNFR2, TREM2, MS4A7, C5AR1, ABCC3, STAB1, LILRB4, MERTK, TMEM119, IL4R, SEMA4D, CLEC10A and SELPLG with individual gene expression level measured by Log₂(TPM+1)≥2.3 in the same biopsy sample. Analysis of PRAD tumor biopsies in the TCGA pool suggests >80% patient tumor biopsies exhibit low expression level of immune checkpoint receptors, PD-1, PD-L1, CTLA-4 and LAG-3(gene expression level, Log₂(TPM+1)≤2.3).

### Example 7—Characterization of αTROP2×αTRAIL-R2 Multi-specific Antibody

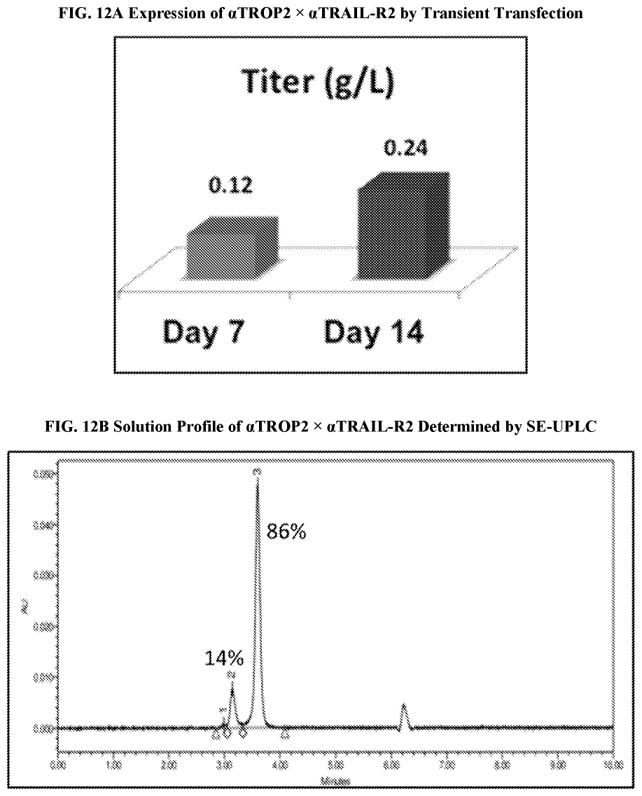

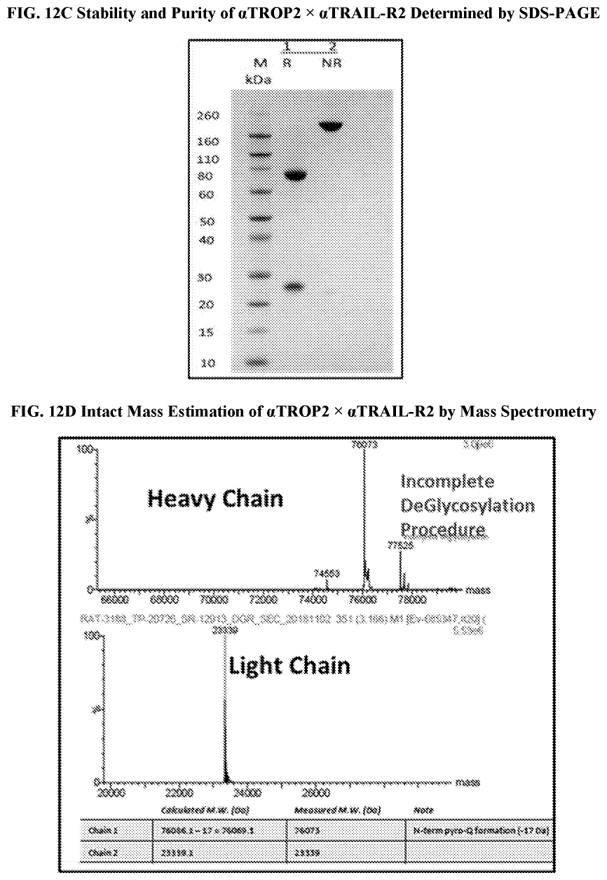

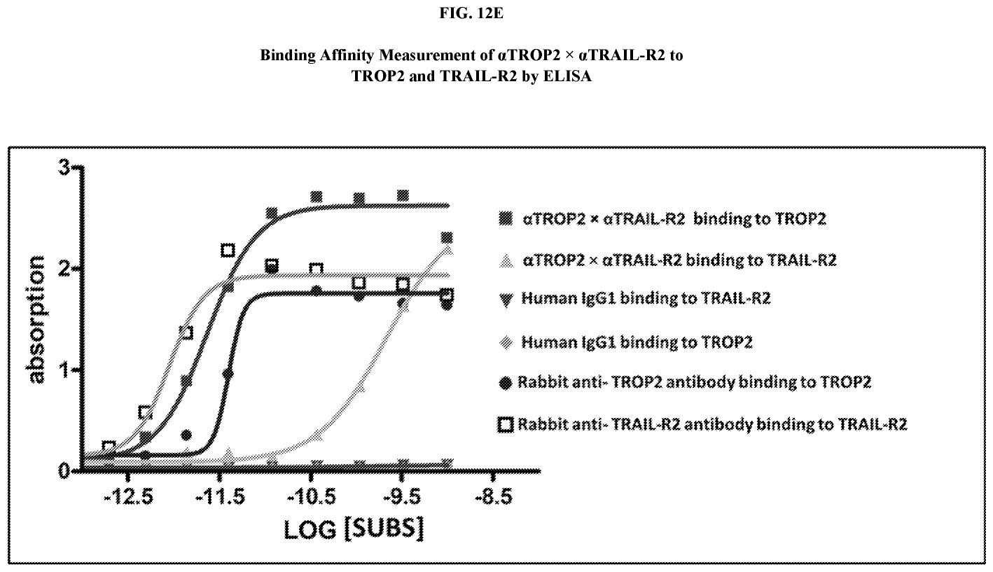

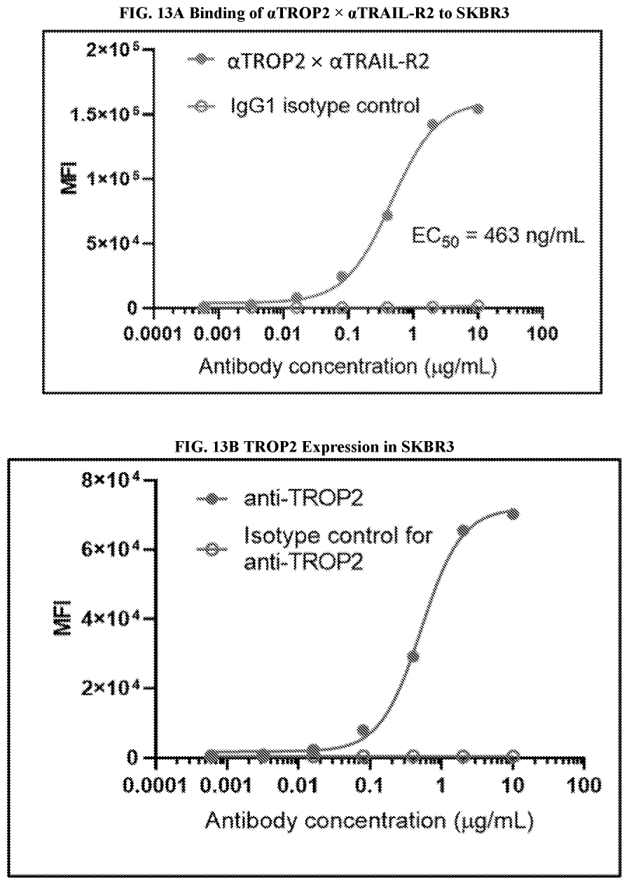

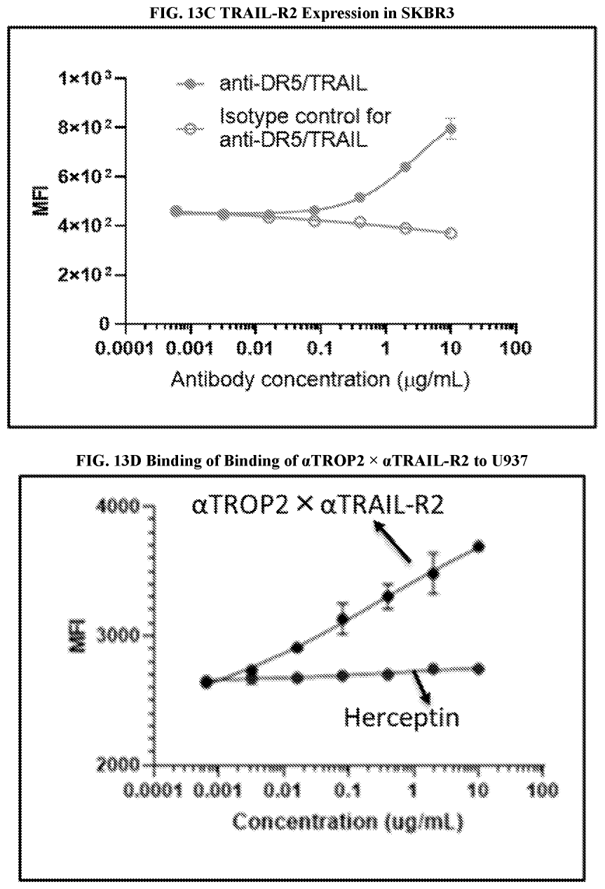

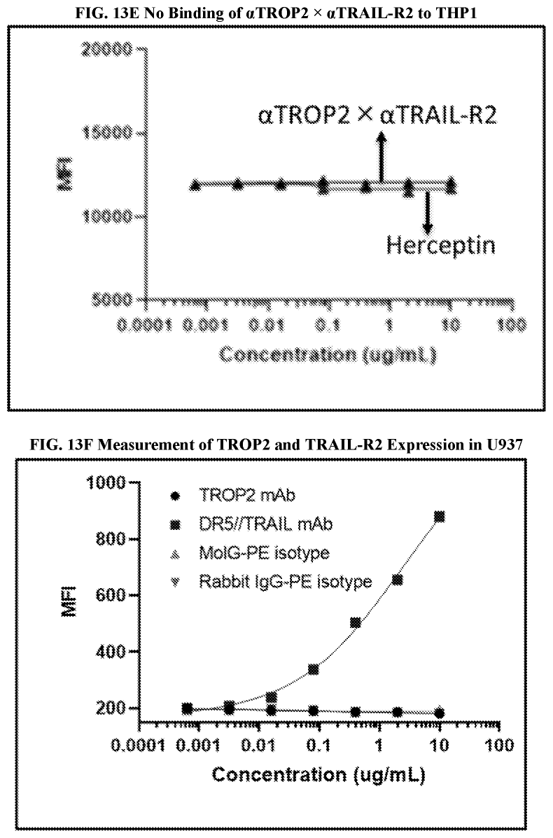

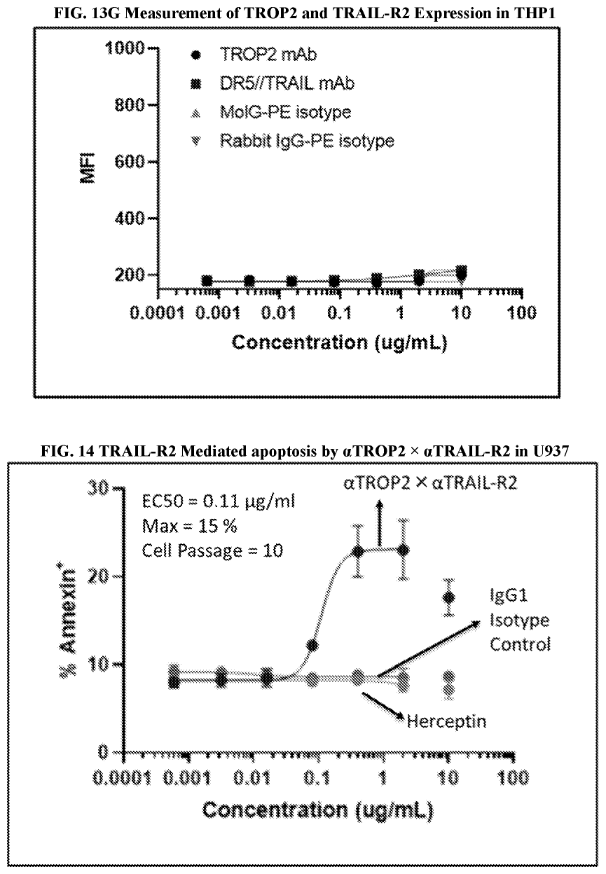

αTROP2×αTRAIL-R2 bispecific antibody was generated according to the scheme in FIG. 1A and comprises SEQ ID NO: 7 and SEQ ID NO: 8, in which the anti-TROP2 binding domain was expressed in the first binding moiety, Fab region, and anti-TRAIL-R2 binding domain was expressed within the second binding moiety, scFv. The construct was transiently expressed in Chinese Hamster Ovary (CHO) mammalian expression system using serum-free chemically defined medium in compliance with current manufacturing practice. A 100 mL transient expression culture yielded a total of ˜23 mg of the multi-specific antibody after Protein A purification with titer as high as 0.24 g/L, FIG. 12A. Analysis of solution profile by Size Exclusion Ultra Performance Liquid Chromatography (SE-UPLC) revealed presence of ˜86% monomer of the intact bispecific antibody with no detection of low molecular weight species, suggesting stability of the construct, as shown in FIG. 12B. Stability and purity of the construct was further confirmed by SDS-PAGE and intact mass analysis under reduced and de-glycosylated conditions where no low molecular weight species was noted, FIG. 12C and FIG. 12D, respectively. Binding affinity to TROP2 and TRAIL-R2 antigens was measured by ELISA in comparison to non-binding human IgG1 isotype (negative control) and binding rabbit anti-TROP2 and anti-TRAIL-R2 antibodies (positive control). The αTROP2×αTRAIL-R2 multi-specific antibody exhibited higher binding affinity towards the tumor associated antigen, TROP2, K_{D}=0.0024 nM, than toward the antigen expressed on immunosuppressive cells, TRAIL-R2, K_{D}=0.22 nM FIG. 12E. The αTROP2×αTRAIL-R2 multi-specific antibody also exhibited target selective binding in cancer cells. In SKBR3, a breast cancer cell line expressing high levels of TROP2 (FIG. 13B) and low but detectable levels of TRAIL-R2 (FIG. 13C) the binding affinity was K_{D}=2.4 nM (0.46 μg/ml), FIG. 13A. In U937 that expresses very low levels of TRAIL-R2 but no TROP2 (FIG. 13F) the αTROP2×αTRAIL-R2 multi-specific antibody exhibited selective binding, FIG. 13D. In THP1 that neither expresses TROP2 nor TRAIL-R2 (FIG. 13G) the αTROP2×αTRAIL-R2 multi-specific antibody exhibited no binding, FIG. 13E. TRAIL-R2 mediated binding of the αTROP2×αTRAIL-R2 multi-specific antibody in U937 and THP1 was detected in the presence of Fc Block reagent that blocked non-specific binding of Fc region of IgG antibodies to Fcγ receptors. High expression of FcγRIA (CD64A) in both U937 and THP1 contributed to binding without the Fc Block reagent in those cell lines. The αTROP2×αTRAIL-R2 multi-specific antibody exhibited dose dependent TRAIL-R2 mediated apoptosis and cell death in U937 in the presence of Fc Block, FIG. 14. A cell killing of 11% and 15% was achieved at IC₅₀=0.65 nM (0.13 μg/ml) in U937. The TRAIL-R2 mediated apoptosis of the αTROP2×αTRAIL-R2 multi-specific antibody was carried out without cross-linking, while studies by several groups had shown that anti-TRAIL-R2 agonist monospecific antibodies were able to induce apoptosis in TRAIL sensitive cancer cell lines only upon cross-linking. The activity of the αTROP2×αTRAIL-R2 multi-specific antibody was higher in cancer cell lines that expressed high levels of TRAIL-R2 with no expression of FcγRIA (CD64A). High expression levels of FcγRIA (CD64A) in U937 binding via Fc region of the antibody contributed to and reduced binding to TRAIL-R2 and subsequently reducing TRAIL-R2 mediated apoptosis.

### Example 8—Characterization of αTROP2×αCD33, αTROP2×αCSF1R and αTROP2×αCD163 Multi-Specific Antibodies

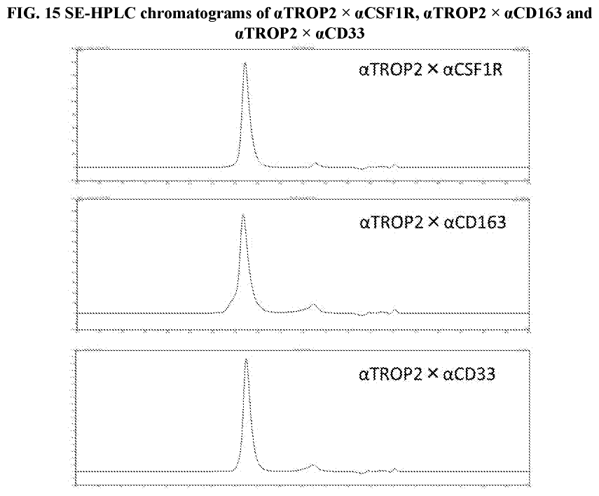

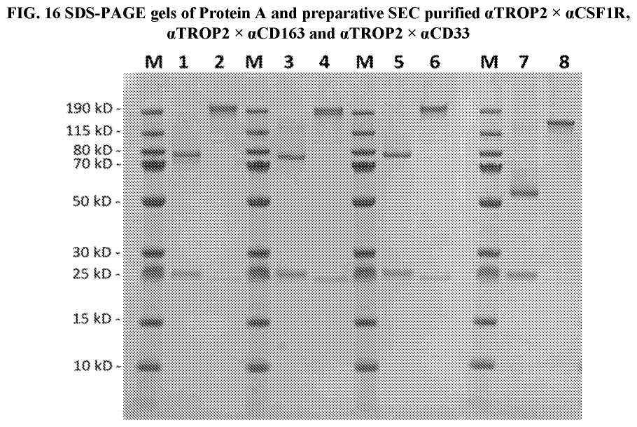

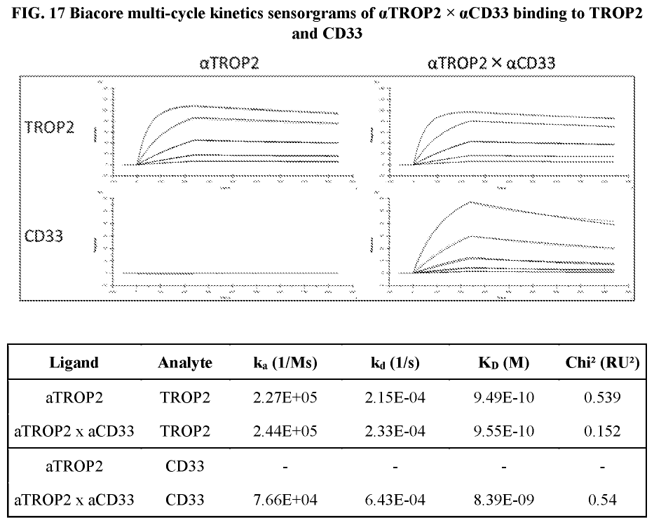

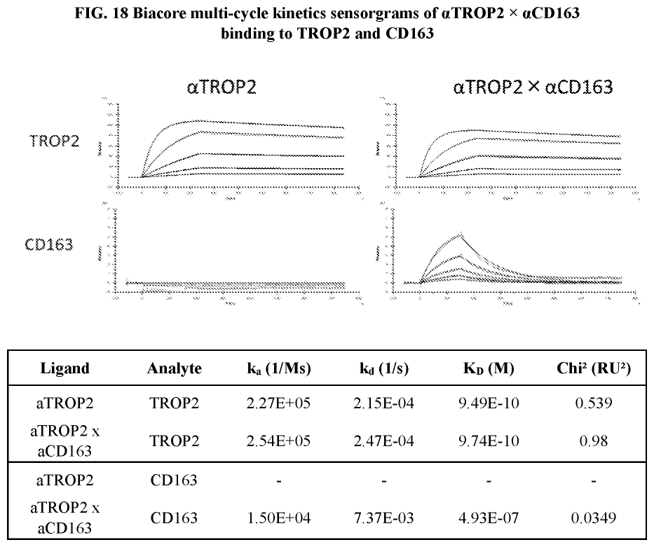

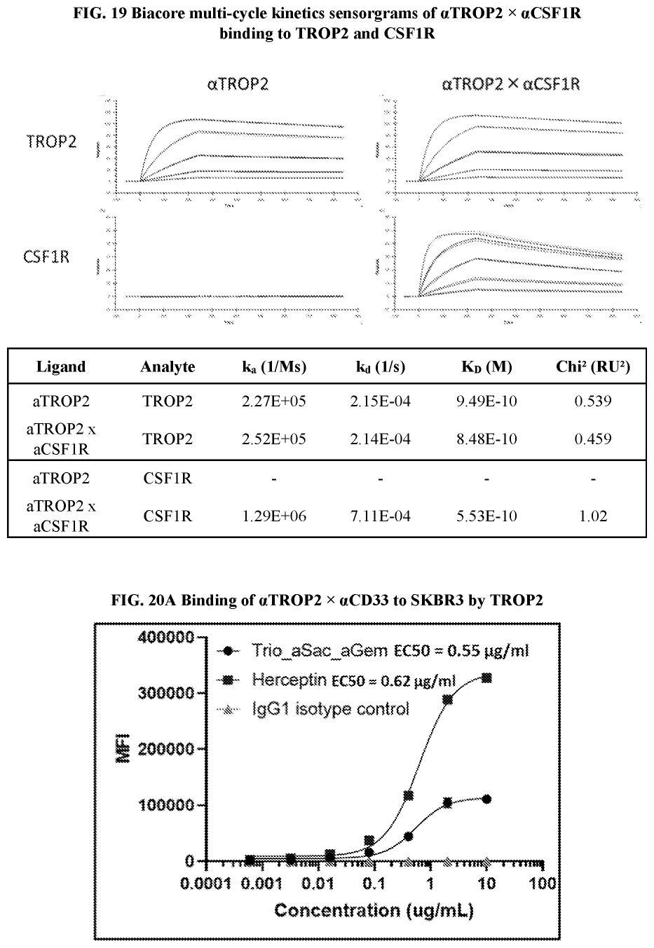

αTROP2×αCD33, αTROP2×αCSF1R and αTROP2×αCD163 bispecific antibodies were generated according to the scheme in FIG. 1A, in which the anti-TROP2 binding domain was expressed in the Fab portion and anti-CD33, anti-CSF1R and anti-CD163 binding domains were expressed in the scFv portion. For αTROP2×αCD33 SEQ ID NOs: 75 and 8 was used; for αTROP2×αCSF1R SEQ ID NOs: 76 and 8 was used; for αTROP2×αCD163 SEQ ID NOs: 77 and 8 was used. The respective construct was transiently expressed in Chinese Hamster Ovary (CHO) mammalian expression system using serum-free chemically defined medium in compliance with current manufacturing practice. A 150 mL transient expression culture yielded a total amount ranging between 4 mg and 10 mg with titers in Day 8 post-transfection as noted: αTROP2×αCD33-0.035 g/L; αTROP2×αCSF1R-0.017 g/L; and αTROP2×αCD163-0.025 g/L. Analysis of solution profile by Size Exclusion High Performance Liquid Chromatography (SE-HPLC) revealed presence of ˜100% monomer of the intact bispecific antibody with no detection of low molecular weight species, suggesting stability of the construct, as shown in FIG. 15. Stability and purity of the construct were further confirmed by SDS-PAGE under reduced and non-reduced conditions where no low molecular weight species were noted, FIG. 16. To assess the binding of the multi-specific antibodies to their respective antigens, multi-cycle kinetics analysis was performed after SEC purification using a Biacore T200 (serial no. 1909913) running Biacore T200 Control Software V2.0.1 and Biacore T200 Evaluation Software V3.0 (Uppsala, Sweden). αTROP2×αCD33 and αTROP2×αCD163 multi-specific antibodies exhibited higher binding affinity towards the tumor associated antigen, TROP2, (K_{D}=0.9 nM), than toward the antigen expressed on immunosuppressive cells, CD33 and CD163, K_{D}=8.4 nM and 493 nM, respectively, FIG. 17, and FIG. 18. The αTROP2×αCSF1R multi-specific antibody exhibited similar binding affinity towards the tumor associated antigen, TROP2, and the antigen expressed on immunosuppressive cells, CSF1R; K_{D}=0.8 nM for TROP2 and K_{D}=0.5 nM for CSF1R, FIG. 19.

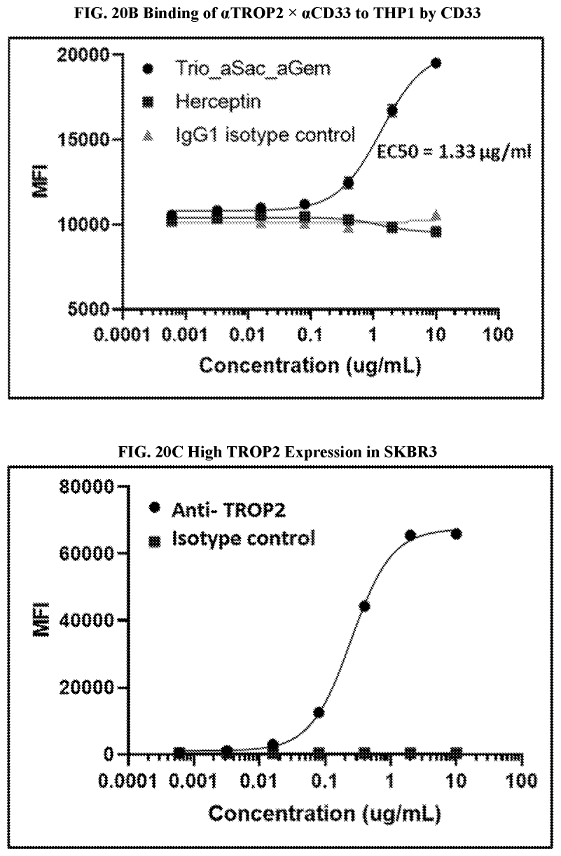

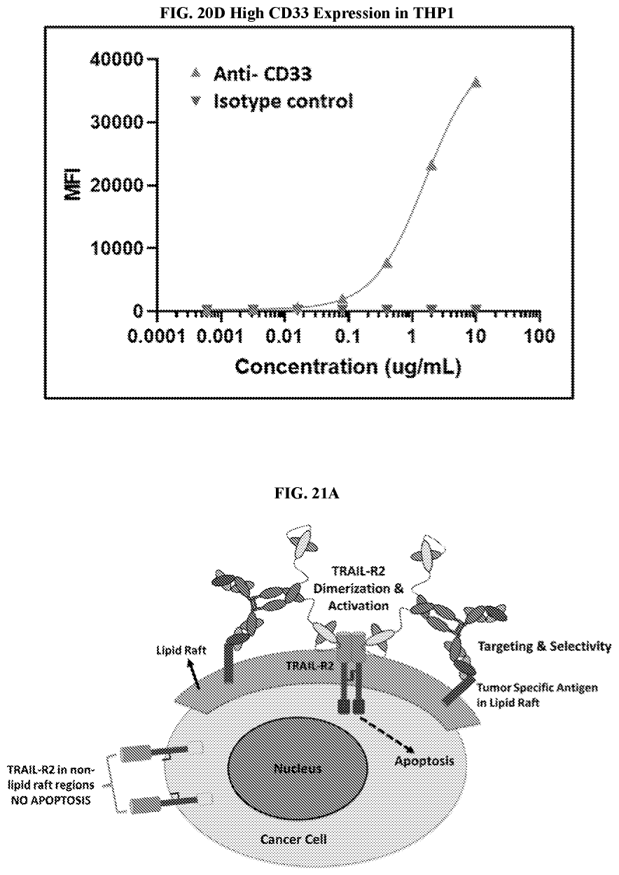

The αTROP2×αCD33 multi-specific antibody also exhibited target selective binding in cancer cells. In SKBR3, a breast cancer cell line expressing high levels of TROP2 (FIGS. 20C and 13B) the binding affinity was K_{D}=2.7 nM (0.55 μg/ml), FIG. 20A. In THP1 that expresses high levels of CD33 but no TROP2 (FIGS. 20D and 13F) the αTROP2×αCD33 multi-specific antibody exhibited selective binding, FIG. 20B. In some instances, CD33 mediated binding of the αTROP2×αCD33 multi-specific antibody in THP1 was detected in the presence of Fc Block reagent that blocked non-specific binding of Fc region of IgG antibodies to Fcγ receptors.

### Example 9—Antibody Humanization

Parental mouse and rabbit antibodies are humanized by grafting CDR or antigen binding sequences from heavy and light chains respectively, onto heavy and light chains of human framework that have high sequence identity with the mouse or rabbit framework sequence. Series of humanized variants are generated by pairing human heavy chain frameworks with human light chain frameworks. The number of humanized variants generated will depend on the number of pairing combinations of human heavy and light chain frameworks. The variants are expressed transiently in HEK293 or CHO or other mammalian expression systems. The supernatant from the expression culture is analyzed for binding to specific human antigen by FACS. The human antigen can be recombinantly expressed on the cell surface of stable mammalian expression cell line.

The binding affinity of the humanized variants is measured in terms of concentration, where lower is the concentration higher is the binding affinity. Usually, humanized antibody variants exhibiting binding affinity in nanomolar range are selected. Furthermore, the selected humanized antibody variants further retain comparable binding affinity profile as the chimera. The selected humanized antibody variants are screened against binding to varied receptor copy numbers from low, medium and high; ideally the selected variants should retain similar binding affinity.

### Example 10—Production of Multi-specific Antibodies

Multi-specific antibodies can be purified using chromatography-based separation methods. The secreted antibodies expressed in mammalian expression system are harvested by separating the cell mass and debris by filtering through a membrane of appropriate size to block cell mass and debris from collecting in the filtrate. The secreted antibody in the filtrate is captured either by batch binding or column binding on a Protein A affinity chromatography resin. The bound antibody is eluted from the resin under acidic conditions pH 2-3 using buffers containing glycine or citrate. The eluted antibody preparation is titrated to pH 5 and diluted 50% by water to reduce conductance. Aggregation and/or impurities are removed from the preparation by cation exchange or HIC chromatography. The antibody is eluted from the resin by gradient of appropriate elution buffer. The eluted fractions are analyzed for monomeric profile by analytic size exclusion chromatography. In addition, the eluted fractions are analyzed for mass analysis by mass-spectrometry. Fractions that matched with the expected molecular weight of the sequence and/or relevant standards and also, revealed a monomeric solution profile by size-exclusion chromatography are pooled together. The pooled multi-specific antibody preparation is formulated onto buffer solutions that are isotonic with human body. For example, the composition can be formulated in a phosphate buffer saline, HEPES buffer saline, Histidine based buffer formulations, Tris-based buffer formulations, citrate-based buffer formulations. Quality control assessments is performed on the final preparation and preparations generated by this strategy resulted in superior quality materials containing <5% aggregate and <5 EU/mg endotoxin level.

### Example 11—Production of Multi-specific Antibodies Conjugated with Toxins

Multi-specific antibodies can be purified using a chromatography-based separation method as described in Example 10. The multi-specific antibody pooled from the chromatography-based separation method can be further conjugated with toxin payload based on conjugation chemistry. The conjugation can be performed by Cys-maleimide conjugation, where the antibody is reduced by reducing agent such as TCEP (Tricarboxyethyl phosphine) or DTT (1,4 Dithiothreitol). The conjugation can be performed in buffer solutions containing 4-6 mM EDTA with buffering capacity spanning between pH 6 and 9. Reducing agent is added to the multi-specific antibody solution reaching a TCEP: antibody ratio of 2-4. Following addition of reducing agent the solution is incubated at 37° C. for 1 hour. The reduced antibody is conjugated to toxin-payloads with maleimide reactive group by adding toxin-payload at a final toxin: reduced Cysteine molar ratio of 1.15. The conjugation reaction can be carried in presence of excipients such as DMSO, trehalose, glycerol, sucrose, etc. with concentrations ranging from 1-20% v/v. The conjugation reaction is performed at 20° C. for 45 minutes-1 hour. The reaction is quenched by adding excess free N(acetyl)-Cysteine 2.3 molar excess than toxin-payload to block the unreacted maleimide group. The quenching reaction can be performed for 20-30 mins at 20° C. A drug-to-antibody ratio (DAR) of 1, 2, 6, and 8 can be accomplished under appropriate conditions. Unconjugated antibodies, unconjugated drug payload-linker, aggregates and impurities can be removed by running the reaction mixture through cation exchange chromatography or hydrophobic interaction chromatography. The antibody drug conjugate can be eluted from the column under a 0-100% gradient of 0.5 M sodium chloride if the method used is cation exchange chromatography. Eluted fractions were analyzed for drug loading and impurity assessments by reverse-phase HPLC, HIC-HPLC, size-exclusion HPLC and mass-spectrometry. Fractions that matched with the expected molecular weight of the sequence and requisite drug-to-antibody ratio and also, revealed a monomeric solution profile by size-exclusion chromatography are pooled together. The pooled multi-specific antibody drug conjugate preparation is formulated onto buffer solutions that are isotonic with human body. For example, the composition of multispecific binding polypeptides described herein can be formulated in a phosphate buffer saline, HEPES buffer saline, Histidine based buffer formulations, Tris-based buffer formulations, citrate-based buffer formulations. Quality control assessments is performed on the final preparation and preparations generated by this strategy resulted in superior quality materials containing <5% aggregate and <5 EU/mg endotoxin level.

### Example 12—Determination of Receptor Expression on Cancer Cell Lines

Expression level of the relevant receptor in cancer cell lines can be estimated by a flow cytometry method. Relevant cell lines expressing the receptor are harvested with Accutase Cell Detachment Solution (BD Bioscience) and assayed for receptor expression using QuantiBRITE PE beads (BD Bioscience) and a PE-conjugated antibody that binds the primary antibody binding the specific receptor. The secondary antibody is conjugated to PE following manufacturer's instructions. Data can be acquired on a FACSCalibur Flow Cytometer (BD) with CellQuest Pro software, with analysis using Flowjo software (Tree Star; Ashland Oreg.).

### Example 13—Cell Killing Potency

Potency of the multi-specific antibodies can be assessed by cell viability in an in vitro cell culture. Specifically the assay determines the number of viable cells present after treatment based on the quantitation of ATP present, which indicates the presence of metabolically active cells. Cell viability can be quantified by using the Cell Titer-Glo assay (Promega) and can be performed according to the manufacturer's instructions. Briefly, cells are first detached from the master culture and seeded in the bottom of each well of 96 well plates (Perkin & Elmer). The number of cells used in seeding can range between 8,000 and 15,000 viable cells per well depending on the cell line. The volume of cell line specific culture medium in each well can vary between 80 and 100 microliters. Cells can be allowed to adhere in a humidified chamber for 3-4 hours at 37° C., 5% CO2. The cells are then treated with the multi-specific antibodies in the proposed invention with concentration varying 0.004 nM to 250 nM. After 72-96 hours viability of the cell is determined by adding 100 microliters per well of Cell Titer-Glo reagent with subsequent mixing on a plate shaker for two minutes at 350 rpm and 10 min incubation in the dark at room temperature. Luminescence can be measured by Synergy 5 reader (Biotek) with a read time of 0.5 seconds per well (sensitivity: 170). Background luminescence in wells with only medium plus the Cell Titer-Glo reagent is subtracted. Data is plotted as percentage of untreated cell viability versus the logarithm of antibody concentration and fitted with 3PL model using Graphpad Prism 5 (GraphPad Software, Inc.). Data from at least three independent experiments are used to calculate the mean IC₅₀±standard deviation (s.d.).

### Example 14—In Vivo Efficacy by Mice Xenograft Studies

A series of murine xenograft studies can be carried out to demonstrate anti-tumor efficacy of multi-specific antibodies in the proposed inventions. Both single dose and repeat dose efficacy studies can be performed on tumor xenograft studies with relevant cell lines. For example, a study can be performed to determine efficacy in xenograft models for triple negative breast cancer utilizing cell lines such as MDA-MB-468, MDA-MB-231, COLO205, SKBR3, U937, THP1, HL-60, KG-1, Shi-1, HNT-34, EOL1, CALU-3, CAPAN-1, PC-3, MCF-7, HCC38, HCC1806, DEL, SUDHL1, GDM1, P31FUJ, or KASUMI6. Immuno-compromised nude (nu/nu) mice, 4-8 weeks old (Taconic Farms) can be engrafted with cancer specific relevant cell lines. A xenograft can be established by making a tumor suspension from stock tumors and mixing with cells harvested from tissue culture. A 0.3 mL injection, containing 20% w/v tumor suspension plus 1×10⁷ cells, is injected subcutaneously into the flank or back of the mice. In some embodiments, a xenograft can be established by harvesting cells from tissue culture and making a final cell suspension by mixing 1:1 with matrigel (BD Bioscience) such that each mouse received a total of 1×10⁷ cells subcutaneously in the flank or back. Tumor volume (TV) is determined by measurements in two dimensions using calipers, with volumes defined as: (L×W×W)/2, where L is the longest dimension of the tumor and W the shortest. Mice is randomized into treatment groups and therapy can be started when tumor volumes are approximately >0.25 cm³. Mice are also weighed periodically by placing on a weighing machine. The dosing strength can be varied from 0.1 mg/kg to a high dose till no toxicity is observed. The dosing frequency is varied based on half-life of the drug and ability to reduce tumor volume. The study protocol and animal handling are performed according to IACUC regulations. Mice are euthanized and deemed to have succumbed to disease once tumors grows >1.0 cm³ in volume. Statistical analysis of tumor growth is based on area under the curve (AUC). Profiles of individual tumor growth can be obtained through linear curve modeling. An f-test is employed to determine equality of variance between groups prior to statistical analysis of growth curves. A two-tailed t-test is applied to assess statistical significance between the various treatment groups and controls, except for the saline control, where a one-tailed t-test is used (significance at P≤0.05). Statistical comparisons of AUC are performed only up to the time that the first animal within a group is euthanized due to disease progression. Survival is analyzed by log-rank test on survival curves generated for each treatment (significance at P≤0.05).

### Example 15—IHC in Tumor Microarrays

Tumor specific microarrays are purchased from vendors specializing in generating microarrays. Antigen retrieval is done by incubating the slides in a Tris/EDTA buffer (DaKo Target Retrieval Solution, pH 9.0; Dako, Denmark), at 95° C. in a NxGen Decloaking Chamber (Biocare Medical; Concord, Calif.) for 30 minutes. The antigen is detected with a goat polyclonal anti-human antigen specific antibody at 10 μg/mL and stained with VECTASTAIN® ABC Kit (Vector Laboratories, Inc.; Burlingame, Calif.). Normal goat antibody is used as the negative control (R&D Systems, Minneapolis, Minn.). Tissues are counterstained with hematoxylin. A formalin-fixed, paraffin-embedded section from a xenograft of human cancer cell line expressing the relevant antigen can serve as a positive control. Scoring is based on the intensity of the stain: >10% of the tumor cells within the specimen, including negative, 1+ (weak), 2+ (moderate), and 3+ (strong).

### Example 16—Measurement of Binding Affinity

Binding kinetic parameters of the multi-specific antibodies are determined by the Octet Red96 system using Octet Data Acquisition software (Forté Bio, Pall). All data are collected at 30° C. in kinetics buffer (KB: PBS pH 7.4, 0.1% BSA, 0.02% Tween-20, Merck) with 1000 rpm orbital senor agitation in a volume of 200 microlitres using black 96-well microplates (Greiner Bio One). The relevant antigens and/or extra-cellular domain are used for the study. Anti-human IgG Fc capture biosensor tips (Forté Bio, Pall) are equilibrated 30 sec in DPBS (Life Technologies). Then, 5 μg/ml antibodies diluted in DPBS are immobilized on biosensor tips for 120 sec, a baseline is recorded for 60 sec in KB followed by stepwise association and dissociation of the analyte for 600 sec and 1200 sec, respectively. Buffer controls are subtracted as background and binding parameters are calculated assuming a 1:1 Langmuir binding model performing global fitting algorithm provided by the Octet data analysis software.

For evaluating simultaneous binding, 5 μg/ml biotinylated antigen A is captured on streptavidin biosensor tips (Forté Bio, Pall) for 40 sec. Biotinylation is performed with the EZ-Link™ Sulfo-NHS-Biotinylation Kit (Thermo Scientific). Biosensors with captured antigen are first blocked with 1% milk powder, 1% BSA, 0.1% Tween® 20 and 10 μg/ml biocytin for 60 sec and then stepwise subjected to 50 nM multi-specific antibody and 50 nM antigen B for 300 sec each. As controls, the non-related isotype control anti-hen egg lysozyme (anti-HEL) or buffer controls can be implemented to exclude unspecific binding.

### Example 17—Pharmacokinetic Studies

The multi-specific antibodies are tested for pharmacokinetic properties. The antibodies are administered by IV bolus injection into the jugular vein via a catheter. The rats are dosed once at strengths ranging from 1 mg/kg to 5 mg/kg. Blood samples are collected from the jugular vein catheter pre-administration and post-administration of drug at designated time points: 0, 2, 24, 48, 96, 168, 240, 384, and 576 hours. Approximately 0.1 mL of whole blood is drawn from the catheter and is collected in a serum separator tube and allowed to clot for 30-60 minutes at room temperature. The serum is separated from the clotted blood by centrifuging at 8000 rpm for 5-10 minutes and is stored at −20° C. The serum is analyzed for the concentration of multi-specific antibodies by an ELISA type assay using an electro-chemi luminescent immunosorbent assay (ECLA). Multi-specific antibodies without drug payloads can be detected in the serum by capture with a specific antigen immobilized on the plate. The captured antibody is then detected by a labeled anti-human Fc or anti-kappa antibody. Multi-specific antibody conjugated with drug toxins are detected with the intact payload where the antibody is captured by an immobilized antigen on the plate. The conjugated drug payload can be detected by polyclonal antibody. Serum concentration versus time graphs is generated in Excel (Microsoft, Redmond, Wash.). The values are reported as mean±standard error of the mean (SEM). Pharmacokinetic analysis on blood clearance based on measured serum concentrations is performed in WinNonlin (Pharsight, St. Louis, Mo.) using non-compartmental analysis.

### Example 18—Lack of Cell Killing in TRAIL-Sensitive MDAMB231 by an Exemplary Multi-Specific Antibody

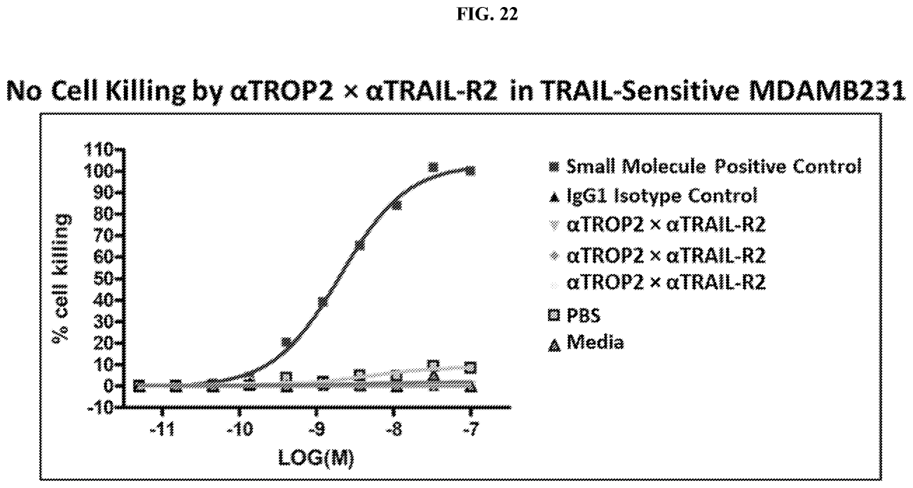

MDAMB231 is a TRAIL-sensitive cell line expressing both TROP2 and TRAIL-R2 on the cell surface. The cell line is killed upon activation of TRAIL-R2 by binding of TRAIL, a ligand of TRAIL-R2. The ability of αTROP2×αTRAIL-R2 in inducing cell death in MDAMB231 upon binding of TRAIL-R2 was determined by measuring the number of viable cells. The number of viable cells present after treatment was based on the quantitation of ATP present, which indicated the presence of metabolically active cells. Cell viability was quantified by using the Cell Titer-Glo assay (Promega) and was performed according to the manufacturer's instructions. Briefly, pre-confluent MDAMB231 cells were seeded in a white opaque 96 well plate (Pierce) at a density of 1,000 cells per well in 100 μl. After ˜16 hours, serial 3-fold dilutions of αTROP2×αTRAIL-R2 and controls (small molecule positive control—MMAE; PBS, media) were prepared so that final concentrations ranged between 100 nM and 0.00508 nM. Cell culture media were removed, and the serial dilutions of the test articles added. The plate was then incubated at 37° C. for three days. After three days, cell killing was assayed using Titer-Glo assay (Promega) according to manufacturer's protocol with a luminometer microplate reader. Analysis of the luminescence readout suggested that there was no concentration dependent change in luminescence for αTROP2×αTRAIL-R2, PBS and media treated cell cultures in comparison to the MMAE treated cell cultures, which exhibited cell killing, IC₅₀=2 nM. This suggest that αTROP2×αTRAIL-R2 did not exhibit any cell killing in MDAMB231 (FIG. 22). The lack of cell killing in MDAMB231 by αTROP2×αTRAIL-R2 is due to preferential binding to TROP2; the binding affinity of αTROP2×αTRAIL-R2 towards TROP2 antigen is 100+ higher than TRAIL-R2 (FIG. 12E). Furthermore, TROP2 is involved in β catenin signaling at cell-cell junction, which is a different subdomain in the plasma membrane. The preferential binding of αTROP2×αTRAIL-R2 to TROP2 at cell-cell junction prevents αTROP2×αTRAIL-R2 from binding to TRAIL-R2 in the lipid raft, and as such, no activation of apoptosis was observed (FIG. 21B).

### Example 19—Determination of Apoptosis and Cell Killing by Selective Activation of TRAIL-R2 in Target Cells by Targeting Tumor Specific Antigen in Lipid Raft

This study utilizes FACS methods to determine the TRAIL-R2 mediated cell killing of target cells with an exemplary multi-specific antibody described herein. The exemplary multi-specific antibody comprises a first binding moiety that binds to a tumor-associated antigen present in the lipid raft of a target cell to recruit and facilitate oligomerization (e.g., dimerization) of the TRAIL-R2 within the lipid raft. The target cells are stained with Annexin V and Propidium Iodide (PI) to detect apoptotic and non-viable cells. Briefly, target cells are first detached from the master culture and seeded in the bottom of each well of 96 well plates (Perkin & Elmer). The number of target cells used in seeding can range between 8,000 and 15,000 viable cells per well depending on the cell line. The volume of cell line specific culture medium in each well can vary between 80 and 100 microliters. Target cells can be allowed to adhere in a humidified chamber for 3-4 hours at 37° C., 5% CO2. The target cells are then treated with the multi-specific antibodies disclosed herein with concentration varying 0.004 nM to 250 nM. After 12-96 hours the target cells are washed and stained with Annexin V and PI to detect apoptotic and necrotic cells. The target cells are washed in PBS and then in binding buffer. The target cells are resuspended in 1× binding buffer at 1-5×10⁶ cells/ml. To 100 μl of the cell suspension 5 μl of fluorochrome-conjugated Annexin V is added. The suspension is incubated at room temperature for 10-15 minutes protected from light. To the cell suspension 2 ml of 1× binding buffer is added and then centrifuged at 400-600 g for 5 minutes at room temperature. The supernatant is discarded. The target cells are then resuspended in 200 μL of 1× binding buffer and added 5 μL of Propidium Iodide Staining Solution and is incubated for 5-15 minutes on ice or at room temperature. The target cells are then analyzed by FACS using BD FACSVerse Instrument. The target cells are analyzed for healthy cells (Annexin V−/PI−), early apoptosis (Annexin V+/PI−) late apoptosis (Annexin V+/PI+), and necrosis (Annexin V−/PI+). The IC₅₀ is calculated by plotting percentage of dead and apoptotic cells against the specific concentration of the test article.

### Example 20—Human Clinical Trial of the Safety and/or Efficacy of αTROP2×αCD33-Val-Cit-MMAE or αTROP2×αCD33-PEG₄-MMAF for TROP2+ Cancers

Objective: To compare the safety and pharmacokinetics of administered dual action bispecific ADC αTROP2×αCD33-Val-Cit-MMAE or αTROP2×αCD33-PEG4-MMAF.

Study Design: This study is a Phase I, single-center, open-label, randomized dose escalation study followed by a Phase II study in TROP2+ patients. Patients who failed IMMU-132 and/or immune checkpoint inhibitor treatments or have not received either of the treatments are eligible. Patients must not have received treatment for their cancer within 14 days of beginning the trial. Treatments include the use of chemotherapy, hematopoietic growth factors, and biologic therapy such as monoclonal antibodies and CAR-Ts. Patients should recover from all toxicities associated with previous treatment. All patients are evaluated for safety and all blood collections for pharmacokinetic analysis are collected as scheduled. All studies are performed with institutional ethics committee approval and patient consent.

Phase I: Patients receive I.V. on days 1 and 30 of 30-day cycle. Doses may be held or modified for toxicity based on assessments as outlined below. Treatment repeats every 30 days in the absence of unacceptable toxicity. Cohorts of 3-6 patients receive escalating doses of αTROP2×αCD33-Val-Cit-MMAE or αTROP2×αCD33-PEG₄-MMAF until the maximum tolerated dose (MTD) is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006). The MTD is the Recommended Phase 2 Dose (RP2D) to be administered during Phase II clinical trial.

Phase II: Patients receive αTROP2×αCD33-Val-Cit-MMAE or αTROP2×αCD33-PEG4-MMAF as in phase I at the RP2D. Treatment regimens involve once every 4 or 8 weeks for 2-6 courses in the absence of disease progression or unacceptable toxicity. After completion of 2 courses of therapy, patients who achieve a complete or partial response may receive additional 4 courses. Patients who maintain stable disease for more than 8 weeks after completion of 6 courses of study therapy may receive an additional 6 courses at the time of disease progression, provided they meet original eligibility criteria.

Blood Sampling: serial blood is drawn by direct vein puncture before and after administration of αTROP2×αCD33-Val-Cit-MMAE or αTROP2×αCD33-PEG₄-MMAF. Venous blood samples (5 mL) for determination of serum concentrations are obtained at about 10 minutes prior to dosing and at approximately the following times after dosing: days 1, 8, and 15. All serum samples are stored at −20° C.

Pharmacokinetics: Patients undergo serum collection for pharmacokinetic evaluation before beginning treatment and at days 1, 8, and 15. Pharmacokinetic parameters are calculated by model independent methods using the latest version of pharmacokinetics evaluation software. The following pharmacokinetics parameters are determined: peak serum concentration (Cmax); time to peak serum concentration (Tmax); area under the concentration-time curve (AUC) from time zero to the last blood sampling time (AUCO-72) calculated with the use of the linear trapezoidal rule; and terminal elimination half-life (T½), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Response rates are determined using the RECIST criteria. (Therasse et al, J. Natl. Cancer Inst. 2000 Feb. 2; 92(3):205-16). Patient response is assessed by imaging with X-ray, CT scans, and MRI. Imaging is performed prior to beginning the study and at the end of the first cycle, with additional imaging performed every four weeks or at the end of subsequent cycles. Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

Embodiment 1: a multi-specific antibody comprising a tumor binding moiety that specifically binds to a tumor-associated antigen and an immune cell binding moiety that specifically binds to an antigen expressed on an immunosuppressive cell.

Embodiment 2: a multi-specific antibody comprising a tumor binding moiety that specifically binds to a tumor-associated antigen and an immune cell binding moiety that specifically binds to an antigen expressed on an immunosuppressive cell, wherein a first binding affinity between the immune cell binding moiety and the antigen expressed on the immunosuppressive cell is less than a second binding affinity between the tumor binding moiety and the tumor-associated antigen.

Embodiment 3: a multi-specific antibody comprising a tumor binding moiety that specifically binds to a tumor-associated antigen and an immune cell binding moiety that specifically binds to an antigen expressed on a Myeloid-derived suppressor cells (MDSC) or a tumor-associated macrophage (TAM).

Embodiment 4: the multi-specific antibody of embodiment 3, wherein the immune cell binding moiety specifically binds to an antigen expressed on a Myeloid-derived suppressor cell (MDSC).

Embodiment 5: the multi-specific antibody of embodiment 3, wherein the immune cell binding moiety specifically binds to an antigen expressed on a TAM, optionally a M2 polarized TAM (M2-TAM).

Embodiment 6: the multi-specific antibody of embodiment 1 or 2, wherein the immunosuppressive cell is a Myeloid-derived suppressor cell (MDSC).

Embodiment 7: the multi-specific antibody of embodiment 1 or 2, wherein the immunosuppressive cell is a tumor-associated macrophage (TAM), optionally a M2 polarized TAM (M2-TAM).

Embodiment 8: the multi-specific antibody of embodiment 1 or 2, wherein the immunosuppressive cell is a T regulator (Treg) cell.

Embodiment 9: the multi-specific antibody of any one of the embodiments 1-8, wherein the tumor-associated antigen is TROP2, HER2, GPC3, GD2, FOLR1, FLT3, BCMA, MUC16, SLC4A4, STEAP1, CD19, CD20, CD22, CD25, CD33, CD38, CD30, CD47, CD123, mesothelin, MT1-MMP, or PSMA.

Embodiment 10: the multi-specific antibody of any one of the embodiments 1-8, wherein the tumor associated antigen is TROP2, GPC3, HER2, FOLR1, CD33, CD38, FLT3, CD30, CD22, or GD2.

Embodiment 11: the multi-specific antibody of any one of the embodiments 1-8, wherein the tumor-associated antigen is TROP2, GPC3, FOLR1, CD33, CD38, or FLT3.

Embodiment 12: the multi-specific antibody of any one of the embodiments 1-8, wherein the tumor-associated antigen is TROP2, CD47, HER2, CD30, CD22, GD2, or FOLR1.

Embodiment 13: the multi-specific antibody of any one of the embodiments 1-12, wherein the antigen expressed on the immunosuppressive cell is TRAIL-R2, CD33, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG.

Embodiment 14: the multi-specific antibody of any one of the embodiments 1-12, wherein the antigen expressed on the immunosuppressive cell is TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9.

Embodiment 15: the multi-specific antibody of any one of the embodiments 1-12, wherein the antigen expressed on the immunosuppressive cell is TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2.

Embodiment 16: the multi-specific antibody of any one of the embodiments 1-12, wherein the antigen expressed on the immunosuppressive cell is TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R.

Embodiment 17: the multi-specific antibody of any one of the embodiments 1-12, wherein the antigen expressed on the immunosuppressive cell is TRAIL-R2, CSF1R, TREM2, C5AR1, LYVE1, MRC1, STAB1, MERTK, SIGLEC1, or IL4R.

Embodiment 18: the multi-specific antibody of any one of the embodiments 1-12, wherein the antigen expressed on the immunosuppressive cell is MARCO, SELPLG, CD163, MS4A7, CD200R, MGL1, or MGL2.

Embodiment 19: the multi-specific antibody of any one of the embodiments 1-12, wherein the antigen expressed on the immunosuppressive cell is CD33, ABCC3, LILRB4, TMEM37, TMEM119, SIGLEC7, or SIGLEC9.

Embodiment 20: the multi-specific antibody of any one of the embodiments 1-12, wherein the antigen expressed on the immunosuppressive cell is SEMA4A, SEMA4D, or TNFR2.

Embodiment 21: the multi-specific antibody of any one of the embodiments 1-12, wherein the antigen expressed on the immunosuppressive cell is TRAIL-R2, CD33, CD163, or CSF1R.

Embodiment 22: the multi-specific antibody of any one of the embodiments 1-12, wherein the antigen expressed on the immunosuppressive cell is CD33, CD163, or CSF1R.

Embodiment 23: the multi-specific antibody of any one of the embodiments 1-22, wherein the multi-specific antibody is a bispecific antibody.

Embodiment 24: the multi-specific antibody of any one of the embodiments 1-23, wherein the multi-specific antibody comprises an Fc portion.

Embodiment 25: the multi-specific antibody of any one of the embodiments 1-24, wherein the multi-specific antibody comprises an Fc region that has been modified to reduce the affinity for human neonatal Fc receptor (FcRn).

Embodiment 26: the multi-specific antibody of any one of the embodiments 1-24, wherein the multi-specific antibody comprises an Fc region comprising a modification to reduce antibody-dependent cellular cytotoxicity (ADCC), wherein the modification optionally comprises L234, L235, P238, or P331, or a combination thereof, wherein L234, L235, P238, and P331 correspond to positions 234, 235, 238, and 331 of a wild-type IgG1, according to the EU numbering convention.

Embodiment 27: the multi-specific antibody of any one of the embodiments 1-24, wherein the multi-specific antibody comprises an Fc region comprising a modification to reduce antibody-dependent cellular cytotoxicity (ADCC), wherein the modification optionally comprises L234, L235, P238, and P331, wherein L234, L235, P238, and P331 correspond to positions 234, 235, 238, and 331 of a wild-type IgG1, according to the EU numbering convention.

Embodiment 28: the multi-specific antibody of embodiment 26 or 27, wherein the Fc region comprises L234A, L235A, P238S, P331S, ora combination thereof.

Embodiment 29: the multi-specific antibody of any one of the embodiments 1-28, wherein the multi-specific antibody comprises an Fc region that has been modified to reduce neutropenia.

Embodiment 30: the multi-specific antibody of embodiment 29, wherein the Fc region comprises a modification at L234, S239, S442, or a combination thereof, wherein L234, S239, and S442 correspond to positions 234, 239, 442 of a wild-type IgG1, according to the EU numbering convention.

Embodiment 31: the multi-specific antibody of embodiment 29 or 30, wherein the Fc region comprises L234F, S239C, S442C, or a combination thereof.

Embodiment 32: the multi-specific antibody of any one of the embodiments 1-31, wherein the multi-specific antibody comprises an Fc region that has been modified to enhance antibody-dependent cellular cytotoxicity (ADCC).

Embodiment 33: the multi-specific antibody of embodiment 32, wherein the Fc region comprises a modification at S239, A330, I332, or a combination thereof, wherein S239, A330, and 1332 correspond to positions 239, 330, and 332 of a wild-type IgG1, according to the EU numbering convention.

Embodiment 34: the multi-specific antibody of embodiment 32 or 33, wherein the Fc region comprises S239D, A330L, I332E, or a combination thereof.

Embodiment 35: the multi-specific antibody of any one of the embodiments 1-34, wherein the multi-specific antibody comprises a modification to a hinge region.

Embodiment 36: the multi-specific antibody of embodiment 35, wherein the hinge region comprises a modification at S228, wherein S228 correspond to position 228 of a wild-type IgG4, according to the EU numbering convention.

Embodiment 37: the multi-specific antibody of embodiment 35 or 36, wherein the hinge region comprises S228P.

Embodiment 38: the multi-specific antibody of any one of the embodiments 1-37, wherein the multi-specific antibody comprises a full-length antibody, an appended antibody, a bispecific fusion protein, or a bispecific antibody conjugate.

Embodiment 39: the multi-specific antibody of any one of the embodiments 1-38, wherein the multi-specific antibody comprises a nanobody, BiTE, diabody, DART, TandAb, scDiabody, scDiabody-CH3, triple body, miniantibody, minibody, TriBi minibody, scFv-CH3 KIH, Fab-scFv, scFv-CH-CL-scFv, F(ab′)2, F(ab′)2-scFv2. scFv-KIH, Fab-scFv-Fc, tetravalent HCAb, scDiabody-Fc, diabody-Fc, tandem scFv-Fc, or intrabody.

Embodiment 40: the multi-specific antibody of any one of the embodiments 1-39, wherein the multi-specific antibody comprises an IgG framework, optionally an IgG1, IgG2, or IgG4 framework, further optionally an IgG1 or IgG4 framework.

Embodiment 41: the multi-specific antibody of any one of the embodiments 1-40, wherein the multi-specific antibody is a humanized antibody.

Embodiment 42: the multi-specific antibody of any one of the embodiments 1-40, wherein the multi-specific antibody is a chimeric antibody.

Embodiment 43: the multi-specific antibody of any one of the embodiments 1-42, wherein the tumor binding moiety comprises a full-length antibody or antigen binding fragment thereof.

Embodiment 44: the multi-specific antibody of embodiment 43, wherein the tumor binding moiety comprises an IgG antibody framework, optionally an IgG1 or IgG4 framework.

Embodiment 45: the multi-specific antibody of embodiment 43 or 44, wherein the tumor binding moiety comprises a full-length antibody.

Embodiment 46: the multi-specific antibody of embodiment 43 or 44, wherein the tumor binding moiety comprises a Fab, F(ab)₂, single-domain antibody, a single chain variable fragment (scFv), or a nanobody.

Embodiment 47: the multi-specific antibody of any one of the embodiments 43-46, wherein the tumor binding moiety is a humanized antibody.

Embodiment 48: the multi-specific antibody of any one of the embodiments 43-46, wherein the tumor binding moiety is a chimeric antibody.

Embodiment 49: the multi-specific antibody of any one of the embodiments 43-48, wherein the tumor binding moiety comprises an immunoglobulin heavy chain variable region comprising an amino acid sequence at least about 90%, 95%, 97%, 98%, 99%, or 100% identical to or consist of the amino acid sequence set forth in SEQ ID NOs: 9, 16, 20, 24, or 28; and an immunoglobulin light chain variable region at least about 90%, 95%, 97%, 98%, 99%, or 100% identical to or consist of the amino acid sequence set forth in SEQ ID NOs: 10, 32, 36, 40, or 44.

Embodiment 50: the multi-specific antibody of any one of the embodiments 43-48, wherein the tumor binding moiety comprises an immunoglobulin heavy chain variable region comprising an amino acid sequence at least about 90%, 95%, 97%, 98%, 99%, or 100% identical to or consist of the amino acid sequence set forth in SEQ ID NO: 9; and an immunoglobulin light chain variable region at least about 90%, 95%, 97%, 98%, 99%, or 100% identical to or consist of the amino acid sequence set forth in SEQ ID NO: 10.

Embodiment 51: the multi-specific antibody of any one of the embodiments 43-48, wherein the tumor binding moiety comprises an immunoglobulin heavy chain variable region comprising an amino acid sequence at least about 90%, 95%, 97%, 98%, 99%, or 100% identical to or consist of the amino acid sequence set forth in SEQ ID NO: 16; and an immunoglobulin light chain variable region at least about 90%, 95%, 97%, 98%, 99%, or 100% identical to or consist of the amino acid sequence set forth in SEQ ID NO: 32.

Embodiment 52: the multi-specific antibody of any one of the embodiments 43-48, wherein the tumor binding moiety comprises an immunoglobulin heavy chain variable region comprising an amino acid sequence at least about 90%, 95%, 97%, 98%, 99%, or 100% identical to or consist of the amino acid sequence set forth in SEQ ID NO: 20; and an immunoglobulin light chain variable region at least about 90%, 95%, 97%, 98%, 99%, or 100% identical to or consist of the amino acid sequence set forth in SEQ ID NO: 36.

Embodiment 53: the multi-specific antibody of any one of the embodiments 43-48, wherein the tumor binding moiety comprises an immunoglobulin heavy chain variable region comprising an amino acid sequence at least about 90%, 95%, 97%, 98%, 99%, or 100% identical to or consist of the amino acid sequence set forth in SEQ ID NO: 24; and an immunoglobulin light chain variable region at least about 90%, 95%, 97%, 98%, 99%, or 100% identical to or consist of the amino acid sequence set forth in SEQ ID NO: 40.

Embodiment 54: the multi-specific antibody of any one of the embodiments 43-48, wherein the tumor binding moiety comprises an immunoglobulin heavy chain variable region comprising an amino acid sequence at least about 90%, 95%, 97%, 98%, 99%, or 100% identical to or consist of the amino acid sequence set forth in SEQ ID NO: 28; and an immunoglobulin light chain variable region at least about 90%, 95%, 97%, 98%, 99%, or 100% identical to or consist of the amino acid sequence set forth in SEQ ID NO: 44.

Embodiment 55: the multi-specific antibody of any one of the embodiments 1-54, wherein the immune cell binding moiety comprises a full-length antibody or antigen binding fragment thereof.

Embodiment 56: the multi-specific antibody of embodiment 55, wherein the immune cell binding moiety comprises an IgG antibody framework, optionally an IgG1 or IgG4 framework.

Embodiment 57: the multi-specific antibody of embodiment 55 or 56, wherein the immune cell binding moiety comprises a full-length antibody.

Embodiment 58: the multi-specific antibody of embodiment 55 or 56, wherein the immune cell binding moiety comprises a Fab, F(ab)₂, single-domain antibody, a single chain variable fragment (scFv), or a nanobody.

Embodiment 59: the multi-specific antibody of any one of the embodiments 55-58, wherein the immune cell binding moiety is a humanized antibody.

Embodiment 60: the multi-specific antibody of any one of the embodiments 55-58, wherein the immune cell binding moiety is a chimeric antibody.

Embodiment 61: the multi-specific antibody of any one of the embodiments 55-60, wherein the immune cell binding moiety comprises an immunoglobulin heavy chain variable region comprising an amino acid sequence at least about 90%, 95%, 97%, 98%, 99%, or 100% identical to the amino acid sequence set forth in SEQ ID NOs: 11, 48, 52, or 59; and an immunoglobulin light chain variable region at least about 90%, 95%, 97%, 98%, 99%, or 100% identical to the amino acid sequence set forth in SEQ ID NOs: 12, 63, 67, or 74.

Embodiment 62: the multi-specific antibody of any one of the embodiments 55-60, wherein the immune cell binding moiety comprises an immunoglobulin heavy chain variable region comprising an amino acid sequence at least about 90%, 95%, 97%, 98%, 99%, or 100% identical to the amino acid sequence set forth in SEQ ID NO: 11; and an immunoglobulin light chain variable region at least about 90%, 95%, 97%, 98%, 99%, or 100% identical to the amino acid sequence set forth in SEQ ID NO: 12.

Embodiment 63: the multi-specific antibody of any one of the embodiments 55-60, wherein the immune cell binding moiety comprises an immunoglobulin heavy chain variable region comprising an amino acid sequence at least about 90%, 95%, 97%, 98%, 99%, or 100% identical to the amino acid sequence set forth in SEQ ID NO: 48; and an immunoglobulin light chain variable region at least about 90%, 95%, 97%, 98%, 99%, or 100% identical to the amino acid sequence set forth in SEQ ID NO: 63.

Embodiment 64: the multi-specific antibody of any one of the embodiments 55-60, wherein the immune cell binding moiety comprises an immunoglobulin heavy chain variable region comprising an amino acid sequence at least about 90%, 95%, 97%, 98%, 99%, or 100% identical to the amino acid sequence set forth in SEQ ID NO: 52; and an immunoglobulin light chain variable region at least about 90%, 95%, 97%, 98%, 99%, or 100% identical to the amino acid sequence set forth in SEQ ID NO: 67.

Embodiment 65: the multi-specific antibody of any one of the embodiments 55-60, wherein the immune cell binding moiety comprises an immunoglobulin heavy chain variable region comprising an amino acid sequence at least about 90%, 95%, 97%, 98%, 99%, or 100% identical to the amino acid sequence set forth in SEQ ID NO: 59; and an immunoglobulin light chain variable region at least about 90%, 95%, 97%, 98%, 99%, or 100% identical to the amino acid sequence set forth in SEQ ID NO: 74.

Embodiment 66: the multi-specific antibody of any one of the embodiments 1-65, wherein the tumor binding moiety comprises an IgG antibody framework, optionally a full-length IgG antibody framework, the immune cell binding moiety is an scFv, and the immune cell binding moiety is coupled to the C-terminus of the tumor binding moiety, optionally recombinantly fused to the C-terminus of the tumor binding moiety.

Embodiment 67: the multi-specific antibody of any one of the embodiments 1-65, wherein the tumor binding moiety comprises an IgG antibody framework, optionally a full-length IgG antibody framework, the immune cell binding moiety is an scFv, and the immune cell binding moiety is coupled to the N-terminus of the tumor binding moiety, optionally recombinantly fused to the N-terminus of the tumor binding moiety.

Embodiment 68: the multi-specific antibody of any one of the embodiments 1-65, wherein the immune cell binding moiety is an IgG antibody framework, optionally a full-length IgG antibody framework, the tumor binding moiety is an scFv, and the tumor binding moiety is coupled to the C-terminus of the immune cell binding moiety, optionally recombinantly fused to the C-terminus of the immune cell binding moiety.

Embodiment 69: the multi-specific antibody of any one of the embodiments 1-65, wherein the immune cell binding moiety is an IgG antibody framework, optionally a full-length IgG antibody framework, the tumor binding moiety is an scFv, and the tumor binding moiety is coupled to the N-terminus of the immune cell binding moiety, optionally recombinantly fused to the N-terminus of the immune cell binding moiety.

Embodiment 70: the multi-specific antibody any one of the embodiments 66-69, wherein the immune cell binding moiety is coupled to the tumor binding moiety by a polypeptide linker.

Embodiment 71: the multi-specific antibody of embodiment 70, wherein the polypeptide linker comprises (Gly4Ser)ₙ, wherein n is an integer from 1 to 10, optionally from 1 to 6, 1 to 4, or 1 to 2, further optionally 1, 2, 3, or 4.

Embodiment 72: the multi-specific antibody of any one of the embodiments 1-71, wherein the antibody comprises an immunoglobulin heavy chain comprising an amino acid sequence at least about 90%, 95%, 97%, 98%, 99%, or 100% identical to or consist of the amino acid sequence set forth in Table 5; and an immunoglobulin light chain at least about 90%, 95%, 97%, 98%, 99%, or 100% identical to or consist of the amino acid sequence set forth in Table 5.

Embodiment 73: the multi-specific antibody of any one of the embodiments 1-71, wherein the antibody comprises an immunoglobulin heavy chain comprising an amino acid sequence at least about 90%, 95%, 97%, 98%, 99%, or 100% identical to or consist of the amino acid sequence set forth in SEQ ID NOs: 7 or 75-77; and an immunoglobulin light chain at least about 90%, 95%, 97%, 98%, 99%, or 100% identical to or consist of the amino acid sequence set forth in SEQ ID NO: 8.

Embodiment 74: the multi-specific antibody of any one of the embodiments 1-71, wherein the antibody comprises an immunoglobulin heavy chain comprising an amino acid sequence at least about 90%, 95%, 97%, 98%, 99%, or 100% identical to or consist of the amino acid sequence set forth in SEQ ID NOs: 79-83; and an immunoglobulin light chain at least about 90%, 95%, 97%, 98%, 99%, or 100% identical to or consist of the amino acid sequence set forth in SEQ ID NO: 80.

Embodiment 75: the multi-specific antibody of any one of the embodiments 1-71, wherein the antibody comprises an immunoglobulin heavy chain comprising an amino acid sequence at least about 90%, 95%, 97%, 98%, 99%, or 100% identical to or consist of the amino acid sequence set forth in SEQ ID NOs: 84-88; and an immunoglobulin light chain at least about 90%, 95%, 97%, 98%, 99%, or 100% identical to or consist of the amino acid sequence set forth in SEQ ID NO: 85.

Embodiment 76: the multi-specific antibody of any one of the embodiments 1-71, wherein the antibody comprises an immunoglobulin heavy chain comprising an amino acid sequence at least about 90%, 95%, 97%, 98%, 99%, or 100% identical to or consist of the amino acid sequence set forth in SEQ ID NOs: 89, 91, or 93; and an immunoglobulin light chain at least about 90%, 95%, 97%, 98%, 99%, or 100% identical to or consist of the amino acid sequence set forth in SEQ ID NO: 90, 92, or 94.

Embodiment 77: the multi-specific antibody of any one of the embodiments 1-76, wherein the multi-specific antibody further comprises at least one payload.

Embodiment 78: the multi-specific antibody of embodiment 77, wherein the at least one payload comprises an auristatin derivative, maytansine, a maytansinoid, a taxane, a calicheamicin, cemadotin, a duocarmycin, a pyrrolobenzodiazepine (PBD), tubulysin, dexamethasone, or dasatinib.

Embodiment 79: the multi-specific antibody of embodiment 78, wherein the auristatin derivative is monomethyl auristatin E (MMAE) or monomethyl auristatin F (MMAF).

Embodiment 80: the multi-specific antibody of embodiment 78, wherein the maytansinoid is DM1, DM2 (mertansine), or DM4.

Embodiment 81: the multi-specific antibody of embodiment 78, wherein the pyrrolobenzodiazepine is a pyrrolobenzodiazepine dimer.

Embodiment 82: the multi-specific antibody of any one of the embodiments 77-81, wherein the at least one payload is attached to the multi-specific antibody via a linker.

Embodiment 83: the multi-specific antibody of embodiment 82, wherein the linker is a cleavable linker.

Embodiment 84: the multi-specific antibody of embodiment 82, wherein the linker is a pH-sensitive linker.

Embodiment 85: the multi-specific antibody of embodiment 82, wherein the linker is an enzyme-sensitive linker.

Embodiment 86: the multi-specific antibody of embodiment 82 or 85, wherein the linker is a protease-sensitive linker.

Embodiment 87: the multi-specific antibody of embodiment 82, wherein the linker is a self-immolative linker.

Embodiment 88: the multi-specific antibody of embodiment 82, wherein the linker is a non-cleavable linker.

Embodiment 89: the multi-specific antibody of any one of the embodiments 82-88, wherein the linker comprises a zero-length linker, a homobifunctional linker, a heterobifunctional linker, a di-peptide linker, a spacer, a maleimide-based conjugating moiety, or a combination thereof.

Embodiment 90: the multi-specific antibody of any one of the embodiments 82-89, wherein the linker comprises a polymer.

Embodiment 91: the multi-specific antibody of embodiment 90, wherein the polymer comprises a linear or branched polyethylene glycol.

Embodiment 92: the multi-specific antibody of embodiment 82, wherein the linker is a peptide.

Embodiment 93: the multi-specific antibody of embodiment 82, wherein the linker is a peptidomimetic linker.

Embodiment 94: the multi-specific antibody of any one of the embodiments 1-93, wherein a ratio of the payload to the multi-specific antibody is about 1:1, about 2:1, about 3:1, about 4:1, about 5:1, about 6:1, about 7:1, about 8:1, about 10:1, or about 12:1.

Embodiment 95: the multi-specific antibody of any one of the embodiments 2 or 6-94, wherein the first binding affinity is less than the second binding affinity by about 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, or higher.

Embodiment 96: a pharmaceutical composition comprising a multi-specific antibody of embodiments 1-95, and a pharmaceutically acceptable excipient.

Embodiment 97: the pharmaceutical composition of embodiment 96, wherein the pharmaceutical composition is formulated for systemic administration.

Embodiment 98: the pharmaceutical composition of embodiment 96, wherein the pharmaceutical composition is formulated for local administration.

Embodiment 99: the pharmaceutical composition of any one of the embodiments 96-98, wherein the pharmaceutical composition is formulated for parenteral administration.

Embodiment 100: the pharmaceutical composition of any one of the embodiments 96-99, wherein the pharmaceutical composition is formulated for subcutaneous, intramuscular, or intravenous administration.

Embodiment 101: a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in Table 5; and optionally an immunoglobulin light chain at least about 90%, 95%, 97%, 98%, 99%, or 100% identical to or consist of the amino acid sequence set forth in Table 5.

Embodiment 102: a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 7 or 75-77; and optionally an immunoglobulin light chain at least about 90%, 95%, 97%, 98%, 99%, or 100% identical to or consist of the amino acid sequence set forth in SEQ ID NO: 8.

Embodiment 103: a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 79-83; and optionally an immunoglobulin light chain at least about 90%, 95%, 97%, 98%, 99%, or 100% identical to or consist of the amino acid sequence set forth in SEQ ID NO: 80.

Embodiment 104: a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 84-88; and optionally an immunoglobulin light chain at least about 90%, 95%, 97%, 98%, 99%, or 100% identical to or consist of the amino acid sequence set forth in SEQ ID NO: 85.

Embodiment 105: a nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 89, 91, or 93; and optionally an immunoglobulin light chain at least about 90%, 95%, 97%, 98%, 99%, or 100% identical to or consist of the amino acid sequence set forth in SEQ ID NO: 90, 92, or 94.

Embodiment 106: a nucleic acid encoding a multi-specific antibody of embodiments 1-95.

Embodiment 107: a vector comprising a nucleic acid of embodiments 101-106.

Embodiment 108: a cell comprising the nucleic acid of embodiments 101-106 or the vector of embodiment 107.

Embodiment 109: a method of treating a disease or condition in a subject in need thereof, comprising administering to the subject a therapeutically effective amount of a multi-specific antibody of embodiments 1-95 or a pharmaceutical composition of embodiments 96-100.

Embodiment 110: the method of embodiment 109, wherein the disease or condition is a cancer.

Embodiment 111: the method of embodiment 110, wherein the cancer is a solid tumor cancer.

Embodiment 112: the method of embodiment 111, wherein the solid tumor cancer is bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer.

Embodiment 113: the method of embodiment 111, wherein the solid tumor cancer is breast cancer.

Embodiment 114: the method of embodiment 113, wherein the breast cancer is luminal A breast cancer, luminal B breast cancer, triple-negative breast cancer, HER2-enriched breast cancer, or normal-like breast cancer.

Embodiment 115: the method of embodiment 113, wherein the breast cancer is ductal carcinoma in situ (DCIS), invasive ductal carcinoma (IDC), invasive lobular carcinoma (ILC), inflammatory breast cancer, lubular carcinoma in situ (LCIS), male breast cancer, Paget's disease of the Nipple, or phyllodes tumors of the breast.

Embodiment 116: the method of embodiment 115, wherein the IDC comprises tubular carcinoma of the breast, medullary carcinoma of the breast, papillary carcinoma of the breast, or cribriform carcinoma of the breast.

Embodiment 117: the method of embodiment 111, wherein the solid tumor cancer is ovarian cancer.

Embodiment 118: the method of embodiment 117, wherein the ovarian cancer is epithelial carcinoma, serous carcinoma, small-cell carcinoma, primary peritoneal carcinoma, clear-cell carcinoma, clear-cell adenocarcinoma, endometrioid, malignant mixed mullerian tumor, mucinous, mucinous adenocarcinoma, pseudomyxoma peritonel, undifferentiated epithelial, malignant Brener tumor, transitional cell carcinoma, sex cord-stromal tumor, granulosa cell tumor, adult granulosa cell tumor, juvenile granulosa cell tumor, Sertoli-Leydig cell tumor, sclerosing stromal tumors, germ cell tumor, dysgerminoma, choriocarcinoma, immature (solid) teratoma, mature teratoma (dermoid cyst), yolk sac tumor (endodermal sinus tumor), embryonal carcinoma, polyembryoma, squamous cell carcinoma, mixed tumors, or low malignant potential tumors.

Embodiment 119: the method of embodiment 111, wherein the solid tumor cancer is lung cancer.

Embodiment 120: the method of embodiment 119, wherein the lung cancer is small cell lung cancer (SCLC) or non-small cell lung cancer (NSCLC).

Embodiment 121: the method of embodiment 120, wherein the NSCLC comprises adenocarcinoma, squamous cell cancer, large cell carcinoma, or undifferentiated non-small cell lung cancer.

Embodiment 122: the method of embodiment 111, wherein the solid tumor cancer is liver cancer.

Embodiment 123: the method of embodiment 122, wherein the liver cancer is hepatocellular carcinoma (HCC), cholangiocarcinoma, liver angiosarcoma, or hepatoblastoma.

Embodiment 124: the method of embodiment 111, wherein the solid tumor cancer is prostate cancer.

Embodiment 125: the method of embodiment 124, wherein the prostate cancer is acinar adenocarcinoma, ductal adenocarcinoma, transitional cell (or urothelial) cancer, squamous cell cancer, small cell prostate cancer, carcinoid in the prostate, or sarcoma in the prostate.

Embodiment 126: the method of embodiment 111, wherein the solid tumor is brain cancer.

Embodiment 127: the method of embodiment 126, wherein the brain cancer is glioblastoma.

Embodiment 128: the method of embodiment 110, wherein the cancer is a hematologic malignancy.

Embodiment 129: the method of embodiment 128, wherein the hematologic malignancy is chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, or acute myeloid leukemia.

Embodiment 130: the method of any one of the embodiments 110-129, wherein the cancer is a metastatic cancer.

Embodiment 131: the method of any one of the embodiments 110-129, wherein the cancer is a relapsed or refractory cancer.

Embodiment 132: the method of embodiment 109, wherein the disease or condition is a liver disease or condition.

Embodiment 133: the method of embodiment 132, wherein the liver disease or condition is non-alcoholic fatty liver disease (NASH) or alcoholic steatohepatitis.

Embodiment 134: the method of any one of the embodiments 109-133, further comprising administering an additional therapeutic agent.

Embodiment 135: the method of embodiment 134, wherein the additional therapeutic agent is a chemotherapeutic agent, radiation, or a combination thereof.

Embodiment 136: the method of any one of the embodiments 109-135, wherein the multi-specific antibody and the additional therapeutic agent are administered simultaneously.

Embodiment 137: the method of any one of the embodiments 109-135, wherein the multi-specific antibody and the additional therapeutic agent are administered sequentially.

Embodiment 138: the method of any one of the embodiments 109-135 or 137, wherein the multi-specific antibody is administered to the subject prior to administering the additional therapeutic agent.

Embodiment 139: the method of any one of the embodiments 109-135 or 137, wherein the additional therapeutic agent is administered to the subject prior to administering the multi-specific antibody.

Embodiment 140: the method of any one of the embodiments 109-139, wherein the multi-specific antibody and the additional therapeutic agent are administered as a combination.

Embodiment 141: the method of any one of the embodiments 109-139, wherein the multi-specific antibody and the additional therapeutic agent are administered as separate dosage forms.

Embodiment 142: the method of any one of the embodiments 109-141, wherein the subject has previously been treated with an immune checkpoint inhibitor treatment.

Embodiment 143: the method of any one of the embodiments 109-142, wherein the subject is insensitive to treatment with an immune checkpoint inhibitor, has failed to respond to treatment with an immune checkpoint inhibitor, or who expresses low level of or does not express an immune checkpoint protein.

Embodiment 144: the method of any one of the embodiments 109-143, wherein the subject has undergone surgery.

Embodiment 145: the method of inducing tumor and immunosuppressive cell kill effect in a target cell population, comprising: contacting the target cell population comprising at least one tumor cell and at least one immunosuppressive cell with a multi-specific antibody of embodiments 1-95 or a pharmaceutical composition of embodiments 96-100 for a time sufficient to induce cell kill effect, thereby killing the at least one tumor cell and the at least one immunosuppressive cell in the target cell population.

Embodiment 146: the method of embodiment 145, wherein the tumor cell is a cell from a solid tumor.

Embodiment 147: the method of embodiment 145, wherein the tumor cell is a cell from a hematological malignancy.

Embodiment 148: the method of embodiment 145 or 146, wherein the tumor cell is from a bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer.

Embodiment 149: the method of embodiment 145 or 147, wherein the tumor cell is from chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, or acute myeloid leukemia.

Embodiment 150: the method of embodiment 145, wherein the immunosuppressive cell is MDSC, a tumor-associated macrophage, or a Treg cell.

Embodiment 151: the method of any one of the embodiments 145-150, wherein the multi-specific antibody of embodiments 1-95 or the pharmaceutical composition of embodiments 96-100 modulates T-cell proliferation, optionally tumor-infiltrating lymphocyte (TIL) proliferation.

Embodiment 152: the method of any one of the embodiments 145-151, wherein the multi-specific antibody of embodiments 1-95 or the pharmaceutical composition of embodiments 96-100 enhances T-cell proliferation, optionally tumor-infiltrating lymphocyte (TIL) proliferation.

Embodiment 153: the method of any one of the embodiments 145-152, wherein the multi-specific antibody of embodiments 1-95 or the pharmaceutical composition of embodiments 96-100 decreases tumor cells in the target cell population by about 20%, 30%, 40%, 50%, 60%, 70%, 80%, 90%, 100%, or more.

Embodiment 154: the method of any one of the embodiments 145-152, wherein the multi-specific antibody of embodiments 1-95 or the pharmaceutical composition of embodiments 96-100 decreases tumor cells in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

Embodiment 155: the method of any one of the embodiments 145-154, wherein the multi-specific antibody of embodiments 1-95 or the pharmaceutical composition of embodiments 96-100 decreases tumor cell proliferation in the target cell population by about 20%, 30%, 40%, 50%, 60%, 70%, 80%, 90%, 100%, or more.

Embodiment 156: the method of any one of the embodiments 145-154, wherein the multi-specific antibody of embodiments 1-95 or the pharmaceutical composition of embodiments 96-100 decreases tumor cell proliferation in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

Embodiment 157: the method of any one of the embodiments 145-156, wherein the multi-specific antibody of embodiments 1-95 or the pharmaceutical composition of embodiments 96-100 decreases immunesuppressive cells in the target cell population by about 20%, 30%, 40%, 50%, 60%, 70%, 80%, 90%, 100%, or more.

Embodiment 158: the method of any one of the embodiments 145-156, wherein the multi-specific antibody of embodiments 1-95 or the pharmaceutical composition of embodiments 96-100 decreases immunesuppressive cells in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

Embodiment 159: the method of any one of the embodiments 145-158, wherein the multi-specific antibody of embodiments 1-95 or the pharmaceutical composition of embodiments 96-100 decreases immunesuppressive cell proliferation in the target cell population by about 20%, 30%, 40%, 50%, 60%, 70%, 80%, 90%, 100%, or more.

Embodiment 160: the method of any one of the embodiments 145-158, wherein the multi-specific antibody of embodiments 1-95 or the pharmaceutical composition of embodiments 96-100 decreases immunesuppressive cell proliferation in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

Embodiment 161: the method of any one of the embodiments 145-160, wherein the target cell population is an in vivo target cell population.

Embodiment 162: the method of any one of the embodiments 145-161, wherein the target cell population is within a tumor microenvironment.

Embodiment 163: the method of any of the preceding embodiments, wherein the subject is a human.

Embodiment 164: the multi-specific antibody of any one of the embodiments 1-95 or the pharmaceutical composition of embodiments 96-100 for use in treating a cancer.

Embodiment 165: the use of embodiment 163, wherein the cancer is bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer.

Embodiment 166: the use of embodiment 164, wherein the breast cancer is luminal A breast cancer, luminal B breast cancer, triple-negative breast cancer, HER2-enriched breast cancer, or normal-like breast cancer.

Embodiment 167: the use of embodiment 163, wherein the cancer is a hematological malignancy.

Embodiment 168: the use of embodiment 166, wherein the hematological malignancy comprises chronic lymphocytic leukemia (CLL), acute myeloid leukemia (AML), diffuse large B-cell lymphoma (DLBCL), chronic myeloid leukemia, or acute myeloid leukemia.

Embodiment 169: the use of embodiment 163, wherein the cancer is refractory to immune checkpoint inhibitor treatment.

Embodiment 170: the multi-specific antibody of any one of embodiments 1-95 or the pharmaceutical composition of embodiments 96-100 for use in treating a liver disease or condition, optionally NASH or alcoholic steatohepatitis.

Embodiment 171: the method for making a cancer treatment comprising contacting the nucleic acid of embodiments 101-106 to a suitable cell line to establish a transfected cell line, culturing the transfected cell line under conditions that promote secretion of the multi-specific antibody, and harvesting the multi-specific antibody from the supernatant of the transfected cell line.

Embodiment 172: the method of embodiment 171, further comprising purifying the multi-specific antibody from the supernatant of the transfected cell line.

Embodiment 173: the method of embodiment 171 or 171, wherein the transfected cell line is stably transfected.

Embodiment 174: the method for making a cancer treatment comprising admixing the multi-specific antibody of any one of embodiments of 1-95 and a pharmaceutically acceptable excipient, carrier, or diluent.

Embodiment 175: the kit comprising a multi-specific antibody of embodiments 1-95, a pharmaceutical composition of embodiments 96-100, a nucleic acid of embodiments 101-106, a vector of embodiment 107, or a cell of embodiment 108.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A multi-specific antibody comprising a tumor binding moiety that specifically binds to a tumor-associated antigen and an immune cell binding moiety that specifically binds to an antigen expressed on an immunosuppressive cell.

2. The multi-specific antibody of claim 1, wherein the immunosuppressive cell is a Myeloid-derived suppressor cell (MDSC).

3. The multi-specific antibody of claim 1, wherein the immunosuppressive cell is a tumor-associated macrophage (TAM), optionally a M2 polarized TAM (M2-TAM).

4. The multi-specific antibody of claim 1, wherein the tumor associated antigen is:
TROP2, HER2, GPC3, GD2, FOLR1, FLT3, BCMA, MUC16, SLC4A4, STEAP1, CD19, CD20, CD22, CD25, CD33, CD38, CD30, CD47, CD123, mesothelin, MT1-MMP, or PSMA;
TROP2, GPC3, HER2, FOLR1, CD33, CD38, FLT3, CD30, CD22, or GD2; or
TROP2, GPC3, FOLR1, CD33, CD38, or FLT3.

5. The multi-specific antibody of claim 1, wherein the antigen expressed on the immunosuppressive cell is:
CD33, TRAIL-R2, CSF1R, SEMA4A, SEMA4D, CD163, MARCO, TNFR2, TREM2, MS4A7, C5AR1, LYVE1, ABCC3, LILRB4, MRC1, STAB1, TMEM37, MERTK, TMEM119, SIGLEC1, SIGLEC7, SIGLEC9, IL4R, MGL1, CD200R, or SELPLG;
TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, MGL2, CD33, ABCC3, LILRB4, TMEM37, TMEM119, SIGLEC7, or SIGLEC9;
TRAIL-R2, CSF1R, MARCO, SELPLG, CD163, TREM2, MS4A7, C5AR1, LYVE1, MRC1, CD200R, STAB1, MERTK, SIGLEC1, IL4R, MGL1, or MGL2;
TRAIL-R2, CSF1R, CD33, TREM2, C5AR1, LYVE1, ABCC3, LILRB4, MRC1, SIGLEC1, STAB1, TMEM37, MERTK, TMEM119, SIGLEC7, SIGLEC9, or IL4R;
TRAIL-R2, CSF1R, TREM2, C5AR1, LYVE1, MRC1, STAB1, MERTK, SIGLEC1, or IL4R;
MARCO, SELPLG, CD163, MS4A7, CD200R, MGL1, or MGL2;
CD33, ABCC3, LILRB4, TMEM37, TMEM119, SIGLEC7, or SIGLEC9;
SEMA4A, SEMA4D, or TNFR2;
TRAIL-R2, CD33, CD163, or CSF1R; or
CD33, CD163, or CSF1R.

6. The multi-specific antibody of claim 1, wherein the tumor binding moiety comprises an IgG antibody framework, optionally an IgG1 or IgG4 framework.

7. The multi-specific antibody of claim 6, wherein the tumor binding moiety comprises a full-length antibody.

8. The multi-specific antibody of claim 6, wherein the tumor binding moiety is a humanized antibody.

9. The multi-specific antibody of claim 6, wherein the tumor binding moiety comprises an immunoglobulin heavy chain variable region comprising an amino acid sequence at least about 90%, 95%, 97%, 98%, 99%, or 100% identical to or consist of the amino acid sequence set forth in SEQ ID NOs: 9, 16, 20, 24, or 28; and an immunoglobulin light chain variable region at least about 90%, 95%, 97%, 98%, 99%, or 100% identical to or consist of the amino acid sequence set forth in SEQ ID NOs: 10, 32, 36, 40, or 44.

10. The multi-specific antibody of claim 1, wherein the immune cell binding moiety comprises an IgG antibody framework, optionally an IgG1 or IgG4 framework.

11. The multi-specific antibody of claim 10, wherein the immune cell binding moiety comprises a Fab, F(ab)₂, single-domain antibody, a single chain variable fragment (scFv), or a nanobody.

12. The multi-specific antibody of claim 10, wherein the immune cell binding moiety is a humanized antibody.

13. The multi-specific antibody of claim 10, wherein the immune cell binding moiety comprises an immunoglobulin heavy chain variable region comprising an amino acid sequence at least about 90%, 95%, 97%, 98%, 99%, or 100% identical to the amino acid sequence set forth in SEQ ID NOs: 11, 48, 52, or 59; and an immunoglobulin light chain variable region at least about 90%, 95%, 97%, 98%, 99%, or 100% identical to the amino acid sequence set forth in SEQ ID NOs: 12, 63, 67, or 74.

14. The multi-specific antibody of claim 1, wherein the tumor binding moiety comprises an IgG antibody framework, optionally a full-length IgG antibody framework, the immune cell binding moiety is an scFv, and the immune cell binding moiety is coupled to the C-terminus of the tumor binding moiety, optionally recombinantly fused to the C-terminus of the tumor binding moiety.

15. The multi-specific antibody of claim 1, wherein the tumor binding moiety comprises an IgG antibody framework, optionally a full-length IgG antibody framework, the immune cell binding moiety is an scFv, and the immune cell binding moiety is coupled to the N-terminus of the tumor binding moiety, optionally recombinantly fused to the N-terminus of the tumor binding moiety.

16. The multi-specific antibody of claim 14 or 15, wherein the immune cell binding moiety is coupled to the tumor binding moiety by a polypeptide linker.

17. The multi-specific antibody of claim 16, wherein the polypeptide linker comprises (Gly4Ser)ₙ, wherein n is an integer from 1 to 10, optionally from 1 to 6, 1 to 4, or 1 to 2, further optionally 1, 2, 3, or 4.

18. The multi-specific antibody of claim 1, wherein the tumor binding moiety has a K_{D} for the tumor associated antigen that is lower than the K_{D} of the immune cell binding moiety for the antigen expressed on the immunosuppressive cell.

19. The multi-specific antibody of claim 1, wherein the multi-specific antibody comprises an Fc region that has been modified to reduce the affinity for human neonatal Fc receptor (FcRn).

20. The multi-specific antibody of claim 1, wherein the multi-specific antibody comprises an Fc region comprising a modification to reduce antibody-dependent cellular cytotoxicity (ADCC), wherein the modification optionally comprises L234, L235, P238, or P331, or a combination thereof, wherein L234, L235, P238, and P331 correspond to positions 234, 235, 238, and 331 of a wild-type IgG1, according to the EU numbering convention.

21. The multi-specific antibody of claim 1, wherein the multi-specific antibody comprises an Fc region that has been modified to reduce neutropenia.

22. The multi-specific antibody of claim 21, wherein the Fc region comprises a modification at L234, S239, S442, or a combination thereof, wherein L234, S239, and S442 correspond to positions 234, 239, 442 of a wild-type IgG1, according to the EU numbering convention.

23. The multi-specific antibody of claim 1, wherein the multi-specific antibody comprises an Fc region that has been modified to enhance antibody-dependent cellular cytotoxicity (ADCC).

24. The multi-specific antibody of claim 23, wherein the Fc region comprises a modification at S239, A330, I332, or a combination thereof, wherein S239, A330, and I332 correspond to positions 239, 330, and 332 of a wild-type IgG1, according to the EU numbering convention.

25. The multi-specific antibody of claim 1, wherein the multi-specific antibody comprises a modification to a hinge region.

26. The multi-specific antibody of claim 25, wherein the hinge region comprises a modification at S228, wherein S228 correspond to position 228 of a wild-type IgG4, according to the EU numbering convention.

27. The multi-specific antibody of claim 1, wherein the antibody comprises:
an immunoglobulin heavy chain comprising an amino acid sequence at least about 90%, 95%, 97%, 98%, 99%, or 100% identical to or consist of the amino acid sequence set forth in Table 5; and an immunoglobulin light chain at least about 90%, 95%, 97%, 98%, 99%, or 100% identical to or consist of the amino acid sequence set forth in Table 5;
an immunoglobulin heavy chain comprising an amino acid sequence at least about 90%, 95%, 97%, 98%, 99%, or 100% identical to or consist of the amino acid sequence set forth in SEQ ID NOs: 7 or 75-77; and an immunoglobulin light chain at least about 90%, 95%, 97%, 98%, 99%, or 100% identical to or consist of the amino acid sequence set forth in SEQ ID NO: 8;
an immunoglobulin heavy chain comprising an amino acid sequence at least about 90%, 95%, 97%, 98%, 99%, or 100% identical to or consist of the amino acid sequence set forth in SEQ ID NOs: 79-83; and an immunoglobulin light chain at least about 90%, 95%, 97%, 98%, 99%, or 100% identical to or consist of the amino acid sequence set forth in SEQ ID NO: 80;
97%, 98%, 99%, or 100% identical to or consist of the amino acid sequence set forth in SEQ ID NOs: 84-88; and an immunoglobulin light chain at least about 90%, 95%, 97%, 98%, 99%, or 100% identical to or consist of the amino acid sequence set forth in SEQ ID NO: 85; or
an immunoglobulin heavy chain comprising an amino acid sequence at least about 90%, 95%, 97%, 98%, 99%, or 100% identical to or consist of the amino acid sequence set forth in SEQ ID NOs: 89, 91, or 93; and an immunoglobulin light chain at least about 90%, 95%, 97%, 98%, 99%, or 100% identical to or consist of the amino acid sequence set forth in SEQ ID NO: 90, 92, or 94.

28. The multi-specific antibody of claim 1, wherein the multi-specific antibody further comprises at least one cytotoxic moiety.

29. The multi-specific antibody of claim 28, wherein the cytotoxic moiety comprises an auristatin, an auristatin derivative, maytansine, a maytansinoid, a taxane, a calicheamicin, cemadotin, a duocarmycin, a pyrrolobenzodiazepine (PBD), tubulysin, dexamethasone, or dasatinib.

30. The multi-specific antibody of claim 29, wherein the auristatin derivative is monomethyl auristatin E (MMAE) or monomethyl auristatin F (MMAF).

31. The multi-specific antibody of claim 29, wherein the maytansinoid is DM1, DM2, or DM4.

32. The multi-specific antibody of claim 29, wherein the pyrrolobenzodiazepine is a pyrrolobenzodiazepine dimer.

33. The multi-specific antibody of claim 29, wherein the at least one cytotoxic moiety is attached to the multi-specific antibody via a linker, optionally a cleavable linker or a non-cleavable linker.

34. A pharmaceutical composition comprising the multi-specific binding polypeptide of any one of claims 1 to 33, and a pharmaceutically acceptable excipient, carrier, or diluent.

35. The pharmaceutical composition of claim 34, wherein the pharmaceutical composition is formulated for parenteral administration, optionally for subcutaneous, intramuscular, or intravenous administration.

36. A nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 7 or 75-77; and optionally an immunoglobulin light chain at least about 90%, 95%, 97%, 98%, 99%, or 100% identical to or consist of the amino acid sequence set forth in SEQ ID NO: 8.

37. A nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 79-83; and optionally an immunoglobulin light chain at least about 90%, 95%, 97%, 98%, 99%, or 100% identical to or consist of the amino acid sequence set forth in SEQ ID NO: 80.

38. A nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 84-88; and optionally an immunoglobulin light chain at least about 90%, 95%, 97%, 98%, 99%, or 100% identical to or consist of the amino acid sequence set forth in SEQ ID NO: 85.

39. A nucleic acid encoding a multi-specific antibody comprising an immunoglobulin heavy chain comprising an amino acid sequence at least about 90%, 95%, 97%, 98%, 99%, or 100% identical to or consist of the amino acid sequence set forth in SEQ ID NOs: 89, 91, or 93; and optionally an immunoglobulin light chain at least about 90%, 95%, 97%, 98%, 99%, or 100% identical to or consist of the amino acid sequence set forth in SEQ ID NO: 90, 92, or 94.

40. A method of treating a disease or condition in a subject in need thereof, the method comprising:
administering to the subject a therapeutically effective amount of a multi-specific antibody of claim 1, a pharmaceutical composition of claim 34, or a nucleic acid of claims 36-39.

41. The method of claim 40, wherein the disease or condition is bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer.

42. The method of claim 41, wherein the breast cancer is luminal A breast cancer, luminal B breast cancer, triple-negative breast cancer, HER2-enriched breast cancer, or normal-like breast cancer.

43. The method of claim 41, wherein the breast cancer is triple-negative breast cancer.

44. The method of claim 40, wherein the disease or condition is a hematological malignancy.

45. The method of claim 44, wherein the hematological malignancy comprises chronic lymphocytic leukemia (CLL), acute myeloid leukemia (AML), diffuse large B-cell lymphoma (DLBCL), chronic myeloid leukemia, or acute myeloid leukemia.

46. The method of claim 40, wherein the disease or condition is a liver disease or condition.

47. The method of claim 46, wherein the liver disease or condition is non-alcoholic fatty liver disease (NASH) or alcoholic steatohepatitis.

48. The method of claim 40, wherein the subject has previously been treated with an immune checkpoint inhibitor treatment.

49. The method of claim 40, wherein the subject is insensitive to treatment with an immune checkpoint inhibitor, has failed to respond to treatment with an immune checkpoint inhibitor, or who expresses low level of or does not express an immune checkpoint protein.

50. A method of inducing tumor and immunosuppressive cell killing effect in a target cell population, comprising:
contacting the target cell population comprising at least one tumor cell and at least one immunosuppressive cell with a multi-specific antibody of claims 1-33, a pharmaceutical composition of claims 34-35, or a nucleic acid of claims 36-39 for a time sufficient to induce cell kill effect, thereby killing the at least one tumor cell and the at least one immunosuppressive cell in the target cell population.

51. The method of claim 50, wherein the tumor cell is a cell from a solid tumor, optionally from a bladder cancer, bone cancer, brain cancer, breast cancer, cervical cancer, cholangiocarcinoma, colorectal cancer, endometrial cancer, esophageal cancer, eye cancer, head and neck cancer, kidney cancer, liver cancer, lung cancer, melanoma, ovarian cancer, pancreatic cancer, prostate cancer, sarcoma, stomach cancer, testicular cancer, or thyroid cancer.

52. The method of claim 50, wherein the tumor cell is a cell from a hematological malignancy.

53. The method of claim 50, wherein the immunosuppressive cell is MDSC, a tumor-associated macrophage, or a Treg cell.

54. The method of any one of the claims 50-53, wherein the multi-specific antibody of claims 1-33, the pharmaceutical composition of claim 34 or 36, or the nucleic acid of claims 36-39 decreases tumor cells, optionally tumor cell proliferation, in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

55. The method of any one of the claims 50-54, wherein the multi-specific antibody of claims 1-33, the pharmaceutical composition of claim 34 or 35, or the nucleic acid of claims 36-39 decreases immunesuppressive cells, optionally immunesuppressive cell proliferation in the target cell population by about 1-fold, 2-fold, 3-fold, 4-fold, 5-fold, 6-fold, 7-fold, 8-fold, 9-fold, 10-fold, 20-fold, 30-fold, 40-fold, 50-fold, or more.

56. The method of any one of the claims 50-55, wherein the multi-specific antibody of claims 1-33, the pharmaceutical composition of claim 34 or 35, or the nucleic acid of claims 36-39 enhances T-cell proliferation, optionally tumor-infiltrating lymphocyte (TIL) proliferation.

57. The method of any one of the claims 50-56, wherein the target cell population is an in vivo target cell population.

58. The method of any one of the claims 50-57, wherein the target cell population is within a tumor microenvironment.

59. A method of inducing immunosuppressive cell killing in a subject in need thereof, comprising administering an antibody-cytotoxin conjugate that specifically binds to an antigen expressed on an immunosuppressive cell, thereby killing the immunosuppressive cell in the subject.

60. A method of activating tumor cell-killing immune cells in a subject in need thereof, comprising administering an antibody-cytotoxin conjugate that specifically binds to an antigen expressed on an immunosuppressive cell, thereby killing the immunosuppressive cell and activating the tumor cell-killing immune cells in the subject.

61. A method of reducing suppression of tumor cell-killing immune cells in a subject in need thereof, comprising administering an antibody-cytotoxin conjugate that specifically binds to an antigen expressed on an immunosuppressive cell, thereby killing the immunosuppressive cell and reducing suppression of the tumor cell-killing immune cells in the subject.

62. The method of any one of the claims 59-61, wherein the immunosuppressive cell is a MDSC, a TAM, or a Treg cell.

63. The method of any one of the claims 59-62, wherein the antibody-cytotoxin conjugate further comprises a tumor-specific binding moiety.

64. The method of any one of the claims 59-63, wherein the antibody-cytotoxin conjugate comprises a multi-specific antibody of claims 1-33.

65. The method of any one of the claims 59-64, wherein the subject has cancer.
